SEQUENCE LISTING (33 sequences)

SEQ ID NO: 1 - DNA, 2053 bp, Homo sapiens
  source 1..2053: mol_type = other DNA
cttccgtcggccattttaggtggtccgcggcggcgccattaaagcgaggaggaggcgagagcggccgccgctggtgcttattcttttttagtgcagcgggagagagcgggagtgtgcgccgcgcgagagtgggaggcgaagggggcaggccagggagaggcgcaggagcctttgcagccacgcgcgcgccttccctgtcttgtgtgcttcgcgaggtagagcgggcgcgcggcagcggcggggattactttgctgctagtttcggttcgcggcagcggcgggtgtagtctcggcggcagcggcggagacactagcactatgtcggaggagcagttcggcggggacggggcggcggcagcggcaacggcggcggtaggcggctcggcgggcgagcaggagggagccatggtggcggcgacacagggggcagcggcggcggcgggaagcggagccgggaccgggggcggaaccgcgtctggaggcaccgaagggggcagcgccgagtcggagggggcgaagattgacgccagtaagaacgaggaggatgaagggaaaatgtttataggaggccttagctgggacactacaaagaaagatctgaaggactacttttccaaatttggtgaagttgtagactgcactctgaagttagatcctatcacagggcgatcaaggggttttggctttgtgctatttaaagaatcggagagtgtagataaggtcatggatcaaaaagaacataaattgaatgggaaggtgattgatcctaaaagggccaaagccatgaaaacaaaagagccggttaaaaaaatttttgttggtggcctttctccagatacacctgaagagaaaataagggagtactttggtggttttggtgaggtggaatccatagagctccccatggacaacaagaccaataagaggcgtgggttctgctttattacctttaaggaagaagaaccagtgaagaagataatggaaaagaaataccacaatgttggtcttagtaaatgtgaaataaaagtagccatgtcgaaggaacaatatcagcaacagcaacagtggggatctagaggaggatttgcaggaagagctcgtggaagaggtggtgaccagcagagtggttatgggaaggtatccaggcgaggtggtcatcaaaatagctacaaaccatactaaattattccatttgcaacttatccccaacaggtggtgaagcagtattttccaatttgaagattcatttgaaggtggctcctgccacctgctaatagcagttcaaactaaattttttgtatcaagtccctgaatggaagtatgacgttgggtccctctgaagtttaattctgagttctcattaaaagaaatttgctttcattgttttatttcttaattgctatgcttcagaatcaatttgtgttttatgccctttcccccagtattgtagagcaagtcttgtgttaaaagcccagtgtgacagtgtcatgatgtagtagtgtcttactggttttttaataaatccttttgtataaaaatgtattggctcttttatcatcagaataggaaaaattgtcatggattcaagttattaaaagcataagtttggaagacaggcttgccgaaattgaggacatgattaaaattgcagtgaagtttgaaatgtttttagcaaaatctaatttttgccataatgtgtcctccctgtccaaattgggaatgacttaatgtcaatttgtttgttggttgttttaataatacttccttatgtagccattaagatttatatgaatattttcccaaatgcccagtttttgcttaatatgtattgtgctttttagaacaaatctggataaatgtgcaaaagtacccctttgcacagatagttaatgttttatgcttccattaaataaaaaggacttaaaatctgttaattataatagaaatgcggctagttcagagagatttttagagctgtggtggacttcatagatgaattcaagtgttgagggaggattaaagaaatatataccgtgtttatgtgtgtgtgctt

SEQ ID NO: 2 - protein, 287 aa, Homo sapiens
MSEEQFGGDGAAAAATAAVGGSAGEQEGAMVAATQGAAAAAGSGAGTGGGTASGGTEGGSAESEGAKIDASKNEEDEGKMFIGGLSWDTTKKDLKDYFSKFGEVVDCTLKLDPITGRSRGFGFVLFKESESVDKVMDQKEHKLNGKVIDPKRAKAMKTKEPVKKIFVGGLSPDTPEEKIREYFGGFGEVESIELPMDNKTNKRRGFCFITFKEEEPVKKIMEKKYHNVGLSKCEIKVAMSKEQYQQQQQWGSRGGFAGRARGRGGDQQSGYGKVSRRGGHQNSYKPY

SEQ ID NO: 3 - DNA, 6607 bp, Mus musculus
  source 1..6607: mol_type = other DNA
ccattttaggtggtccgcggcggcgccattaaagcgaggaggaggcgagagtggccgccgctgctacttcattcttttttttttcagtgcagccggggagagcgagagagcgcgctgcgcgagagtgggaggcgaggggggcaggccggggagaggcgcaggagcccttgcagccacgcgcgcgccttgtctagggtgcctcgcgaggtagagcgggcatcgcgcggcggcggcggggattactttgctgctagtttcggttcgcggcggcggcggcgtcggcgggtgtcgtcttcggcggcggcagtagcactatgtcggaggagcagttcggaggggacggggcggcggcggcggcaacggcggcggtaggcggctcggcgggcgagcaggagggagccatggtggcggcggcggcgcaggggccggcggcggcggcgggaagcgggagcggcggcggcggctctgcggccggaggcaccgaaggaggcagcgccgaggcagagggagccaagatcgacgccagtaagaacgaggaggatgaagggaaaatgtttataggaggccttagctgggacaccacaaagaaagatctgaaggactacttttccaaatttggtgaagttgtagactgcactctgaagttagatcctatcacagggcgatcaaggggttttggctttgtgctatttaaagagtcggagagtgtagataaggtcatggatcagaaagaacataaattgaatgggaaagtcattgatcctaaaagggccaaagccatgaaaacaaaagagcctgtcaaaaaaatttttgttggtggcctttctccagacacacctgaagaaaaaataagagagtactttggtggttttggtgaggttgaatccatagagctccctatggacaacaagaccaataagaggcgtgggttctgttttattacctttaaggaagaggagccagtgaagaagataatggaaaagaaataccacaatgttggtcttagtaaatgtgaaataaaagtagccatgtcaaaggaacagtatcagcagcagcagcagtggggatctagaggagggtttgcaggcagagctcgcggaagaggtggagatcagcagagtggttatgggaaagtatccaggcgaggtggacatcaaaatagctacaaaccatactaaattattccatttgcaacttatccccaacaggtggtgaagcagtattttccaatttgaagattcatttgaaggtggctcctgccacctgctaatagcagttcaaactaaattttttctatcaagttcctgaatggaagtatgacgttgggtccctctgaagtttaattctgagttctcattaaaagaatttgctttcattgttttatttcttaattgctatgcttcagtatcaatttgtgttttatgccccccctcccccccagtattgtagagcaagtcttgtgttaaaaaaagcccagtgtgacagtgtcatgatgtagtagtgtcttactggttttttaataaatccttttgtataaaaatgtattggctcttttatcatcagaataggaggaagtgaaatactacaaatgtttgtcttggattcaagtcactagaagcataaatttgaggggataaaaacaacggtaaactttgtctgaaagagggcatggttaaaaatgtagtgaattttaaatgtttttagcaaaatttgattttgcccaagaatccctgtctgaattggaaatgacttaatgtagtcaatgtgcttgttggttgtcttaatattacttctgtagccattaagttttatgagtaacttcccaaatacccacgtttttctttatatgtattgtgctttttaaaaacaaatctggaaaaatgggcaagaacatttgcagacaattgtttttaagcttccattaaataaaaaaaatgtggacttaaggaaatctattaatttaaatagaactgcagctagtttagagagtatttttttcttaaagctttggtgtaattagggaagattttaaaaaatgcatagtgtttatttgtatgtgtgctctttttttaagtcaatttttggggggttggtctgttaactgagtctaggatttaaaggtaagatgttcctagaaatcttgtcatcccaaaggggcgggcgctaaggtgaaacttcagggttcagtcagggtcactgctttatgtgtgaaatcactcaaattggtaagtctcttatgttagcattcaggacattgatttcaacttggatggacaatttatagttactactgaattgtgtgttaatgtgttcagtcctggtaagttttcagtttgatcagttagttggaagcagacttgaagagctgttagtcacgtgagccatgggtgcagtcgatctgtggtcagatgcctgagtctgtgatagtgaattgtgtctaaagacattttaatgataaaagtcagtgctgtaaagttgaaagttcatgagagacatacaatgagggctgcagcccatttttaaaaacattataatacaaaagtatgcacatttgtttacatatccctgcctttgtattacagtggcaggtttgtgtacttaaactgggaaagcctcagatctatgattacctggcctatcatagaaagtgtctaaataaatcactctgtcaattgaatacattagtattagctagcatacttcattatgcctgttttccataaataccacaccaaaaacttgcttggggcagtttgagcctagttcatgagctgctatcagattggtcttgatcctatataataggccaaatgtctgtaaacagctgtgctggtggaatgtagaaagtcactgcactcagattcaacttcctgattggaagtcatcacagtgtgattaaacattttcacaaagaatagtagataaataacttggtttttaatgttaactttgtttccattaagtcacatttaaaaacttatcctcacgcctacctgagttaattatctgttgacctagatatctttctggccactcactgacttatttcttgaacttttgccatttgcataaatcttgtcagctttgttcttgattatgcattgtccaggctgagctagttgtctttccaggaatccctttgtctctgaattaggtcctttgtttcctaaatcatcctgcttgtttggcacaagtcttcccaggccagtgagacctccgtgtcctctcagcaccataggggtaggtaaccctggttaggctggacaggggtttgctgagggagtttgttcatttgaatctaggtcttacatgacgtctttcaaatagggtttttaccttgacactaaactgtccagtctaagcagttctgcaaaatgtgagggaattatgaacttcttcctgcagtgggtttttatggttttggtttgttttttgttgttttggttctttgttgagccctggacaaaaacttccctagttctggtttctacaatttaaattaaaaacagaattcatcttagaatttttcaccctcttccccaactattctaatcaatcttaagtatgcccttcatcttttttccttcctaaggcttttactgatagtgtaattccgtactcttcaaccctgggaaggctgaagtggattcttgagctcatttcaaggctgacctgggtgttggcaagaacccagcttagaacaaacacatgcaaggccatcttaccttacatcctgttgcttggacttcttcctgctcaaagtttttagtggatgctaagtgatctttgcttccactgaggagtggaacactttagaatgaacctctagatagatatttttattgtctggtgagggttactggagtttcccaccctgcctgaagggtgaatctggcttacagtgttctcatctcaaagggaagaaggcagatggctgtgtccagagagagccatcacagtttgcttcagagacactagaatgggctggaagatctagtggtcttaatcagacttgaaacctggcctttcttcattacccatatgtctaccagtacttgggctaacacttaagccattagggcctttgtaggggtgttttgagaccccctccatgctaacaaatatacaggtttcttaacatttgctcataaacttgtaaagcttactttctcttaatccaccccacatttaacaagccctggtacttagaatttcagaagagtaatggcaggtaggtgtgtgtgtgtgtgtgtgtgtgtgtgtgtgtgtgtgtgtgtgagagagagagagagagagagagagagagagagagagagagagagagagagagagaagtttgtggaaaatcaggtaatgacagctcatccttttagaattgtacttcagaatagaaacatttggtgggctgttaggtagctttgattacttgtgggtagacctgctagtattgccagtcctcaagcaatgagctttctgtatcttgtttactagatatatactaccaggtgagtcatttcctggggttctgttttcttttaaaatctttccctaaacttaatatgtattaaaaagtctggcttttcagtccattctttgtgcactgggatggcaattgcttcattatatgacaattgctgttcccaagtcagaattcagtgtgctgatttgacatcagttcgtcccgaataagttcctgttaccaggatttacattcagcacattagaaacttgttggtgtgcttttattcttggagcattttccttagactaccttccactttgagtgctctgtttaggatgttgaggtgttaggattcttgacagccagaaagactgaacccactatctgggcacagtgttcgtgttgctctataaatgtatgctttttttgatttggggttgttttacctacattgtcaaactagatccatgcttaacagtgataatgaaggctttttgtttgttttgtttgtgggtcctccccccccccccaagacagggtttctctgtaggctgtcctagaacttgttcttttttaaccaaaatttggcaaggctgaaaatggaatcctataatcaatgctggccacattaaagttaatagttgagaagtcttgtctgaatttccttgggcaaaaagattctagccagttcaataccctgttgtgcaaattcaatttgctgttataatttgctctcagttatcagttggaaggaggttaattctaatgtacttggaagaggcctgtagaccatctataactgcatcagttgtacagcgttgttgcctgggattctctagttcacataaactcccaagtcttagccgtggtgatggctacagtgtggaagatggtgagcattctagtgagtatcgcgatgacggcagtaaagagcagcaggcagccgtggctgggctcactgaccgtggctgtaagttacggaggcagcacacacttctgtacacacctctcatcagttaccggagtcattgcattgcggactaactggctgactcaagttgtcttgctactgaagtcttgagttggtctcatgcatttaccctgttgacttgagcaccttaaagtcgaaaggatgtctggttgtggctttattgtaaacagccttaggtaaagaggggagtatatcggttaggaaggtgaaaaatgatacttccaagttcagtgggaaaccctgggtttatcccccagcttaagaaagaatgcctaacaatgtttcagaattagattctgtggaaggtgagggtgttagaacagtccaaatttgttattgtagacttgcagtgggaggaatttttaaatatacagatcagtcgacactcattaacttcactgataaaggtggaaacggatgtggcaacacttctaagttcatttgtatatgtttgtaatttgattggttgtattctgttgcactctagaatttgaaggcaaggttacctctgctttttaatttttttttttttaaagaaagaaaaaacactgaaagaaacttcaaaagatctgttaatgctaatacctgaatgtggcatttaacatgtcatggaaactgctttgaataaatacttgagaaaaggaatgaaataattgccgtttttgttgttgagtgaatgggtgtggtttaatgagcgtaatcatttttataaaacagctgtgagactgaagtggaatccttattaaatgtggaaaatggcctttgaggattacagtagagattcaactaagagagtaaataaagcttgaaactaattcgttgtaaattgcttctacaatcattgctctatatagcatgctattgccaatcagttttatgtattaagacctatcagcatgtcttttttaggttgacctcattttaaattataagatgctctctgtaccgttttaacatttccaggatttattctttctaggcaaattccactggactgtttccattgtagaagcttccttatagattcttcaaatgaagcttacagtgtgctttcttggggttttgatttgcactaaattttattttctgaaagatcacttatgtttataatgtagtgctttgtcttaacaattaaactttccagcactcatgca

SEQ ID NO: 4 - protein, 287 aa, Mus musculus
MSEEQFGGDGAAAAATAAVGGSAGEQEGAMVAAAAQGPAAAAGSGSGGGGSAAGGTEGGSAEAEGAKIDASKNEEDEGKMFIGGLSWDTTKKDLKDYFSKFGEVVDCTLKLDPITGRSRGFGFVLFKESESVDKVMDQKEHKLNGKVIDPKRAKAMKTKEPVKKIFVGGLSPDTPEEKIREYFGGFGEVESIELPMDNKTNKRRGFCFITFKEEEPVKKIMEKKYHNVGLSKCEIKVAMSKEQYQQQQQWGSRGGFAGRARGRGGDQQSGYGKVSRRGGHQNSYKPY

SEQ ID NO: 5 - DNA, 2110 bp, Homo sapiens
  source 1..2110: mol_type = other DNA
cttccgtcggccattttaggtggtccgcggcggcgccattaaagcgaggaggaggcgagagcggccgccgctggtgcttattcttttttagtgcagcgggagagagcgggagtgtgcgccgcgcgagagtgggaggcgaagggggcaggccagggagaggcgcaggagcctttgcagccacgcgcgcgccttccctgtcttgtgtgcttcgcgaggtagagcgggcgcgcggcagcggcggggattactttgctgctagtttcggttcgcggcagcggcgggtgtagtctcggcggcagcggcggagacactagcactatgtcggaggagcagttcggcggggacggggcggcggcagcggcaacggcggcggtaggcggctcggcgggcgagcaggagggagccatggtggcggcgacacagggggcagcggcggcggcgggaagcggagccgggaccgggggcggaaccgcgtctggaggcaccgaagggggcagcgccgagtcggagggggcgaagattgacgccagtaagaacgaggaggatgaaggccattcaaactcctccccacgacactctgaagcagcgacggcacagcgggaagaatggaaaatgtttataggaggccttagctgggacactacaaagaaagatctgaaggactacttttccaaatttggtgaagttgtagactgcactctgaagttagatcctatcacagggcgatcaaggggttttggctttgtgctatttaaagaatcggagagtgtagataaggtcatggatcaaaaagaacataaattgaatgggaaggtgattgatcctaaaagggccaaagccatgaaaacaaaagagccggttaaaaaaatttttgttggtggcctttctccagatacacctgaagagaaaataagggagtactttggtggttttggtgaggtggaatccatagagctccccatggacaacaagaccaataagaggcgtgggttctgctttattacctttaaggaagaagaaccagtgaagaagataatggaaaagaaataccacaatgttggtcttagtaaatgtgaaataaaagtagccatgtcgaaggaacaatatcagcaacagcaacagtggggatctagaggaggatttgcaggaagagctcgtggaagaggtggtgaccagcagagtggttatgggaaggtatccaggcgaggtggtcatcaaaatagctacaaaccatactaaattattccatttgcaacttatccccaacaggtggtgaagcagtattttccaatttgaagattcatttgaaggtggctcctgccacctgctaatagcagttcaaactaaattttttgtatcaagtccctgaatggaagtatgacgttgggtccctctgaagtttaattctgagttctcattaaaagaaatttgctttcattgttttatttcttaattgctatgcttcagaatcaatttgtgttttatgccctttcccccagtattgtagagcaagtcttgtgttaaaagcccagtgtgacagtgtcatgatgtagtagtgtcttactggttttttaataaatccttttgtataaaaatgtattggctcttttatcatcagaataggaaaaattgtcatggattcaagttattaaaagcataagtttggaagacaggcttgccgaaattgaggacatgattaaaattgcagtgaagtttgaaatgtttttagcaaaatctaatttttgccataatgtgtcctccctgtccaaattgggaatgacttaatgtcaatttgtttgttggttgttttaataatacttccttatgtagccattaagatttatatgaatattttcccaaatgcccagtttttgcttaatatgtattgtgctttttagaacaaatctggataaatgtgcaaaagtacccctttgcacagatagttaatgttttatgcttccattaaataaaaaggacttaaaatctgttaattataatagaaatgcggctagttcagagagatttttagagctgtggtggacttcatagatgaattcaagtgttgagggaggattaaagaaatatataccgtgtttatgtgtgtgtgctt

SEQ ID NO: 6 - protein, 306 aa, Homo sapiens
MSEEQFGGDGAAAAATAAVGGSAGEQEGAMVAATQGAAAAAGSGAGTGGGTASGGTEGGSAESEGAKIDASKNEEDEGHSNSSPRHSEAATAQREEWKMFIGGLSWDTTKKDLKDYFSKFGEVVDCTLKLDPITGRSRGFGFVLFKESESVDKVMDQKEHKLNGKVIDPKRAKAMKTKEPVKKIFVGGLSPDTPEEKIREYFGGFGEVESIELPMDNKTNKRRGFCFITFKEEEPVKKIMEKKYHNVGLSKCEIKVAMSKEQYQQQQQWGSRGGFAGRARGRGGDQQSGYGKVSRRGGHQNSYKPY

SEQ ID NO: 7 - DNA, 6664 bp, Mus musculus
  source 1..6664: mol_type = other DNA
ccattttaggtggtccgcggcggcgccattaaagcgaggaggaggcgagagtggccgccgctgctacttcattcttttttttttcagtgcagccggggagagcgagagagcgcgctgcgcgagagtgggaggcgaggggggcaggccggggagaggcgcaggagcccttgcagccacgcgcgcgccttgtctagggtgcctcgcgaggtagagcgggcatcgcgcggcggcggcggggattactttgctgctagtttcggttcgcggcggcggcggcgtcggcgggtgtcgtcttcggcggcggcagtagcactatgtcggaggagcagttcggaggggacggggcggcggcggcggcaacggcggcggtaggcggctcggcgggcgagcaggagggagccatggtggcggcggcggcgcaggggccggcggcggcggcgggaagcgggagcggcggcggcggctctgcggccggaggcaccgaaggaggcagcgccgaggcagagggagccaagatcgacgccagtaagaacgaggaggatgaaggccattcaaactcctccccacgacacactgaagcagcggcggcacagcgggaagaatggaaaatgtttataggaggccttagctgggacaccacaaagaaagatctgaaggactacttttccaaatttggtgaagttgtagactgcactctgaagttagatcctatcacagggcgatcaaggggttttggctttgtgctatttaaagagtcggagagtgtagataaggtcatggatcagaaagaacataaattgaatgggaaagtcattgatcctaaaagggccaaagccatgaaaacaaaagagcctgtcaaaaaaatttttgttggtggcctttctccagacacacctgaagaaaaaataagagagtactttggtggttttggtgaggttgaatccatagagctccctatggacaacaagaccaataagaggcgtgggttctgttttattacctttaaggaagaggagccagtgaagaagataatggaaaagaaataccacaatgttggtcttagtaaatgtgaaataaaagtagccatgtcaaaggaacagtatcagcagcagcagcagtggggatctagaggagggtttgcaggcagagctcgcggaagaggtggagatcagcagagtggttatgggaaagtatccaggcgaggtggacatcaaaatagctacaaaccatactaaattattccatttgcaacttatccccaacaggtggtgaagcagtattttccaatttgaagattcatttgaaggtggctcctgccacctgctaatagcagttcaaactaaattttttctatcaagttcctgaatggaagtatgacgttgggtccctctgaagtttaattctgagttctcattaaaagaatttgctttcattgttttatttcttaattgctatgcttcagtatcaatttgtgttttatgccccccctcccccccagtattgtagagcaagtcttgtgttaaaaaaagcccagtgtgacagtgtcatgatgtagtagtgtcttactggttttttaataaatccttttgtataaaaatgtattggctcttttatcatcagaataggaggaagtgaaatactacaaatgtttgtcttggattcaagtcactagaagcataaatttgaggggataaaaacaacggtaaactttgtctgaaagagggcatggttaaaaatgtagtgaattttaaatgtttttagcaaaatttgattttgcccaagaatccctgtctgaattggaaatgacttaatgtagtcaatgtgcttgttggttgtcttaatattacttctgtagccattaagttttatgagtaacttcccaaatacccacgtttttctttatatgtattgtgctttttaaaaacaaatctggaaaaatgggcaagaacatttgcagacaattgtttttaagcttccattaaataaaaaaaatgtggacttaaggaaatctattaatttaaatagaactgcagctagtttagagagtatttttttcttaaagctttggtgtaattagggaagattttaaaaaatgcatagtgtttatttgtatgtgtgctctttttttaagtcaatttttggggggttggtctgttaactgagtctaggatttaaaggtaagatgttcctagaaatcttgtcatcccaaaggggcgggcgctaaggtgaaacttcagggttcagtcagggtcactgctttatgtgtgaaatcactcaaattggtaagtctcttatgttagcattcaggacattgatttcaacttggatggacaatttatagttactactgaattgtgtgttaatgtgttcagtcctggtaagttttcagtttgatcagttagttggaagcagacttgaagagctgttagtcacgtgagccatgggtgcagtcgatctgtggtcagatgcctgagtctgtgatagtgaattgtgtctaaagacattttaatgataaaagtcagtgctgtaaagttgaaagttcatgagagacatacaatgagggctgcagcccatttttaaaaacattataatacaaaagtatgcacatttgtttacatatccctgcctttgtattacagtggcaggtttgtgtacttaaactgggaaagcctcagatctatgattacctggcctatcatagaaagtgtctaaataaatcactctgtcaattgaatacattagtattagctagcatacttcattatgcctgttttccataaataccacaccaaaaacttgcttggggcagtttgagcctagttcatgagctgctatcagattggtcttgatcctatataataggccaaatgtctgtaaacagctgtgctggtggaatgtagaaagtcactgcactcagattcaacttcctgattggaagtcatcacagtgtgattaaacattttcacaaagaatagtagataaataacttggtttttaatgttaactttgtttccattaagtcacatttaaaaacttatcctcacgcctacctgagttaattatctgttgacctagatatctttctggccactcactgacttatttcttgaacttttgccatttgcataaatcttgtcagctttgttcttgattatgcattgtccaggctgagctagttgtctttccaggaatccctttgtctctgaattaggtcctttgtttcctaaatcatcctgcttgtttggcacaagtcttcccaggccagtgagacctccgtgtcctctcagcaccataggggtaggtaaccctggttaggctggacaggggtttgctgagggagtttgttcatttgaatctaggtcttacatgacgtctttcaaatagggtttttaccttgacactaaactgtccagtctaagcagttctgcaaaatgtgagggaattatgaacttcttcctgcagtgggtttttatggttttggtttgttttttgttgttttggttctttgttgagccctggacaaaaacttccctagttctggtttctacaatttaaattaaaaacagaattcatcttagaatttttcaccctcttccccaactattctaatcaatcttaagtatgcccttcatcttttttccttcctaaggcttttactgatagtgtaattccgtactcttcaaccctgggaaggctgaagtggattcttgagctcatttcaaggctgacctgggtgttggcaagaacccagcttagaacaaacacatgcaaggccatcttaccttacatcctgttgcttggacttcttcctgctcaaagtttttagtggatgctaagtgatctttgcttccactgaggagtggaacactttagaatgaacctctagatagatatttttattgtctggtgagggttactggagtttcccaccctgcctgaagggtgaatctggcttacagtgttctcatctcaaagggaagaaggcagatggctgtgtccagagagagccatcacagtttgcttcagagacactagaatgggctggaagatctagtggtcttaatcagacttgaaacctggcctttcttcattacccatatgtctaccagtacttgggctaacacttaagccattagggcctttgtaggggtgttttgagaccccctccatgctaacaaatatacaggtttcttaacatttgctcataaacttgtaaagcttactttctcttaatccaccccacatttaacaagccctggtacttagaatttcagaagagtaatggcaggtaggtgtgtgtgtgtgtgtgtgtgtgtgtgtgtgtgtgtgtgtgtgagagagagagagagagagagagagagagagagagagagagagagagagagagagaagtttgtggaaaatcaggtaatgacagctcatccttttagaattgtacttcagaatagaaacatttggtgggctgttaggtagctttgattacttgtgggtagacctgctagtattgccagtcctcaagcaatgagctttctgtatcttgtttactagatatatactaccaggtgagtcatttcctggggttctgttttcttttaaaatctttccctaaacttaatatgtattaaaaagtctggcttttcagtccattctttgtgcactgggatggcaattgcttcattatatgacaattgctgttcccaagtcagaattcagtgtgctgatttgacatcagttcgtcccgaataagttcctgttaccaggatttacattcagcacattagaaacttgttggtgtgcttttattcttggagcattttccttagactaccttccactttgagtgctctgtttaggatgttgaggtgttaggattcttgacagccagaaagactgaacccactatctgggcacagtgttcgtgttgctctataaatgtatgctttttttgatttggggttgttttacctacattgtcaaactagatccatgcttaacagtgataatgaaggctttttgtttgttttgtttgtgggtcctccccccccccccaagacagggtttctctgtaggctgtcctagaacttgttcttttttaaccaaaatttggcaaggctgaaaatggaatcctataatcaatgctggccacattaaagttaatagttgagaagtcttgtctgaatttccttgggcaaaaagattctagccagttcaataccctgttgtgcaaattcaatttgctgttataatttgctctcagttatcagttggaaggaggttaattctaatgtacttggaagaggcctgtagaccatctataactgcatcagttgtacagcgttgttgcctgggattctctagttcacataaactcccaagtcttagccgtggtgatggctacagtgtggaagatggtgagcattctagtgagtatcgcgatgacggcagtaaagagcagcaggcagccgtggctgggctcactgaccgtggctgtaagttacggaggcagcacacacttctgtacacacctctcatcagttaccggagtcattgcattgcggactaactggctgactcaagttgtcttgctactgaagtcttgagttggtctcatgcatttaccctgttgacttgagcaccttaaagtcgaaaggatgtctggttgtggctttattgtaaacagccttaggtaaagaggggagtatatcggttaggaaggtgaaaaatgatacttccaagttcagtgggaaaccctgggtttatcccccagcttaagaaagaatgcctaacaatgtttcagaattagattctgtggaaggtgagggtgttagaacagtccaaatttgttattgtagacttgcagtgggaggaatttttaaatatacagatcagtcgacactcattaacttcactgataaaggtggaaacggatgtggcaacacttctaagttcatttgtatatgtttgtaatttgattggttgtattctgttgcactctagaatttgaaggcaaggttacctctgctttttaatttttttttttttaaagaaagaaaaaacactgaaagaaacttcaaaagatctgttaatgctaatacctgaatgtggcatttaacatgtcatggaaactgctttgaataaatacttgagaaaaggaatgaaataattgccgtttttgttgttgagtgaatgggtgtggtttaatgagcgtaatcatttttataaaacagctgtgagactgaagtggaatccttattaaatgtggaaaatggcctttgaggattacagtagagattcaactaagagagtaaataaagcttgaaactaattcgttgtaaattgcttctacaatcattgctctatatagcatgctattgccaatcagttttatgtattaagacctatcagcatgtcttttttaggttgacctcattttaaattataagatgctctctgtaccgttttaacatttccaggatttattctttctaggcaaattccactggactgtttccattgtagaagcttccttatagattcttcaaatgaagcttacagtgtgctttcttggggttttgatttgcactaaattttattttctgaaagatcacttatgtttataatgtagtgctttgtcttaacaattaaactttccagcactcatgca

SEQ ID NO: 8 - protein, 306 aa, Mus musculus
MSEEQFGGDGAAAAATAAVGGSAGEQEGAMVAAAAQGPAAAAGSGSGGGGSAAGGTEGGSAEAEGAKIDASKNEEDEGHSNSSPRHTEAAAAQREEWKMFIGGLSWDTTKKDLKDYFSKFGEVVDCTLKLDPITGRSRGFGFVLFKESESVDKVMDQKEHKLNGKVIDPKRAKAMKTKEPVKKIFVGGLSPDTPEEKIREYFGGFGEVESIELPMDNKTNKRRGFCFITFKEEEPVKKIMEKKYHNVGLSKCEIKVAMSKEQYQQQQQWGSRGGFAGRARGRGGDQQSGYGKVSRRGGHQNSYKPY

SEQ ID NO: 9 - DNA, 2200 bp, Homo sapiens
  source 1..2200: mol_type = other DNA
cttccgtcggccattttaggtggtccgcggcggcgccattaaagcgaggaggaggcgagagcggccgccgctggtgcttattcttttttagtgcagcgggagagagcgggagtgtgcgccgcgcgagagtgggaggcgaagggggcaggccagggagaggcgcaggagcctttgcagccacgcgcgcgccttccctgtcttgtgtgcttcgcgaggtagagcgggcgcgcggcagcggcggggattactttgctgctagtttcggttcgcggcagcggcgggtgtagtctcggcggcagcggcggagacactagcactatgtcggaggagcagttcggcggggacggggcggcggcagcggcaacggcggcggtaggcggctcggcgggcgagcaggagggagccatggtggcggcgacacagggggcagcggcggcggcgggaagcggagccgggaccgggggcggaaccgcgtctggaggcaccgaagggggcagcgccgagtcggagggggcgaagattgacgccagtaagaacgaggaggatgaagggaaaatgtttataggaggccttagctgggacactacaaagaaagatctgaaggactacttttccaaatttggtgaagttgtagactgcactctgaagttagatcctatcacagggcgatcaaggggttttggctttgtgctatttaaagaatcggagagtgtagataaggtcatggatcaaaaagaacataaattgaatgggaaggtgattgatcctaaaagggccaaagccatgaaaacaaaagagccggttaaaaaaatttttgttggtggcctttctccagatacacctgaagagaaaataagggagtactttggtggttttggtgaggtggaatccatagagctccccatggacaacaagaccaataagaggcgtgggttctgctttattacctttaaggaagaagaaccagtgaagaagataatggaaaagaaataccacaatgttggtcttagtaaatgtgaaataaaagtagccatgtcgaaggaacaatatcagcaacagcaacagtggggatctagaggaggatttgcaggaagagctcgtggaagaggtggtggccccagtcaaaactggaaccagggatatagtaactattggaatcaaggctatggcaactatggatataacagccaaggttacggtggttatggaggatatgactacactggttacaacaactactatggatatggtgattatagcaaccagcagagtggttatgggaaggtatccaggcgaggtggtcatcaaaatagctacaaaccatactaaattattccatttgcaacttatccccaacaggtggtgaagcagtattttccaatttgaagattcatttgaaggtggctcctgccacctgctaatagcagttcaaactaaattttttgtatcaagtccctgaatggaagtatgacgttgggtccctctgaagtttaattctgagttctcattaaaagaaatttgctttcattgttttatttcttaattgctatgcttcagaatcaatttgtgttttatgccctttcccccagtattgtagagcaagtcttgtgttaaaagcccagtgtgacagtgtcatgatgtagtagtgtcttactggttttttaataaatccttttgtataaaaatgtattggctcttttatcatcagaataggaaaaattgtcatggattcaagttattaaaagcataagtttggaagacaggcttgccgaaattgaggacatgattaaaattgcagtgaagtttgaaatgtttttagcaaaatctaatttttgccataatgtgtcctccctgtccaaattgggaatgacttaatgtcaatttgtttgttggttgttttaataatacttccttatgtagccattaagatttatatgaatattttcccaaatgcccagtttttgcttaatatgtattgtgctttttagaacaaatctggataaatgtgcaaaagtacccctttgcacagatagttaatgttttatgcttccattaaataaaaaggacttaaaatctgttaattataatagaaatgcggctagttcagagagatttttagagctgtggtggacttcatagatgaattcaagtgttgagggaggattaaagaaatatataccgtgtttatgtgtgtgtgctt

SEQ ID NO: 10 - protein, 336 aa, Homo sapiens
MSEEQFGGDGAAAAATAAVGGSAGEQEGAMVAATQGAAAAAGSGAGTGGGTASGGTEGGSAESEGAKIDASKNEEDEGKMFIGGLSWDTTKKDLKDYFSKFGEVVDCTLKLDPITGRSRGFGFVLFKESESVDKVMDQKEHKLNGKVIDPKRAKAMKTKEPVKKIFVGGLSPDTPEEKIREYFGGFGEVESIELPMDNKTNKRRGFCFITFKEEEPVKKIMEKKYHNVGLSKCEIKVAMSKEQYQQQQQWGSRGGFAGRARGRGGGPSQNWNQGYSNYWNQGYGNYGYNSQGYGGYGGYDYTGYNNYYGYGDYSNQQSGYGKVSRRGGHQNSYKPY

SEQ ID NO: 11 - DNA, 6754 bp, Mus musculus
  source 1..6754: mol_type = other DNA
ccattttaggtggtccgcggcggcgccattaaagcgaggaggaggcgagagtggccgccgctgctacttcattcttttttttttcagtgcagccggggagagcgagagagcgcgctgcgcgagagtgggaggcgaggggggcaggccggggagaggcgcaggagcccttgcagccacgcgcgcgccttgtctagggtgcctcgcgaggtagagcgggcatcgcgcggcggcggcggggattactttgctgctagtttcggttcgcggcggcggcggcgtcggcgggtgtcgtcttcggcggcggcagtagcactatgtcggaggagcagttcggaggggacggggcggcggcggcggcaacggcggcggtaggcggctcggcgggcgagcaggagggagccatggtggcggcggcggcgcaggggccggcggcggcggcgggaagcgggagcggcggcggcggctctgcggccggaggcaccgaaggaggcagcgccgaggcagagggagccaagatcgacgccagtaagaacgaggaggatgaagggaaaatgtttataggaggccttagctgggacaccacaaagaaagatctgaaggactacttttccaaatttggtgaagttgtagactgcactctgaagttagatcctatcacagggcgatcaaggggttttggctttgtgctatttaaagagtcggagagtgtagataaggtcatggatcagaaagaacataaattgaatgggaaagtcattgatcctaaaagggccaaagccatgaaaacaaaagagcctgtcaaaaaaatttttgttggtggcctttctccagacacacctgaagaaaaaataagagagtactttggtggttttggtgaggttgaatccatagagctccctatggacaacaagaccaataagaggcgtgggttctgttttattacctttaaggaagaggagccagtgaagaagataatggaaaagaaataccacaatgttggtcttagtaaatgtgaaataaaagtagccatgtcaaaggaacagtatcagcagcagcagcagtggggatctagaggagggtttgcaggcagagctcgcggaagaggtggaggccccagtcaaaactggaaccagggatatagtaactattggaatcaaggctatggcaactatggatataacagccaaggttacggaggttatggaggatatgactacactggttacaacaactactatggatatggtgattatagcaatcagcagagtggttatgggaaagtatccaggcgaggtggacatcaaaatagctacaaaccatactaaattattccatttgcaacttatccccaacaggtggtgaagcagtattttccaatttgaagattcatttgaaggtggctcctgccacctgctaatagcagttcaaactaaattttttctatcaagttcctgaatggaagtatgacgttgggtccctctgaagtttaattctgagttctcattaaaagaatttgctttcattgttttatttcttaattgctatgcttcagtatcaatttgtgttttatgccccccctcccccccagtattgtagagcaagtcttgtgttaaaaaaagcccagtgtgacagtgtcatgatgtagtagtgtcttactggttttttaataaatccttttgtataaaaatgtattggctcttttatcatcagaataggaggaagtgaaatactacaaatgtttgtcttggattcaagtcactagaagcataaatttgaggggataaaaacaacggtaaactttgtctgaaagagggcatggttaaaaatgtagtgaattttaaatgtttttagcaaaatttgattttgcccaagaatccctgtctgaattggaaatgacttaatgtagtcaatgtgcttgttggttgtcttaatattacttctgtagccattaagttttatgagtaacttcccaaatacccacgtttttctttatatgtattgtgctttttaaaaacaaatctggaaaaatgggcaagaacatttgcagacaattgtttttaagcttccattaaataaaaaaaatgtggacttaaggaaatctattaatttaaatagaactgcagctagtttagagagtatttttttcttaaagctttggtgtaattagggaagattttaaaaaatgcatagtgtttatttgtatgtgtgctctttttttaagtcaatttttggggggttggtctgttaactgagtctaggatttaaaggtaagatgttcctagaaatcttgtcatcccaaaggggcgggcgctaaggtgaaacttcagggttcagtcagggtcactgctttatgtgtgaaatcactcaaattggtaagtctcttatgttagcattcaggacattgatttcaacttggatggacaatttatagttactactgaattgtgtgttaatgtgttcagtcctggtaagttttcagtttgatcagttagttggaagcagacttgaagagctgttagtcacgtgagccatgggtgcagtcgatctgtggtcagatgcctgagtctgtgatagtgaattgtgtctaaagacattttaatgataaaagtcagtgctgtaaagttgaaagttcatgagagacatacaatgagggctgcagcccatttttaaaaacattataatacaaaagtatgcacatttgtttacatatccctgcctttgtattacagtggcaggtttgtgtacttaaactgggaaagcctcagatctatgattacctggcctatcatagaaagtgtctaaataaatcactctgtcaattgaatacattagtattagctagcatacttcattatgcctgttttccataaataccacaccaaaaacttgcttggggcagtttgagcctagttcatgagctgctatcagattggtcttgatcctatataataggccaaatgtctgtaaacagctgtgctggtggaatgtagaaagtcactgcactcagattcaacttcctgattggaagtcatcacagtgtgattaaacattttcacaaagaatagtagataaataacttggtttttaatgttaactttgtttccattaagtcacatttaaaaacttatcctcacgcctacctgagttaattatctgttgacctagatatctttctggccactcactgacttatttcttgaacttttgccatttgcataaatcttgtcagctttgttcttgattatgcattgtccaggctgagctagttgtctttccaggaatccctttgtctctgaattaggtcctttgtttcctaaatcatcctgcttgtttggcacaagtcttcccaggccagtgagacctccgtgtcctctcagcaccataggggtaggtaaccctggttaggctggacaggggtttgctgagggagtttgttcatttgaatctaggtcttacatgacgtctttcaaatagggtttttaccttgacactaaactgtccagtctaagcagttctgcaaaatgtgagggaattatgaacttcttcctgcagtgggtttttatggttttggtttgttttttgttgttttggttctttgttgagccctggacaaaaacttccctagttctggtttctacaatttaaattaaaaacagaattcatcttagaatttttcaccctcttccccaactattctaatcaatcttaagtatgcccttcatcttttttccttcctaaggcttttactgatagtgtaattccgtactcttcaaccctgggaaggctgaagtggattcttgagctcatttcaaggctgacctgggtgttggcaagaacccagcttagaacaaacacatgcaaggccatcttaccttacatcctgttgcttggacttcttcctgctcaaagtttttagtggatgctaagtgatctttgcttccactgaggagtggaacactttagaatgaacctctagatagatatttttattgtctggtgagggttactggagtttcccaccctgcctgaagggtgaatctggcttacagtgttctcatctcaaagggaagaaggcagatggctgtgtccagagagagccatcacagtttgcttcagagacactagaatgggctggaagatctagtggtcttaatcagacttgaaacctggcctttcttcattacccatatgtctaccagtacttgggctaacacttaagccattagggcctttgtaggggtgttttgagaccccctccatgctaacaaatatacaggtttcttaacatttgctcataaacttgtaaagcttactttctcttaatccaccccacatttaacaagccctggtacttagaatttcagaagagtaatggcaggtaggtgtgtgtgtgtgtgtgtgtgtgtgtgtgtgtgtgtgtgtgtgagagagagagagagagagagagagagagagagagagagagagagagagagagagaagtttgtggaaaatcaggtaatgacagctcatccttttagaattgtacttcagaatagaaacatttggtgggctgttaggtagctttgattacttgtgggtagacctgctagtattgccagtcctcaagcaatgagctttctgtatcttgtttactagatatatactaccaggtgagtcatttcctggggttctgttttcttttaaaatctttccctaaacttaatatgtattaaaaagtctggcttttcagtccattctttgtgcactgggatggcaattgcttcattatatgacaattgctgttcccaagtcagaattcagtgtgctgatttgacatcagttcgtcccgaataagttcctgttaccaggatttacattcagcacattagaaacttgttggtgtgcttttattcttggagcattttccttagactaccttccactttgagtgctctgtttaggatgttgaggtgttaggattcttgacagccagaaagactgaacccactatctgggcacagtgttcgtgttgctctataaatgtatgctttttttgatttggggttgttttacctacattgtcaaactagatccatgcttaacagtgataatgaaggctttttgtttgttttgtttgtgggtcctccccccccccccaagacagggtttctctgtaggctgtcctagaacttgttcttttttaaccaaaatttggcaaggctgaaaatggaatcctataatcaatgctggccacattaaagttaatagttgagaagtcttgtctgaatttccttgggcaaaaagattctagccagttcaataccctgttgtgcaaattcaatttgctgttataatttgctctcagttatcagttggaaggaggttaattctaatgtacttggaagaggcctgtagaccatctataactgcatcagttgtacagcgttgttgcctgggattctctagttcacataaactcccaagtcttagccgtggtgatggctacagtgtggaagatggtgagcattctagtgagtatcgcgatgacggcagtaaagagcagcaggcagccgtggctgggctcactgaccgtggctgtaagttacggaggcagcacacacttctgtacacacctctcatcagttaccggagtcattgcattgcggactaactggctgactcaagttgtcttgctactgaagtcttgagttggtctcatgcatttaccctgttgacttgagcaccttaaagtcgaaaggatgtctggttgtggctttattgtaaacagccttaggtaaagaggggagtatatcggttaggaaggtgaaaaatgatacttccaagttcagtgggaaaccctgggtttatcccccagcttaagaaagaatgcctaacaatgtttcagaattagattctgtggaaggtgagggtgttagaacagtccaaatttgttattgtagacttgcagtgggaggaatttttaaatatacagatcagtcgacactcattaacttcactgataaaggtggaaacggatgtggcaacacttctaagttcatttgtatatgtttgtaatttgattggttgtattctgttgcactctagaatttgaaggcaaggttacctctgctttttaatttttttttttttaaagaaagaaaaaacactgaaagaaacttcaaaagatctgttaatgctaatacctgaatgtggcatttaacatgtcatggaaactgctttgaataaatacttgagaaaaggaatgaaataattgccgtttttgttgttgagtgaatgggtgtggtttaatgagcgtaatcatttttataaaacagctgtgagactgaagtggaatccttattaaatgtggaaaatggcctttgaggattacagtagagattcaactaagagagtaaataaagcttgaaactaattcgttgtaaattgcttctacaatcattgctctatatagcatgctattgccaatcagttttatgtattaagacctatcagcatgtcttttttaggttgacctcattttaaattataagatgctctctgtaccgttttaacatttccaggatttattctttctaggcaaattccactggactgtttccattgtagaagcttccttatagattcttcaaatgaagcttacagtgtgctttcttggggttttgatttgcactaaattttattttctgaaagatcacttatgtttataatgtagtgctttgtcttaacaattaaactttccagcactcatgca

SEQ ID NO: 12 - protein, 336 aa, Mus musculus
MSEEQFGGDGAAAAATAAVGGSAGEQEGAMVAAAAQGPAAAAGSGSGGGGSAAGGTEGGSAEAEGAKIDASKNEEDEGKMFIGGLSWDTTKKDLKDYFSKFGEVVDCTLKLDPITGRSRGFGFVLFKESESVDKVMDQKEHKLNGKVIDPKRAKAMKTKEPVKKIFVGGLSPDTPEEKIREYFGGFGEVESIELPMDNKTNKRRGFCFITFKEEEPVKKIMEKKYHNVGLSKCEIKVAMSKEQYQQQQQWGSRGGFAGRARGRGGGPSQNWNQGYSNYWNQGYGNYGYNSQGYGGYGGYDYTGYNNYYGYGDYSNQQSGYGKVSRRGGHQNSYKPY

SEQ ID NO: 13 - DNA, 2257 bp, Homo sapiens
  source 1..2257: mol_type = other DNA
cttccgtcggccattttaggtggtccgcggcggcgccattaaagcgaggaggaggcgagagcggccgccgctggtgcttattcttttttagtgcagcgggagagagcgggagtgtgcgccgcgcgagagtgggaggcgaagggggcaggccagggagaggcgcaggagcctttgcagccacgcgcgcgccttccctgtcttgtgtgcttcgcgaggtagagcgggcgcgcggcagcggcggggattactttgctgctagtttcggttcgcggcagcggcgggtgtagtctcggcggcagcggcggagacactagcactatgtcggaggagcagttcggcggggacggggcggcggcagcggcaacggcggcggtaggcggctcggcgggcgagcaggagggagccatggtggcggcgacacagggggcagcggcggcggcgggaagcggagccgggaccgggggcggaaccgcgtctggaggcaccgaagggggcagcgccgagtcggagggggcgaagattgacgccagtaagaacgaggaggatgaaggccattcaaactcctccccacgacactctgaagcagcgacggcacagcgggaagaatggaaaatgtttataggaggccttagctgggacactacaaagaaagatctgaaggactacttttccaaatttggtgaagttgtagactgcactctgaagttagatcctatcacagggcgatcaaggggttttggctttgtgctatttaaagaatcggagagtgtagataaggtcatggatcaaaaagaacataaattgaatgggaaggtgattgatcctaaaagggccaaagccatgaaaacaaaagagccggttaaaaaaatttttgttggtggcctttctccagatacacctgaagagaaaataagggagtactttggtggttttggtgaggtggaatccatagagctccccatggacaacaagaccaataagaggcgtgggttctgctttattacctttaaggaagaagaaccagtgaagaagataatggaaaagaaataccacaatgttggtcttagtaaatgtgaaataaaagtagccatgtcgaaggaacaatatcagcaacagcaacagtggggatctagaggaggatttgcaggaagagctcgtggaagaggtggtggccccagtcaaaactggaaccagggatatagtaactattggaatcaaggctatggcaactatggatataacagccaaggttacggtggttatggaggatatgactacactggttacaacaactactatggatatggtgattatagcaaccagcagagtggttatgggaaggtatccaggcgaggtggtcatcaaaatagctacaaaccatactaaattattccatttgcaacttatccccaacaggtggtgaagcagtattttccaatttgaagattcatttgaaggtggctcctgccacctgctaatagcagttcaaactaaattttttgtatcaagtccctgaatggaagtatgacgttgggtccctctgaagtttaattctgagttctcattaaaagaaatttgctttcattgttttatttcttaattgctatgcttcagaatcaatttgtgttttatgccctttcccccagtattgtagagcaagtcttgtgttaaaagcccagtgtgacagtgtcatgatgtagtagtgtcttactggttttttaataaatccttttgtataaaaatgtattggctcttttatcatcagaataggaaaaattgtcatggattcaagttattaaaagcataagtttggaagacaggcttgccgaaattgaggacatgattaaaattgcagtgaagtttgaaatgtttttagcaaaatctaatttttgccataatgtgtcctccctgtccaaattgggaatgacttaatgtcaatttgtttgttggttgttttaataatacttccttatgtagccattaagatttatatgaatattttcccaaatgcccagtttttgcttaatatgtattgtgctttttagaacaaatctggataaatgtgcaaaagtacccctttgcacagatagttaatgttttatgcttccattaaataaaaaggacttaaaatctgttaattataatagaaatgcggctagttcagagagatttttagagctgtggtggacttcatagatgaattcaagtgttgagggaggattaaagaaatatataccgtgtttatgtgtgtgtgctt

SEQ ID NO: 14 - protein, 355 aa, Homo sapiens
MSEEQFGGDGAAAAATAAVGGSAGEQEGAMVAATQGAAAAAGSGAGTGGGTASGGTEGGSAESEGAKIDASKNEEDEGHSNSSPRHSEAATAQREEWKMFIGGLSWDTTKKDLKDYFSKFGEVVDCTLKLDPITGRSRGFGFVLFKESESVDKVMDQKEHKLNGKVIDPKRAKAMKTKEPVKKIFVGGLSPDTPEEKIREYFGGFGEVESIELPMDNKTNKRRGFCFITFKEEEPVKKIMEKKYHNVGLSKCEIKVAMSKEQYQQQQQWGSRGGFAGRARGRGGGPSQNWNQGYSNYWNQGYGNYGYNSQGYGGYGGYDYTGYNNYYGYGDYSNQQSGYGKVSRRGGHQNSYKPY

SEQ ID NO: 15 - DNA, 6811 bp, Mus musculus
  source 1..6811: mol_type = other DNA
ccattttaggtggtccgcggcggcgccattaaagcgaggaggaggcgagagtggccgccgctgctacttcattcttttttttttcagtgcagccggggagagcgagagagcgcgctgcgcgagagtgggaggcgaggggggcaggccggggagaggcgcaggagcccttgcagccacgcgcgcgccttgtctagggtgcctcgcgaggtagagcgggcatcgcgcggcggcggcggggattactttgctgctagtttcggttcgcggcggcggcggcgtcggcgggtgtcgtcttcggcggcggcagtagcactatgtcggaggagcagttcggaggggacggggcggcggcggcggcaacggcggcggtaggcggctcggcgggcgagcaggagggagccatggtggcggcggcggcgcaggggccggcggcggcggcgggaagcgggagcggcggcggcggctctgcggccggaggcaccgaaggaggcagcgccgaggcagagggagccaagatcgacgccagtaagaacgaggaggatgaaggccattcaaactcctccccacgacacactgaagcagcggcggcacagcgggaagaatggaaaatgtttataggaggccttagctgggacaccacaaagaaagatctgaaggactacttttccaaatttggtgaagttgtagactgcactctgaagttagatcctatcacagggcgatcaaggggttttggctttgtgctatttaaagagtcggagagtgtagataaggtcatggatcagaaagaacataaattgaatgggaaagtcattgatcctaaaagggccaaagccatgaaaacaaaagagcctgtcaaaaaaatttttgttggtggcctttctccagacacacctgaagaaaaaataagagagtactttggtggttttggtgaggttgaatccatagagctccctatggacaacaagaccaataagaggcgtgggttctgttttattacctttaaggaagaggagccagtgaagaagataatggaaaagaaataccacaatgttggtcttagtaaatgtgaaataaaagtagccatgtcaaaggaacagtatcagcagcagcagcagtggggatctagaggagggtttgcaggcagagctcgcggaagaggtggaggccccagtcaaaactggaaccagggatatagtaactattggaatcaaggctatggcaactatggatataacagccaaggttacggaggttatggaggatatgactacactggttacaacaactactatggatatggtgattatagcaatcagcagagtggttatgggaaagtatccaggcgaggtggacatcaaaatagctacaaaccatactaaattattccatttgcaacttatccccaacaggtggtgaagcagtattttccaatttgaagattcatttgaaggtggctcctgccacctgctaatagcagttcaaactaaattttttctatcaagttcctgaatggaagtatgacgttgggtccctctgaagtttaattctgagttctcattaaaagaatttgctttcattgttttatttcttaattgctatgcttcagtatcaatttgtgttttatgccccccctcccccccagtattgtagagcaagtcttgtgttaaaaaaagcccagtgtgacagtgtcatgatgtagtagtgtcttactggttttttaataaatccttttgtataaaaatgtattggctcttttatcatcagaataggaggaagtgaaatactacaaatgtttgtcttggattcaagtcactagaagcataaatttgaggggataaaaacaacggtaaactttgtctgaaagagggcatggttaaaaatgtagtgaattttaaatgtttttagcaaaatttgattttgcccaagaatccctgtctgaattggaaatgacttaatgtagtcaatgtgcttgttggttgtcttaatattacttctgtagccattaagttttatgagtaacttcccaaatacccacgtttttctttatatgtattgtgctttttaaaaacaaatctggaaaaatgggcaagaacatttgcagacaattgtttttaagcttccattaaataaaaaaaatgtggacttaaggaaatctattaatttaaatagaactgcagctagtttagagagtatttttttcttaaagctttggtgtaattagggaagattttaaaaaatgcatagtgtttatttgtatgtgtgctctttttttaagtcaatttttggggggttggtctgttaactgagtctaggatttaaaggtaagatgttcctagaaatcttgtcatcccaaaggggcgggcgctaaggtgaaacttcagggttcagtcagggtcactgctttatgtgtgaaatcactcaaattggtaagtctcttatgttagcattcaggacattgatttcaacttggatggacaatttatagttactactgaattgtgtgttaatgtgttcagtcctggtaagttttcagtttgatcagttagttggaagcagacttgaagagctgttagtcacgtgagccatgggtgcagtcgatctgtggtcagatgcctgagtctgtgatagtgaattgtgtctaaagacattttaatgataaaagtcagtgctgtaaagttgaaagttcatgagagacatacaatgagggctgcagcccatttttaaaaacattataatacaaaagtatgcacatttgtttacatatccctgcctttgtattacagtggcaggtttgtgtacttaaactgggaaagcctcagatctatgattacctggcctatcatagaaagtgtctaaataaatcactctgtcaattgaatacattagtattagctagcatacttcattatgcctgttttccataaataccacaccaaaaacttgcttggggcagtttgagcctagttcatgagctgctatcagattggtcttgatcctatataataggccaaatgtctgtaaacagctgtgctggtggaatgtagaaagtcactgcactcagattcaacttcctgattggaagtcatcacagtgtgattaaacattttcacaaagaatagtagataaataacttggtttttaatgttaactttgtttccattaagtcacatttaaaaacttatcctcacgcctacctgagttaattatctgttgacctagatatctttctggccactcactgacttatttcttgaacttttgccatttgcataaatcttgtcagctttgttcttgattatgcattgtccaggctgagctagttgtctttccaggaatccctttgtctctgaattaggtcctttgtttcctaaatcatcctgcttgtttggcacaagtcttcccaggccagtgagacctccgtgtcctctcagcaccataggggtaggtaaccctggttaggctggacaggggtttgctgagggagtttgttcatttgaatctaggtcttacatgacgtctttcaaatagggtttttaccttgacactaaactgtccagtctaagcagttctgcaaaatgtgagggaattatgaacttcttcctgcagtgggtttttatggttttggtttgttttttgttgttttggttctttgttgagccctggacaaaaacttccctagttctggtttctacaatttaaattaaaaacagaattcatcttagaatttttcaccctcttccccaactattctaatcaatcttaagtatgcccttcatcttttttccttcctaaggcttttactgatagtgtaattccgtactcttcaaccctgggaaggctgaagtggattcttgagctcatttcaaggctgacctgggtgttggcaagaacccagcttagaacaaacacatgcaaggccatcttaccttacatcctgttgcttggacttcttcctgctcaaagtttttagtggatgctaagtgatctttgcttccactgaggagtggaacactttagaatgaacctctagatagatatttttattgtctggtgagggttactggagtttcccaccctgcctgaagggtgaatctggcttacagtgttctcatctcaaagggaagaaggcagatggctgtgtccagagagagccatcacagtttgcttcagagacactagaatgggctggaagatctagtggtcttaatcagacttgaaacctggcctttcttcattacccatatgtctaccagtacttgggctaacacttaagccattagggcctttgtaggggtgttttgagaccccctccatgctaacaaatatacaggtttcttaacatttgctcataaacttgtaaagcttactttctcttaatccaccccacatttaacaagccctggtacttagaatttcagaagagtaatggcaggtaggtgtgtgtgtgtgtgtgtgtgtgtgtgtgtgtgtgtgtgtgtgagagagagagagagagagagagagagagagagagagagagagagagagagagagaagtttgtggaaaatcaggtaatgacagctcatccttttagaattgtacttcagaatagaaacatttggtgggctgttaggtagctttgattacttgtgggtagacctgctagtattgccagtcctcaagcaatgagctttctgtatcttgtttactagatatatactaccaggtgagtcatttcctggggttctgttttcttttaaaatctttccctaaacttaatatgtattaaaaagtctggcttttcagtccattctttgtgcactgggatggcaattgcttcattatatgacaattgctgttcccaagtcagaattcagtgtgctgatttgacatcagttcgtcccgaataagttcctgttaccaggatttacattcagcacattagaaacttgttggtgtgcttttattcttggagcattttccttagactaccttccactttgagtgctctgtttaggatgttgaggtgttaggattcttgacagccagaaagactgaacccactatctgggcacagtgttcgtgttgctctataaatgtatgctttttttgatttggggttgttttacctacattgtcaaactagatccatgcttaacagtgataatgaaggctttttgtttgttttgtttgtgggtcctccccccccccccaagacagggtttctctgtaggctgtcctagaacttgttcttttttaaccaaaatttggcaaggctgaaaatggaatcctataatcaatgctggccacattaaagttaatagttgagaagtcttgtctgaatttccttgggcaaaaagattctagccagttcaataccctgttgtgcaaattcaatttgctgttataatttgctctcagttatcagttggaaggaggttaattctaatgtacttggaagaggcctgtagaccatctataactgcatcagttgtacagcgttgttgcctgggattctctagttcacataaactcccaagtcttagccgtggtgatggctacagtgtggaagatggtgagcattctagtgagtatcgcgatgacggcagtaaagagcagcaggcagccgtggctgggctcactgaccgtggctgtaagttacggaggcagcacacacttctgtacacacctctcatcagttaccggagtcattgcattgcggactaactggctgactcaagttgtcttgctactgaagtcttgagttggtctcatgcatttaccctgttgacttgagcaccttaaagtcgaaaggatgtctggttgtggctttattgtaaacagccttaggtaaagaggggagtatatcggttaggaaggtgaaaaatgatacttccaagttcagtgggaaaccctgggtttatcccccagcttaagaaagaatgcctaacaatgtttcagaattagattctgtggaaggtgagggtgttagaacagtccaaatttgttattgtagacttgcagtgggaggaatttttaaatatacagatcagtcgacactcattaacttcactgataaaggtggaaacggatgtggcaacacttctaagttcatttgtatatgtttgtaatttgattggttgtattctgttgcactctagaatttgaaggcaaggttacctctgctttttaatttttttttttttaaagaaagaaaaaacactgaaagaaacttcaaaagatctgttaatgctaatacctgaatgtggcatttaacatgtcatggaaactgctttgaataaatacttgagaaaaggaatgaaataattgccgtttttgttgttgagtgaatgggtgtggtttaatgagcgtaatcatttttataaaacagctgtgagactgaagtggaatccttattaaatgtggaaaatggcctttgaggattacagtagagattcaactaagagagtaaataaagcttgaaactaattcgttgtaaattgcttctacaatcattgctctatatagcatgctattgccaatcagttttatgtattaagacctatcagcatgtcttttttaggttgacctcattttaaattataagatgctctctgtaccgttttaacatttccaggatttattctttctaggcaaattccactggactgtttccattgtagaagcttccttatagattcttcaaatgaagcttacagtgtgctttcttggggttttgatttgcactaaattttattttctgaaagatcacttatgtttataatgtagtgctttgtcttaacaattaaactttccagcactcatgca

SEQ ID NO: 16 - protein, 355 aa, Mus musculus
MSEEQFGGDGAAAAATAAVGGSAGEQEGAMVAAAAQGPAAAAGSGSGGGGSAAGGTEGGSAEAEGAKIDASKNEEDEGHSNSSPRHTEAAAAQREEWKMFIGGLSWDTTKKDLKDYFSKFGEVVDCTLKLDPITGRSRGFGFVLFKESESVDKVMDQKEHKLNGKVIDPKRAKAMKTKEPVKKIFVGGLSPDTPEEKIREYFGGFGEVESIELPMDNKTNKRRGFCFITFKEEEPVKKIMEKKYHNVGLSKCEIKVAMSKEQYQQQQQWGSRGGFAGRARGRGGGPSQNWNQGYSNYWNQGYGNYGYNSQGYGGYGGYDYTGYNNYYGYGDYSNQQSGYGKVSRRGGHQNSYKPY

SEQ ID NO: 17 - DNA, 600 bp, synthetic construct
  source 1..600: mol_type = other DNA
agccagcctcagtttcccctccactcagtccctaggaggaaggggcgcccaagcgcgggtttctggggttagactgccctccattgcaattggtccttctcccggcctctgcttcctccagctcacagggtatctgctcctcctggagccacaccttggttccccgaggtgccgctgggactcgggtaggggtgagggcccagggggcacagggggagccgagggccacaggaagggctggtggctgaaggagactcaggggccaggggacggtggcttctacgtgcttgggacgttcccagccaccgtcccatgttcccggcggggggccagctgtccccaccgccagcccaactcagcacttggtcagggtatcagcttggtgggggggcgtgagcccagcccctggggcggctcagcccatacaaggccatggggctgggcgcaaagcatgcctgggttcagggtgggtatggtgcgggagcagggaggtgagaggctcagctgccctccagaactcctccctggggacaacccctcccagccaatagcacagcctaggtccccctatataaggccacggctgctggcccttccttt

SEQ ID NO: 18 - DNA, 600 bp, synthetic construct
  source 1..600: mol_type = other DNA
ctgaggctcagggctagctcgcccatagacatacatggcaggcaggctttggccaggatccctccgcctgccaggcgtctccctgccctcccttcctgcctagagacccccaccctcaagcctggctggtctttgcctgagacccaaacctcttcgacttcaagagaatatttaggaacaaggtggtttagggcctttcctgggaacaggccttgaccctttaagaaatgacccaaagtctctccttgaccaaaaaggggaccctcaaactaaagggaagcctctcttctgctgtctcccctgaccccactcccccccaccccaggacgaggagataaccagggctgaaagaggcccgcctgggggctgcagacatgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccct

SEQ ID NO: 19 - DNA, 600 bp, synthetic construct
  source 1..600: mol_type = other DNA
ggagttccaggggcgtaaaggagagggagttcgccttccttcccttcctgagactcaggagtgactgcttctccaatcctcccaagcccaccactccacacgactccctcttcccggtagtcgcaagtgggagtttggggatctgagcaaagaacccgaagaggagttgaaatattggaagtcagcagtcaggcaccttcccgagcgcccagggcgctcagagtggacatggttggggaggcctttgggacaggtgcggttcccggagcgcaggcgcacacatgcacccaccggcgaacgcggtgaccctcgccccaccccatcccctccggcgggcaactgggtcgggtcaggaggggcaaacccgctagggagacactccatatacggcccggcccgcgttacctgggaccgggccaacccgctccttctttggtcaacgcaggggacccgggcgggggcccaggccgcgaaccggccgagggagggggctctagtgcccaacacccaaatatggctcgagaagggcagcgacattcctgcggggtggcgcggagggaatgcccgcgggctatataaaacctgagcagagggacaagc

SEQ ID NO: 20 - DNA, 600 bp, synthetic construct
  source 1..600: mol_type = other DNA
agaaacctgtggtctagaggcggggcggggccgatggaggcaacgcacgcccccgcaggcgcccaggccacgccctctgccgcagcattcggtgaaacctgcgttccgagaacttctgaaaactttatctgggggccttcgagaaggctcagacagtaagggtgcatgctgccaatcctgaggagctgagttcgatccctgagaccttcagggtggacagagacggactcccacatgttgttttctgacttctacatgtgtccagtcatacatacacaaatatggaataaacagatggctcatcaggtaagagtgctggctgcttttgcagaggacccaggttcgatttccagaacccacatgtcggctcaaaatcatctgtaattccagttccagggagatccagcactttcttccagggcctccacagacacacataaaataaagataaaaatctccaaaaaatattgttttaataattacaacctgaagaccttgcacaactattcctggctgagaagatggtaagggcgctagctgccaagcttgacagcctgagtttcatctccaagaaccatgaaaactgactcctgggaatta

SEQ ID NO: 21 - DNA, 600 bp, synthetic construct
  source 1..600: mol_type = other DNA
ggaagcagaaggccaacattcctcccaagggaaactgaggctcagagttaaaacccaggtatcagtgatatgcatgtgccccggccagggtcactctctgactaaccggtacctaccctacaggcctacctagagactcttttgaaaggatggtagagacctgtccgggctttgcccacagtcgttggaaacctcagcattttctaggcaacttgtgcgaataaaacacttcgggggtccttcttgttcattccaataacctaaaacctctcctcggagaaaatagggggcctcaaacaaacgaaattctctagcccgctttccccaggataaggcaggcatccaaatggaaaaaaaggggccggccgggggtctcctgtcagctccttgccctgtgaaacccagcaggcctgcctgtcttctgtcctcttggggctgtccaggggcgcaggcctcttgcgggggagctggcctccccgccccctcgcctgtggccgcccttttcctggcaggacagagggatcctgcagctgtcaggggaggggcgccggggggtgatgtcaggagggctacaaatagtgcagacagctaaggggctcc

SEQ ID NO: 22 - DNA, 601 bp, synthetic construct
  source 1..601: mol_type = other DNA
ggggtgatgtgtgtcagatctctggattgggggagcttcaaagtgggaaagaaaatggagttcaaatgtggggcttattttccatccctacctggagcccatgactcctcccggctcacctgaccacagggctacctcccctgagcttaagcatcaaggcttagtagtctgagttaagdaacccataaatggggtgcattgtggcaggtcagcaatcgtgtgtccaggtgggcagaactggggagacctttcaaacaggtaaatcttgggaagtacagaccagcagtctgcaaagcagtgacctttggcccagcacagcccttccgtgagccttggagccagttgggaggggcagacagctggggatactctccatatacggcctggtccggtcctagctacctgggccagggccagtcctctccttctttggtcagtgcaggagacccgggcggggacccaggctgagaaccagccgaaggaagggactctagtgcccgacacccaaatatggcttgggaagggcagcaacattccttcggggcggtgtggggagagctcccgggactatataaaaacctgtgcaaggggacaggcggtc

SEQ ID NO: 23 - DNA, 770 bp, synthetic construct
  source 1..770: mol_type = other DNA
ctagaagctgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcac

SEQ ID NO: 24 - DNA, 383 bp, synthetic construct
  source 1..383: mol_type = other DNA
cgagctccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccccgggctgcaggaattc

SEQ ID NO: 25 - DNA, 82 bp, synthetic construct
  source 1..82: mol_type = other DNA
gtgagtatctcagggatccagacatggggatatgggaggtgcctctgatcccagggctcactgtgggtctctctgttcacag

SEQ ID NO: 26 - DNA, 133 bp, synthetic construct
  source 1..133: mol_type = other DNA
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 27 - DNA, 97 bp, synthetic construct
  source 1..97: mol_type = other DNA
gtaagtttagtctttttgtcttttatttcaggtcccggatccggtggtggtgcaaatcaaagaactgctcctcagtggatgttgcctttacttctag

SEQ ID NO: 28 - DNA, 7051 bp, synthetic construct
  source 1..7051: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctaaggcaattggccactacgggtctaggctgcccatgtaaggaggcaaggcctggggacacccgagatgcctggttataattaaccccaacacctgctgccccccccccccaacacctgctgcctgagcctgagcggttaccccaccccggtgcctgggtcttaggctctgtacaccatggaggagaagctcgctctaaaaataaccctgtccctggtggatcgccactacgggtctaggctgcccatgtaaggaggcaaggcctggggacacccgagatgcctggttataattaaccccaacacctgctgccccccccccccaacacctgctgcctgagcctgagcggttaccccaccccggtgcctgggtcttaggctctgtacaccatggaggagaagctcgctctaaaaataaccctgtccctggtggatcgccactacgggtctaggctgcccatgtaaggaggcaaggcctggggacacccgagatgcctggttataattaaccccaacacctgctgccccccccccccaacacctgctgcctgagcctgagcggttaccccaccccggtgcctgggtcttaggctctgtacaccatggaggagaagctcgctctaaaaataaccctgtccctggtggatccctccctggggacagcccctcctggctagtcacaccctgtaggctcctctatataacccaggggcacaggggctgcccccgggtcaccgctagccaaagcttctcgaggctggctagttaagctatcaacaagtttgtacagaaaagcaggctttaaaggaaccaattcagtcgacgctagcaagcttggtaccggatccgaattccaccatgtcggaggagcagttcggaggggacggggcggcggcggcggcaacggcggcggtaggcggctcggcgggcgagcaggagggagccatggtggcggcggcggcgcaggggccggcggcggcggcgggaagcgggagcggcggcggcggctctgcggccggaggcaccgaaggaggcagcgccgaggcagagggagccaagatcgacgccagtaagaacgaggaggatgaaggccattcaaactcctccccacgacacactgaagcagcggcggcacagcgggaagaatggaaaatgtttataggaggccttagctgggacaccacaaagaaagatctgaaggactacttttccaaatttggtgaagttgtagactgcactctgaagttagatcctatcacagggcgatcaaggggttttggctttgtgctatttaaagagtcggagagtgtagataaggtcatggatcagaaagaacataaattgaatgggaaagtcattgatcctaaaagggccaaagccatgaaaacaaaagagcctgtcaaaaaaatttttgttggtggcctttctccagacacacctgaagaaaaaataagagagtactttggtggttttggtgaggttgaatccatagagctccctatggacaacaagaccaataagaggcgtgggttctgttttattacctttaaggaagaggagccagtgaagaagataatggaaaagaaataccacaatgttggtcttagtaaatgtgaaataaaagtagccatgtcaaaggaacagtatcagcagcagcagcagtggggatctagaggagggtttgcaggcagagctcgcggaagaggtggagatcagcagagtggttatgggaaagtatccaggcgaggtggacatcaaaatagctacaaaccatactaagatatcgcggccgcctcgaggactacaaggatgacgatgacaaggattacaaagacgacgatgataaggactataaggatgatgacgacaaataatagcaattcctcgacgactgcatagggttacccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatggccacaactagtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagtttaaactctagacccagctttcttgtacaaagtggttgatctagagggcccgtaactagttgagcggccgcaactcgagactctagaggttaatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgaaacccgctgactagacgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggccgcgggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 29 - protein, 738 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 30 - DNA, 129 bp, synthetic construct
  source 1..129: mol_type = other DNA
cgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 31 - DNA, 130 bp, synthetic construct
  source 1..130: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 32 - DNA, 921 bp, synthetic construct
  source 1..921: mol_type = other DNA
atgtcggaggagcagttcggaggggacggggcggcggcggcggcaacggcggcggtaggcggctcggcgggcgagcaggagggagccatggtggcggcggcggcgcaggggccggcggcggcggcgggaagcgggagcggcggcggcggctctgcggccggaggcaccgaaggaggcagcgccgaggcagagggagccaagatcgacgccagtaagaacgaggaggatgaaggccattcaaactcctccccacgacacactgaagcagcggcggcacagcgggaagaatggaaaatgtttataggaggccttagctgggacaccacaaagaaagatctgaaggactacttttccaaatttggtgaagttgtagactgcactctgaagttagatcctatcacagggcgatcaaggggttttggctttgtgctatttaaagagtcggagagtgtagataaggtcatggatcagaaagaacataaattgaatgggaaagtcattgatcctaaaagggccaaagccatgaaaacaaaagagcctgtcaaaaaaatttttgttggtggcctttctccagacacacctgaagaaaaaataagagagtactttggtggttttggtgaggttgaatccatagagctccctatggacaacaagaccaataagaggcgtgggttctgttttattacctttaaggaagaggagccagtgaagaagataatggaaaagaaataccacaatgttggtcttagtaaatgtgaaataaaagtagccatgtcaaaggaacagtatcagcagcagcagcagtggggatctagaggagggtttgcaggcagagctcgcggaagaggtggagatcagcagagtggttatgggaaagtatccaggcgaggtggacatcaaaatagctacaaaccatactaa

SEQ ID NO: 33 - DNA, 714 bp, synthetic construct
  source 1..714: mol_type = other DNA
ccactacgggtctaggctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggatccactacgggtctatgctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggaccactacgggtctaggctgcccatgtaaggaggcaaggcctggggacacccgagatgcctggttataattaaccccaacacctgctgccccccccccccaacacctgctgcctgagcctgagcggttaccccaccccggtgcctgggtcttaggctctgtacaccatggaggagaagctcgctctaaaaataaccctgtccctggtcctccctggggacagcccctcctggctagtcacaccctgtaggctcctctatataacccaggggcacaggggctgcccccgggtcac

(19) United States
(12) Patent Application Publication
(10) Pub. No.: US 2025/0186619 A1
(43) Pub. Date: Jun. 12, 2025

(54) ADENO-ASSOCIATED VIRAL VECTOR COMPOSITIONS AND METHODS OF PROMOTING MUSCLE REGENERATION

(71) Applicant: NEW YORK UNIVERSITY, New York, NY (US)

(72) Inventors: Dounia ABBADI, New York, NY (US); Robert J. SCHNEIDER, New York, NY (US)

(21) Appl. No.: 18/580,078

(22) PCT Filed: Jul. 19, 2022
(86) PCT No.: PCT/US2022/073910

§ 371 Date: Jan. 17, 2024

Related U.S. Application Data
(60) Provisional application No. 63/223,480, filed on Jul. 19, 2021.

(51) Int. Cl. A61K 48/00; C12N 15/86

(52) U.S. Cl. CPC A61K 48/0058; C12N 15/86; C12N 2750/14122; C12N 2750/14143

(57) ABSTRACT

Provided are compositions comprising, and methods of administering, gene therapy vectors, particularly recombinant AAV vectors encoding AUF1 proteins for expression in muscle cells for restoring or increasing muscle mass, muscle function or performance, and/or reducing or reversing muscle atrophy. The compositions and methods may be used for the treatment of subject suffering from sarcopenia, muscle degenerative disease or traumatic injury.

### GOVERNMENT SUPPORT

This invention was made with government support under RO1 AR074430-01 awarded by the National Institutes of Health. The government has certain rights in the invention.

### 1. FIELD OF THE INVENTION

The present invention relates to compositions and methods of treating conditions associated with loss of muscle or muscle performance or promoting muscle formation by administration of doses of gene therapy vectors, such as AAV gene therapy vectors, in which the transgene encodes an AUF1.

### 2. BACKGROUND

Muscle wasting diseases represent a major source of human disease. They can be genetic in origin (primarily muscular dystrophies), related to aging (sarcopenia), or the result of traumatic muscle injury, among others. There are few treatment options available for individuals with myopathies, or those who have suffered severe muscle trauma, or the loss of muscle mass with aging (known as sarcopenia). The physiology of myopathies is well understood and founded on a common pathogenesis of relentless cycles of muscle degeneration and regeneration, typically leading to functional exhaustion of muscle stem (satellite) cells and their progenitor cells that fail to reactivate, and at times their loss as well (Carlson & Conboy, “Loss of Stem Cell Regenerative Capacity Within Aged Niches,” Aging Cell 6(3):371-82 (2007); Shefer et al., “Satellite-cell Pool Size Does Matter: Defining the Myogenic Potency of Aging Skeletal Muscle,” Dev. Biol. 294(1):50-66 (2006); Bernet et al., “p38 MAPK Signaling Underlies a Cell-autonomous Loss of Stem Cell Self-renewal in Skeletal Muscle of Aged Mice,” Nat. Med. 20(3):265-71 (2014); and Dumont et al., “Intrinsic and Extrinsic Mechanisms Regulating Satellite Cell Function,” Development 142(9):1572-1581 (2015)).

Age-related skeletal muscle loss and atrophy is characterized by the progressive loss of muscle mass, strength, and endurance with age. It can be a significant source of frailty, increased fractures, and mortality in the elderly population (Vermeiren et al., “Frailty and the Prediction of Negative Health Outcomes: A Meta-Analysis,” J. Am. Med. Dir. Assoc. 17(12):1163.el-1163.e17 (2016) and Buford, T. W., “Sarcopenia: Relocating the Forest among the Trees,” Toxicol. Pathol. 45(7):957-960 (2017)). Although different strategies have been investigated to counter muscle loss and atrophy, regular resistance exercise is the most effective in slowing muscle loss and atrophy, but compliance and physical limitations are significant barriers (Wilkinson et al., “The Age-Related loss of Skeletal Muscle Mass and Function: Measurement and Physiology of Muscle Fibre Atrophy and Muscle Fibre Loss in Humans,” Ageing Res. Rev. 47:123-132 (2018)). Consequently, with an aging global population, therapeutic strategies need to be developed to reverse age-related muscle decline.

Muscle regeneration is initiated by skeletal muscle stem (satellite) cells that reside between striated muscle fibers (myofibers), which are the contractile cellular bundles, and the basal lamina that surrounds them (Carlson & Conboy, “Loss of Stem Cell Regenerative Capacity within Aged Niches,” Aging Cell 6(3):371-382 (2007) and Schiaffino & Reggiani, “Fiber Types in Mammalian Skeletal Muscles,” Physiol. Rev. 91(4):1447-1531 (2011)). Upon physical injury to muscle, the anatomical niche is disrupted, normally quiescent satellite cells become activated and proliferate asymmetrically. Some satellite cells reconstitute the stem cell population while most others differentiate and fuse to form new myofibers (Hindi et al., “Signaling Mechanisms in Mammalian Myoblast Fusion,” Sci. Signal. 6(272):re2 (2013)). Studies have demonstrated the singular importance of the satellite cell/myoblast population in muscle regeneration (Shefer et al., “Satellite-cell Pool Size Does Matter: Defining the Myogenic Potency of Aging Skeletal Muscle,” Dev. Biol. 294(1):50-66 (2006); Dumont et al., “Intrinsic and Extrinsic Mechanisms Regulating Satellite Cell Function,” Development 142(9):1572-1581 (2015); Briggs & Morgan, “Recent Progress in Satellite Cell/Myoblast Engraftment—Relevance for Therapy, FEBS J. 280(17):4281-93 (2013); Morgan & Zammit, “Direct Effects of the Pathogenic Mutation on Satellite Cell Function in Muscular Dystrophy,” Exp. Cell Res. 316(18):3100-8 (2010); and Relaix & Zammit, “Satellite Cells are Essential for Skeletal Muscle Regeneration: The Cell on the Edge Returns Centre Stage,” Development 139(16):2845-56 (2012)).

Myofibers are divided into two types that display different contractile and metabolic properties: slow-twitch (Type I) and fast-twitch (Type II). Slow- and fast-twitch myofibers are defined according to their contraction speed, metabolism, and type of myosin gene expressed (Schiaffino & Reggiani, “Fiber Types in Mammalian Skeletal Muscles,” Physiol. Rev. 91(4):1447-1531 (2011) and Bassel-Duby & Olson, “Signaling Pathways in Skeletal Muscle Remodeling,” Annu. Rev. Biochem. 75:19-37 (2006)). Slow-twitch myofibers are rich in mitochondria, preferentially utilize oxidative metabolism, and provide resistance to fatigue at the expense of speed of contraction. Fast-twitch myofibers more readily atrophy in response to nutrient deprivation, traumatic damage, advanced age-related loss (sarcopenia), and cancer-mediated cachexia, whereas slow-twitch myofibers are more resilient (Wang & Pessin, “Mechanisms for Fiber-Type Specificity of Skeletal Muscle Atrophy,” Curr. Opin. Clin. Nutr. Metab. Care 16(3):243-250 (2013); Tonkin et al., “SIRT1 Signaling as Potential Modulator of Skeletal Muscle Diseases,” Curr. Opin. Pharmacol. 12(3):372-376 (2012); and Arany, Z, “PGC-1 Coactivators and Skeletal Muscle Adaptations in Health and Disease,” Curr. Opin. Genet. Dev. 18(5):426-434 (2008)). Peroxisome proliferator-activated receptor gamma co-activator 1-alpha (PGC1α or Ppargc1) is a major physiological regulator of mitochondrial biogenesis and Type I myofiber specification (Lin et al., “Transcriptional Co-Activator PGC-1 Alpha Drives the Formation of Slow-Twitch Muscle Fibres,” Nature 418 (6899):797-801 (2002)). PGC1α stimulates mitochondrial biogenesis and oxidative metabolism through increased expression of nuclear respiratory factors (NRFs) such as NRF1 and 2 that stimulate mitochondrial biosynthesis, mitochondria transcription factor A (Tfam), and in addition to mitochondrial biosynthesis, also promote slow myofiber formation through increased expression of Mef2 proteins (Lin et al., “Transcriptional Co-Activator PGC-1 Alpha Drives the Formation of Slow-Twitch Muscle Fibres,” Nature 418 (6899):797-801 (2002); Lai et al., “Effect of Chronic Contractile Activity on mRNA Stability in Skeletal Muscle,” Am. J. Physiol. Cell. Physiol. 299(1):C155-163 (2010); Ekstrand et al., “Mitochondrial Transcription Factor A Regulates mtDNA Copy Number in Mammals,” Hum. Mol. Genet. 13(9):935-944 (2004); and Scarpulla, RC, “Transcriptional Paradigms in Mammalian Mitochondrial Biogenesis and Function,” Physiol. Rev. 88(2): 611-638 (2008)). Importantly, PGC1α protects muscle from atrophy due to disuse, certain myopathies, starvation, sarcopenia, cachexia, and other causes (Wiggs, M. P., “Can Endurance Exercise Preconditioning Prevention Disuse Muscle Atrophy?,” Front. Physiol. 6:63 (2015); Wing et al., “Proteolysis in Illness-Associated Skeletal Muscle Atrophy: From Pathways to Networks,” Crit. Rev. Clin. Lab. Sci. 48(2):49-70 (2011); Bost & Kaminski, “The Metabolic Modulator PGC-1alpha in Cancer,” Am. J. Cancer Res. 9(2):198-211 (2019); and Dos Santos et al., “The Effect of Exercise on Skeletal Muscle Glucose Uptake in type 2 Diabetes: An Epigenetic Perspective,” Metabolism 64(12):1619-1628 (2015)).

Skeletal muscle can remodel between slow- and fast-twitch myofibers in response to physiological stimuli, load bearing, atrophy, disease, and injury (Bassel-Duby & Olson, “Signaling Pathways in Skeletal Muscle Remodeling,” Annu. Rev. Biochem. 75:19-37 (2006)), involving transcriptional, metabolic, and post-transcriptional control mechanisms (Schiaffino & Reggiani, “Fiber Types in Mammalian Skeletal Muscles,” Physiol. Rev. 91(4):1447-1531 (2011) and Robinson & Dilworth, “Epigenetic Regulation of Adult Myogenesis,” Curr. Top Dev. Biol. 126:235-284 (2018)). The ability to selectively promote slow-twitch muscle has been a long-standing goal, because endurance slow-twitch Type I myofibers provide greater resistance to muscle atrophy (Talbot & Maves, “Skeletal Muscle Fiber Type: Using Insights from Muscle Developmental Biology to Dissect Targets for Susceptibility and Resistance to Muscle Disease,” Wiley Interdiscip. Rev. Dev. Biol. 5(4):518-534 (2016)), and could be an effective therapy for sarcopenia, Duchenne Muscular Dystrophy, cachexia, and other muscle wasting diseases (Selsby et al., “Rescue of Dystrophic Skeletal Muscle By PGC-1alpha Involves A Fast To Slow Fiber Type Shift In The Mdx Mouse,” PLoS One 7(1):e30063 (2012); von Maltzahn et al., “Wnt7a Treatment Ameliorates Muscular Dystrophy,” Proc. Natl. Acad. Sci. USA 109(50):20614-20619 (2012); and Ljubicic et al., “The Therapeutic Potential Of Skeletal Muscle Plasticity In Duchenne Muscular Dystrophy: Phenotypic Modifiers As Pharmacologic Targets,” FASEB J. 28(2):548-568 (2014)).

Duchenne Muscular Dystrophy (“DMD”) is one of the most severe disorders of muscle degeneration known as myopathies. Inherited in an X-linked recessive manner, the disorder is caused by mutations in the dystrophin gene, resulting in a near-absence of expression of the protein, which plays a key role in stabilization of muscle cell membranes (Bonilla et al., “Duchenne Muscular Dystrophy: Deficiency of Dystrophin at the Muscle Cell Surface,” Cell 54(4):447-452 (1988) and Hoffman et al., “Dystrophin: The Protein Product of the Duchenne Muscular Dystrophy Locus,” Cell 51(6):919-928 (1987)). Consequently, only males with the mutation are afflicted with DMD, which affects 1 in 3500 live births. There are no cures for DMD, and currently approved approaches involve limited use of corticosteroids to dampen inflammatory immune responses, a secondary exacerbating effect of muscle atrophy. While the inflammatory response is generally beneficial in normal muscle wound repair and regeneration, in DMD the response is no longer self-limiting due to the chronic nature of muscle damage. This results in exacerbation of necrosis of existing muscle and depletion of muscle fibers (myofibers) with replacement by connective and adipose tissue (Carnwath & Shotton, “Muscular Dystrophy in the mdx Mouse: Histopathology of the Soleus and Extensor Digitorum Longus Muscles,” J. Neurol. Sci. 80(1):39-54 (1987); Tanabe et al., “Skeletal Muscle Pathology in X Chromosome-Linked Muscular Dystrophy (mdx) Mouse,” Acta Neuropathol. 69(1-2):91-95 (1986); and Fairclough et al., “Pharmacologically Targeting the Primary Defect and Downstream Pathology in Duchenne Muscular Dystrophy,” Curr. Gene Ther. 12(3):206-244 (2012)). While steroids can provide short-term increased muscle strength, long-term treatment is ultimately ineffective and can exacerbate disease. Steroids do not target the underlying cause of disease. There is therefore an urgent need for pharmacologic approaches that address the primary underlying cause of DMD: loss of muscle fiber strength, loss of muscle stem cells, loss of muscle regenerative capacity, and attenuation of the exacerbating destructive effects of the pathological immune response on muscle health and integrity (Fairclough et al., “Pharmacologically Targeting the Primary Defect and Downstream Pathology in Duchenne Muscular Dystrophy,” Curr. Gene Ther. 12(3):206-244 (2012)).

Dystrophin functions to assemble the dystroglycan complex at the sarcolemma, which connects the extracellular matrix to the cytoplasmic intermediate filaments of the muscle cell, providing physical strength and structural integrity to muscle fibers which are readily damaged in the absence of dystrophin (Yiu & Kornberg, “Duchenne Muscular Dystrophy,” Neurol. India 56(3):236-247 (2008)). Dystrophin-defective myofibers are very easily damaged by minor stresses and micro-tears in DMD. This triggers continuous cycles of muscle repair and regeneration, depletes the muscle stem cell population, and provokes a destructive immune response that increases with age (Yiu & Kornberg, “Duchenne Muscular Dystrophy,” Neurol. India 56(3):236-247 (2008); Smythe et al., “Age Influences The Early Events of Skeletal Muscle Regeneration: Studies of Whole Muscle Grafts Transplanted Between Young (8 Weeks) and Old (13-21 Months) Mice,” Exp. Gerontol. 43(6):550-562 (2008); Heslop et al., “Evidence for a Myogenic Stem Cell that is Exhausted in Dystrophic Muscle,” J. Cell Sci. 113(Pt 12):2299-32208 (2000); Cros et al., “Muscle Hypertrophy in Duchenne Muscular Dystrophy. A Pathological and Morphometric Study,” J. Neurol. 236(1):43-47 (1989); and Abdel-Salam et al., “Markers of Degeneration and Regeneration in Duchenne Muscular Dystrophy,” Acta Myol. 28(3):94-100 (2009)). In this regard, it has been shown that the progressive loss of muscle and its regenerative capacity in DMD results from exhaustion (inability to activate) and depletion of the muscle stem cell population (i.e., satellite cells) (Carlson & Conboy, “Loss of Stem Cell Regenerative Capacity Within Aged Niches,” Aging Cell 6(3):371-82 (2007); Kudryashova et al., “Satellite Cell Senescence Underlies Myopathy in a Mouse Model of Limb-girdle Muscular Dystrophy 2H,” J. Clin. Invest. 122(5):1764-76 (2012); Gopinath & Rando, “Stem Cell Review Series: Aging of the Skeletal Muscle Stem Cell Niche,” Aging Cell 7(4):590-8 (2008); Morgan & Zammit, “Direct Effects of the Pathogenic Mutation on Satellite Cell Function in Muscular Dystrophy,” Exp. Cell Res. 316(18):3100-8 (2010); and Collins et al., “Stem Cell Function, Self-renewal, and Behavioral Heterogeneity of Cells From the Adult Muscle Satellite Cell Niche,” Cell 122(2):289-301 (2005)). Typically, in normal muscle, the small pool of satellite cells that do not differentiate following injury repopulate muscle and re-enter the quiescent state in their niche, only to be activated again upon muscle damage to differentiate and fuse into myofibers. The niche is defined both structurally and morphologically as sites where satellite cells reside adjacent to muscle fibers, in which quiescence is maintained by the structural integrity of the micro-environment, identified by laminin and other structural proteins (Carlson & Conboy, “Loss of Stem Cell Regenerative Capacity Within Aged Niches,” Aging Cell 6(3):371-82 (2007); Dumont et al., “Intrinsic and Extrinsic Mechanisms Regulating Satellite Cell Function,” Development 142(9):1572-1581 (2015); Gopinath & Rando, “Stem Cell Review Series: Aging of the Skeletal Muscle Stem Cell Niche,” Aging Cell 7(4):590-8 (2008); Seale & Rudnicki, “A New Look at the Origin, Function, and “Stem-cell” Status of Muscle Satellite Cells,” Dev. Biol. 218(2):115-24 (2000); Briggs & Morgan, “Recent Progress in Satellite Cell/Myoblast Engraftment—Relevance for Therapy, FEBS J 280(17):4281-93 (2013); Collins et al., “Stem Cell Function, Self-renewal, and Behavioral Heterogeneity of Cells From the Adult Muscle Satellite Cell Niche,” Cell 122(2):289-301 (2005); and Murphy et al., “Satellite Cells, Connective Tissue Fibroblasts and Their Interactions are Crucial for Muscle Regeneration,” Development 138(17):3625-37 (2011)). Studies suggest that it is the continuous damage to muscle in DMD that destroys this satellite cell niche, preventing these stem cells from renewing and ultimately leading to their functional exhaustion and cessation of muscle repair.

The myogenesis program is controlled by genes that encode myogenic regulatory factors (MRFs) (Mok & Sweetman, “Many Routes to the Same Destination: Lessons From Skeletal Muscle Development,” Reproduction 141(3):301-12 (2011)), which orchestrate differentiation of the activated satellite cell to become myoblasts, arrest their proliferation, cause them to differentiate, and fuse with multi-nucleated myofibers (Mok & Sweetman, “Many Routes to the Same Destination: Lessons From Skeletal Muscle Development,” Reproduction 141(3):301-12 (2011)). Unique expression markers identify and stage skeletal muscle regeneration. PAX7 is a transcription factor expressed by quiescent and early activated satellite cells (Brack, A. S., “Pax7 is Back,” Skelet. Muscle 4(1):24 (2014) and Gunther, S., et al., “Myf5-positive Satellite Cells Contribute to Pax7-dependent Long-term Maintenance of Adult Muscle Stem Cells,” Cell Stem Cell 13(5):590-601 (2013)).

As satellite cells age, they lose their ability to maintain a quiescent population (Dumont et al., “Intrinsic and Extrinsic Mechanisms Regulating Satellite Cell Function,” Development 142(9):1572-1581 (2015)), and become depleted or functionally exhausted, a primary cause of sarcopenia (muscle loss) with aging and in myopathic diseases (Bernet et al., “p38 MAPK Signaling Underlies a Cell-autonomous Loss of Stem Cell Self-renewal in Skeletal Muscle of Aged Mice,” Nat. Med. 20(3):265-71 (2014); Dumont et al., “Intrinsic and Extrinsic Mechanisms Regulating Satellite Cell Function,” Development 142(9):1572-1581 (2015); Kudryashova et al., “Satellite Cell Senescence Underlies Myopathy in a Mouse Model of Limb-girdle Muscular Dystrophy 2H,” J. Clin. Invest. 122(5):1764-76 (2012); and Silva et al., “Inhibition of Stat3 Activation Suppresses Caspase-3 and the Ubiquitin-proteasome System, Leading to Preservation of Muscle Mass in Cancer Cachexia,” J. Biol. Chem. 290(17):11177-87 (2015)).

Thus, there remains an urgent need for effective therapeutic options that address the primary underlying cause myopathic diseases (e.g., sarcopenia, Duchenne muscular dystrophy, traumatic muscle injury), which include, e.g., loss of muscle fiber strength, loss of muscle stem cells, loss of muscle regenerative capacity, and attenuation of the exacerbating destructive effects of the pathological immune response on muscle health and integrity.

Cultured or laboratory-based meat production provides an alternative to slaughtered animals, particularly chicken, beef or pork, as a source of meat. Technologies currently used for cultured meat production suffer from the inability to increase the presence of slow-twitch (dark) muscle fibers in cultured meat (myotubes or myofibers), and contain instead a large proportion or are entirely composed of fast-twitch myotubes or myofibers. Slow twitch muscle is generally considered more flavorful and desirable, but methods have not been developed to reliably enhance the slow twitch muscle composition in cultured muscle.

The present application is directed to overcoming these and other deficiencies in the art.

### 3. SUMMARY OF THE INVENTION

As shown in the Examples herein, supplementing AUF1 protein in muscle cells, for example, by viral vector gene transfer increases skeletal muscle mass and fiber formation in AUF1 knock out mice, and mouse models of muscular dystrophy and muscle injury, promotes increase in slow-twitch muscle fiber, enhances exercise endurance, reduces biomarkers of muscle atrophy and inflammation in age-related muscle loss, and stabilizes the sarcolemma by increasing expression of components of the dystrophin glycoprotein complex (DGC) (also known as the Dystrophin Associated Protein Complex (DAPC)) and/or participation of those components in the DGC.

Accordingly, provided are methods and pharmaceutical compositions for promoting muscle regeneration, restoring or increasing muscle mass, muscle function or performance, and/or reducing or reversing muscle atrophy by increasing the levels of AUF1 in muscle cells in a subject in need thereof. Methods and compositions are provided for administering AUF1 protein or nucleic acid that encodes and expresses AUF1 protein in muscle cells, such as DNA, mRNA, plasmid DNA or viral vectors encoding AUF1. Provided are therapeutic compositions comprising, and methods of administering, gene therapy vectors, particularly recombinant AAV vectors, comprising genomes with transgenes encoding an AUF1 protein (mouse or human p37AUF1, p40AUF1, p42UAUF1, or p45AUF1) operably linked to regulatory elements that promote AUF1 expression in muscle cells for restoring or increasing muscle mass, muscle function or performance, and/or reducing or reversing muscle atrophy. In embodiments, the gene therapy vectors are delivered to the subject in need such that the AUF1 protein is expressed in muscle cells of the subject.

In an embodiment, provided is a method of and pharmaceutical compositions for use in stabilizing sarcolemma in a subject comprising administering to the subject a pharmaceutical composition comprising a therapeutically effective amount of AUF1 or a nucleic acid encoding AUF1 and a pharmaceutically acceptable carrier. In an embodiment, the method of stabilizing the sarcolemma comprises administering to the subject a vector comprising a nucleic acid molecule encoding an AUF1 protein or a functional fragment thereof, operatively coupled to the muscle cell-specific promoter such that AUF1 is expressed in muscle cells of the patient. In embodiments methods are provided for increasing the expression of one or more components of the DGC and/or participation in the DGC, including one or more of α-sarcoglycan, β-sarcoglycan, δ-sarcoglycan, γ-sarcoglycan, ε-sarcoglycan, ζ-sarcoglycan, α-dystroglycan, β-dystroglycan, sarcospan, α-syntrophin, β-syntrophin, α-dystrobrevin, β-dystrobrevin, Caveolin-3, or nNOS.

In embodiments, stabilization of the sarcolemma is compared (at, for example, 1 month, 2 months, 3 months. 4 months, 5 months or 6 months after administration) to normal muscle (or reference normal or diseased muscle) or muscle of the subject prior (e.g. 2 weeks, 1 month or 2 months prior) to administration of the therapeutic (including “pre-treatment levels” being measured within 1 day, 1 week, 2 weeks or 1 month prior to therapeutic administration or other appropriate time period for assessing a baseline value), wherein the stabilization provides for 20%, 30%, 40%, 50%, 60%, 70%, 80%, 90% or 100% or greater (2 fold, 3 fold or more) reduction in markers of sarcolemma integrity, including, for example, serum creatine kinase levels, 20%, 30%, 40%, 50%, 60%, 70%, 80%, 90% or 100% or greater (2 fold, 3 fold or more) reduction in markers of muscle atrophy (for example, biomarkers as disclosed herein), 20%, 30%, 40%, 50%, 60%, 70%, 80%, 90% or 100% or greater (2 fold, 3 fold or more) increase in utrophin levels or a member of the dystrophin sarcoglycan complex, 20%, 30%, 40%, 50%, 60%, 70%, 80%, 90% or 100% or greater (2 fold, 3 fold or more) increase compared to normal muscle or muscle of the subject prior to administration of the therapeutic of muscle mass, or muscle function, or performance using methods known in the art for assessing muscle mass, muscle function or muscle performance.

In certain embodiments, provided are methods of increasing utrophin (including at least 20%, 30%, 40%, 50%, 60%, 70%, 80%, 90% or 100% or more) in a dystrophin glycoprotein complex (DGC) in a subject comprising administering AUF1 or a nucleic acid encoding AUF1 to the subject, including as a vector comprising a muscle cell-specific promoter and a nucleic acid molecule encoding an AU-rich mRNA binding factor 1 (AUF1) protein or a functional fragment thereof, wherein the nucleic acid molecule is operatively coupled to the muscle cell-specific promoter. In certain methods, the subject has a mutated dystrophin. And, in further embodiments, the method promotes replacement of the mutated dystrophin with utrophin in the DGC.

Further provided are methods of and pharmaceutical compositions for use in increasing muscle mass or treating sarcopenia in a subject having age-related muscle loss comprising administering to the subject a therapeutically effective amount of an AUF1 protein or a nucleic acid encoding an AUF1 protein, including a vector comprising a nucleic acid molecule encoding an AUF1 protein or a functional fragment thereof operatively coupled to the muscle cell-specific promoter. In certain embodiments, the subject is over 65 years old, over 75 years old, over 85 years old or over 90 years old. In embodiments, increasing muscle mass is compared to normal muscle or muscle of the subject prior (e.g. 2 weeks, 1 month or 2 months prior) to administration of the therapeutic, wherein the muscle mass increases for 20%, 30%, 40%, 50%, 60%, 70%, 80%, 90% or 100% or greater (2 fold, 3 fold or more) within 1 month, 2 months, 3 months, 4 months, 5 months or at least 6 months of administration of the therapeutic.

In an embodiment, provided are methods of and pharmaceutical compositions for use in treating a dystrophinopathy in a subject comprising administering to the subject a therapeutically effective amount of an AUF1 protein or a nucleic acid encoding an AUF1 protein, including a nucleic acid molecule encoding an AUF1 protein or a functional fragment thereof operatively coupled to the muscle cell-specific promoter. The dystrophinopathy may be Duchenne muscular dystrophy (DMD), Becker muscular dystrophy (BMD), X-linked dilated cardiomyopathy or limb-girdle muscular dystrophy.

In embodiments, provided are methods of and pharmaceutical compositions for use in increasing healing of traumatic muscle injury in a subject in need thereof comprising administering to the subject an AUF1 protein or nucleic acid encoding an AUF1 protein, including a vector comprising a nucleic acid encoding an AUF1 protein or a functional fragment thereof operatively coupled to the muscle cell-specific promoter. Healing of traumatic muscle injury can be assessed at within 1 month, 2 months, 3 months, 4 months, 5 months or at least 6 months of administration of the therapeutic using methods known in the art for assessing increased muscle mass, healing, strength and performance as well as monitoring of creatine kinase levels. In embodiments, healing of traumatic muscle injury is assessed at, for example, 1 month, 2 months, 3 months. 4 months, 5 months or 6 months after administration wherein the healing provides for 20%, 30%, 40%, 50%, 60%, 70%, 80%, 90% or 100% or greater (2 fold, 3 fold or more) reduction in markers of muscle leakiness, including, for example, serum creatine kinase levels, 20%, 30%, 40%, 50%, 60%, 70%, 80%, 90% or 100% or greater (2 fold, 3 fold or more) increase compared to reference injured muscle or muscle of the subject prior to administration of the therapeutic of muscle mass, or muscle function, or performance using methods known in the art for assessing muscle mass, muscle function or muscle performance.

In the embodiments, the administration of AUF1 or nucleic acid encoding AUF1 increases muscle mass, increase muscle strength, reduce expression of biomarkers of muscle atrophy, enhance muscle performance, increase muscle stamina, increase muscle resistance to fatigue and/or increase proportion of slow twitch fibers to fast twitch fibers.

The AUF1 administered may be one or more of p37AUF1, p40AUF1, p42AUF1, or p45AUF1. The muscle cell-specific promoter may be a muscle creatine kinase (MCK) promoter, a C5-12 promoter, a CK6-CK9 promoter, a dMCK promoter, a tMCK promoter, a smooth muscle 22 (SM22) promoter, a myo-3 promoter, a SPc5-12 promoter, a creatine kinase (CK) 8 promoter, a creatine kinase (CK) 8e promoter, a U6 promoter, a H1 promoter, a desmin promoter, a Pitx3 promoter, a skeletal alpha-actin promoter, a MHCK7 promoter, and a Sp-301 promoter. In embodiments, the nucleic acid encoding the AUF1 is delivered in a viral vector, including an rAAV viral particle, which, in a particular embodiment is an AAV8 serotype. The rAAV may be administered intravenously or intramuscularly, and may be administered at a dose of 1×10¹³ to 1×10¹⁴ genome copies/kg. In embodiments, the administration is a single dose of the gene therapy therapeutic.

Also provided are methods of producing synthetic meat or cultured or synthetic muscle tubes, fibers or tissue comprising contacting cultured muscle cells with AUF1 or a nucleic acid encoding and expressing AUF1, wherein the AUF1 is present in an amount sufficient to induce slow twitch muscle fibers in the cultured muscle cells; and growing the muscle cells under conditions and for a time sufficient to produce synthetic meat or cultured or synthetic muscle tubes, fibers or tissue, wherein the synthetic meat or cultured or synthetic muscle tubes, fibers or tissue comprises a greater proportion of slow twitch muscle fibers than synthetic meat produced in the absence of AUF1. The method may be used to increase slow twitch muscle fibers in synthetic meat or cultured or synthetic muscle tubes, fibers or tissue comprising contacting cultured muscle cells with AUF1 or a nucleic acid encoding and expressing AUF1, wherein the AUF1 is present in an amount sufficient to induce slow twitch muscle fibers in the cultured muscle cells; and growing the muscle cells under conditions and for a time sufficient to produce synthetic meat or cultured or synthetic muscle tubes, fibers or tissue having a greater proportion of slow twitch muscle fibers than synthetic meat or cultured or synthetic muscle tubes, fibers or tissue produced in the absence of AUF1. In embodiments, the muscle cells are sheep, goat, pig, cow, buffalo, chicken, duck, or goose muscle cells. In additional embodiments, the muscle cells are cultured in two-dimensional monolayer systems or three-dimensional complex muscle structure systems, which may be a consumable matrix.

### 4. BRIEF DESCRIPTION OF THE FIGURES

FIGS. 1A-1N show AUF1 supplementation in skeletal muscle improves exercise endurance in 12 and 28 month old mice. FIG. 1A. Relative expression of auf1 mRNA in the TA, gastrocnemius, EDL and soleus muscles normalized to invariant tbp mRNA at 3 and 12 months of age in wild type (WT) mice. FIG. 1B. Representative immunoblot and quantification of AUF1 protein levels in the TA muscle of WT mice at 3, 12 and 18 months. GAPDH is a loading control. n=2 mice chosen at random per group. FIG. 1C. Representative staining of AAV GFP control and AAV AUF1/GFP positive myofibers in TA muscle 40 d post-administration. FIG. 1D. Quantification of GFP positive myofibers in TA muscle 40 d post-AAV administration. n=5 mice. FIG. 1E. Relative fold increased expression of auf1 mRNA in gastrocnemius, TA, EDL and soleus muscles 40 d post-AAV administration. n=8-9 mice. FIGS. 1F-1J Strength and exercise endurance in 3 and 12 month old mice and 40 d post-AAV administration: (FIG. 1F) Grid hanging time, (FIG. 1G) maximum speed, (FIG. 1H) work performance, (FIG. 1I) time to exhaustion, (FIG. 1J) distance to exhaustion. n=5-9 mice. FIGS. 1K-1N Strength and exercise endurance 6 months post-AAV administration in 18 month old mice: (FIG. 1K) maximum speed, (FIG. 1L) work performance, (FIG. 1M) time to exhaustion, (FIG. 1N) distance to exhaustion. n=4 mice. Mean±SEM from 5 or more independent studies. *P<0.05, **P<0.01 by unpaired Mann-Whitney U test.

FIGS. 2A-20 show AUF1 gene therapy induces muscle mass along with an increase in myofiber capacity. FIGS. 2A-B are graphs showing muscle weight relative to total body weight 40 d post-AAV administration for gastrocnemius and TA muscles, respectively. n=8-9 mice. FIGS. 2C-D are graphs showing frequency distribution of gastrocnemius myofiber CSA and mean area at 40 d post-AAV administration. n=6 mice/group. FIGS. 2E-F are graphs showing frequency distribution of TA muscle CSA and mean area at 40 d post-AAV administration. n=5 mice. FIG. 2G show TA muscle CSA at 40 d post-AAV administration for GFP⁻ myofibers. n=5 mice. FIG. 2H presents photographic images showing representative immunostain of slow myofiber (red) and nuclei (DAPI blue) in gastrocnemius muscle at 40 d post-therapy. Scale bar: 200 m. FIGS. 2I-2K are graphs showing slow myofibers per field and mean CSA, respectively of slow and fast myofibers in gastrocnemius muscle at 40 d post-AAV administration. FIG. 2L shows representative immunostain of slow myofiber (red) and nuclei (blue) in soleus muscle 40 d after AAV AUF1-GFP or AAV GFP administration. FIG. 2M shows mean cross surface area (CSA) of slow-twitch soleus muscle myofiber 40 d after AAV AUF1 or AAV GFP administration. n=3 mice per group. FIG. 2N shows mean soleus weight in GFP control and AUF1-GFP AAV8 treated animals 40 d post-gene transfer. n=4 mice per group. FIG. 2O shows representative immunostaining and quantification of different myofibers in the soleus muscle, 6 months post-AUF1 gene transfer in 12 month old mice. Scale bar, 100 mm.

FIGS. 3A-3L show molecular markers of skeletal muscle myogenesis in AAV8 AUF1-GFP gene transferred mice. FIGS. 3A-B are graphs showing relative myh7 mRNA levels in gastrocnemius (FIG. 3A) and soleus (FIG. 3B) muscles normalized to invariant nuclear TATA-box binding protein (tbp) mRNA at 40 d post-gene transfer. FIGS. 3C-D are graphs showing relative fast myosin mRNA levels in gastrocnemius (FIG. 3C) and soleus (FIG. 3D) muscles normalized to tbp mRNA at 40 d gene transfer. FIG. 3E is a graph showing expression levels of non-mitochondrial mRNAs (pparg, six1) and mitochondrial mRNA in gastrocnemius muscle at 40 d post-gene transfer. FIG. 3F is a graph showing the level of mitochondrial mRNA for acadvl and tfam in gastrocnemius and EDL muscles at 40 d post-gene transfer. FIGS. 3G and H are graphs showing nrf1 and nrf2 mRNA levels in gastrocnemius muscle and soleus muscle, respectively, 40 d after gene transfer. FIG. 3I is a pair of graphs showing mitochondrial DNA content in the gastrocnemius muscle 40 d and 6 months after gene transfer. FIG. 3J is a graph showing mitochondrial DNA content in the soleus muscle 40 d after gene transfer. Red histogram, AAV AUF1-GFP. Black histogram, AAV GFP. FIG. 3K provides representative images of succinate dehydrogenase (SDH) enzyme activity in TA, EDL and gastrocnemius muscles from mice 40 d post-administration of AAV8 GFP or AAV8 AUF1-GFP. FIG. 3L shows quantitation of SDH positive myofibers per field for TA, EDL and gastrocnemius muscles corresponding to (K). n=3 mice per muscle, 5 fields chosen at random. Mean±SEM from 3 or more independent studies. *P<0.05; **P<0.01, ***P<0.001 by unpaired Mann-Whitney U test.

FIGS. 4A-4K show AUF1 promotes slow-twitch fiber myogenesis by stabilizing pgc1α mRNA. FIG. 4A is a pair of graphs showing relative auf1 mRNA expression in 3 and 12 month old WT mice in TA, gastrocnemius, EDL, and soleus muscles. n=5-7 mice. FIG. 4B shows representative immunofluorescence staining of AUF1 expression in slow myofibers in 3 month old mice. FIG. 4C shows representative immunoblot of AUF1 protein level and quantification in TA, gastrocnemius, EDL and soleus muscle in 3 month old mice. FIG. 4D is a graph showing relative myh7 mRNA expression in 3 month old mouse TA, gastrocnemius, EDL, and soleus muscles. FIG. 4E shows relative pgc1α mRNA expression and protein levels in WT C2C12 myoblasts and AUF1 KO myoblasts. FIG. 4F is a pair of graphs showing relative pgc1α mRNA expression in TA, gastrocnemius, and EDL muscles 40 d post-treatment, and in gastrocnemius at 6 months post gene transfer in 12 month old mice. FIG. 4G is a representative immunoblot of two AAV8-GFP control and AAV8-AUF1 GFP animals (left) and quantification of AUF1 and PGC1α in three animals per group (right) at 6 months after treatment. FIG. 4H shows Pgc1a mRNA immunoprecipitation with endogenous AUF1 protein in myoblasts 48 h after myotube induction of differentiation in WT C2C12 cells. n=5. (I) pgc1a mRNA decay rate in WT and AUF1 KO C2C12 cells. FIG. 4I is a graph showing Pgc1α mRNA immunoprecipitation with endogenous AUF1 protein in myoblasts 48 h after myotube induction of differentiation in WT C2C12 cells. n=3. FIGS. 4J and K show results with C2C12 cells overexpressing AUF1 transfected with plasmids expressing luciferase reporters without (pIS1) and with (pIS1 pgc1α 3′UTR) the pgc1α 3′UTR AREs. Cells were harvested at 36 h, equal protein amounts analyzed by immunoblot, luciferase activity determined and luciferase mRNA levels quantified. Mean±SEM from 3 or more independent studies. FIGS. 4 A and B: ****P<0.001 by Kruskall-Wallis test. All otherpanels by unpaired Mann-Whitney U test *P<0.05, **P<0.01, ***P<0.001. a, TA 3 vs. 12 month, *; b, gastrocnemius 3 vs. 12 month, **; c, EDL 3 vs. 12 month, **; d, soleus 3 vs. 12 month, **.

FIGS. 5A-5H show loss of AUF1 expression induces atrophy of slow-twitch myofibers. FIG. 5A is a graph showing body weight of WT and AUF1 KO mice at 3 months. FIG. 5B shows TA, gastrocnemius, EDL, and soleus muscle mass in 3 month old WT and AUF1 KO mice. Representative image of WT and AUF1 KO soleus muscles shown. FIG. 5C shows photographic images of a representative immunostain of slow (top) or fast (bottom) myosin (red) and laminin (green) in the soleus muscle from 3 month old WT and AUF1 KO mice. Scale bar: 200 m. FIGS. 5D and 5E are graphs showing slow-twitch myofibers per field of percentage and number, respectively, in 3 month old WT and AUF1 KO mice. FIGS. 5F and 5G are graphs showing fast-twitch myofibers per field of percentage and number, respectively, in 3 month old WT and AUF1 KO mice. FIG. 5H is a graph showing mean soleus slow- and fast-twitch myofiber CSA in 3 month old WT and AUF1 KO mice, n=6-7 mice.

FIGS. 6A-6I show AUF1 deletion induces slow- and fast-twitch muscle atrophy at 6 months of age. FIG. 6A is a graph showing body weight of WT and AUF1 KO mice at 6 months, n=5-6 mice. FIG. 6B shows TA, EDL, gastrocnemius, and soleus muscle weight in 6 month old WT and AUF1 KO mice. FIG. 6C shows representative photographic images of excised muscles from 6 month old WT and AUF1 KO mice. FIG. 6D are photographic images showing representative immunostain of slow myosin (red) and laminin (green) in soleus muscle from 6 month old WT and AUF1 KO mice. Scale bar: 500 m. FIG. 6E is a graph showing mean CSA of slow- and fast-twitch myofibers in soleus muscle of 6 month old WT and AUF1 KO mice. FIG. 6F is a graph showing percentage of slow-twitch myofibers in 6 month old WT and AUF1 KO mice in soleus muscle. FIG. 6G is a pair of photographic images showing representative staining of slow myosin (red) and laminin (green) in 6 month old WT and AUF1 KO gastrocnemius muscle. Nuclei were stained by DAPI (blue), scale bar, 200 m. FIG. 6H is a graph showing the number of slow-twitch myofibers per field in gastrocnemius muscle of 6 month old WT and AUF1 KO mice. n=4 mice per group. FIG. 6I is a graph showing mean gastrocnemius myofiber CSA of slow- and fast-twitch myofibers in 6 month old WT and AUF1 KO mice. n=4 mice per group. Mean±SEM from 4 or more independent studies. *P<0.05, **P<0.01 by unpaired Mann-Whitney U test.

FIGS. 7A-7I show AUF1 supplementation in skeletal muscle increased Pax7 expression in muscle and reduces markers of muscle atrophy improves exercise endurance in 12-month old (middle-aged) and 18 month old mice. FIG. 7A presents graphs showing TA, gastrocnemius, EDL muscle mass, and soleus in 3, 12, and 18 month old WT mice normalized to total body weight. FIG. 7B is an immunoblot of AUF1 and β-tubulin in TA muscle 40 d after AAV8 administration. FIG. 7C shows representative immunofluorescence staining of TA muscle at 40 day post-administration of AAV8 GFP control or AAV8 AUF1 GFP vectors, shown is DAPI staining, AUF1, laminin α2 to highlight myofibers and merged images. White arrows point to nuclear AUF1; yellow arrows point to sarcoplasmic AUF1. FIG. 7D is a graph showing auf1 mRNA expression normalized to invariant gapdh mRNA in various organs of 12 month old mice, 40 d after AAV8 AUF1-GFP or AAV8 GFP control administration. FIG. 7F shows representative Pax7, GFP and DAPI staining in TA muscle in 12 month old mice 40 d after AAV8 AUF1-GFP or AAV8 GFP control vector administration. Scale bar, 100 m. Quantification of Pax7 mRNA expression normalized to invariant TBP mRNA, in TA muscle of 12 month old mice 40 d after AAV8 AUF1-GFP or AAV8 GFP control vector administration. n=8-9 per mice group. FIG. 7G is a graph showing relative expression of Trim63 and Fbxo32 mRNAs in TA muscle normalized to TBP mRNA 40 d after AAV administration. FIG. 7H is a graph showing relative expression of Trim63 and Fbxo32 mRNAs in gastrocnemius muscle normalized to TBP mRNA 40 d after AAV administration. FIG. 7I shows representative co-immunostaining of Pax7 (red) and Myf5 (purple) showing activated satellite cells in 12 month old TA muscle of mice at 40 days following AUF1 gene transfer. Mean±SEM from 3 or more independent studies. FIG. 7A-B: *P<0.05, **P<0.01 by Kruskall-Wallis test. All other panels *P<0.05, **P<0.01 by unpaired Mann-Whitney U test.

FIG. 8A-8E show AUF1 controls myosin and MEF2C expression. The graphs of FIG. 8A show relative expression of fast and slow myosin mRNAs normalized to gapdh mRNA in differentiating (48 h) wild type myotubes and AUF1 knock out C2C12 cells. n=5 mice per group. FIG. 8B is a graph showing mef2c mRNA expression normalized to TBP mRNA in gastrocnemius muscle 40 days after AAV AUF1-GFP or AAV GFP injection. n=5 mice per group. FIG. 8C is a graph showing mef2c mRNA expression normalized to TBP mRNA in gastrocnemius muscle 6 months after AAV AUF1-GFP or AAV GFP injection. n=5 mice per group. FIG. 8D shows representative protein levels in the gastrocnemius muscle from two mice chosen at random at 6 months after AAV GFP (GFP) or AAV AUF-1GFP (AUF1) administration. Mean±SEM from 5 or more independent studies. *P<0.05 by unpaired Mann-Whitney U test. Ns, not significant. FIG. 8E shows a schematic of the Renilla luciferase (RLuc) reporter construct in plasmid pIS1 containing either the plasmid 3′UTR without ARE sequences, or as shown, the AU-rich 3′UTR of the pcg1α mRNA. Red, UA-rich elements, blue, U-rich elements. Insertion sites are indicated.

FIGS. 9A-9G show AUF1 deletion induces slow-twitch muscle atrophy at a young age. FIG. 9A shows representative photographic images of TA, EDL, and gastrocnemius muscles in 3 month old WT and AUF1 KO mice. FIG. 9B shows representative immunostain images of slow and fast myosin (red) myofibers in the soleus of WT and AUF1 KO mice. DAPI stain (blue) of nuclei, laminin (green) stain of extracellular matrix. Scale bar: 500 m. FIG. 9C shows photographic images of representative stains of slow myosin (red) and laminin (green) in 3 month old WT and AUF1 KO gastrocnemius muscle (scale bar, 200 m). FIGS. 9D-E are graphs showing percentage and number, respectively, of slow-twitch myofibers per field in gastrocnemius muscle of 3 month old WT and AUF1 KO mice. FIG. 9F is a graph showing mean gastrocnemius muscle area of slow- and fast-twitch myofibers in 3 month old WT and AUF1 KO mice. n=4 mice per group. FIG. 9G shows levels of PGC1α, AUF1, and control GAPDH protein in gastrocnemius and soleus muscles of 3 month old WT and AUF1 KO mice. Each lane corresponds to one mouse. Lower band in AUF1 gastrocnemius muscle lanes is a non-specific protein. Mean±SEM from 3 or more independent studies. *P<0.05 by unpaired Mann-Whitney U test. ns, (not significant).

FIGS. 10A-10C illustrate the development of AAV8 expression vectors. FIG. 10A is a schematic illustration of the development of AAV8 expression vectors. The cDNA of the murine p40^{AUF1} cDNA was cloned into an AAV8 vector under the tMCK promoter (AAV8-tMCK-AUF1-IRES-eGFP) (Vector Biolabs). The tMCK promoter was generated by the addition of a triple tandem of 2RS5 enhancer sequences (3-Ebox) ligated to the truncated regulation region of the MCK (muscle creatine kinase) promoter, which induces high muscle specificity (Blankinship et al., “Efficient Transduction of Skeletal Muscle Using Vectors Based on Adeno-associated Virus Serotype 6,” Mol. Ther. 10(4):671-8 (2004), which is hereby incorporated by reference in its entirety). AAV8 vectors express AUF1 and GFP (AUF1-GFP, with GFP translated from the same mRNA by the HCV IRES), or as a control only GFP. Expression of both genes is controlled by the creatine kinase tMCK promoter that is selectively active in skeletal muscle cells. The AAV8-tMCK-IRES-eGFP construct was used as a control vector. FIG. 10B shows the amino acid sequence of the encoded p40^{AUF}1 isoform (SEQ ID NO:6) expressed in transduced cells by the AAV8 vector in FIG. 10A. FIG. 10C shows the nucleotide sequence (SEQ ID NO:32) of the coding region of the p40^{AUF1} isoform.

FIGS. 11A-11B show AAV8 transduction frequency in mdx mice. AAV8 AUF1-GFP and AAV8 GFP control vector-treated mdx mice displayed similar vector transduction and retention rates, shown by tibialis anterior (TA) muscle GFP staining. FIG. 11A shows representative photographic images of GFP immunofluorescence staining of TA muscle (green) to highlight AAV8 transduction efficiency and laminin-α2 staining (red) to highlight muscle fiber architecture and integrity. FIG. 11B is a graph showing quantification of 3 animals per condition for AAV8 GFP transduction in TA muscle. There is no statistical difference (ns) in transduction efficiency between control AAV8 GFP and treatment AAV8 AUF1 GFP groups.

FIGS. 12A-12F show AUF1 gene therapy enhances muscle mass and endurance in mdx mice. One month old C57BL/10ScSn male DMD mice (herein mdx mice, JACS) were administered 2×10¹¹ genome copies of AAV8 AUF1-GFP or control AAV8 GFP as a single retro-orbital injection of 50 μl containing 2.5×10¹¹ AAV particles. Two months following AAV8 administration, mdx mice transduced with AAV8 AUF1-GFP or AAV8 GFP as a control were tested by standard procedures for exercise performance (see Examples, infra). FIG. 12A is a graph showing mdx control mice receiving only AAV8 GFP at three months old had an average body weight of 29 gm compared to 30 gm for wild type (WT) C57BL mice. In contrast, when compared to control AAV8 GFP treated mdx mice, AAV8 AUF1-GFP supplemented mdx mice had an average body weight of 31 gm, a significant increase compared to control mdx mice. FIG. 12B is a graph showing when normalized to body weight and at 2 months post-gene therapy transduction, AAV8 AUF1-GFP treated mdx mice demonstrated a 10% increase in tibialis anterior (TA) muscle mass, an 11% increase in extensor digitorum longus (EDL) muscle mass, and an 8.5% increase in gastrocnemius muscle mass. There was no difference in soleus muscle mass. Compared to control AAV8 GFP treated mdx mice, AUF1 supplemented mdx mice showed a ˜40% improvement in grid hanging time (FIG. 12C), a measure of limb-girdle skeletal muscle strength and endurance. When tested by treadmill, AAV AUF1-GFP mdx mice displayed 16% higher maximum speed (FIG. 12D), a 35% greater time to exhaustion (FIG. 12E), and a 37% increased distance to exhaustion (FIG. 12F). These data demonstrate a substantial and statistically significant increase in exercise performance and endurance in mdx mice as a result of AUF1 gene transfer. All results are expressed as the mean±SEM. Two group comparisons were analyzed by the unpaired Mann-Whitney test. *, P<0.05.

FIGS. 13A-13D show AUF1 gene therapy does not increase WT muscle mass or endurance. Normal WT C57BL mice, the same background as mdx mice, were administered at 1 month of age AAV8 GFP control or AAV8 AUF1-GFP at 2×10¹¹ genome copies by retro-orbital injection as described in FIGS. 12A-12F. Mice were analyzed at 3 months post-gene transfer. These data are in contrast to the significant increase in muscle mass and exercise endurance found in mdx mice. Rather, WT mice administered with AAV8 AUF1-GFP compared to control AAV8 GFP mice of the same genetic background, show no statistically significant increase in body weight (FIG. 13A), treadmill time to exhaustion (FIG. 13B), maximum speed (FIG. 13C), and distance to exhaustion (FIG. 13D). All results are expressed as the mean±SEM. Two group comparisons were analyzed by the unpaired Mann-Whitney test. No results were found to be significantly different at P<0.05.

FIG. 14 shows AAV8 AUF1 gene therapy reduces serum creatine kinase levels in mdx mice. mdx mice at 1 month old were administered AAV8 AUF1-GFP or control AAV8 GFP as described in FIGS. 12A-12F. At 3 months, mice were tested for levels of serum creatine kinase (CK) activity, a measure of sarcolemma leakiness and muscle atrophy. Top: Raw data showing serum CD activity results for WT control, mdx mice treated with AAV8 GFP vector alone, and mdx mice treated with AAV8 AUF1 GFP. Bottom: Quantification of three replicate studies of 3 mice each. Control AAV8 GFP mdx mice displayed high levels of serum CK activity, mdx mice that received AAV8 AUF1-GFP gene therapy were reduced in serum CK activity by more than 4-fold, a highly significant reduction. WT C57BL mice had no detectable level of serum CK activity. ND, not detected. All results are expressed as the mean±SEM. Two group comparisons were analyzed by the unpaired Mann-Whitney test. **, P<0.01; *** P<0.001.

FIGS. 15A-15B show AAV8 AUF1 gene therapy reduces muscle necrosis and fibrosis in mdx mouse diaphragm. mdx mice at 1 month old were administered AAV8 AUF1-GFP or control AAV8 GFP as described in FIGS. 12A-12F. At 3 months, diaphragms were reduced from AAV8 GFP control and AAV8 AUF1-GFP mice, embedded FFPE and stained with H&E (FIG. 15A). The percent degenerative diaphragm muscle was scored and found to be reduced by 74% by AUF1 gene transfer. WT C57BL mouse diaphragm served as a control. Diaphragm muscle from mdx mice was stained with Masson Trichome to quantify muscle fibrosis (FIG. 15B). Shown are representative muscle sections. AUF1 gene transfer reduced fibrosis by 2-fold compared to control AAV8 GFP treated animals. All results are expressed as the mean±SEM. Two group comparisons were analyzed by the unpaired Mann-Whitney test. **, P<0.01. Otherwise analyzed by Fisher Exact test as indicated.

FIGS. 16A-16B show AAV8 AUF1 gene therapy reduces muscle immune cell invasion. mdx mice at 1 month old were administered AAV8 AUF1-GFP or control AAV8 GFP as described in FIGS. 12A-12F. At 3 months, diaphragms were resected from AAV8 GFP control and AAV8 AUF1-GFP treated mice, embedded in FFPE, and stained with an antibody to the macrophage biomarker CD68 coupled with the red fluorescence marker Alexa Fluor 555. Representative images show strong reduction in macrophage CD68 staining in AAV8 AUF1-GFP treated animals compared to AAV8 GFP controls (FIG. 16A). Quantification of 5 fields per specimen from 3 mice per group for CD68 staining (FIG. 16B). All results are expressed as the mean±SEM. Two group comparisons were analyzed by the unpaired Mann-Whitney test. *, P<0.05.

FIGS. 17A-17E show AAV8 AUF1 gene therapy suppresses expression of embryonic myosin heavy chain (eMHC) in mdx mice. eMHC is a clinical marker of muscle degeneration in DMD. mdx mice at 1 month old were administered AAV8 AUF1-GFP or control AAV8 GFP as described in FIGS. 12A-12F. At 3 months, diaphragm muscle was removed, fixed in FFPE, and stained with antibodies to eMHC (green), nuclei (DAPI, blue), and laminin (red). Immunofluorescence was carried out and representative images shown compared to WT C57BL6 mice (FIG. 17A). AAV8 AUF1-GFP gene transfer strongly reduced eHMC expression in diaphragm. High magnification of diaphragm stained as in FIG. 17A showing strong reduction in eMHC expression by AUF1 gene transfer (FIG. 17B). Quantification of eMHC staining in myofibers, showing a 75% reduction in eMHC expression by AUF1 gene transfer (FIG. 17C). The percent of centro-nuclei per myofiber/field was quantified, a measure of normal muscle fiber maturation (FIG. 17D). AUF1 gene transfer reduced the percentage of centro-nuclei by 52% compared to AAV8 GFP controls. Myofiber cross sectional area (CSA) was quantified (FIG. 17E). AUF1 gene transfer strongly increased the CSA of the larger myofibers, indicative of mature regenerative muscle. All results are expressed as the mean±SEM. Two group comparisons were analyzed by the unpaired Mann-Whitney test. Multiple group comparisons were performed using one-way analysis of variance (ANOVA). The non-parametric Kruskal-Wallis test followed by the Dunn's comparison of pairs was used to analyze groups when suitable. *, P<0.05; *** P<0.001.

FIGS. 18A-18C show AAV8 AUF1 gene transfer increases expression of endogenous utrophin-A in mdx mice. mdx mice at 1 month old were administered AAV8 AUF1-GFP or control AAV8 GFP as described in FIGS. 12A-12F. The gastrocnemius muscle was removed at 3 months, fixed in FFPE, and stained with DAPI (blue for nuclei, antibodies to utrophin (red) and laminin (green) (FIG. 18A). Representative images from 3 mice for each group are shown. AUF1 gene therapy strongly increased expression of utrophin and showed evidence for normalization of myofiber integrity (laminin staining). Immunoblot analysis for utrophin, AUF1, and GAPDH (invariant control) proteins was conducted on the gastrocnemius muscle of 3 AAV8 GFP and 3 AAV8 AUF1-GFP mdx mice at 3 months (FIG. 18B). Gastrocnemius utrophin protein levels were increased by an average of 20-fold in animals receiving AUF1 gene therapy. AUF1 protein levels were increased an average of 3-4 fold. Utrophin mRNA levels were quantified by qRT-PCR and normalized to invariant TBP mRNA (FIG. 18C). There was no statistically significant difference between samples. n=3 animals for each condition.

FIGS. 19A-19C show AAV8 AUF1 gene transfer increases expression of satellite cell activation gene Pax7, key muscle regeneration genes pgc1α and mef2c, slow twitch determination genes and mitochondrial DNA content in mdx mice. mdx mice at 1 month old were administered AAV8 AUF1-GFP or control AAV8 GFP as described in FIGS. 12A-12F. The gastrocnemius muscle was removed at 3 months, mRNA extracted and quantified by qRT-PCR relative to invariant tbp mRNA. AUF1 gene therapy increased expression of pgc1α, mef2c, and Pax7 mRNAs in the gastrocnemius of mdx mice relative to controls receiving vector alone (FIG. 19A). Wild type non-mdx animals (WT) served as a control for normal muscle levels in age-matched animals. AAV8 AUF1 gene therapy restored near WT levels or exceeded WT levels of gene expression. AUF1 gene therapy increased expression of slow-twitch lineage determination myosin mRNAs in the gastrocnemius muscle in mdx animals relative to controls receiving vector alone (FIG. 19B). AAV8 AUF1 gene therapy restored near WT levels or exceeded WT levels of gene expression. AUF1 gene therapy increased expression of mitochondrial DNA in the gastrocnemius muscle of mdx mice, consistent with increased slow-twitch muscle mass (FIG. 19C). All results are expressed as the mean±SEM. Two group comparisons were analyzed by the unpaired Mann-Whitney test. *, P<0.05; **, P<0.01; *** P<0.001.

FIG. 20 shows genome-wide transcriptomic and translatomic studies demonstrate AUF1 activation of C2C12 myoblast muscle fiber development. Proliferating C2C12 mouse cardiac myoblasts were transduced with lentivirus control vectors or lentivirus vectors expressing p45 AUF1, and induced to differentiate into myotubes by culturing in differentiation medium as described in the Examples infra. Proliferating myoblasts were used because they are activated in p38 MAPK and other signaling pathways that promote myogenesis, which is representative of the activated state and population of muscle cells following muscle damage from wounding, or the state of muscle in myogenic diseases, such as chronic regenerative attempts that occur in Duchene Muscular Dystrophy (DMD). Overview of the experimental approach. At 48 h, when myotubes begin to form, polyribosomes were separated by sucrose sedimentation corresponding to poorly translated (2 & 3 ribosome) fraction and well translated (≥4 polysome) fractions, total mRNA and mRNA in polyribosome fractions were independently purified (polyA+ fraction devoid of rRNA), bacterial libraries were generated and subjected to deep sequencing using RNAseq, in two independent studies. Genome-wide mRNA abundance used log 2 ratios of translated/total mRNA. Procedures and bioinformatic pipeline used for analysis are described in the Examples infra.

FIGS. 21A-21B show AUF1 supplementation stimulates expression of major muscle development pathways and decreases expression of inflammatory cytokine, inflammation, cell proliferation, cell death, and anti-muscle regeneration pathways. Data from FIG. 20 genome-wide mRNA expression and translation analysis. Major upregulated pathways at the levels of transcription, translation, or both with AUF1 supplementation in C2C12 myoblasts (FIG. 21A). Analyzed by KEGG. Major downregulated pathways at the levels of transcription, translation, or both with AUF1 supplementation in C2C12 myoblasts (FIG. 21B). Analyzed by KEGG.

FIGS. 22A-22B show AUF1 supplementation of C2C12 myoblasts upregulates pathways for major biological processes and molecular functions in muscle development and regeneration. Data from FIG. 20 genome-wide mRNA expression and translation analysis. Major upregulated biological processes at the levels of transcription, translation, or both with AUF1 supplementation in C2C12 myoblasts (FIG. 22A). Analyzed by KEGG. Major upregulated molecular functions at the levels of transcription, translation, or both with AUF1 supplementation in C2C12 myoblasts (FIG. 22B). Analyzed by KEGG.

FIGS. 23A-23B show AUF1 supplementation of C2C12 myoblasts decreases muscle inflammation, inflammatory cytokine, and signaling pathways that oppose muscle regeneration. Data from FIG. 20 genome-wide mRNA expression and translation analysis. Major downregulated biological processes at the levels of transcription, translation, or both with AUF1 supplementation in C2C12 myoblasts (FIG. 23A). Analyzed by KEGG. Major downregulated molecular functions at the levels of transcription, translation, or both with AUF1 supplementation in C2C12 myoblasts (FIG. 23B). Analyzed by KEGG.

FIG. 24 shows AUF1 supplementation of C2C12 myoblasts decreases expression of muscle genes associated with development of fibrosis. Data from FIG. 20 genome-wide mRNA expression and translation analysis. Major downregulated pathways and functions at the levels of transcription, translation, or both with AUF1 supplementation in C2C12 myoblasts. Analyzed by KEGG.

FIGS. 25A-25D show lentivirus transduction of injured TA muscle with p45 AUF1 in mice activates satellite cells and reduces biomarkers of muscle atrophy. A lentivirus vector was developed expressing cDNA for p45 AUF1 under control of the CMV promoter (Abbadi et al., “Muscle Development and Regeneration Controlled by AUF1-mediated Stage-specific Degradation of Fate-determining Checkpoint mRNAs,” Proc. Nat'l. Acad. Sci. USA 116:11285-90 (2019), which is hereby incorporated by reference in its entirety). Three month old male mice were administered an intramuscular injection of 50 μl of filtered 1.2% BaCl₂ in sterile saline with control lentivirus vector or with lentivirus AUF1 vector (1×10⁸ genome copies) (total volume 100 l) into the left Tibialis Anterior (TA) muscle (FIG. 25A). The right TA muscle remained uninjured as a control. Mice were sacrificed at 7 days post-injection. TA muscles were excised, weighed, and normalized to mouse body weight in grams. TA injury reduced TA weight by 27% which was restored to near-uninjured levels by concurrent AUF1 gene therapy. In FIG. 25B, immunoblot analysis of AUF1 normalized to invariant GAPDH protein for TA muscle at 7 days post-lentivirus p45 AUF1 administration as in FIG. 25A. Shown is a representative uninjured, two injured, and injured TA muscles with concurrent p45 AUF1 gene therapy from independent animals. Lentivirus p45 AUF1 gene transfer strongly increased levels of the p45^{AUF1} isoform but not p42^{AUF1} and p40^{AUF1} that were not encoded (p37^{AUF1} is undetectable). In FIG. 25C, TA muscles as in FIG. 25A were probed by qRT-PCR for Pax7 mRNA levels, a biomarker of muscle satellite (stem) cell activation, and normalized to invariant TATA-box binding protein (TBP) mRNA. AUF1 gene therapy increased Pax7 expression by >3-fold. In FIG. 25D, TA muscles as in FIG. 25A were probed by qRT-PCR for expression of muscle atrophy biomarker genes TRIM63 and Fbxo32, normalized to TBP mRNA. TA muscle injury strongly induced expression of TRIM63 and Fbxo32 mRNA, which were downregulated to uninjured TA muscle levels by p45^{AUF1} gene therapy, indicating strong cessation of muscle injury due to AUF1 intramuscular administration. No statistical difference (ns). All results are expressed as the mean±SEM with at least three independent trials of 3 or more animals per condition. Two group comparisons were analyzed by the unpaired Mann-Whitney test. *, P<0.05; **, P<0.01; ***, P<0.001.

FIGS. 26A-26D show p45 AUF1 lentivirus transduction enhances expression of muscle regeneration factors (MRFs) following TA muscle injury. Three month old male mice were injured in the TA muscle with BaCl₂ and administered with an intramuscular injection of control lentivirus vector or lentivirus AUF1 vector (see FIGS. 25A-D). Mice were sacrificed at 7 days post-injection. TA muscles were probed by qRT-PCR for identified mRNAs normalized to invariant TBP mRNA. In FIG. 26A, myogenin and MyoD mRNA levels, biomarkers of myoblast activation, differentiation, and muscle regeneration (Zammit, “Function of the Myogenic Regulatory Factors Myf5, MyoD, Myogenin and MRF4 in Skeletal Muscle, Satellite Cells and Regenerative Myogenesis,” Semin. Cell. Dev. Biol. 72:19-32 (2017), which is hereby incorporated by reference in its entirety), were increased ˜2-fold by AUF1 gene therapy relative to injured control vector specimens. In FIG. 25B, myh8 mRNA, an embryonic myosin only expressed in adult muscle during muscle regeneration and a marker of co-expression of utrophin (Guiraud et al., “Embryonic Myosin is a Regeneration Marker to Monitor Utrophin-based Therapies for DMD,” Hum. Mol. Genet. 28:307-19 (2019), which is hereby incorporated by reference in its entirety), was increased in expression by 5-fold in injured muscle with AUF1 gene therapy relative to injured control vector specimens. In FIG. 26C, myh7 mRNA, a myosin that specifies slow-twitch muscle (Zammit, “Function of the Myogenic Regulatory Factors Myf5, MyoD, Myogenin and MRF4 in Skeletal Muscle, Satellite Cells and Regenerative Myogenesis,” Semin. Cell. Dev. Biol. 72:19-32 (2017), which is hereby incorporated by reference in its entirety), was increased in expression by -2-fold in injured muscle with AUF1 gene therapy relative to injured control vector specimens. In FIG. 26D, myh4 mRNA, a myosin that specifies fast-twitch muscle (Zammit, “Function of the Myogenic Regulatory Factors Myf5, MyoD, Myogenin and MRF4 in Skeletal Muscle, Satellite Cells and Regenerative Myogenesis,” Semin. Cell. Dev. Biol. 72:19-32 (2017), which is hereby incorporated by reference in its entirety), was increased in expression by -2-fold in injured muscle with AUF1 gene therapy relative to injured control vector specimens. All results are expressed as the mean±SEM with at least three independent trials of 3 or more animals per condition. Two group comparisons were analyzed by the unpaired Mann-Whitney test. *, P<0.05; **, P<0.01.

FIGS. 27A-27D show p45 AUF1 lentivirus gene therapy promotes rapid regeneration of injured muscle. Three month old male mice were injured in the TA muscle with BaCl₂, and administered with an intramuscular injection of control lentivirus vector or lentivirus AUF1 vector, as in FIGS. 25A-25D. Mice were sacrificed at 3 days and 7 days post-injury. FIG. 27A shows photographic images of muscle fibers provide evidence for accelerated but normal muscle regeneration of myofibers in animals administered lentiviral AUF1 gene therapy. TA muscle in OCT was sectioned and stained for immunofluorescence microscopy analysis for Laminin alpha 2 (red), Nuclei are stained with DAPI (blue). Note the disrupted myofiber architecture and high level of central nuclei in the injured TA muscle treated with vector alone compared to the injured TA muscle administered lentiviral AUF1 gene therapy, consistent with accelerated muscle regeneration and mature myofibers. Scale bar, 200 m. FIG. 27B is a graph showing the percent muscle loss (atrophy) or gain (increase in mass) determined for the injured TA muscle compared to uninjured control or injured muscle receiving control lentivirus vector or lentivirus p45 AUF1, measured at sacrifice at 3 days and 7 days post-injury. Injured TA muscle receiving sham gene therapy sustained a 20% loss in mass by day 3 following injury, which only very slightly improved by day 7. In contrast, injured TA muscle receiving AUF1 gene therapy showed a trend to less atrophy by day 3, which was almost fully recovered by day 7, demonstrating near normal mass. FIG. 27C is a graph showing high levels of myotube central nuclei are a marker of immature myofiber development (Yin et al., “Satellite Cells and the Muscle Stem Cell Niche,” Physiol. Rev. 93:23-67 (2013) and Schiaffino & Reggiani, “Fiber Types in Mammalian Skeletal Muscles,” Physiol. Rev. 91:1447-531 (2011), which are hereby incorporated by reference in their entirety). TA muscle analyzed at day 7 post-injury administered p45 AUF1 gene therapy were reduced by half in the percent of myofibers with central nuclei compared to vector only control injured muscle. This is consistent with accelerated muscle regeneration provided by AUF1 gene transfer. FIG. 27D is a graph showing a wider cross-sectional area of myofibers (cross-sectional area, CSA) with low numbers of central nuclei are indicative of mature myofiber development (Yin et al., “Satellite Cells and the Muscle Stem Cell Niche,” Physiol. Rev. 93:23-67 (2013) and Schiaffino & Reggiani, “Fiber Types in Mammalian Skeletal Muscles,” Physiol. Rev. 91:1447-531 (2011), which are hereby incorporated by reference in their entirety). AUF1 gene transfer in injured TA muscle produced a striking increase in CSA with reduced central nuclei per myofiber, consistent with generation of mature myofibers. All results are expressed as the mean±SEM with at least three independent trials of 3 or more animals per condition. Two group comparisons were analyzed by the unpaired Mann-Whitney test. *, P<0.05; **, P<0.01, ***, P<0.001.

FIGS. 28A-28F show AUF1 is essential to promote repair of injured muscle, and can provide injury protection benefit when delivered by AAV8 gene transfer. FIG. 28A is a schematic illustration of an AUF1 conditional knockout mouse developed as an aspect of the technology described herein. Shown is a schematic of the exon 3 LoxP site insertions in the AUF1 gene. Lox sites were cloned to flank exon 3 of AUF1, which is maintained in all 4 AUF1 isoforms and contains the RNA binding domain. AUF1^{Fox/Flox} mice were derived, siblings mated to homogeneic purity generated, then mated with a Pax7cre ERT2 (B6; 129-Pax7^{tm2.1(cre/ER)Fan}/J mouse) (Jackson Labs). This provides cre recombinase induction by tamoxifen administration only in PAX7⁺ expressing muscle satellite and myoblast cells. FIG. 28B is a graph showing results of three month old mice induced for cre expression with 5 daily i.p. injections of tamoxifen (3 mg/kg). There was no change in body weight of cre-induced mice. FIG. 28C is a graph showing weight of non-injured skeletal muscles in mice were not significantly different in uninduced and tamoxifen induced cre mice. FIG. 28D shows tamoxifen induction of cre for 3 months specifically deletes the auf1 gene in skeletal muscle and abolishes skeletal muscle AUF1 protein expression. A representative immunoblot is shown for AUF1 levels in TA skeletal muscle and kidney, normalized to invariant GAPDH in control AUF1^{Flox/Flox} and AUF1^{Flox/Flox}×PAX7^{cre} ERT2 mice after 5 days of cre induction and analyzed at day 7. There is no evidence for expression of AUF1 after Pax7-specific cre induction in muscle, whereas abundant AUF1 is present in kidney. FIG. 28E is a graph showing one month old AUF1^{Fox/Flox}×PAX7^{cre} ERT2 mice were either sham injected or injected with tamoxifen for 5 days as above, then maintained on a diet that included oral tamoxifen for 5 months daily at 500 mg/kg (Envigo). Wild type (WT) BL6 mice and AUF1^{Flox/Flox}×PAX7^{cre}ERT2 mice were either not induced for cre-expression (labeled AUF^{fl/fl/Pax7}) or induced for 5 months and deleted in the AUF1 gene (labeled ΔAUF1^{fl/fl/Pax7}). One set of ΔAUF1^{fl/fl/Pax7} mice induced for cre expression for 5 months were also administered at 1 month of age with 2.0×10¹¹ AAV8 AUF1 particles (2×10¹¹ genome copies) by single retro-orbital injection of 50 μl. All mice were then injured by 1.2% BaCl₂ injection in the TA muscle, as described in FIGS. 25A-D. AUF1 is controlled by the creatine kinase tMCK promoter that is selectively active in skeletal muscle cells. TA muscle was excised at 7 days post-BaCl₂ injection and the percent of muscle atrophy determined by weight. TA muscle of AUF1^{Flox/Flox}×PAX7^{cre}ERT2 mice expressing AUF1 and WT mice expressing AUF1 (not induced for cre) showed 16-18% atrophy that was not statistically different. In contrast, deletion of the AUF1 gene caused strongly increased atrophy of the TA muscle, doubling atrophy levels to 35%. However, animals deleted for the AUF1 gene but prophylactically administered AAV8 AUF1 gene therapy demonstrated dramatically reduced levels of TA muscle atrophy, averaging ˜3%. FIG. 28F is a graph showing AUF1 control and cre-induced skeletal muscle AUF1 deleted mice were tested at 5 months for grip strength, a measure of limb-girdle skeletal muscle strength and endurance. AUF1 deleted mice showed a ˜50% reduction in grip strength. Collectively, these data demonstrate that AUF1 is essential for maintenance of muscle strength and muscle regeneration following injury, and that AUF1 gene therapy provides a remarkable ability to promote muscle regeneration and protect muscle from extensive damage despite traumatic injury. All results are expressed as the mean±SEM with at least three independent trials of 3 or more animals per condition. Two group comparisons were analyzed by the unpaired Mann-Whitney test. *, P<0.05.

FIGS. 29A and 29B. FIG. 29A shows that supplemental expression of AUF1 (p40) accelerates the formation of myotubes from wild type C2C12 myoblast cells in culture. FIG. 29B provides RNA sequence analysis of the wild type C2C12 myoblast cultures after differentiation into myotubes as compared to vector control samples. Results show AUF1 supplementation stimulates expression of slow muscle specification genes.

FIGS. 30A-E. FIG. 30A shows that prophylactic administration of AAV8-mAUF1 significantly decreases the percent of muscle atrophy compared to WT control mice measured at 7 d and 14 d post-BaCl₂ induction of muscle necrosis. FIG. 30B is a histogram that quantifies centrally located nuclei compared to mean cross-sectional area (csa) of muscle fibers. Results show that the greatest central nuclei with the greatest csa is in muscle of mAUF1-treated animals at 14 d post-injury. FIGS. 30C-E are raw data plots used to derive the summary histogram shown in FIG. 30B.

### 5. DETAILED DESCRIPTION

Provided are methods and compositions for promoting muscle regeneration, restoring or increasing muscle mass, muscle function or performance, and/or reducing or reversing muscle atrophy by increasing the levels of AUF1 in muscle cells in a subject in need thereof. Methods and compositions are provided for administering AUF1 protein or nucleic acid that encodes and expresses AUF1 protein in muscle cells, such as DNA, mRNA, plasmid DNA or viral vectors encoding AUF1. Provided are compositions comprising, and methods of administering, gene therapy vectors, particularly recombinant AAV vectors, comprising genomes with transgenes encoding AUF1 proteins operably linked to regulatory elements that promote AUF1 expression in muscle cells for restoring or increasing muscle mass, muscle function or performance, and/or reducing or reversing muscle atrophy. Such methods include stabilizing the sarcolemma of the muscle cell by reducing leakiness (for example, as measured by creatine kinase levels), increasing expression of β-sarcoglycan or utrophin or other components of the dystrophin-glycoprotein complex (including α-dystroglycan, β-dystroglycan, α-sarcoglycan, β-sarcoglycan, δ-sarcoglycan, γ-sarcoglycan, ε-Sarcoglycan, ζ-sarcoglycan, α-dystroglycan, β-dystroglycan, sarcospan, α-syntrophin, β-syntrophin, α-dystrobrevin, β-dystrobrevin, caveolin-3, or nNOS) and/or their presence in the dystrophin-glycoprotein complex of muscle cells by providing AUF1 protein, including by gene therapy methods, such as AAV gene therapy. Other methods provided include treatment, prevention or amelioration of the symptoms of muscle wasting including sarcopenia, including in the elderly, traumatic muscle injury, and diseases or disorders associated with a lack or loss of muscle mass, function or performance, such as, but not limited to dystrophinopathies and other related muscle diseases or disorders. Such methods include promoting an increase in muscle cell mass, number of muscle fibers, size of muscle fibers, muscle cell regeneration, reduction in or reverse of muscle cell atrophy, satellite cell activation and differentiation, improvement in muscle cell function (for example, by increasing mitochondrial oxidative capacity), and increasing proportion of slow twitch fiber in muscle (including by conversion of fast to slow twitch muscle fibers). Other methods disclosed herein include methods of producing synthetic or cultured meat by promoting muscle cell formation, for example, in vitro in cell culture and particularly producing slow twitch muscle fibers, including converting fast twitch to slow twitch muscle fibers in the cultured muscle cells, thereby providing an improved cultured meat product. In addition, other methods disclosed herein include methods of producing synthetic muscle tubes, muscle fiber and muscle by promoting muscle cell formation, for example, in vitro in cell culture and particularly producing slow twitch muscle fibers, including converting fast twitch to slow twitch muscle fibers in the cultured muscle cells, thereby providing an improved cultured muscle composition and uses thereof.

Such methods maybe carried out by administration of AUF1 (including mouse or human p37AUF1, p40AUF1, p42AUF1 or p45AUF1), including administering a gene therapy vector, such as a lentiviral vector or a recombinant AAV gene therapy vector comprising a nucleic acid encoding the mouse or human AUF1, or a functional fragment thereof, operably linked to regulatory elements promoting AUF1 expression in muscle cells. Compositions comprising rAAV comprising a genome comprising a transgene encoding human AUF1, or a functional fragment thereof, operably linked to regulatory elements that promote expression of the AUF1 encoding nucleic acid in muscle cells are further provided.

### 5.1.Definitions

The term “vector” is used interchangeably with “expression vector.” The term “vector” may refer to viral or non-viral, prokaryotic or eukaryotic, DNA or RNA sequences that are capable of being transfected into a cell, referred to as “host cell,” so that all or a part of the sequences are transcribed. It is not necessary for the transcript to be expressed. It is also not necessary for a vector to comprise a transgene having a coding sequence. Vectors are frequently assembled as composites of elements derived from different viral, bacterial, or mammalian genes. Vectors contain various coding and non-coding sequences, such as sequences coding for selectable markers, sequences that facilitate their propagation in bacteria, or one or more transcription units that are expressed only in certain cell types. For example, mammalian expression vectors often contain both prokaryotic sequences that facilitate the propagation of the vector in bacteria and one or more eukaryotic transcription units that are expressed only in eukaryotic cells. It will be appreciated by those skilled in the art that the design of the expression vector can depend on such factors as the choice of the host cell to be transformed, the level of expression of protein desired, etc.

The term “promoter” is used interchangeably with “promoter element” and “promoter sequence.” Likewise, the term “enhancer” is used interchangeably with “enhancer element” and “enhancer sequence.” The term “promoter” refers to a minimal sequence of a transgene that is sufficient to initiate transcription of a coding sequence of the transgene. Promoters may be constitutive or inducible. A constitutive promoter is considered to be a strong promoter if it drives expression of a transgene at a level comparable to that of the cytomegalovirus promoter (CMV) (Boshart et al., “A Very Strong Enhancer is Located Upstream of an Immediate Early Gene of Human Cytomegalovirus,” Cell 41:521 (1985), which is hereby incorporated by reference in its entirety). Promoters may be synthetic, modified, or hybrid promoters. Promoters may be coupled with other regulatory sequences/elements which, when bound to appropriate intracellular regulatory factors, enhance (“enhancers”) or repress (“repressors”) promoter-dependent transcription. A promoter, enhancer, or repressor, is said to be “operably linked” to a transgene when such element(s) control(s) or affect(s) transgene transcription rate or efficiency. For example, a promoter sequence located proximally to the 5′ end of a transgene coding sequence is usually operably linked with the transgene. As used herein, the term “regulatory elements” is used interchangeably with “regulatory sequences” and refers to promoters, enhancers, and other expression control elements, or any combination of such elements.

Promoters are positioned 5′ (upstream) to the genes that they control. Many eukaryotic promoters contain two types of recognition sequences: TATA box and the upstream promoter elements. The TATA box, located 25-30 bp upstream of the transcription initiation site, is thought to be involved in directing RNA polymerase II to begin RNA synthesis at the correct site. In contrast, the upstream promoter elements determine the rate at which transcription is initiated. These elements can act regardless of their orientation, but they must be located within 100 to 200 bp upstream of the TATA box.

Enhancer elements can stimulate transcription up to 1000-fold from linked homologous or heterologous promoters. Enhancer elements often remain active even if their orientation is reversed (Li et al., “High Level Desmin Expression Depends on a Muscle-Specific Enhancer,” J. Bio. Chem. 266(10):6562-6570 (1991), which is hereby incorporated by reference in its entirety). Furthermore, unlike promoter elements, enhancers can be active when placed downstream from the transcription initiation site, e.g., within an intron, or even at a considerable distance from the promoter (Yutzey et al., “An Internal Regulatory Element Controls Troponin I Gene Expression,” Mol. Cell. Bio. 9(4):1397-1405 (1989), which is hereby incorporated by reference in its entirety).

The term “muscle cell-specific” refers to the capability of regulatory elements, such as promoters and enhancers, to drive expression of an operatively linked nucleic acid molecule (e.g., a nucleic acid molecule encoding an AU-rich mRNA binding factor 1 (AUF1) protein or a functional fragment thereof) exclusively or preferentially in muscle cells or muscle tissue.

The term “AAV” or “adeno-associated virus” refers to a Dependoparvovirus within the Parvoviridae genus of viruses. The AAV can be an AAV derived from a naturally occurring “wild-type” virus, an AAV derived from a rAAV genome packaged into a capsid comprising capsid proteins encoded by a naturally occurring cap gene and/or from a rAAV genome packaged into a capsid comprising capsid proteins encoded by a non-naturally occurring capsid cap gene. An example of the latter includes a rAAV having a capsid protein having a modified sequence and/or a peptide insertion into the amino acid sequence of the naturally-occurring capsid.

The term “rAAV” refers to a “recombinant AAV.” In some embodiments, a recombinant AAV has an AAV genome in which part or all of the rep and cap genes have been replaced with heterologous sequences, including a transgene, such as nucleotide sequence encoding AUF1, and regulatory elements for expression of the transgene.

The term “rep-cap helper plasmid” refers to a plasmid that provides the viral rep and cap gene function and aids the production of AAVs from rAAV genomes lacking functional rep and/or the cap gene sequences.

The term “cap gene” refers to the nucleic acid sequences that encode capsid proteins that form or help form the capsid coat of the virus. For AAV, the capsid protein may be VP1, VP2, or VP3.

The term “rep gene” refers to the nucleic acid sequences that encode the non-structural protein needed for replication and production of virus.

The terms “nucleic acids” and “nucleotide sequences” include DNA molecules (e.g., cDNA or genomic DNA), RNA molecules (e.g., mRNA), combinations of DNA and RNA molecules or hybrid DNA/RNA molecules, and analogs of DNA or RNA molecules. Such analogs can be generated using, for example, nucleotide analogs, which include, but are not limited to, inosine or tritylated bases. Such analogs can also comprise DNA or RNA molecules comprising modified backbones that lend beneficial attributes to the molecules such as, for example, nuclease resistance or an increased ability to cross cellular membranes. The nucleic acids or nucleotide sequences can be single-stranded, double-stranded, may contain both single-stranded and double-stranded portions, and may contain triple-stranded portions, but preferably is double-stranded DNA.

Amino acid residues as disclosed herein can be modified by conservative substitutions to maintain, or substantially maintain, overall polypeptide structure and/or function. As used herein, “conservative amino acid substitution” indicates that: hydrophobic amino acids (i.e., Ala, Cys, Gly, Pro, Met, Val, lie, and Leu) can be substituted with other hydrophobic amino acids; hydrophobic amino acids with bulky side chains (i.e., Phe, Tyr, and Trp) can be substituted with other hydrophobic amino acids with bulky side chains; amino acids with positively charged side chains (i.e., Arg, His, and Lys) can be substituted with other amino acids with positively charged side chains; amino acids with negatively charged side chains (i.e., Asp and Glu) can be substituted with other amino acids with negatively charged side chains; and amino acids with polar uncharged side chains (i.e., Ser, Thr, Asn, and Gln) can be substituted with other amino acids with polar uncharged side chains.

The terms “subject”, “host”, and “patient” are used interchangeably. A subject may be a mammal such as a non-primate (e.g., cows, pigs, horses, cats, dogs, rats etc.) or a primate (e.g., monkey and human), and includes a human.

The terms “therapeutic agent” refers to any agent which can be used in treating, managing, or ameliorating symptoms associated with a disease or disorder, where the disease or disorder is associated with a function to be provided by a transgene. A “therapeutically effective amount” refers to the amount of agent, (e.g., an amount of product expressed by the transgene) that provides at least one therapeutic benefit in the treatment or management of the target disease or disorder, when administered to a subject suffering therefrom. Further, a therapeutically effective amount with respect to an agent of the invention means that amount of agent alone, or when in combination with other therapies, that provides at least one therapeutic benefit in the treatment or management of the disease or disorder.

The term “prophylactic agent” refers to any agent which can be used in the prevention, reducing the likelihood of, delay, or slowing down of the progression of a disease or disorder, where the disease or disorder is associated with a function to be provided by a transgene. A “prophylactically effective amount” refers to the amount of the prophylactic agent (e.g., an amount of product expressed by the transgene) that provides at least one prophylactic benefit in the prevention or delay of the target disease or disorder, when administered to a subject predisposed thereto. A prophylactically effective amount also may refer to the amount of agent sufficient to prevent, reduce the likelihood of, or delay the occurrence of the target disease or disorder; or slow the progression of the target disease or disorder; the amount sufficient to delay or minimize the onset of the target disease or disorder; or the amount sufficient to prevent or delay the recurrence or spread thereof. A prophylactically effective amount also may refer to the amount of agent sufficient to prevent or delay the exacerbation of symptoms of a target disease or disorder. Further, a prophylactically effective amount with respect to a prophylactic agent of the invention means that amount of prophylactic agent alone, or when in combination with other agents, that provides at least one prophylactic benefit in the prevention or delay of the disease or disorder.

A prophylactic agent of the invention can be administered to a subject “pre-disposed” to a target disease or disorder. A subject that is “pre-disposed” to a disease or disorder is one that shows symptoms associated with the development of the disease or disorder, or that has a genetic makeup, environmental exposure, or other risk factor for such a disease or disorder, but where the symptoms are not yet at the level to be diagnosed as the disease or disorder. For example, a patient with a family history of a disease associated with a missing gene (to be provided by a transgene) may qualify as one predisposed thereto. Further, a patient with a dormant tumor that persists after removal of a primary tumor may qualify as one predisposed to recurrence of a tumor.

As used herein, the terms “promote,” “promotion,” and “promoting” refer to an increase in an activity, response, condition, or other biological parameter, including the production, presence, expression, or function of cells, biomolecules or bioactive molecules. The terms “promote,” “promotion,” and “promoting include, but are not limited to, initiation of an activity, response, or condition, as well as initiation of the production, presence, or expression of cells, biomolecules, or bioactive molecules. The terms “promote,” “promotion,” and “promoting” may also include measurably increasing an activity, response, or condition, or measurably increasing the production, presence, expression, or function of cells, biomolecules, or bioactive molecules, as compared to a native or control level.

### 5.2. AU-Rich mRNA Binding Factor 1 Transgenes

Provided are nucleic acids, including transgenes, encoding AUF1s, including the p37, p40, p42 and p45 isoforms of human and mouse AUF1, or therapeutically functional fragments thereof, and vectors and viral particles, including rAAVs, containing same and methods of using same in methods of treatment, prevention or amelioration of symptoms of conditions associated with loss of muscle mass or performance or where an increase in muscle mass or performance is desired or useful, as well as methods of producing synthetic meat.

Genes involved in rapid response to cell stimuli are highly regulated and typically encode mRNAs that are selectively and rapidly degraded to quickly terminate protein expression and reprogram the cell (Moore et al., “Physiological Networks and Disease Functions of RNA-binding Protein AUF1,” Wiley Interdiscip. Rev. RNA 5(4):549-64 (2014), which is hereby incorporated by reference in its entirety). These include growth factors, inflammatory cytokines (Moore et al., “Physiological Networks and Disease Functions of RNA-binding Protein AUF1,” Wiley Interdiscip Rev RNA 5(4):549-64 (2014) and Zhang et al., “Purification, Characterization, and cDNA Cloning of an AU-rich Element RNA-binding Protein, AUF1,” Mol. Cell. Biol. 13(12):7652-65 (1993), which are hereby incorporated by reference in their entirety), and tissue stem cell fate-determining mRNAs (Chenette et al., “Targeted mRNA Decay by RNA Binding Protein AUF1 Regulates Adult Muscle Stem Cell Fate, Promoting Skeletal Muscle Integrity,” Cell Rep. 16(5):1379-90 (2016), which is hereby incorporated by reference in its entirety) that have very short half-lives of 5-30 minutes.

Short-lived mRNAs typically contain an AU-rich element (“ARE”) in the 3′ untranslated region (“3′UTR”) of the mRNA, having the repeated sequence AUUUA (Moore et al., “Physiological Networks and Disease Functions of RNA-binding Protein AUF1,” Wiley Interdiscip Rev. RNA 5(4):549-64 (2014), which is hereby incorporated by reference in its entirety), which confers rapid decay. The ARE serves as a binding site for regulatory proteins known as AU-rich binding proteins (AUBPs) that control the stability and in some cases the translation of the mRNA (Moore et al., “Physiological Networks and Disease Functions of RNA-binding Protein AUF1,” Wiley Interdiscip. Rev. RNA 5(4):549-64 (2014); Zhang et al., “Purification, Characterization, and cDNA Cloning of an AU-rich Element RNA-binding Protein, AUF1,” Mol. Cell. Biol. 13(12):7652-65 (1993); and Halees et al., “ARED Organism: Expansion of ARED Reveals AU-rich Element Cluster Variations Between Human And Mouse,” Nucleic Acids Res 36(Database issue):D137-40 (2008), which are hereby incorporated by reference in their entirety).

AU-rich mRNA binding factor 1 (AUF1; HNRNPD) binds with high affinity to repeated AU-rich elements (“AREs”) located in the 3′ untranslated region (“3′ UTR”) found in approximately 5% of mRNAs. Although AUF1 typically targets ARE-mRNAs for rapid degradation, while not as well understood, it can oppositely stabilize and increase the translation of some ARE-mRNAs (Moore et al., “Physiological Networks and Disease Functions of RNA-Binding Protein AUF1,” Wiley Interdiscip. Rev. RNA 5(4):549-564 (2014), which is hereby incorporated by reference in its entirety). It was previously reported that mice with AUF1 deficiency undergo an accelerated loss of muscle mass due to an inability to carry out the myogenesis program (Chenette et al., “Targeted mRNA Decay by RNA Binding Protein AUF1 Regulates Adult Muscle Stem Cell Fate, Promoting Skeletal Muscle Integrity,” Cell Rep. 16(5):1379-90 (2016), which is hereby incorporated by reference in its entirety). It was also found that AUF1 expression is severely reduced with age in skeletal muscle, and this significantly contributes to loss and atrophy of muscle, loss of muscle mass, and reduced strength (Abbadi et al., “Muscle Development and Regeneration Controlled by AUF1-mediated Stage-specific Degradation of Fate-determining Checkpoint mRNAs,” Proc. Natl. Acad. Sci. USA 116(23):11285-11290 (2019), which is hereby incorporated by reference in its entirety). It was also found that AUF1 controls all major stages of skeletal muscle development, starting with satellite cell activation and lineage commitment, by selectively targeting for rapid degradation the major differentiation checkpoint mRNAs that block entry into each next step of muscle development.

AUF1 has four related protein isoforms identified by their molecular weight (p37^{AUF1}, p40^{AUF1}, p42^{AUF1}, p45^{AUF1}) derived by differential splicing of a single pre-mRNA (Moore et al., “Physiological Networks and Disease Functions of RNA-Binding Protein AUF1,” Wiley Interdiscip. Rev. RNA 5(4):549-564 (2014); Chen & Shyu, “AU-Rich Elements: Characterization and Importance in mRNA Degradation,” Trends Biochem. Sci. 20(11):465-470 (1995); and Kim et al., “Emerging Roles of RNA and RNA-Binding Protein Network in Cancer Cells,” BMB Rep. 42(3):125-130 (2009), which are hereby incorporated by reference in their entirety). Each of these four isoforms include two centrally-positioned, tandemly arranged RNA recognition motifs (“RRMs”) which mediate RNA binding (DeMaria et al., “Structural Determinants in AUF 1 Required for High Affinity Binding to A+U-rich Elements,” J. Biol. Chem. 272:27635-27643 (1997), which is hereby incorporated by reference in its entirety).

The general organization of an RRM is a β-α-β-β-α-β RNA binding platform of anti-parallel P-sheets backed by the a-helices (Zucconi & Wilson, “Modulation of Neoplastic Gene Regulatory Pathways by the RNA-binding Factor AUF1,” Front. Biosci. 16:2307-2325 (2013); Nagai et al., “The RNP Domain: A Sequence-specific RNA-binding Domain Involved in Processing and Transport of RNA,” Trends Biochem. Sci. 20:235-240 (1995), which are hereby incorporated by reference in their entirety). Structures of individual AUF1 RRM domains resolved by NMR are largely consistent with this overall tertiary fold (Zucconi & Wilson, “Modulation of Neoplastic Gene Regulatory Pathways by the RNA-binding Factor AUF1,” Front. Biosci. 16:2307-2325 (2013); Nagata et al., “Structure and Interactions with RNA of the N-terminal UUAG-specific RNA-binding Domain of hnRNP DO,” J. Mol. Biol. 287:221-237 (1999); and Katahira et al., “Structure of the C-terminal RNA-binding Domain of hnRNP D0 (AUF1), its Interactions with RNA and DNA, and Change in Backbone Dynamics Upon Complex Formation with DNA,” J. Mol. Biol. 311:973-988 (2001), which are hereby incorporated by reference in their entirety).

Mutations and/or polymorphisms in AUF1 are linked to human limb girdle muscular dystrophy (LGMD) type 1G (Chenette et al., “Targeted mRNA Decay by RNA Binding Protein AUF1 Regulates Adult Muscle Stem Cell Fate, Promoting Skeletal Muscle Integrity,” Cell Rep. 16(5):1379-1390 (2016), which is hereby incorporated by reference in its entirety), suggesting a critical requirement for AUF1 in post-natal skeletal muscle regeneration and maintenance.

The term “fragment” or “portion” when used herein with respect to a given polypeptide sequence (e.g., AUF1), refers to a contiguous stretch of amino acids of the given polypeptide's sequence that is shorter than the given polypeptide's full-length sequence. A fragment of a polypeptide may be defined by its first position and its final position, in which the first and final positions each correspond to a position in the sequence of the given full-length polypeptide. The sequence position corresponding to the first position is situated N-terminal to the sequence position corresponding to the final position. The sequence of the fragment or portion is the contiguous amino acid sequence or stretch of amino acids in the given polypeptide that begins at the sequence position corresponding to the first position and ends at the sequence position corresponding to the final position. Functional or active fragments are fragments that retain functional characteristics, e.g., of the native sequence or other reference sequence. Typically, active fragments are fragments that retain substantially the same activity as the wild-type protein. A fragment may, for example, contain a functionally important domain, such as a domain that is important for receptor or ligand binding. Functional fragments are at least 10, 15, 20, 50, 75, 100, 150, 200, 250 or 300 contiguous amino acids of a full length AUF1 (including the p37, p40, p42 or p45 isoforms thereof) and retain one or more AUF1 functions.

Accordingly, in certain embodiments, functional fragments of AUF1 as described herein include at least one RNA recognition domain (“RRM”) domain. In certain embodiments, functional fragments of AUF1 as described herein include two RRM domains.

AUF1 or functional fragments thereof as described herein may be derived from a mammalian AUF1. In one embodiment, the AUF1 or functional fragment thereof is a human AUF1 or functional fragment thereof. In another embodiment, the AUF1 or functional fragment thereof is a murine AUF1 or a functional fragment thereof. The AUF1 protein according to embodiments described herein may include one or more of the AUF1 isoforms p37^{AUF1}, p40^{AUF1}, p42^{AUF1}, and p45^{AUF1}. The GenBank accession numbers corresponding to the nucleotide and amino acid sequences of each human and mouse isoform is found in Table 1 below, each of which is hereby incorporated by reference in its entirety.

TABLE 1
Summary of GenBank Accession Numbers of AUF1 Sequences

| | Human | | Mouse | |
|---|---|---|---|---|
| Isoform | Nucleotide | Amino Acid | Nucleotide | Amino Acid |
| p37^{AUF1} | NM_001003810.2 | NP_001003810.1 | NM_001077267.2 | NP_001070735.1 |
| | (SEQ ID NO: 1) | (SEQ ID NO: 2) | (SEQ ID NO: 3) | (SEQ ID NO: 4) |
| p40^{AUF1} | NM_002138.3 | NP_002129.2 | NM_007516.3 | NP_031542.2 |
| | (SEQ ID NO: 5) | (SEQ ID NO: 6) | (SEQ ID NO: 7) | (SEQ ID NO: 8) |
| p42^{AUF1} | NM_031369.2 | NP_112737.1 | NM_001077266.2 | NP_001070734.1 |
| | (SEQ ID NO: 9) | (SEQ ID NO: 10) | (SEQ ID NO: 11) | (SEQ ID NO: 12) |
| p45^{AUF1} | NM_031370.2 | NP_112738.1 | NM_001077265.2 | NP_001070733.1 |
| | (SEQ ID NO: 13) | (SEQ ID NO: 14) | (SEQ ID NO: 15) | (SEQ ID NO: 16) |

The sequences referred to in Table 1 are reproduced below.

The human p37^{AUF1} nucleotide sequence of GenBank Accession No. NM_001003810.1
(SEQ ID NO: 1) is as follows:

| | |
|---|---|
| CTTCCGTCGG CCATTTTAGG TGGTCCGCGG CGGCGCCATT AAAGCGAGGA GGAGGCGAGA | 60 |
| GCGGCCGCCG CTGGTGCTTA TTCTTTTTTA GTGCAGCGGG AGAGAGCGGG AGTGTGCGCC | 120 |
| GCGCGAGAGT GGGAGGCGAA GGGGGCAGGC CAGGGAGAGG CGCAGGAGCC TTTGCAGCCA | 180 |
| CGCGCGCGCC TTCCCTGTCT TGTGTGCTTC GCGAGGTAGA GCGGGCGCGC GGCAGCGGCG | 240 |
| GGGATTACTT TGCTGCTAGT TTCGGTTCGC GGCAGCGGCG GGTGTAGTCT CGGCGGCAGC | 300 |
| GGCGGAGACA CTAGCACTAT GTCGGAGGAG CAGTTCGGCG GGGACGGGGC GGCGGCAGCG | 360 |
| GCAACGGCGG CGGTAGGCGG CTCGGCGGGC GAGCAGGAGG GAGCCATGGT GGCGGCGACA | 420 |
| CAGGGGGCAG CGGCGGCGGC GGGAAGCGGA GCCGGGACCG GGGGCGGAAC CGCGTCTGGA | 480 |
| GGCACCGAAG GGGGCAGCGC CGAGTCGGAG GGGGCGAAGA TTGACGCCAG TAAGAACGAG | 540 |
| GAGGATGAAG GGAAAATGTT TATAGGAGGC CTTAGCTGGG ACACTACAAA GAAAGATCTG | 600 |
| AAGGACTACT TTTCCAAATT TGGTGAAGTT GTAGACTGCA CTCTGAAGTT AGATCCTATC | 660 |
| ACAGGGCGAT CAAGGGGTTT TGGCTTTGTG CTATTTAAAG AATCGGAGAG TGTAGATAAG | 720 |
| GTCATGGATC AAAAAGAACA TAAATTGAAT GGGAAGGTGA TTGATCCTAA AAGGGCCAAA | 780 |
| GCCATGAAAA CAAAAGAGCC GGTTAAAAAA ATTTTTGTTG GTGGCCTTTC TCCAGATACA | 840 |
| CCTGAAGAGA AAATAAGGGA GTACTTTGGT GGTTTTGGTG AGGTGGAATC CATAGAGCTC | 900 |
| CCCATGGACA ACAAGACCAA TAAGAGGCGT GGGTTCTGCT TTATTACCTT TAAGGAAGAA | 960 |
| GAACCAGTGA AGAAGATAAT GGAAAAGAAA TACCACAATG TTGGTCTTAG TAAATGTGAA | 1020 |
| ATAAAAGTAG CCATGTCGAA GGAACAATAT CAGCAACAGC AACAGTGGGG ATCTAGAGGA | 1080 |
| GGATTTGCAG GAAGAGCTCG TGGAAGAGGT GGTGACCAGC AGAGTGGTTA TGGGAAGGTA | 1140 |
| TCCAGGCGAG GTGGTCATCA AAATAGCTAC AAACCATACT AAATTATTCC ATTTGCAACT | 1200 |
| TATCCCCAAC AGGTGGTGAA GCAGTATTTT CCAATTTGAA GATTCATTTG AAGGTGGCTC | 1260 |
| CTGCCACCTG CTAATAGCAG TTCAAACTAA ATTTTTTGTA TCAAGTCCCT GAATGGAAGT | 1320 |
| ATGACGTTGG GTCCCTCTGA AGTTTAATTC TGAGTTCTCA TTAAAAGAAA TTTGCTTTCA | 1380 |
| TTGTTTTATT TCTTAATTGC TATGCTTCAG AATCAATTTG TGTTTTATGC CCTTTCCCCC | 1440 |
| AGTATTGTAG AGCAAGTCTT GTGTTAAAAG CCCAGTGTGA CAGTGTCATG ATGTAGTAGT | 1500 |
| GTCTTACTGG TTTTTTAATA AATCCTTTTG TATAAAAATG TATTGGCTCT TTTATCATCA | 1560 |
| GAATAGGAAA AATTGTCATG GATTCAAGTT ATTAAAAGCA TAAGTTTGGA AGACAGGCTT | 1620 |
| GCCGAAATTG AGGACATGAT TAAAATTGCA GTGAAGTTTG AAATGTTTTT AGCAAAATCT | 1680 |
| AATTTTTGCC ATAATGTGTC CTCCCTGTCC AAATTGGGAA TGACTTAATG TCAATTTGTT | 1740 |
| TGTTGGTTGT TTTAATAATA CTTCCTTATG TAGCCATTAA GATTTATATG AATATTTTCC | 1800 |
| CAAATGCCCA GTTTTTGCTT AATATGTATT GTGCTTTTTA GAACAAATCT GGATAAATGT | 1860 |
| GCAAAAGTAC CCCTTTGCAC AGATAGTTAA TGTTTTATGC TTCCATTAAA TAAAAAGGAC | 1920 |
| TTAAAATCTG TTAATTATAA TAGAAATGCG GCTAGTTCAG AGAGATTTTT AGAGCTGTGG | 1980 |
| TGGACTTCAT AGATGAATTC AAGTGTTGAG GGAGGATTAA AGAAATATAT ACCGTGTTTA | 2040 |
| TGTGTGTGTG CTT | |

The human p37^{AUF1} amino acid sequence of GenBank Accession No. NP_001003810.1
(SEQ ID NO: 2) is as follows:

| | |
|---|---|
| MSEEQFGGDG AAAAATAAVG GSAGEQEGAM VAATQGAAAA AGSGAGTGGG TASGGTEGGS | 60 |
| AESEGAKIDA SKNEEDEGKM FIGGLSWDTT KKDLKDYFSK FGEVVDCTLK LDPITGRSRG | 120 |
| FGFVLFKESE SVDKVMDQKE HKLNGKVIDP KRAKAMKTKE PVKKIFVGGL SPDTPEEKIR | 180 |
| EYFGGFGEVE SIELPMDNKT NKRRGFCFIT FKEEEPVKKI MEKKYHNVGL SKCEIKVAMS | 240 |
| KEQYQQQQQW GSRGGFAGRA RGRGGDQQSG YGKVSRRGGH QNSYKPY | |

The human p40^{AUF1} nucleotide sequence of GenBank Accession No. NM_002138.3
(SEQ ID NO: 5) is as follows:

| | |
|---|---|
| CTTCCGTCGG CCATTTTAGG TGGTCCGCGG CGGCGCCATT AAAGCGAGGA GGAGGCGAGA | 60 |
| GCGGCCGCCG CTGGTGCTTA TTCTTTTTTA GTGCAGCGGG AGAGAGCGGG AGTGTGCGCC | 120 |
| GCGCGAGAGT GGGAGGCGAA GGGGGCAGGC CAGGGAGAGG CGCAGGAGCC TTTGCAGCCA | 180 |
| CGCGCGCGCC TTCCCTGTCT TGTGTGCTTC GCGAGGTAGA GCGGGCGCGC GGCAGCGGCG | 240 |
| GGGATTACTT TGCTGCTAGT TTCGGTTCGC GGCAGCGGCG GGTGTAGTCT CGGCGGCAGC | 300 |
| GGCGGAGACA CTAGCACTAT GTCGGAGGAG CAGTTCGGCG GGGACGGGGC GGCGGCAGCG | 360 |
| GCAACGGCGG CGGTAGGCGG CTCGGCGGGC GAGCAGGAGG GAGCCATGGT GGCGGCGACA | 420 |
| CAGGGGGCAG CGGCGGCGGC GGGAAGCGGA GCCGGGACCG GGGGCGGAAC CGCGTCTGGA | 480 |
| GGCACCGAAG GGGGCAGCGC CGAGTCGGAG GGGGCGAAGA TTGACGCCAG TAAGAACGAG | 540 |
| GAGGATGAAG GCCATTCAAA CTCCTCCCCA CGACACTCTG AAGCAGCGAC GGCACAGCGG | 600 |
| GAAGAATGGA AAATGTTTAT AGGAGGCCTT AGCTGGGACA CTACAAAGAA AGATCTGAAG | 660 |
| GACTACTTTT CCAAATTTGG TGAAGTTGTA GACTGCACTC TGAAGTTAGA TCCTATCACA | 720 |
| GGGCGATCAA GGGGTTTTGG CTTTGTGCTA TTTAAAGAAT CGGAGAGTGT AGATAAGGTC | 780 |
| ATGGATCAAA AAGAACATAA ATTGAATGGG AAGGTGATTG ATCCTAAAAG GGCCAAAGCC | 840 |
| ATGAAAACAA AAGAGCCGGT TAAAAAAATT TTTGTTGGTG GCCTTTCTCC AGATACACCT | 900 |
| GAAGAGAAAA TAAGGGAGTA CTTTGGTGGT TTTGGTGAGG TGGAATCCAT AGAGCTCCCC | 960 |
| ATGGACAACA AGACCAATAA GAGGCGTGGG TTCTGCTTTA TTACCTTTAA GGAAGAAGAA | 1020 |
| CCAGTGAAGA AGATAATGGA AAAGAAATAC CACAATGTTG GTCTTAGTAA ATGTGAAATA | 1080 |
| AAAGTAGCCA TGTCGAAGGA ACAATATCAG CAACAGCAAC AGTGGGGATC TAGAGGAGGA | 1140 |
| TTTGCAGGAA GAGCTCGTGG AAGAGGTGGT GACCAGCAGA GTGGTTATGG GAAGGTATCC | 1200 |
| AGGCGAGGTG GTCATCAAAA TAGCTACAAA CCATACTAAA TTATTCCATT TGCAACTTAT | 1260 |
| CCCCAACAGG TGGTGAAGCA GTATTTTCCA ATTTGAAGAT TCATTTGAAG GTGGCTCCTG | 1320 |
| CCACCTGCTA ATAGCAGTTC AAACTAAATT TTTTGTATCA AGTCCCTGAA TGGAAGTATG | 1380 |
| ACGTTGGGTC CCTCTGAAGT TTAATTCTGA GTTCTCATTA AAAGAAATTT GCTTTCATTG | 1440 |
| TTTTATTTCT TAATTGCTAT GCTTCAGAAT CAATTTGTGT TTTATGCCCT TTCCCCCAGT | 1500 |
| ATTGTAGAGC AAGTCTTGTG TTAAAAGCCC AGTGTGACAG TGTCATGATG TAGTAGTGTC | 1560 |
| TTACTGGTTT TTTAATAAAT CCTTTTGTAT AAAAATGTAT TGGCTCTTTT ATCATCAGAA | 1620 |
| TAGGAAAAAT TGTCATGGAT TCAAGTTATT AAAAGCATAA GTTTGGAAGA CAGGCTTGCC | 1680 |
| GAAATTGAGG ACATGATTAA AATTGCAGTG AAGTTTGAAA TGTTTTTAGC AAAATCTAAT | 1740 |
| TTTTGCCATA ATGTGTCCTC CCTGTCCAAA TTGGGAATGA CTTAATGTCA ATTTGTTTGT | 1800 |
| TGGTTGTTTT AATAATACTT CCTTATGTAG CCATTAAGAT TTATATGAAT ATTTTCCCAA | 1860 |
| ATGCCCAGTT TTTGCTTAAT ATGTATTGTG CTTTTTAGAA CAAATCTGGA TAAATGTGCA | 1920 |
| AAAGTACCCC TTTGCACAGA TAGTTAATGT TTTATGCTTC CATTAAATAA AAAGGACTTA | 1980 |
| AAATCTGTTA ATTATAATAG AAATGCGGCT AGTTCAGAGA GATTTTTAGA GCTGTGGTGG | 2040 |
| ACTTCATAGA TGAATTCAAG TGTTGAGGGA GGATTAAAGA AATATATACC GTGTTTATGT | 2100 |
| GTGTGTGCTT | |

The human p40^{AUF1} amino acid sequence of GenBank Accession No. NP_002129.2
(SEQ ID NO: 6) is as follows:

| | |
|---|---|
| MSEEQFGGDG AAAAATAAVG GSAGEQEGAM VAATQGAAAA AGSGAGTGGG TASGGTEGGS | 60 |
| AESEGAKIDA SKNEEDEGHS NSSPRHSEAA TAQREEWKMF IGGLSWDTTK KDLKDYFSKF | 120 |
| GEVVDCTLKL DPITGRSRGF GFVLFKESES VDKVMDQKEH KLNGKVIDPK RAKAMKTKEP | 180 |
| VKKIFVGGLS PDTPEEKIRE YFGGFGEVES IELPMDNKIN KRRGFCFITF KEEEPVKKIM | 240 |
| EKKYHNVGLS KCEIKVAMSK EQYQQQQQWG SRGGFAGRAR GRGGDQQSGY GKVSRRGGHQ | 300 |

NSYKPY
The human p42^{AUF1} nucleotide sequence of GenBank Accession No. NM_031369.2
(SEQ ID NO: 9) is as follows:

| | |
|---|---|
| CTTCCGTCGG CCATTTTAGG TGGTCCGCGG CGGCGCCATT AAAGCGAGGA GGAGGCGAGA | 60 |
| GCGGCCGCCG CTGGTGCTTA TTCTTTTTTA GTGCAGCGGG AGAGAGCGGG AGTGTGCGCC | 120 |
| GCGCGAGAGT GGGAGGCGAA GGGGGCAGGC CAGGGAGAGG CGCAGGAGCC TTTGCAGCCA | 180 |
| CGCGCGCGCC TTCCCTGTCT TGTGTGCTTC GCGAGGTAGA GCGGGCGCGC GGCAGCGGCG | 240 |
| GGGATTACTT TGCTGCTAGT TTCGGTTCGC GGCAGCGGCG GGTGTAGTCT CGGCGGCAGC | 300 |
| GGCGGAGACA CTAGCACTAT GTCGGAGGAG CAGTTCGGCG GGGACGGGGC GGCGGCAGCG | 360 |
| GCAACGGCGG CGGTAGGCGG CTCGGCGGGC GAGCAGGAGG GAGCCATGGT GGCGGCGACA | 420 |
| CAGGGGGCAG CGGCGGCGGC GGGAAGCGGA GCCGGGACCG GGGGCGGAAC CGCGTCTGGA | 480 |
| GGCACCGAAG GGGGCAGCGC CGAGTCGGAG GGGGCGAAGA TTGACGCCAG TAAGAACGAG | 540 |
| GAGGATGAAG GGAAAATGTT TATAGGAGGC CTTAGCTGGG ACACTACAAA GAAAGATCTG | 600 |
| AAGGACTACT TTTCCAAATT TGGTGAAGTT GTAGACTGCA CTCTGAAGTT AGATCCTATC | 660 |
| ACAGGGCGAT CAAGGGGTTT TGGCTTTGTG CTATTTAAAG AATCGGAGAG TGTAGATAAG | 720 |
| GTCATGGATC AAAAAGAACA TAAATTGAAT GGGAAGGTGA TTGATCCTAA AAGGGCCAAA | 780 |
| GCCATGAAAA CAAAAGAGCC GGTTAAAAAA ATTTTTGTTG GTGGCCTTTC TCCAGATACA | 840 |
| CCTGAAGAGA AAATAAGGGA GTACTTTGGT GGTTTTGGTG AGGTGGAATC CATAGAGCTC | 900 |
| CCCATGGACA ACAAGACCAA TAAGAGGCGT GGGTTCTGCT TTATTACCTT TAAGGAAGAA | 960 |
| GAACCAGTGA AGAAGATAAT GGAAAAGAAA TACCACAATG TTGGTCTTAG TAAATGTGAA | 1020 |
| ATAAAAGTAG CCATGTCGAA GGAACAATAT CAGCAACAGC AACAGTGGGG ATCTAGAGGA | 1080 |
| GGATTTGCAG GAAGAGCTCG TGGAAGAGGT GGTGGCCCCA GTCAAAACTG GAACCAGGGA | 1140 |
| TATAGTAACT ATTGGAATCA AGGCTATGGC AACTATGGAT ATAACAGCCA AGGTTACGGT | 1200 |
| GGTTATGGAG GATATGACTA CACTGGTTAC AACAACTACT ATGGATATGG TGATTATAGC | 1260 |
| AACCAGCAGA GTGGTTATGG GAAGGTATCC AGGCGAGGTG GTCATCAAAA TAGCTACAAA | 1320 |
| CCATACTAAA TTATTCCATT TGCAACTTAT CCCCAACAGG TGGTGAAGCA GTATTTTCCA | 1380 |
| ATTTGAAGAT TCATTTGAAG GTGGCTCCTG CCACCTGCTA ATAGCAGTTC AAACTAAATT | 1440 |
| TTTTGTATCA AGTCCCTGAA TGGAAGTATG ACGTTGGGTC CCTCTGAAGT TTAATTCTGA | 1500 |
| GTTCTCATTA AAAGAAATTT GCTTTCATTG TTTTATTTCT TAATTGCTAT GCTTCAGAAT | 1560 |
| CAATTTGTGT TTTATGCCCT TTCCCCCAGT ATTGTAGAGC AAGTCTTGTG TTAAAAGCCC | 1620 |
| AGTGTGACAG TGTCATGATG TAGTAGTGTC TTACTGGTTT TTTAATAAAT CCTTTTGTAT | 1680 |
| AAAAATGTAT TGGCTCTTTT ATCATCAGAA TAGGAAAAAT TGTCATGGAT TCAAGTTATT | 1740 |
| AAAAGCATAA GTTTGGAAGA CAGGCTTGCC GAAATTGAGG ACATGATTAA AATTGCAGTG | 1800 |
| AAGTTTGAAA TGTTTTTAGC AAAATCTAAT TTTTGCCATA ATGTGTCCTC CCTGTCCAAA | 1860 |
| TTGGGAATGA CTTAATGTCA ATTTGTTTGT TGGTTGTTTT AATAATACTT CCTTATGTAG | 1920 |
| CCATTAAGAT TTATATGAAT ATTTTCCCAA ATGCCCAGTT TTTGCTTAAT ATGTATTGTG | 1980 |
| CTTTTTAGAA CAAATCTGGA TAAATGTGCA AAAGTACCCC TTTGCACAGA TAGTTAATGT | 2040 |
| TTTATGCTTC CATTAAATAA AAAGGACTTA AAATCTGTTA ATTATAATAG AAATGCGGCT | 2100 |
| AGTTCAGAGA GATTTTTAGA GCTGTGGTGG ACTTCATAGA TGAATTCAAG TGTTGAGGGA | 2160 |
| GGATTAAAGA AATATATACC GTGTTTATGT GTGTGTGCTT | |

The human p42^{AUF1} amino acid sequence of GenBank Accession No. NP_112737.1
(SEQ ID NO: 10) is as follows:

| | |
|---|---|
| MSEEQFGGDG AAAAATAAVG GSAGEQEGAM VAATQGAAAA AGSGAGTGGG TASGGTEGGS | 61 |
| AESEGAKIDA SKNEEDEGKM FIGGLSWDTT KKDLKDYFSK FGEVVDCTLK LDPITGRSRG | 121 |
| FGFVLFKESE SVDKVMDQKE HKLNGKVIDP KRAKAMKTKE PVKKIFVGGL SPDTPEEKIR | 181 |
| EYFGGFGEVE SIELPMDNKT NKRRGFCFIT FKEEEPVKKI MEKKYHNVGL SKCEIKVAMS | 241 |
| KEQYQQQQQW GSRGGFAGRA RGRGGGPSQN WNQGYSNYWN QGYGNYGYNS QGYGGYGGYD | 301 |
| YTGYNNYYGY GDYSNQQSGY GKVSRRGGHQ NSYKPY | |

The human p45^{AUF1} nucleotide sequence of GenBank Accession No. NM_031370.2
(SEQ ID NO: 13) is as follows:

| | |
|---|---|
| CTTCCGTCGG CCATTTTAGG TGGTCCGCGG CGGCGCCATT AAAGCGAGGA GGAGGCGAGA | 60 |
| GCGGCCGCCG CTGGTGCTTA TTCTTTTTTA GTGCAGCGGG AGAGAGCGGG AGTGTGCGCC | 120 |
| GCGCGAGAGT GGGAGGCGAA GGGGGCAGGC CAGGGAGAGG CGCAGGAGCC TTTGCAGCCA | 180 |
| CGCGCGCGCC TTCCCTGTCT TGTGTGCTTC GCGAGGTAGA GCGGGCGCGC GGCAGCGGCG | 240 |
| GGGATTACTT TGCTGCTAGT TTCGGTTCGC GGCAGCGGCG GGTGTAGTCT CGGCGGCAGC | 300 |
| GGCGGAGACA CTAGCACTAT GTCGGAGGAG CAGTTCGGCG GGGACGGGGC GGCGGCAGCG | 360 |
| GCAACGGCGG CGGTAGGCGG CTCGGCGGGC GAGCAGGAGG GAGCCATGGT GGCGGCGACA | 420 |
| CAGGGGGCAG CGGCGGCGGC GGGAAGCGGA GCCGGGACCG GGGGCGGAAC CGCGTCTGGA | 480 |
| GGCACCGAAG GGGGCAGCGC CGAGTCGGAG GGGGCGAAGA TTGACGCCAG TAAGAACGAG | 540 |
| GAGGATGAAG GCCATTCAAA CTCCTCCCCA CGACACTCTG AAGCAGCGAC GGCACAGCGG | 600 |
| GAAGAATGGA AAATGTTTAT AGGAGGCCTT AGCTGGGACA CTACAAAGAA AGATCTGAAG | 660 |
| GACTACTTTT CCAAATTTGG TGAAGTTGTA GACTGCACTC TGAAGTTAGA TCCTATCACA | 720 |
| GGGCGATCAA GGGGTTTTGG CTTTGTGCTA TTTAAAGAAT CGGAGAGTGT AGATAAGGTC | 780 |
| ATGGATCAAA AAGAACATAA ATTGAATGGG AAGGTGATTG ATCCTAAAAG GGCCAAAGCC | 840 |
| ATGAAAACAA AAGAGCCGGT TAAAAAAATT TTTGTTGGTG GCCTTTCTCC AGATACACCT | 900 |
| GAAGAGAAAA TAAGGGAGTA CTTTGGTGGT TTTGGTGAGG TGGAATCCAT AGAGCTCCCC | 960 |
| ATGGACAACA AGACCAATAA GAGGCGTGGG TTCTGCTTTA TTACCTTTAA GGAAGAAGAA | 1020 |
| CCAGTGAAGA AGATAATGGA AAAGAAATAC CACAATGTTG GTCTTAGTAA ATGTGAAATA | 1080 |
| AAAGTAGCCA TGTCGAAGGA ACAATATCAG CAACAGCAAC AGTGGGGATC TAGAGGAGGA | 1140 |
| TTTGCAGGAA GAGCTCGTGG AAGAGGTGGT GGCCCCAGTC AAAACTGGAA CCAGGGATAT | 1200 |
| AGTAACTATT GGAATCAAGG CTATGGCAAC TATGGATATA ACAGCCAAGG TTACGGTGGT | 1260 |
| TATGGAGGAT ATGACTACAC TGGTTACAAC AACTACTATG GATATGGTGA TTATAGCAAC | 1320 |
| CAGCAGAGTG GTTATGGGAA GGTATCCAGG CGAGGTGGTC ATCAAAATAG CTACAAACCA | 1380 |
| TACTAAATTA TTCCATTTGC AACTTATCCC CAACAGGTGG TGAAGCAGTA TTTTCCAATT | 1440 |
| TGAAGATTCA TTTGAAGGTG GCTCCTGCCA CCTGCTAATA GCAGTTCAAA CTAAATTTTT | 1500 |
| TGTATCAAGT CCCTGAATGG AAGTATGACG TTGGGTCCCT CTGAAGTTTA ATTCTGAGTT | 1560 |
| CTCATTAAAA GAAATTTGCT TTCATTGTTT TATTTCTTAA TTGCTATGCT TCAGAATCAA | 1620 |
| TTTGTGTTTT ATGCCCTTTC CCCCAGTATT GTAGAGCAAG TCTTGTGTTA AAAGCCCAGT | 1680 |
| GTGACAGTGT CATGATGTAG TAGTGTCTTA CTGGTTTTTT AATAAATCCT TTTGTATAAA | 1740 |
| AATGTATTGG CTCTTTTATC ATCAGAATAG GAAAAATTGT CATGGATTCA AGTTATTAAA | 1800 |
| AGCATAAGTT TGGAAGACAG GCTTGCCGAA ATTGAGGACA TGATTAAAAT TGCAGTGAAG | 1860 |
| TTTGAAATGT TTTTAGCAAA ATCTAATTTT TGCCATAATG TGTCCTCCCT GTCCAAATTG | 1920 |
| GGAATGACTT AATGTCAATT TGTTTGTTGG TTGTTTTAAT AATACTTCCT TATGTAGCCA | 1980 |
| TTAAGATTTA TATGAATATT TTCCCAAATG CCCAGTTTTT GCTTAATATG TATTGTGCTT | 2040 |
| TTTAGAACAA ATCTGGATAA ATGTGCAAAA GTACCCCTTT GCACAGATAG TTAATGTTTT | 2100 |
| ATGCTTCCAT TAAATAAAAA GGACTTAAAA TCTGTTAATT ATAATAGAAA TGCGGCTAGT | 2160 |
| TCAGAGAGAT TTTTAGAGCT GTGGTGGACT TCATAGATGA ATTCAAGTGT TGAGGGAGGA | 2220 |
| TTAAAGAAAT ATATACCGTG TTTATGTGTG TGTGCTT | |

The human p45^{AUF1} amino acid sequence of GenBank Accession No. NP_112738.1
(SEQ ID NO: 14) is as follows:

| | |
|---|---|
| MSEEQFGGDG AAAAATAAVG GSAGEQEGAM VAATQGAAAA AGSGAGTGGG TASGGTEGGS | 60 |
| AESEGAKIDA SKNEEDEGHS NSSPRHSEAA TAQREEWKMF IGGLSWDTTK KDLKDYFSKF | 120 |
| GEVVDCTLKL DPITGRSRGF GFVLFKESES VDKVMDQKEH KLNGKVIDPK RAKAMKTKEP | 180 |
| VKKIFVGGLS PDTPEEKIRE YFGGFGEVES IELPMDNKIN KRRGFCFITF KEEEPVKKIM | 240 |
| EKKYHNVGLS KCEIKVAMSK EQYQQQQQWG SRGGFAGRAR GRGGGPSQNW NQGYSNYWNQ | 300 |
| GYGNYGYNSQ GYGGYGGYDY TGYNNYYGYG DYSNQQSGYG KVSRRGGHQN SYKPY | |

The mouse p37^{AUF1} nucleotide sequence of GenBank Accession No. NM_001077267.2
(SEQ ID NO: 3) is as follows:

| | |
|---|---|
| CCATTTTAGG TGGTCCGCGG CGGCGCCATT AAAGCGAGGA GGAGGCGAGA GTGGCCGCCG | 60 |
| CTGCTACTTC ATTCTTTTTT TTTTCAGTGC AGCCGGGGAG AGCGAGAGAG CGCGCTGCGC | 120 |
| GAGAGTGGGA GGCGAGGGGG GCAGGCCGGG GAGAGGCGCA GGAGCCCTTG CAGCCACGCG | 180 |
| CGCGCCTTGT CTAGGGTGCC TCGCGAGGTA GAGCGGGCAT CGCGCGGCGG CGGCGGGGAT | 240 |
| TACTTTGCTG CTAGTTTCGG TTCGCGGCGG CGGCGGCGTC GGCGGGTGTC GTCTTCGGCG | 300 |
| GCGGCAGTAG CACTATGTCG GAGGAGCAGT TCGGAGGGGA CGGGGCGGCG GCGGCGGCAA | 360 |
| CGGCGGCGGT AGGCGGCTCG GCGGGCGAGC AGGAGGGAGC CATGGTGGCG GCGGCGGCGC | 420 |
| AGGGGCCGGC GGCGGCGGCG GGAAGCGGGA GCGGCGGCGG CGGCTCTGCG GCCGGAGGCA | 480 |
| CCGAAGGAGG CAGCGCCGAG GCAGAGGGAG CCAAGATCGA CGCCAGTAAG AACGAGGAGG | 540 |
| ATGAAGGGAA AATGTTTATA GGAGGCCTTA GCTGGGACAC CACAAAGAAA GATCTGAAGG | 600 |
| ACTACTTTTC CAAATTTGGT GAAGTTGTAG ACTGCACTCT GAAGTTAGAT CCTATCACAG | 660 |
| GGCGATCAAG GGGTTTTGGC TTTGTGCTAT TTAAAGAGTC GGAGAGTGTA GATAAGGTCA | 720 |
| TGGATCAGAA AGAACATAAA TTGAATGGGA AAGTCATTGA TCCTAAAAGG GCCAAAGCCA | 780 |
| TGAAAACAAA AGAGCCTGTC AAAAAAATTT TTGTTGGTGG CCTTTCTCCA GACACACCTG | 840 |
| AAGAAAAAAT AAGAGAGTAC TTTGGTGGTT TTGGTGAGGT TGAATCCATA GAGCTCCCTA | 900 |
| TGGACAACAA GACCAATAAG AGGCGTGGGT TCTGTTTTAT TACCTTTAAG GAAGAGGAGC | 960 |
| CAGTGAAGAA GATAATGGAA AAGAAATACC ACAATGTTGG TCTTAGTAAA TGTGAAATAA | 1020 |
| AAGTAGCCAT GTCAAAGGAA CAGTATCAGC AGCAGCAGCA GTGGGGATCT AGAGGAGGGT | 1080 |
| TTGCAGGCAG AGCTCGCGGA AGAGGTGGAG ATCAGCAGAG TGGTTATGGG AAAGTATCCA | 1140 |
| GGCGAGGTGG ACATCAAAAT AGCTACAAAC CATACTAAAT TATTCCATTT GCAACTTATC | 1200 |
| CCCAACAGGT GGTGAAGCAG TATTTTCCAA TTTGAAGATT CATTTGAAGG TGGCTCCTGC | 1260 |
| CACCTGCTAA TAGCAGTTCA AACTAAATTT TTTCTATCAA GTTCCTGAAT GGAAGTATGA | 1320 |
| CGTTGGGTCC CTCTGAAGTT TAATTCTGAG TTCTCATTAA AAGAATTTGC TTTCATTGTT | 1380 |
| TTATTTCTTA ATTGCTATGC TTCAGTATCA ATTTGTGTTT TATGCCCCCC CTCCCCCCCA | 1440 |
| GTATTGTAGA GCAAGTCTTG TGTTAAAAAA AGCCCAGTGT GACAGTGTCA TGATGTAGTA | 1500 |
| GTGTCTTACT GGTTTTTTAA TAAATCCTTT TGTATAAAAA TGTATTGGCT CTTTTATCAT | 1560 |
| CAGAATAGGA GGAAGTGAAA TACTACAAAT GTTTGTCTTG GATTCAAGTC ACTAGAAGCA | 1620 |
| TAAATTTGAG GGGATAAAAA CAACGGTAAA CTTTGTCTGA AAGAGGGCAT GGTTAAAAAT | 1680 |
| GTAGTGAATT TTAAATGTTT TTAGCAAAAT TTGATTTTGC CCAAGAATCC CTGTCTGAAT | 1740 |
| TGGAAATGAC TTAATGTAGT CAATGTGCTT GTTGGTTGTC TTAATATTAC TTCTGTAGCC | 1800 |
| ATTAAGTTTT ATGAGTAACT TCCCAAATAC CCACGTTTTT CTTTATATGT ATTGTGCTTT | 1860 |
| TTAAAAACAA ATCTGGAAAA ATGGGCAAGA ACATTTGCAG ACAATTGTTT TTAAGCTTCC | 1920 |
| ATTAAATAAA AAAAATGTGG ACTTAAGGAA ATCTATTAAT TTAAATAGAA CTGCAGCTAG | 1980 |
| TTTAGAGAGT ATTTTTTTCT TAAAGCTTTG GTGTAATTAG GGAAGATTTT AAAAAATGCA | 2040 |
| TAGTGTTTAT TTGTATGTGT GCTCTTTTTT TAAGTCAATT TTTGGGGGGT TGGTCTGTTA | 2100 |
| ACTGAGTCTA GGATTTAAAG GTAAGATGTT CCTAGAAATC TTGTCATCCC AAAGGGGCGG | 2160 |
| GCGCTAAGGT GAAACTTCAG GGTTCAGTCA GGGTCACTGC TTTATGTGTG AAATCACTCA | 2220 |
| AATTGGTAAG TCTCTTATGT TAGCATTCAG GACATTGATT TCAACTTGGA TGGACAATTT | 2280 |
| ATAGTTACTA CTGAATTGTG TGTTAATGTG TTCAGTCCTG GTAAGTTTTC AGTTTGATCA | 2340 |
| GTTAGTTGGA AGCAGACTTG AAGAGCTGTT AGTCACGTGA GCCATGGGTG CAGTCGATCT | 2400 |
| GTGGTCAGAT GCCTGAGTCT GTGATAGTGA ATTGTGTCTA AAGACATTTT AATGATAAAA | 2460 |
| GTCAGTGCTG TAAAGTTGAA AGTTCATGAG AGACATACAA TGAGGGCTGC AGCCCATTTT | 2520 |
| TAAAAACATT ATAATACAAA AGTATGCACA TTTGTTTACA TATCCCTGCC TTTGTATTAC | 2580 |
| AGTGGCAGGT TTGTGTACTT AAACTGGGAA AGCCTCAGAT CTATGATTAC CTGGCCTATC | 2640 |
| ATAGAAAGTG TCTAAATAAA TCACTCTGTC AATTGAATAC ATTAGTATTA GCTAGCATAC | 2700 |
| TTCATTATGC CTGTTTTCCA TAAATACCAC ACCAAAAACT TGCTTGGGGC AGTTTGAGCC | 2760 |
| TAGTTCATGA GCTGCTATCA GATTGGTCTT GATCCTATAT AATAGGCCAA ATGTCTGTAA | 2820 |
| ACAGCTGTGC TGGTGGAATG TAGAAAGTCA CTGCACTCAG ATTCAACTTC CTGATTGGAA | 2880 |
| GTCATCACAG TGTGATTAAA CATTTTCACA AAGAATAGTA GATAAATAAC TTGGTTTTTA | 2940 |
| ATGTTAACTT TGTTTCCATT AAGTCACATT TAAAAACTTA TCCTCACGCC TACCTGAGTT | 3000 |
| AATTATCTGT TGACCTAGAT ATCTTTCTGG CCACTCACTG ACTTATTTCT TGAACTTTTG | 3060 |
| CCATTTGCAT AAATCTTGTC AGCTTTGTTC TTGATTATGC ATTGTCCAGG CTGAGCTAGT | 3120 |
| TGTCTTTCCA GGAATCCCTT TGTCTCTGAA TTAGGTCCTT TGTTTCCTAA ATCATCCTGC | 3180 |
| TTGTTTGGCA CAAGTCTTCC CAGGCCAGTG AGACCTCCGT GTCCTCTCAG CACCATAGGG | 3240 |
| GTAGGTAACC CTGGTTAGGC TGGACAGGGG TTTGCTGAGG GAGTTTGTTC ATTTGAATCT | 3300 |
| AGGTCTTACA TGACGTCTTT CAAATAGGGT TTTTACCTTG ACACTAAACT GTCCAGTCTA | 3360 |
| AGCAGTTCTG CAAAATGTGA GGGAATTATG AACTTCTTCC TGCAGTGGGT TTTTATGGTT | 3420 |
| TTGGTTTGTT TTTTGTTGTT TTGGTTCTTT GTTGAGCCCT GGACAAAAAC TTCCCTAGTT | 3480 |
| CTGGTTTCTA CAATTTAAAT TAAAAACAGA ATTCATCTTA GAATTTTTCA CCCTCTTCCC | 3540 |
| CAACTATTCT AATCAATCTT AAGTATGCCC TTCATCTTTT TTCCTTCCTA AGGCTTTTAC | 3600 |
| TGATAGTGTA ATTCCGTACT CTTCAACCCT GGGAAGGCTG AAGTGGATTC TTGAGCTCAT | 3660 |
| TTCAAGGCTG ACCTGGGTGT TGGCAAGAAC CCAGCTTAGA ACAAACACAT GCAAGGCCAT | 3720 |
| CTTACCTTAC ATCCTGTTGC TTGGACTTCT TCCTGCTCAA AGTTTTTAGT GGATGCTAAG | 3780 |
| TGATCTTTGC TTCCACTGAG GAGTGGAACA CTTTAGAATG AACCTCTAGA TAGATATTTT | 3840 |
| TATTGTCTGG TGAGGGTTAC TGGAGTTTCC CACCCTGCCT GAAGGGTGAA TCTGGCTTAC | 3900 |
| AGTGTTCTCA TCTCAAAGGG AAGAAGGCAG ATGGCTGTGT CCAGAGAGAG CCATCACAGT | 3960 |
| TTGCTTCAGA GACACTAGAA TGGGCTGGAA GATCTAGTGG TCTTAATCAG ACTTGAAACC | 4020 |
| TGGCCTTTCT TCATTACCCA TATGTCTACC AGTACTTGGG CTAACACTTA AGCCATTAGG | 4080 |
| GCCTTTGTAG GGGTGTTTTG AGACCCCCTC CATGCTAACA AATATACAGG TTTCTTAACA | 4140 |
| TTTGCTCATA AACTTGTAAA GCTTACTTTC TCTTAATCCA CCCCACATTT AACAAGCCCT | 4200 |
| GGTACTTAGA ATTTCAGAAG AGTAATGGCA GGTAGGTGTG TGTGTGTGTG TGTGTGTGTG | 4260 |
| TGTGTGTGTG TGTGTGTGAG AGAGAGAGAG AGAGAGAGAG AGAGAGAGAG AGAGAGAGAG | 4320 |
| AGAGAGAGAG AGAAGTTTGT GGAAAATCAG GTAATGACAG CTCATCCTTT TAGAATTGTA | 4380 |
| CTTCAGAATA GAAACATTTG GTGGGCTGTT AGGTAGCTTT GATTACTTGT GGGTAGACCT | 4440 |
| GCTAGTATTG CCAGTCCTCA AGCAATGAGC TTTCTGTATC TTGTTTACTA GATATATACT | 4500 |
| ACCAGGTGAG TCATTTCCTG GGGTTCTGTT TTCTTTTAAA ATCTTTCCCT AAACTTAATA | 4560 |
| TGTATTAAAA AGTCTGGCTT TTCAGTCCAT TCTTTGTGCA CTGGGATGGC AATTGCTTCA | 4620 |
| TTATATGACA ATTGCTGTTC CCAAGTCAGA ATTCAGTGTG CTGATTTGAC ATCAGTTCGT | 4680 |
| CCCGAATAAG TTCCTGTTAC CAGGATTTAC ATTCAGCACA TTAGAAACTT GTTGGTGTGC | 4740 |
| TTTTATTCTT GGAGCATTTT CCTTAGACTA CCTTCCACTT TGAGTGCTCT GTTTAGGATG | 4800 |
| TTGAGGTGTT AGGATTCTTG ACAGCCAGAA AGACTGAACC CACTATCTGG GCACAGTGTT | 4860 |
| CGTGTTGCTC TATAAATGTA TGCTTTTTTT GATTTGGGGT TGTTTTACCT ACATTGTCAA | 4920 |
| ACTAGATCCA TGCTTAACAG TGATAATGAA GGCTTTTTGT TTGTTTTGTT TGTGGGTCCT | 4980 |
| CCCCCCCCCC CCAAGACAGG GTTTCTCTGT AGGCTGTCCT AGAACTTGTT CTTTTTTAAC | 5040 |
| CAAAATTTGG CAAGGCTGAA AATGGAATCC TATAATCAAT GCTGGCCACA TTAAAGTTAA | 5100 |
| TAGTTGAGAA GTCTTGTCTG AATTTCCTTG GGCAAAAAGA TTCTAGCCAG TTCAATACCC | 5160 |
| TGTTGTGCAA ATTCAATTTG CTGTTATAAT TTGCTCTCAG TTATCAGTTG GAAGGAGGTT | 5220 |
| AATTCTAATG TACTTGGAAG AGGCCTGTAG ACCATCTATA ACTGCATCAG TTGTACAGCG | 5280 |
| TTGTTGCCTG GGATTCTCTA GTTCACATAA ACTCCCAAGT CTTAGCCGTG GTGATGGCTA | 5340 |
| CAGTGTGGAA GATGGTGAGC ATTCTAGTGA GTATCGCGAT GACGGCAGTA AAGAGCAGCA | 5400 |
| GGCAGCCGTG GCTGGGCTCA CTGACCGTGG CTGTAAGTTA CGGAGGCAGC ACACACTTCT | 5460 |
| GTACACACCT CTCATCAGTT ACCGGAGTCA TTGCATTGCG GACTAACTGG CTGACTCAAG | 5520 |
| TTGTCTTGCT ACTGAAGTCT TGAGTTGGTC TCATGCATTT ACCCTGTTGA CTTGAGCACC | 5580 |
| TTAAAGTCGA AAGGATGTCT GGTTGTGGCT TTATTGTAAA CAGCCTTAGG TAAAGAGGGG | 5640 |
| AGTATATCGG TTAGGAAGGT GAAAAATGAT ACTTCCAAGT TCAGTGGGAA ACCCTGGGTT | 5700 |
| TATCCCCCAG CTTAAGAAAG AATGCCTAAC AATGTTTCAG AATTAGATTC TGTGGAAGGT | 5760 |
| GAGGGTGTTA GAACAGTCCA AATTTGTTAT TGTAGACTTG CAGTGGGAGG AATTTTTAAA | 5820 |
| TATACAGATC AGTCGACACT CATTAACTTC ACTGATAAAG GTGGAAACGG ATGTGGCAAC | 5880 |
| ACTTCTAAGT TCATTTGTAT ATGTTTGTAA TTTGATTGGT TGTATTCTGT TGCACTCTAG | 5940 |
| AATTTGAAGG CAAGGTTACC TCTGCTTTTT AATTTTTTTT TTTTTAAAGA AAGAAAAAAC | 6000 |
| ACTGAAAGAA ACTTCAAAAG ATCTGTTAAT GCTAATACCT GAATGTGGCA TTTAACATGT | 6060 |
| CATGGAAACT GCTTTGAATA AATACTTGAG AAAAGGAATG AAATAATTGC CGTTTTTGTT | 6120 |
| GTTGAGTGAA TGGGTGTGGT TTAATGAGCG TAATCATTTT TATAAAACAG CTGTGAGACT | 6180 |
| GAAGTGGAAT CCTTATTAAA TGTGGAAAAT GGCCTTTGAG GATTACAGTA GAGATTCAAC | 6240 |
| TAAGAGAGTA AATAAAGCTT GAAACTAATT CGTTGTAAAT TGCTTCTACA ATCATTGCTC | 6300 |
| TATATAGCAT GCTATTGCCA ATCAGTTTTA TGTATTAAGA CCTATCAGCA TGTCTTTTTT | 6360 |
| AGGTTGACCT CATTTTAAAT TATAAGATGC TCTCTGTACC GTTTTAACAT TTCCAGGATT | 6420 |
| TATTCTTTCT AGGCAAATTC CACTGGACTG TTTCCATTGT AGAAGCTTCC TTATAGATTC | 6480 |
| TTCAAATGAA GCTTACAGTG TGCTTTCTTG GGGTTTTGAT TTGCACTAAA TTTTATTTTC | 6540 |
| TGAAAGATCA CTTATGTTTA TAATGTAGTG CTTTGTCTTA ACAATTAAAC TTTCCAGCAC | 6600 |

TCATGCA
The mouse p37^{AUF1} amino acid sequence of GenBank Accession No. NP_001070735.1
(SEQ ID NO: 4) is as follows:

| | |
|---|---|
| MSEEQFGGDG AAAAATAAVG GSAGEQEGAM VAAAAQGPAA AAGSGSGGGG SAAGGTEGGS | 60 |
| AEAEGAKIDA SKNEEDEGKM FIGGLSWDTT KKDLKDYFSK FGEVVDCTLK LDPITGRSRG | 120 |
| FGFVLFKESE SVDKVMDQKE HKLNGKVIDP KRAKAMKTKE PVKKIFVGGL SPDTPEEKIR | 180 |
| EYFGGFGEVE SIELPMDNKT NKRRGFCFIT FKEEEPVKKI MEKKYHNVGL SKCEIKVAMS | 240 |
| KEQYQQQQQW GSRGGFAGRA RGRGGDQQSG YGKVSRRGGH QNSYKPY | |

The mouse p40^{AUF1} nucleotide sequence of GenBank Accession No. NM_007516.3
(SEQ ID NO: 7) is as follows:

| | |
|---|---|
| CCATTTTAGG TGGTCCGCGG CGGCGCCATT AAAGCGAGGA GGAGGCGAGA GTGGCCGCCG | 60 |
| CTGCTACTTC ATTCTTTTTT TTTTCAGTGC AGCCGGGGAG AGCGAGAGAG CGCGCTGCGC | 120 |
| GAGAGTGGGA GGCGAGGGGG GCAGGCCGGG GAGAGGCGCA GGAGCCCTTG CAGCCACGCG | 180 |
| CGCGCCTTGT CTAGGGTGCC TCGCGAGGTA GAGCGGGCAT CGCGCGGCGG CGGCGGGGAT | 240 |
| TACTTTGCTG CTAGTTTCGG TTCGCGGCGG CGGCGGCGTC GGCGGGTGTC GTCTTCGGCG | 300 |
| GCGGCAGTAG CACTATGTCG GAGGAGCAGT TCGGAGGGGA CGGGGCGGCG GCGGCGGCAA | 360 |
| CGGCGGCGGT AGGCGGCTCG GCGGGCGAGC AGGAGGGAGC CATGGTGGCG GCGGCGGCGC | 420 |
| AGGGGCCGGC GGCGGCGGCG GGAAGCGGGA GCGGCGGCGG CGGCTCTGCG GCCGGAGGCA | 480 |
| CCGAAGGAGG CAGCGCCGAG GCAGAGGGAG CCAAGATCGA CGCCAGTAAG AACGAGGAGG | 540 |
| ATGAAGGCCA TTCAAACTCC TCCCCACGAC ACACTGAAGC AGCGGCGGCA CAGCGGGAAG | 600 |
| AATGGAAAAT GTTTATAGGA GGCCTTAGCT GGGACACCAC AAAGAAAGAT CTGAAGGACT | 660 |
| ACTTTTCCAA ATTTGGTGAA GTTGTAGACT GCACTCTGAA GTTAGATCCT ATCACAGGGC | 720 |
| GATCAAGGGG TTTTGGCTTT GTGCTATTTA AAGAGTCGGA GAGTGTAGAT AAGGTCATGG | 780 |
| ATCAGAAAGA ACATAAATTG AATGGGAAAG TCATTGATCC TAAAAGGGCC AAAGCCATGA | 840 |
| AAACAAAAGA GCCTGTCAAA AAAATTTTTG TTGGTGGCCT TTCTCCAGAC ACACCTGAAG | 900 |
| AAAAAATAAG AGAGTACTTT GGTGGTTTTG GTGAGGTTGA ATCCATAGAG CTCCCTATGG | 960 |
| ACAACAAGAC CAATAAGAGG CGTGGGTTCT GTTTTATTAC CTTTAAGGAA GAGGAGCCAG | 1020 |
| TGAAGAAGAT AATGGAAAAG AAATACCACA ATGTTGGTCT TAGTAAATGT GAAATAAAAG | 1080 |
| TAGCCATGTC AAAGGAACAG TATCAGCAGC AGCAGCAGTG GGGATCTAGA GGAGGGTTTG | 1140 |
| CAGGCAGAGC TCGCGGAAGA GGTGGAGATC AGCAGAGTGG TTATGGGAAA GTATCCAGGC | 1200 |
| GAGGTGGACA TCAAAATAGC TACAAACCAT ACTAAATTAT TCCATTTGCA ACTTATCCCC | 1260 |
| AACAGGTGGT GAAGCAGTAT TTTCCAATTT GAAGATTCAT TTGAAGGTGG CTCCTGCCAC | 1320 |
| CTGCTAATAG CAGTTCAAAC TAAATTTTTT CTATCAAGTT CCTGAATGGA AGTATGACGT | 1380 |
| TGGGTCCCTC TGAAGTTTAA TTCTGAGTTC TCATTAAAAG AATTTGCTTT CATTGTTTTA | 1440 |
| TTTCTTAATT GCTATGCTTC AGTATCAATT TGTGTTTTAT GCCCCCCCTC CCCCCCAGTA | 1500 |
| TTGTAGAGCA AGTCTTGTGT TAAAAAAAGC CCAGTGTGAC AGTGTCATGA TGTAGTAGTG | 1560 |
| TCTTACTGGT TTTTTAATAA ATCCTTTTGT ATAAAAATGT ATTGGCTCTT TTATCATCAG | 1620 |
| AATAGGAGGA AGTGAAATAC TACAAATGTT TGTCTTGGAT TCAAGTCACT AGAAGCATAA | 1680 |
| ATTTGAGGGG ATAAAAACAA CGGTAAACTT TGTCTGAAAG AGGGCATGGT TAAAAATGTA | 1740 |
| GTGAATTTTA AATGTTTTTA GCAAAATTTG ATTTTGCCCA AGAATCCCTG TCTGAATTGG | 1800 |
| AAATGACTTA ATGTAGTCAA TGTGCTTGTT GGTTGTCTTA ATATTACTTC TGTAGCCATT | 1860 |
| AAGTTTTATG AGTAACTTCC CAAATACCCA CGTTTTTCTT TATATGTATT GTGCTTTTTA | 1920 |
| AAAACAAATC TGGAAAAATG GGCAAGAACA TTTGCAGACA ATTGTTTTTA AGCTTCCATT | 1980 |
| AAATAAAAAA AATGTGGACT TAAGGAAATC TATTAATTTA AATAGAACTG CAGCTAGTTT | 2040 |
| AGAGAGTATT TTTTTCTTAA AGCTTTGGTG TAATTAGGGA AGATTTTAAA AAATGCATAG | 2100 |
| TGTTTATTTG TATGTGTGCT CTTTTTTTAA GTCAATTTTT GGGGGGTTGG TCTGTTAACT | 2160 |
| GAGTCTAGGA TTTAAAGGTA AGATGTTCCT AGAAATCTTG TCATCCCAAA GGGGGGGGCG | 2220 |
| CTAAGGTGAA ACTTCAGGGT TCAGTCAGGG TCACTGCTTT ATGTGTGAAA TCACTCAAAT | 2280 |
| TGGTAAGTCT CTTATGTTAG CATTCAGGAC ATTGATTTCA ACTTGGATGG ACAATTTATA | 2340 |
| GTTACTACTG AATTGTGTGT TAATGTGTTC AGTCCTGGTA AGTTTTCAGT TTGATCAGTT | 2400 |
| AGTTGGAAGC AGACTTGAAG AGCTGTTAGT CACGTGAGCC ATGGGTGCAG TCGATCTGTG | 2460 |
| GTCAGATGCC TGAGTCTGTG ATAGTGAATT GTGTCTAAAG ACATTTTAAT GATAAAAGTC | 2520 |
| AGTGCTGTAA AGTTGAAAGT TCATGAGAGA CATACAATGA GGGCTGCAGC CCATTTTTAA | 2580 |
| AAACATTATA ATACAAAAGT ATGCACATTT GTTTACATAT CCCTGCCTTT GTATTACAGT | 2640 |
| GGCAGGTTTG TGTACTTAAA CTGGGAAAGC CTCAGATCTA TGATTACCTG GCCTATCATA | 2700 |
| GAAAGTGTCT AAATAAATCA CTCTGTCAAT TGAATACATT AGTATTAGCT AGCATACTTC | 2760 |
| ATTATGCCTG TTTTCCATAA ATACCACACC AAAAACTTGC TTGGGGCAGT TTGAGCCTAG | 2820 |
| TTCATGAGCT GCTATCAGAT TGGTCTTGAT CCTATATAAT AGGCCAAATG TCTGTAAACA | 2880 |
| GCTGTGCTGG TGGAATGTAG AAAGTCACTG CACTCAGATT CAACTTCCTG ATTGGAAGTC | 2940 |
| ATCACAGTGT GATTAAACAT TTTCACAAAG AATAGTAGAT AAATAACTTG GTTTTTAATG | 3000 |
| TTAACTTTGT TTCCATTAAG TCACATTTAA AAACTTATCC TCACGCCTAC CTGAGTTAAT | 3060 |
| TATCTGTTGA CCTAGATATC TTTCTGGCCA CTCACTGACT TATTTCTTGA ACTTTTGCCA | 3120 |
| TTTGCATAAA TCTTGTCAGC TTTGTTCTTG ATTATGCATT GTCCAGGCTG AGCTAGTTGT | 3180 |
| CTTTCCAGGA ATCCCTTTGT CTCTGAATTA GGTCCTTTGT TTCCTAAATC ATCCTGCTTG | 3240 |
| TTTGGCACAA GTCTTCCCAG GCCAGTGAGA CCTCCGTGTC CTCTCAGCAC CATAGGGGTA | 3300 |
| GGTAACCCTG GTTAGGCTGG ACAGGGGTTT GCTGAGGGAG TTTGTTCATT TGAATCTAGG | 3360 |
| TCTTACATGA CGTCTTTCAA ATAGGGTTTT TACCTTGACA CTAAACTGTC CAGTCTAAGC | 3420 |
| AGTTCTGCAA AATGTGAGGG AATTATGAAC TTCTTCCTGC AGTGGGTTTT TATGGTTTTG | 3480 |
| GTTTGTTTTT TGTTGTTTTG GTTCTTTGTT GAGCCCTGGA CAAAAACTTC CCTAGTTCTG | 3540 |
| GTTTCTACAA TTTAAATTAA AAACAGAATT CATCTTAGAA TTTTTCACCC TCTTCCCCAA | 3600 |
| CTATTCTAAT CAATCTTAAG TATGCCCTTC ATCTTTTTTC CTTCCTAAGG CTTTTACTGA | 3660 |
| TAGTGTAATT CCGTACTCTT CAACCCTGGG AAGGCTGAAG TGGATTCTTG AGCTCATTTC | 3720 |
| AAGGCTGACC TGGGTGTTGG CAAGAACCCA GCTTAGAACA AACACATGCA AGGCCATCTT | 3780 |
| ACCTTACATC CTGTTGCTTG GACTTCTTCC TGCTCAAAGT TTTTAGTGGA TGCTAAGTGA | 3840 |
| TCTTTGCTTC CACTGAGGAG TGGAACACTT TAGAATGAAC CTCTAGATAG ATATTTTTAT | 3900 |
| TGTCTGGTGA GGGTTACTGG AGTTTCCCAC CCTGCCTGAA GGGTGAATCT GGCTTACAGT | 3960 |
| GTTCTCATCT CAAAGGGAAG AAGGCAGATG GCTGTGTCCA GAGAGAGCCA TCACAGTTTG | 4020 |
| CTTCAGAGAC ACTAGAATGG GCTGGAAGAT CTAGTGGTCT TAATCAGACT TGAAACCTGG | 4080 |
| CCTTTCTTCA TTACCCATAT GTCTACCAGT ACTTGGGCTA ACACTTAAGC CATTAGGGCC | 4140 |
| TTTGTAGGGG TGTTTTGAGA CCCCCTCCAT GCTAACAAAT ATACAGGTTT CTTAACATTT | 4200 |
| GCTCATAAAC TTGTAAAGCT TACTTTCTCT TAATCCACCC CACATTTAAC AAGCCCTGGT | 4260 |
| ACTTAGAATT TCAGAAGAGT AATGGCAGGT AGGTGTGTGT GTGTGTGTGT GTGTGTGTGT | 4320 |
| GTGTGTGTGT GTGTGAGAGA GAGAGAGAGA GAGAGAGAGA GAGAGAGAGA GAGAGAGAGA | 4380 |
| GAGAGAGAGA AGTTTGTGGA AAATCAGGTA ATGACAGCTC ATCCTTTTAG AATTGTACTT | 4440 |
| CAGAATAGAA ACATTTGGTG GGCTGTTAGG TAGCTTTGAT TACTTGTGGG TAGACCTGCT | 4500 |
| AGTATTGCCA GTCCTCAAGC AATGAGCTTT CTGTATCTTG TTTACTAGAT ATATACTACC | 4560 |
| AGGTGAGTCA TTTCCTGGGG TTCTGTTTTC TTTTAAAATC TTTCCCTAAA CTTAATATGT | 4620 |
| ATTAAAAAGT CTGGCTTTTC AGTCCATTCT TTGTGCACTG GGATGGCAAT TGCTTCATTA | 4680 |
| TATGACAATT GCTGTTCCCA AGTCAGAATT CAGTGTGCTG ATTTGACATC AGTTCGTCCC | 4740 |
| GAATAAGTTC CTGTTACCAG GATTTACATT CAGCACATTA GAAACTTGTT GGTGTGCTTT | 4800 |
| TATTCTTGGA GCATTTTCCT TAGACTACCT TCCACTTTGA GTGCTCTGTT TAGGATGTTG | 4860 |
| AGGTGTTAGG ATTCTTGACA GCCAGAAAGA CTGAACCCAC TATCTGGGCA CAGTGTTCGT | 4920 |
| GTTGCTCTAT AAATGTATGC TTTTTTTGAT TTGGGGTTGT TTTACCTACA TTGTCAAACT | 4980 |
| AGATCCATGC TTAACAGTGA TAATGAAGGC TTTTTGTTTG TTTTGTTTGT GGGTCCTCCC | 5040 |
| CCCCCCCCCA AGACAGGGTT TCTCTGTAGG CTGTCCTAGA ACTTGTTCTT TTTTAACCAA | 5100 |
| AATTTGGCAA GGCTGAAAAT GGAATCCTAT AATCAATGCT GGCCACATTA AAGTTAATAG | 5160 |
| TTGAGAAGTC TTGTCTGAAT TTCCTTGGGC AAAAAGATTC TAGCCAGTTC AATACCCTGT | 5220 |
| TGTGCAAATT CAATTTGCTG TTATAATTTG CTCTCAGTTA TCAGTTGGAA GGAGGTTAAT | 5280 |
| TCTAATGTAC TTGGAAGAGG CCTGTAGACC ATCTATAACT GCATCAGTTG TACAGCGTTG | 5340 |
| TTGCCTGGGA TTCTCTAGTT CACATAAACT CCCAAGTCTT AGCCGTGGTG ATGGCTACAG | 5400 |
| TGTGGAAGAT GGTGAGCATT CTAGTGAGTA TCGCGATGAC GGCAGTAAAG AGCAGCAGGC | 5460 |
| AGCCGTGGCT GGGCTCACTG ACCGTGGCTG TAAGTTACGG AGGCAGCACA CACTTCTGTA | 5520 |
| CACACCTCTC ATCAGTTACC GGAGTCATTG CATTGCGGAC TAACTGGCTG ACTCAAGTTG | 5580 |
| TCTTGCTACT GAAGTCTTGA GTTGGTCTCA TGCATTTACC CTGTTGACTT GAGCACCTTA | 5640 |
| AAGTCGAAAG GATGTCTGGT TGTGGCTTTA TTGTAAACAG CCTTAGGTAA AGAGGGGAGT | 5700 |
| ATATCGGTTA GGAAGGTGAA AAATGATACT TCCAAGTTCA GTGGGAAACC CTGGGTTTAT | 5760 |
| CCCCCAGCTT AAGAAAGAAT GCCTAACAAT GTTTCAGAAT TAGATTCTGT GGAAGGTGAG | 5820 |
| GGTGTTAGAA CAGTCCAAAT TTGTTATTGT AGACTTGCAG TGGGAGGAAT TTTTAAATAT | 5880 |
| ACAGATCAGT CGACACTCAT TAACTTCACT GATAAAGGTG GAAACGGATG TGGCAACACT | 5940 |
| TCTAAGTTCA TTTGTATATG TTTGTAATTT GATTGGTTGT ATTCTGTTGC ACTCTAGAAT | 6000 |
| TTGAAGGCAA GGTTACCTCT GCTTTTTAAT TTTTTTTTTT TTAAAGAAAG AAAAAACACT | 6060 |
| GAAAGAAACT TCAAAAGATC TGTTAATGCT AATACCTGAA TGTGGCATTT AACATGTCAT | 6120 |
| GGAAACTGCT TTGAATAAAT ACTTGAGAAA AGGAATGAAA TAATTGCCGT TTTTGTTGTT | 6180 |
| GAGTGAATGG GTGTGGTTTA ATGAGCGTAA TCATTTTTAT AAAACAGCTG TGAGACTGAA | 6240 |
| GTGGAATCCT TATTAAATGT GGAAAATGGC CTTTGAGGAT TACAGTAGAG ATTCAACTAA | 6300 |
| GAGAGTAAAT AAAGCTTGAA ACTAATTCGT TGTAAATTGC TTCTACAATC ATTGCTCTAT | 6360 |
| ATAGCATGCT ATTGCCAATC AGTTTTATGT ATTAAGACCT ATCAGCATGT CTTTTTTAGG | 6420 |
| TTGACCTCAT TTTAAATTAT AAGATGCTCT CTGTACCGTT TTAACATTTC CAGGATTTAT | 6480 |
| TCTTTCTAGG CAAATTCCAC TGGACTGTTT CCATTGTAGA AGCTTCCTTA TAGATTCTTC | 6540 |
| AAATGAAGCT TACAGTGTGC TTTCTTGGGG TTTTGATTTG CACTAAATTT TATTTTCTGA | 6600 |
| AAGATCACTT ATGTTTATAA TGTAGTGCTT TGTCTTAACA ATTAAACTTT CCAGCACTCA | 6660 |

TGCA
The mouse p40^{AUF1} amino acid sequence of GenBank Accession No. NP_031542.2 (SEQ
ID NO: 8) is as follows:

| | |
|---|---|
| MSEEQFGGDG AAAAATAAVG GSAGEQEGAM VAAAAQGPAA AAGSGSGGGG SAAGGTEGGS | 60 |
| AEAEGAKIDA SKNEEDEGHS NSSPRHTEAA AAQREEWKMF IGGLSWDTTK KDLKDYFSKF | 120 |
| GEVVDCTLKL DPITGRSRGF GFVLFKESES VDKVMDQKEH KLNGKVIDPK RAKAMKTKEP | 180 |
| VKKIFVGGLS PDTPEEKIRE YFGGFGEVES IELPMDNKTN KRRGFCFITF KEEEPVKKIM | 240 |
| EKKYHNVGLS KCEIKVAMSK EQYQQQQQWG SRGGFAGRAR GRGGDQQSGY GKVSRRGGHQ | 300 |
| NSYKPY | |

The mouse p42^{AUF1} nucleotide sequence of GenBank Accession No. NM_001077266.2
(SEQ ID NO: 11) is as follows:

| | |
|---|---|
| CCATTTTAGG TGGTCCGCGG CGGCGCCATT AAAGCGAGGA GGAGGCGAGA GTGGCCGCCG | 60 |
| CTGCTACTTC ATTCTTTTTT TTTTCAGTGC AGCCGGGGAG AGCGAGAGAG CGCGCTGCGC | 120 |
| GAGAGTGGGA GGCGAGGGGG GCAGGCCGGG GAGAGGCGCA GGAGCCCTTG CAGCCACGCG | 180 |
| CGCGCCTTGT CTAGGGTGCC TCGCGAGGTA GAGCGGGCAT CGCGCGGCGG CGGCGGGGAT | 240 |
| TACTTTGCTG CTAGTTTCGG TTCGCGGCGG CGGCGGCGTC GGCGGGTGTC GTCTTCGGCG | 300 |
| GCGGCAGTAG CACTATGTCG GAGGAGCAGT TCGGAGGGGA CGGGGCGGCG GCGGCGGCAA | 360 |
| CGGCGGCGGT AGGCGGCTCG GCGGGCGAGC AGGAGGGAGC CATGGTGGCG GCGGCGGCGC | 420 |
| AGGGGCCGGC GGCGGCGGCG GGAAGCGGGA GCGGCGGCGG CGGCTCTGCG GCCGGAGGCA | 480 |
| CCGAAGGAGG CAGCGCCGAG GCAGAGGGAG CCAAGATCGA CGCCAGTAAG AACGAGGAGG | 540 |
| ATGAAGGGAA AATGTTTATA GGAGGCCTTA GCTGGGACAC CACAAAGAAA GATCTGAAGG | 600 |
| ACTACTTTTC CAAATTTGGT GAAGTTGTAG ACTGCACTCT GAAGTTAGAT CCTATCACAG | 660 |
| GGCGATCAAG GGGTTTTGGC TTTGTGCTAT TTAAAGAGTC GGAGAGTGTA GATAAGGTCA | 720 |
| TGGATCAGAA AGAACATAAA TTGAATGGGA AAGTCATTGA TCCTAAAAGG GCCAAAGCCA | 780 |
| TGAAAACAAA AGAGCCTGTC AAAAAAATTT TTGTTGGTGG CCTTTCTCCA GACACACCTG | 840 |
| AAGAAAAAAT AAGAGAGTAC TTTGGTGGTT TTGGTGAGGT TGAATCCATA GAGCTCCCTA | 900 |
| TGGACAACAA GACCAATAAG AGGCGTGGGT TCTGTTTTAT TACCTTTAAG GAAGAGGAGC | 960 |
| CAGTGAAGAA GATAATGGAA AAGAAATACC ACAATGTTGG TCTTAGTAAA TGTGAAATAA | 1020 |
| AAGTAGCCAT GTCAAAGGAA CAGTATCAGC AGCAGCAGCA GTGGGGATCT AGAGGAGGGT | 1080 |
| TTGCAGGCAG AGCTCGCGGA AGAGGTGGAG GCCCCAGTCA AAACTGGAAC CAGGGATATA | 1140 |
| GTAACTATTG GAATCAAGGC TATGGCAACT ATGGATATAA CAGCCAAGGT TACGGAGGTT | 1200 |
| ATGGAGGATA TGACTACACT GGTTACAACA ACTACTATGG ATATGGTGAT TATAGCAATC | 1260 |
| AGCAGAGTGG TTATGGGAAA GTATCCAGGC GAGGTGGACA TCAAAATAGC TACAAACCAT | 1320 |
| ACTAAATTAT TCCATTTGCA ACTTATCCCC AACAGGTGGT GAAGCAGTAT TTTCCAATTT | 1380 |
| GAAGATTCAT TTGAAGGTGG CTCCTGCCAC CTGCTAATAG CAGTTCAAAC TAAATTTTTT | 1440 |
| CTATCAAGTT CCTGAATGGA AGTATGACGT TGGGTCCCTC TGAAGTTTAA TTCTGAGTTC | 1500 |
| TCATTAAAAG AATTTGCTTT CATTGTTTTA TTTCTTAATT GCTATGCTTC AGTATCAATT | 1560 |
| TGTGTTTTAT GCCCCCCCTC CCCCCCAGTA TTGTAGAGCA AGTCTTGTGT TAAAAAAAGC | 1620 |
| CCAGTGTGAC AGTGTCATGA TGTAGTAGTG TCTTACTGGT TTTTTAATAA ATCCTTTTGT | 1680 |
| ATAAAAATGT ATTGGCTCTT TTATCATCAG AATAGGAGGA AGTGAAATAC TACAAATGTT | 1740 |
| TGTCTTGGAT TCAAGTCACT AGAAGCATAA ATTTGAGGGG ATAAAAACAA CGGTAAACTT | 1800 |
| TGTCTGAAAG AGGGCATGGT TAAAAATGTA GTGAATTTTA AATGTTTTTA GCAAAATTTG | 1860 |
| ATTTTGCCCA AGAATCCCTG TCTGAATTGG AAATGACTTA ATGTAGTCAA TGTGCTTGTT | 1920 |
| GGTTGTCTTA ATATTACTTC TGTAGCCATT AAGTTTTATG AGTAACTTCC CAAATACCCA | 1980 |
| CGTTTTTCTT TATATGTATT GTGCTTTTTA AAAACAAATC TGGAAAAATG GGCAAGAACA | 2040 |
| TTTGCAGACA ATTGTTTTTA AGCTTCCATT AAATAAAAAA AATGTGGACT TAAGGAAATC | 2100 |
| TATTAATTTA AATAGAACTG CAGCTAGTTT AGAGAGTATT TTTTTCTTAA AGCTTTGGTG | 2160 |
| TAATTAGGGA AGATTTTAAA AAATGCATAG TGTTTATTTG TATGTGTGCT CTTTTTTTAA | 2220 |
| GTCAATTTTT GGGGGGTTGG TCTGTTAACT GAGTCTAGGA TTTAAAGGTA AGATGTTCCT | 2280 |
| AGAAATCTTG TCATCCCAAA GGGGGGGGCG CTAAGGTGAA ACTTCAGGGT TCAGTCAGGG | 2340 |
| TCACTGCTTT ATGTGTGAAA TCACTCAAAT TGGTAAGTCT CTTATGTTAG CATTCAGGAC | 2400 |
| ATTGATTTCA ACTTGGATGG ACAATTTATA GTTACTACTG AATTGTGTGT TAATGTGTTC | 2460 |
| AGTCCTGGTA AGTTTTCAGT TTGATCAGTT AGTTGGAAGC AGACTTGAAG AGCTGTTAGT | 2520 |
| CACGTGAGCC ATGGGTGCAG TCGATCTGTG GTCAGATGCC TGAGTCTGTG ATAGTGAATT | 2580 |
| GTGTCTAAAG ACATTTTAAT GATAAAAGTC AGTGCTGTAA AGTTGAAAGT TCATGAGAGA | 2640 |
| CATACAATGA GGGCTGCAGC CCATTTTTAA AAACATTATA ATACAAAAGT ATGCACATTT | 2700 |
| GTTTACATAT CCCTGCCTTT GTATTACAGT GGCAGGTTTG TGTACTTAAA CTGGGAAAGC | 2760 |
| CTCAGATCTA TGATTACCTG GCCTATCATA GAAAGTGTCT AAATAAATCA CTCTGTCAAT | 2820 |
| TGAATACATT AGTATTAGCT AGCATACTTC ATTATGCCTG TTTTCCATAA ATACCACACC | 2880 |
| AAAAACTTGC TTGGGGCAGT TTGAGCCTAG TTCATGAGCT GCTATCAGAT TGGTCTTGAT | 2940 |
| CCTATATAAT AGGCCAAATG TCTGTAAACA GCTGTGCTGG TGGAATGTAG AAAGTCACTG | 3000 |
| CACTCAGATT CAACTTCCTG ATTGGAAGTC ATCACAGTGT GATTAAACAT TTTCACAAAG | 3060 |
| AATAGTAGAT AAATAACTTG GTTTTTAATG TTAACTTTGT TTCCATTAAG TCACATTTAA | 3120 |
| AAACTTATCC TCACGCCTAC CTGAGTTAAT TATCTGTTGA CCTAGATATC TTTCTGGCCA | 3180 |
| CTCACTGACT TATTTCTTGA ACTTTTGCCA TTTGCATAAA TCTTGTCAGC TTTGTTCTTG | 3240 |
| ATTATGCATT GTCCAGGCTG AGCTAGTTGT CTTTCCAGGA ATCCCTTTGT CTCTGAATTA | 3300 |
| GGTCCTTTGT TTCCTAAATC ATCCTGCTTG TTTGGCACAA GTCTTCCCAG GCCAGTGAGA | 3360 |
| CCTCCGTGTC CTCTCAGCAC CATAGGGGTA GGTAACCCTG GTTAGGCTGG ACAGGGGTTT | 3420 |
| GCTGAGGGAG TTTGTTCATT TGAATCTAGG TCTTACATGA CGTCTTTCAA ATAGGGTTTT | 3480 |
| TACCTTGACA CTAAACTGTC CAGTCTAAGC AGTTCTGCAA AATGTGAGGG AATTATGAAC | 3540 |
| TTCTTCCTGC AGTGGGTTTT TATGGTTTTG GTTTGTTTTT TGTTGTTTTG GTTCTTTGTT | 3600 |
| GAGCCCTGGA CAAAAACTTC CCTAGTTCTG GTTTCTACAA TTTAAATTAA AAACAGAATT | 3660 |
| CATCTTAGAA TTTTTCACCC TCTTCCCCAA CTATTCTAAT CAATCTTAAG TATGCCCTTC | 3720 |
| ATCTTTTTTC CTTCCTAAGG CTTTTACTGA TAGTGTAATT CCGTACTCTT CAACCCTGGG | 3780 |
| AAGGCTGAAG TGGATTCTTG AGCTCATTTC AAGGCTGACC TGGGTGTTGG CAAGAACCCA | 3840 |
| GCTTAGAACA AACACATGCA AGGCCATCTT ACCTTACATC CTGTTGCTTG GACTTCTTCC | 3900 |
| TGCTCAAAGT TTTTAGTGGA TGCTAAGTGA TCTTTGCTTC CACTGAGGAG TGGAACACTT | 3960 |
| TAGAATGAAC CTCTAGATAG ATATTTTTAT TGTCTGGTGA GGGTTACTGG AGTTTCCCAC | 4020 |
| CCTGCCTGAA GGGTGAATCT GGCTTACAGT GTTCTCATCT CAAAGGGAAG AAGGCAGATG | 4080 |
| GCTGTGTCCA GAGAGAGCCA TCACAGTTTG CTTCAGAGAC ACTAGAATGG GCTGGAAGAT | 4140 |
| CTAGTGGTCT TAATCAGACT TGAAACCTGG CCTTTCTTCA TTACCCATAT GTCTACCAGT | 4200 |
| ACTTGGGCTA ACACTTAAGC CATTAGGGCC TTTGTAGGGG TGTTTTGAGA CCCCCTCCAT | 4260 |
| GCTAACAAAT ATACAGGTTT CTTAACATTT GCTCATAAAC TTGTAAAGCT TACTTTCTCT | 4320 |
| TAATCCACCC CACATTTAAC AAGCCCTGGT ACTTAGAATT TCAGAAGAGT AATGGCAGGT | 4380 |
| AGGTGTGTGT GTGTGTGTGT GTGTGTGTGT GTGTGTGTGT GTGTGAGAGA GAGAGAGAGA | 4440 |
| GAGAGAGAGA GAGAGAGAGA GAGAGAGAGA GAGAGAGAGA AGTTTGTGGA AAATCAGGTA | 4500 |
| ATGACAGCTC ATCCTTTTAG AATTGTACTT CAGAATAGAA ACATTTGGTG GGCTGTTAGG | 4560 |
| TAGCTTTGAT TACTTGTGGG TAGACCTGCT AGTATTGCCA GTCCTCAAGC AATGAGCTTT | 4620 |
| CTGTATCTTG TTTACTAGAT ATATACTACC AGGTGAGTCA TTTCCTGGGG TTCTGTTTTC | 4680 |
| TTTTAAAATC TTTCCCTAAA CTTAATATGT ATTAAAAAGT CTGGCTTTTC AGTCCATTCT | 4740 |
| TTGTGCACTG GGATGGCAAT TGCTTCATTA TATGACAATT GCTGTTCCCA AGTCAGAATT | 4800 |
| CAGTGTGCTG ATTTGACATC AGTTCGTCCC GAATAAGTTC CTGTTACCAG GATTTACATT | 4860 |
| CAGCACATTA GAAACTTGTT GGTGTGCTTT TATTCTTGGA GCATTTTCCT TAGACTACCT | 4920 |
| TCCACTTTGA GTGCTCTGTT TAGGATGTTG AGGTGTTAGG ATTCTTGACA GCCAGAAAGA | 4980 |
| CTGAACCCAC TATCTGGGCA CAGTGTTCGT GTTGCTCTAT AAATGTATGC TTTTTTTGAT | 5040 |
| TTGGGGTTGT TTTACCTACA TTGTCAAACT AGATCCATGC TTAACAGTGA TAATGAAGGC | 5100 |
| TTTTTGTTTG TTTTGTTTGT GGGTCCTCCC CCCCCCCCCA AGACAGGGTT TCTCTGTAGG | 5160 |
| CTGTCCTAGA ACTTGTTCTT TTTTAACCAA AATTTGGCAA GGCTGAAAAT GGAATCCTAT | 5220 |
| AATCAATGCT GGCCACATTA AAGTTAATAG TTGAGAAGTC TTGTCTGAAT TTCCTTGGGC | 5280 |
| AAAAAGATTC TAGCCAGTTC AATACCCTGT TGTGCAAATT CAATTTGCTG TTATAATTTG | 5340 |
| CTCTCAGTTA TCAGTTGGAA GGAGGTTAAT TCTAATGTAC TTGGAAGAGG CCTGTAGACC | 5400 |
| ATCTATAACT GCATCAGTTG TACAGCGTTG TTGCCTGGGA TTCTCTAGTT CACATAAACT | 5460 |
| CCCAAGTCTT AGCCGTGGTG ATGGCTACAG TGTGGAAGAT GGTGAGCATT CTAGTGAGTA | 5520 |
| TCGCGATGAC GGCAGTAAAG AGCAGCAGGC AGCCGTGGCT GGGCTCACTG ACCGTGGCTG | 5580 |
| TAAGTTACGG AGGCAGCACA CACTTCTGTA CACACCTCTC ATCAGTTACC GGAGTCATTG | 5640 |
| CATTGCGGAC TAACTGGCTG ACTCAAGTTG TCTTGCTACT GAAGTCTTGA GTTGGTCTCA | 5700 |
| TGCATTTACC CTGTTGACTT GAGCACCTTA AAGTCGAAAG GATGTCTGGT TGTGGCTTTA | 5760 |
| TTGTAAACAG CCTTAGGTAA AGAGGGGAGT ATATCGGTTA GGAAGGTGAA AAATGATACT | 5820 |
| TCCAAGTTCA GTGGGAAACC CTGGGTTTAT CCCCCAGCTT AAGAAAGAAT GCCTAACAAT | 5880 |
| GTTTCAGAAT TAGATTCTGT GGAAGGTGAG GGTGTTAGAA CAGTCCAAAT TTGTTATTGT | 5940 |
| AGACTTGCAG TGGGAGGAAT TTTTAAATAT ACAGATCAGT CGACACTCAT TAACTTCACT | 6000 |
| GATAAAGGTG GAAACGGATG TGGCAACACT TCTAAGTTCA TTTGTATATG TTTGTAATTT | 6060 |
| GATTGGTTGT ATTCTGTTGC ACTCTAGAAT TTGAAGGCAA GGTTACCTCT GCTTTTTAAT | 6120 |
| TTTTTTTTTT TTAAAGAAAG AAAAAACACT GAAAGAAACT TCAAAAGATC TGTTAATGCT | 6180 |
| AATACCTGAA TGTGGCATTT AACATGTCAT GGAAACTGCT TTGAATAAAT ACTTGAGAAA | 6240 |
| AGGAATGAAA TAATTGCCGT TTTTGTTGTT GAGTGAATGG GTGTGGTTTA ATGAGCGTAA | 6300 |
| TCATTTTTAT AAAACAGCTG TGAGACTGAA GTGGAATCCT TATTAAATGT GGAAAATGGC | 6360 |
| CTTTGAGGAT TACAGTAGAG ATTCAACTAA GAGAGTAAAT AAAGCTTGAA ACTAATTCGT | 6420 |
| TGTAAATTGC TTCTACAATC ATTGCTCTAT ATAGCATGCT ATTGCCAATC AGTTTTATGT | 6480 |
| ATTAAGACCT ATCAGCATGT CTTTTTTAGG TTGACCTCAT TTTAAATTAT AAGATGCTCT | 6540 |
| CTGTACCGTT TTAACATTTC CAGGATTTAT TCTTTCTAGG CAAATTCCAC TGGACTGTTT | 6600 |
| CCATTGTAGA AGCTTCCTTA TAGATTCTTC AAATGAAGCT TACAGTGTGC TTTCTTGGGG | 6660 |
| TTTTGATTTG CACTAAATTT TATTTTCTGA AAGATCACTT ATGTTTATAA TGTAGTGCTT | 6720 |
| TGTCTTAACA ATTAAACTTT CCAGCACTCA TGCA | |

The mouse p42^{AUF1} amino acid sequence of GenBank Accession No. NP_001070734.1
(SEQ ID NO: 12) is as follows:

| | |
|---|---|
| MSEEQFGGDG AAAAATAAVG GSAGEQEGAM VAAAAQGPAA AAGSGSGGGG SAAGGTEGGS | 60 |
| AEAEGAKIDA SKNEEDEGKM FIGGLSWDTT KKDLKDYFSK FGEVVDCTLK LDPITGRSRG | 120 |
| FGFVLFKESE SVDKVMDQKE HKLNGKVIDP KRAKAMKTKE PVKKIFVGGL SPDTPEEKIR | 180 |
| EYFGGFGEVE SIELPMDNKT NKRRGFCFIT FKEEEPVKKI MEKKYHNVGL SKCEIKVAMS | 240 |
| KEQYQQQQQW GSRGGFAGRA RGRGGGPSQN WNQGYSNYWN QGYGNYGYNS QGYGGYGGYD | 300 |
| YTGYNNYYGY GDYSNQQSGY GKVSRRGGHQ NSYKPY | |

The mouse p45^{AUF1} nucleotide sequence of GenBank Accession No. NM_001077265.2
(SEQ ID NO: 15) is as follows:

| | |
|---|---|
| CCATTTTAGG TGGTCCGCGG CGGCGCCATT AAAGCGAGGA GGAGGCGAGA GTGGCCGCCG | 60 |
| CTGCTACTTC ATTCTTTTTT TTTTCAGTGC AGCCGGGGAG AGCGAGAGAG CGCGCTGCGC | 120 |
| GAGAGTGGGA GGCGAGGGGG GCAGGCCGGG GAGAGGCGCA GGAGCCCTTG CAGCCACGCG | 180 |
| CGCGCCTTGT CTAGGGTGCC TCGCGAGGTA GAGCGGGCAT CGCGCGGCGG CGGCGGGGAT | 240 |
| TACTTTGCTG CTAGTTTCGG TTCGCGGCGG CGGCGGCGTC GGCGGGTGTC GTCTTCGGCG | 300 |
| GCGGCAGTAG CACTATGTCG GAGGAGCAGT TCGGAGGGGA CGGGGCGGCG GCGGCGGCAA | 360 |
| CGGCGGCGGT AGGCGGCTCG GCGGGCGAGC AGGAGGGAGC CATGGTGGCG GCGGCGGCGC | 420 |
| AGGGGCCGGC GGCGGCGGCG GGAAGCGGGA GCGGCGGCGG CGGCTCTGCG GCCGGAGGCA | 480 |
| CCGAAGGAGG CAGCGCCGAG GCAGAGGGAG CCAAGATCGA CGCCAGTAAG AACGAGGAGG | 540 |
| ATGAAGGCCA TTCAAACTCC TCCCCACGAC ACACTGAAGC AGCGGCGGCA CAGCGGGAAG | 600 |
| AATGGAAAAT GTTTATAGGA GGCCTTAGCT GGGACACCAC AAAGAAAGAT CTGAAGGACT | 660 |
| ACTTTTCCAA ATTTGGTGAA GTTGTAGACT GCACTCTGAA GTTAGATCCT ATCACAGGGC | 720 |
| GATCAAGGGG TTTTGGCTTT GTGCTATTTA AAGAGTCGGA GAGTGTAGAT AAGGTCATGG | 780 |
| ATCAGAAAGA ACATAAATTG AATGGGAAAG TCATTGATCC TAAAAGGGCC AAAGCCATGA | 840 |
| AAACAAAAGA GCCTGTCAAA AAAATTTTTG TTGGTGGCCT TTCTCCAGAC ACACCTGAAG | 900 |
| AAAAAATAAG AGAGTACTTT GGTGGTTTTG GTGAGGTTGA ATCCATAGAG CTCCCTATGG | 960 |
| ACAACAAGAC CAATAAGAGG CGTGGGTTCT GTTTTATTAC CTTTAAGGAA GAGGAGCCAG | 1020 |
| TGAAGAAGAT AATGGAAAAG AAATACCACA ATGTTGGTCT TAGTAAATGT GAAATAAAAG | 1080 |
| TAGCCATGTC AAAGGAACAG TATCAGCAGC AGCAGCAGTG GGGATCTAGA GGAGGGTTTG | 1140 |
| CAGGCAGAGC TCGCGGAAGA GGTGGAGGCC CCAGTCAAAA CTGGAACCAG GGATATAGTA | 1200 |
| ACTATTGGAA TCAAGGCTAT GGCAACTATG GATATAACAG CCAAGGTTAC GGAGGTTATG | 1260 |
| GAGGATATGA CTACACTGGT TACAACAACT ACTATGGATA TGGTGATTAT AGCAATCAGC | 1320 |
| AGAGTGGTTA TGGGAAAGTA TCCAGGCGAG GTGGACATCA AAATAGCTAC AAACCATACT | 1380 |
| AAATTATTCC ATTTGCAACT TATCCCCAAC AGGTGGTGAA GCAGTATTTT CCAATTTGAA | 1440 |
| GATTCATTTG AAGGTGGCTC CTGCCACCTG CTAATAGCAG TTCAAACTAA ATTTTTTCTA | 1500 |
| TCAAGTTCCT GAATGGAAGT ATGACGTTGG GTCCCTCTGA AGTTTAATTC TGAGTTCTCA | 1560 |
| TTAAAAGAAT TTGCTTTCAT TGTTTTATTT CTTAATTGCT ATGCTTCAGT ATCAATTTGT | 1620 |
| GTTTTATGCC CCCCCTCCCC CCCAGTATTG TAGAGCAAGT CTTGTGTTAA AAAAAGCCCA | 1680 |
| GTGTGACAGT GTCATGATGT AGTAGTGTCT TACTGGTTTT TTAATAAATC CTTTTGTATA | 1740 |
| AAAATGTATT GGCTCTTTTA TCATCAGAAT AGGAGGAAGT GAAATACTAC AAATGTTTGT | 1800 |
| CTTGGATTCA AGTCACTAGA AGCATAAATT TGAGGGGATA AAAACAACGG TAAACTTTGT | 1860 |
| CTGAAAGAGG GCATGGTTAA AAATGTAGTG AATTTTAAAT GTTTTTAGCA AAATTTGATT | 1920 |
| TTGCCCAAGA ATCCCTGTCT GAATTGGAAA TGACTTAATG TAGTCAATGT GCTTGTTGGT | 1980 |
| TGTCTTAATA TTACTTCTGT AGCCATTAAG TTTTATGAGT AACTTCCCAA ATACCCACGT | 2040 |
| TTTTCTTTAT ATGTATTGTG CTTTTTAAAA ACAAATCTGG AAAAATGGGC AAGAACATTT | 2100 |
| GCAGACAATT GTTTTTAAGC TTCCATTAAA TAAAAAAAAT GTGGACTTAA GGAAATCTAT | 2160 |
| TAATTTAAAT AGAACTGCAG CTAGTTTAGA GAGTATTTTT TTCTTAAAGC TTTGGTGTAA | 2220 |
| TTAGGGAAGA TTTTAAAAAA TGCATAGTGT TTATTTGTAT GTGTGCTCTT TTTTTAAGTC | 2280 |
| AATTTTTGGG GGGTTGGTCT GTTAACTGAG TCTAGGATTT AAAGGTAAGA TGTTCCTAGA | 2340 |
| AATCTTGTCA TCCCAAAGGG GCGGGCGCTA AGGTGAAACT TCAGGGTTCA GTCAGGGTCA | 2400 |
| CTGCTTTATG TGTGAAATCA CTCAAATTGG TAAGTCTCTT ATGTTAGCAT TCAGGACATT | 2460 |
| GATTTCAACT TGGATGGACA ATTTATAGTT ACTACTGAAT TGTGTGTTAA TGTGTTCAGT | 2520 |
| CCTGGTAAGT TTTCAGTTTG ATCAGTTAGT TGGAAGCAGA CTTGAAGAGC TGTTAGTCAC | 2580 |
| GTGAGCCATG GGTGCAGTCG ATCTGTGGTC AGATGCCTGA GTCTGTGATA GTGAATTGTG | 2640 |
| TCTAAAGACA TTTTAATGAT AAAAGTCAGT GCTGTAAAGT TGAAAGTTCA TGAGAGACAT | 2700 |
| ACAATGAGGG CTGCAGCCCA TTTTTAAAAA CATTATAATA CAAAAGTATG CACATTTGTT | 2760 |
| TACATATCCC TGCCTTTGTA TTACAGTGGC AGGTTTGTGT ACTTAAACTG GGAAAGCCTC | 2820 |
| AGATCTATGA TTACCTGGCC TATCATAGAA AGTGTCTAAA TAAATCACTC TGTCAATTGA | 2880 |
| ATACATTAGT ATTAGCTAGC ATACTTCATT ATGCCTGTTT TCCATAAATA CCACACCAAA | 2940 |
| AACTTGCTTG GGGCAGTTTG AGCCTAGTTC ATGAGCTGCT ATCAGATTGG TCTTGATCCT | 3000 |
| ATATAATAGG CCAAATGTCT GTAAACAGCT GTGCTGGTGG AATGTAGAAA GTCACTGCAC | 3060 |
| TCAGATTCAA CTTCCTGATT GGAAGTCATC ACAGTGTGAT TAAACATTTT CACAAAGAAT | 3120 |
| AGTAGATAAA TAACTTGGTT TTTAATGTTA ACTTTGTTTC CATTAAGTCA CATTTAAAAA | 3180 |
| CTTATCCTCA CGCCTACCTG AGTTAATTAT CTGTTGACCT AGATATCTTT CTGGCCACTC | 3240 |
| ACTGACTTAT TTCTTGAACT TTTGCCATTT GCATAAATCT TGTCAGCTTT GTTCTTGATT | 3300 |
| ATGCATTGTC CAGGCTGAGC TAGTTGTCTT TCCAGGAATC CCTTTGTCTC TGAATTAGGT | 3360 |
| CCTTTGTTTC CTAAATCATC CTGCTTGTTT GGCACAAGTC TTCCCAGGCC AGTGAGACCT | 3420 |
| CCGTGTCCTC TCAGCACCAT AGGGGTAGGT AACCCTGGTT AGGCTGGACA GGGGTTTGCT | 3480 |
| GAGGGAGTTT GTTCATTTGA ATCTAGGTCT TACATGACGT CTTTCAAATA GGGTTTTTAC | 3540 |
| CTTGACACTA AACTGTCCAG TCTAAGCAGT TCTGCAAAAT GTGAGGGAAT TATGAACTTC | 3600 |
| TTCCTGCAGT GGGTTTTTAT GGTTTTGGTT TGTTTTTTGT TGTTTTGGTT CTTTGTTGAG | 3660 |
| CCCTGGACAA AAACTTCCCT AGTTCTGGTT TCTACAATTT AAATTAAAAA CAGAATTCAT | 3720 |
| CTTAGAATTT TTCACCCTCT TCCCCAACTA TTCTAATCAA TCTTAAGTAT GCCCTTCATC | 3780 |
| TTTTTTCCTT CCTAAGGCTT TTACTGATAG TGTAATTCCG TACTCTTCAA CCCTGGGAAG | 3840 |
| GCTGAAGTGG ATTCTTGAGC TCATTTCAAG GCTGACCTGG GTGTTGGCAA GAACCCAGCT | 3900 |
| TAGAACAAAC ACATGCAAGG CCATCTTACC TTACATCCTG TTGCTTGGAC TTCTTCCTGC | 3960 |
| TCAAAGTTTT TAGTGGATGC TAAGTGATCT TTGCTTCCAC TGAGGAGTGG AACACTTTAG | 4020 |
| AATGAACCTC TAGATAGATA TTTTTATTGT CTGGTGAGGG TTACTGGAGT TTCCCACCCT | 4080 |
| GCCTGAAGGG TGAATCTGGC TTACAGTGTT CTCATCTCAA AGGGAAGAAG GCAGATGGCT | 4140 |
| GTGTCCAGAG AGAGCCATCA CAGTTTGCTT CAGAGACACT AGAATGGGCT GGAAGATCTA | 4200 |
| GTGGTCTTAA TCAGACTTGA AACCTGGCCT TTCTTCATTA CCCATATGTC TACCAGTACT | 4260 |
| TGGGCTAACA CTTAAGCCAT TAGGGCCTTT GTAGGGGTGT TTTGAGACCC CCTCCATGCT | 4320 |
| AACAAATATA CAGGTTTCTT AACATTTGCT CATAAACTTG TAAAGCTTAC TTTCTCTTAA | 4380 |
| TCCACCCCAC ATTTAACAAG CCCTGGTACT TAGAATTTCA GAAGAGTAAT GGCAGGTAGG | 4440 |
| TGTGTGTGTG TGTGTGTGTG TGTGTGTGTG TGTGTGTGTG TGAGAGAGAG AGAGAGAGAG | 4500 |
| AGAGAGAGAG AGAGAGAGAG AGAGAGAGAG AGAGAGAAGT TTGTGGAAAA TCAGGTAATG | 4560 |
| ACAGCTCATC CTTTTAGAAT TGTACTTCAG AATAGAAACA TTTGGTGGGC TGTTAGGTAG | 4620 |
| CTTTGATTAC TTGTGGGTAG ACCTGCTAGT ATTGCCAGTC CTCAAGCAAT GAGCTTTCTG | 4680 |
| TATCTTGTTT ACTAGATATA TACTACCAGG TGAGTCATTT CCTGGGGTTC TGTTTTCTTT | 4740 |
| TAAAATCTTT CCCTAAACTT AATATGTATT AAAAAGTCTG GCTTTTCAGT CCATTCTTTG | 4800 |
| TGCACTGGGA TGGCAATTGC TTCATTATAT GACAATTGCT GTTCCCAAGT CAGAATTCAG | 4860 |
| TGTGCTGATT TGACATCAGT TCGTCCCGAA TAAGTTCCTG TTACCAGGAT TTACATTCAG | 4920 |
| CACATTAGAA ACTTGTTGGT GTGCTTTTAT TCTTGGAGCA TTTTCCTTAG ACTACCTTCC | 4980 |
| ACTTTGAGTG CTCTGTTTAG GATGTTGAGG TGTTAGGATT CTTGACAGCC AGAAAGACTG | 5040 |
| AACCCACTAT CTGGGCACAG TGTTCGTGTT GCTCTATAAA TGTATGCTTT TTTTGATTTG | 5100 |
| GGGTTGTTTT ACCTACATTG TCAAACTAGA TCCATGCTTA ACAGTGATAA TGAAGGCTTT | 5160 |
| TTGTTTGTTT TGTTTGTGGG TCCTCCCCCC CCCCCCAAGA CAGGGTTTCT CTGTAGGCTG | 5220 |
| TCCTAGAACT TGTTCTTTTT TAACCAAAAT TTGGCAAGGC TGAAAATGGA ATCCTATAAT | 5280 |
| CAATGCTGGC CACATTAAAG TTAATAGTTG AGAAGTCTTG TCTGAATTTC CTTGGGCAAA | 5340 |
| AAGATTCTAG CCAGTTCAAT ACCCTGTTGT GCAAATTCAA TTTGCTGTTA TAATTTGCTC | 5400 |
| TCAGTTATCA GTTGGAAGGA GGTTAATTCT AATGTACTTG GAAGAGGCCT GTAGACCATC | 5460 |
| TATAACTGCA TCAGTTGTAC AGCGTTGTTG CCTGGGATTC TCTAGTTCAC ATAAACTCCC | 5520 |
| AAGTCTTAGC CGTGGTGATG GCTACAGTGT GGAAGATGGT GAGCATTCTA GTGAGTATCG | 5580 |
| CGATGACGGC AGTAAAGAGC AGCAGGCAGC CGTGGCTGGG CTCACTGACC GTGGCTGTAA | 5640 |
| GTTACGGAGG CAGCACACAC TTCTGTACAC ACCTCTCATC AGTTACCGGA GTCATTGCAT | 5700 |
| TGCGGACTAA CTGGCTGACT CAAGTTGTCT TGCTACTGAA GTCTTGAGTT GGTCTCATGC | 5760 |
| ATTTACCCTG TTGACTTGAG CACCTTAAAG TCGAAAGGAT GTCTGGTTGT GGCTTTATTG | 5820 |
| TAAACAGCCT TAGGTAAAGA GGGGAGTATA TCGGTTAGGA AGGTGAAAAA TGATACTTCC | 5880 |
| AAGTTCAGTG GGAAACCCTG GGTTTATCCC CCAGCTTAAG AAAGAATGCC TAACAATGTT | 5940 |
| TCAGAATTAG ATTCTGTGGA AGGTGAGGGT GTTAGAACAG TCCAAATTTG TTATTGTAGA | 6000 |
| CTTGCAGTGG GAGGAATTTT TAAATATACA GATCAGTCGA CACTCATTAA CTTCACTGAT | 6060 |
| AAAGGTGGAA ACGGATGTGG CAACACTTCT AAGTTCATTT GTATATGTTT GTAATTTGAT | 6120 |
| TGGTTGTATT CTGTTGCACT CTAGAATTTG AAGGCAAGGT TACCTCTGCT TTTTAATTTT | 6180 |
| TTTTTTTTTA AAGAAAGAAA AAACACTGAA AGAAACTTCA AAAGATCTGT TAATGCTAAT | 6240 |
| ACCTGAATGT GGCATTTAAC ATGTCATGGA AACTGCTTTG AATAAATACT TGAGAAAAGG | 6300 |
| AATGAAATAA TTGCCGTTTT TGTTGTTGAG TGAATGGGTG TGGTTTAATG AGCGTAATCA | 6360 |
| TTTTTATAAA ACAGCTGTGA GACTGAAGTG GAATCCTTAT TAAATGTGGA AAATGGCCTT | 6420 |
| TGAGGATTAC AGTAGAGATT CAACTAAGAG AGTAAATAAA GCTTGAAACT AATTCGTTGT | 6480 |
| AAATTGCTTC TACAATCATT GCTCTATATA GCATGCTATT GCCAATCAGT TTTATGTATT | 6540 |
| AAGACCTATC AGCATGTCTT TTTTAGGTTG ACCTCATTTT AAATTATAAG ATGCTCTCTG | 6600 |
| TACCGTTTTA ACATTTCCAG GATTTATTCT TTCTAGGCAA ATTCCACTGG ACTGTTTCCA | 6660 |
| TTGTAGAAGC TTCCTTATAG ATTCTTCAAA TGAAGCTTAC AGTGTGCTTT CTTGGGGTTT | 6720 |
| TGATTTGCAC TAAATTTTAT TTTCTGAAAG ATCACTTATG TTTATAATGT AGTGCTTTGT | 6780 |
| CTTAACAATT AAACTTTCCA GCACTCATGC A | |

The mouse p45^{AUF1} amino acid sequence of GenBank Accession No. NP_001070733.1
(SEQ ID NO: 16) is as follows:

| | |
|---|---|
| MSEEQFGGDG AAAAATAAVG GSAGEQEGAM VAAAAQGPAA AAGSGSGGGG SAAGGTEGGS | 60 |
| AEAEGAKIDA SKNEEDEGHS NSSPRHTEAA AAQREEWKMF IGGLSWDTTK KDLKDYFSKF | 120 |
| GEVVDCTLKL DPITGRSRGF GFVLFKESES VDKVMDQKEH KLNGKVIDPK RAKAMKTKEP | 180 |
| VKKIFVGGLS PDTPEEKIRE YFGGFGEVES IELPMDNKTN KRRGFCFITF KEEEPVKKIM | 240 |
| EKKYHNVGLS KCEIKVAMSK EQYQQQQQWG SRGGFAGRAR GRGGGPSQNW NQGYSNYWNQ | 300 |
| GYGNYGYNSQ GYGGYGGYDY TGYNNYYGYG DYSNQQSGYG KVSRRGGHQN SYKPY | |

It is noted that the sequences described herein may be described with reference to accession numbers, for example, as provided in Table 1, that include, e.g., a coding sequence or protein sequence with or without additional sequence elements or portions (e.g., leader sequences, tags, immature portions, regulatory regions, etc.). Thus, reference to such sequence accession numbers or corresponding sequence identification numbers refers to either the sequence fully described therein or some portion thereof (e.g., that portion encoding a protein or polypeptide of interest to the technology described herein (e.g., AUF1 or a functional fragment thereof); the mature protein sequence that is described within a longer amino acid sequence; a regulatory region of interest (e.g., promoter sequence or regulatory element) disclosed within a longer sequence described herein; etc.). Likewise, variants and isoforms of accession numbers and corresponding sequence identification numbers described herein are also contemplated.

Accordingly, in certain embodiments, the AUF1 protein referred to herein has an amino acid sequence as set forth in Table 1 and the sequences disclosed herein, or is a functional fragment thereof. In certain embodiments, the AUF1 is a p37, p40, p42 or p45 form of human AUF1 and has an amino acid sequence of SEQ ID NO: 2, 6, 10 or 14, respectively. In other embodiments, the AUF1 is a p37, p40, p42 or p45 form of mouse AUF1 and has an amino acid sequence of SEQ ID NO: 4, 8, 12 or 16, respectively. In certain embodiments, the AUF1 has 90%, 95% or 99% sequence identity to the amino acid sequence of SEQ ID NO: 2, 6, 10 or 14 and has AUF1 functional activity. In certain embodiments, the AUF1 has 90%, 95% or 99% sequence identity to the amino acid sequence of SEQ ID NO: 4, 8, 12 or 16 and has AUF1 functional activity. In one embodiment, the functional fragment as referred to herein includes an amino acid sequence that has at least 80%, at least 85%, at least 90%, at least 95%, at least 97%, or at least 99% amino acid sequence identity to amino acid sequence of SEQ ID NO: 2, 6, 10 or 14 for human AUF1 or in other embodiments to the amino acid sequence of SEQ ID NO: 4, 8, 12 or 16 for mouse AUF1.

Also provided are nucleic acids comprising nucleotide sequences encoding a human AUF1 protein, or functional fragment thereof, for example, the nucleotide sequences of SEQ ID NO: 1, 5, 9, or 13. Also provided are nucleic acids comprising nucleotide sequences having 80%, 85%, 90%, 95%, or 99% sequence identity to one of the nucleotide sequences of SEQ ID NO: 1, 5, 9, or 13 and encoding a human AUF1 protein having an amino acid sequence of SEQ ID NO: 2, 6, 10 or 14, or a functional fragment thereof. Also provided are nucleic acids comprising nucleotide sequences having 80%, 85%, 90%, 95%, or 99% sequence identity to one of the nucleotide sequences of SEQ ID NO: 3, 7, 11, or 15 and encoding a mouse AUF1 protein having an amino acid sequence of SEQ ID NO: 4, 8, 12 or 16, or a functional fragment thereof.

In some embodiments, the AAV vectors and viral particles described herein comprise a nucleic acid molecule comprising a nucleotide sequence set forth in Table 1 (or described herein), or portions thereof that encode a functional fragment of an AUF1 protein as described supra, particularly in an expression cassette as described herein for expression in the cells of a subject, particularly, muscle cells of a subject.

As described in more detail below, provided are compositions comprising vectors encoding an AUF1 protein that may be useful in gene therapy, which includes both ex vivo and in vivo techniques. Thus, host cells can be genetically engineered ex vivo with a nucleic acid molecule that encodes an AUF1 or functional fragment thereof that is expressed in the host cell, with the engineered cells then being provided to a patient to be treated. Delivery of the active agent of a composition described herein in vivo may involve a process that effectively introduces a molecule of interest (e.g., AUF1 protein or a functional fragment thereof) into the cells or tissue being treated. In the case of polypeptide-based active agents, this can be carried out directly or, alternatively, by transfecting transcriptionally active DNA into living cells such that the active polypeptide coding sequence is expressed and the polypeptide is produced by cellular machinery. Transcriptionally active DNA may be delivered into the cells or tissue, e.g., muscle, being treated using transfection methods including, but not limited to, electroporation, microinjection, calcium phosphate coprecipitation, DEAE dextran facilitated transfection, cationic liposomes, retroviruses or gene therapy viral vectors such as AAV or adenoviral vectors. In certain embodiments, the DNA to be transfected is cloned into a vector and, in certain embodiments, a gene therapy vector, such as an rAAV vector, and is operably linked to regulatory sequences which promote expression of the AUF1 in muscle cells.

Alternatively, cells can be engineered in vivo by administration of the polynucleotide using techniques known in the art. For example, by direct injection of a “naked” polynucleotide (Felgner et al., “Gene Therapeutics,” Nature 349:351-352 (1991); U.S. Pat. No. 5,679,647; Wolff et al., “The Mechanism of Naked DNA Uptake and Expression,” Adv. Genet. 54:3-20 (2005), which are hereby incorporated by reference in their entirety) or a polynucleotide formulated in a composition with one or more other targeting elements which facilitate uptake of the polynucleotide by a cell. Alternatively, nucleic acids encoding an AUF1 protein, or functional fragment thereof, operably linked to a regulatory element, particularly for expression in muscle cells, may be introduced into cells in vivo using gene therapy methods described in further detail herein, for example, by a viral vector, such as a recombinant AAV viral particle.

Host cells that can be used with the vectors described herein include, without limitation, myocytes. The term “myocyte,” as used herein, refers a cell that has been differentiated from a progenitor myoblast such that it is capable of expressing muscle-specific phenotype under appropriate conditions. Terminally differentiated myocytes fuse with one another to form myotubes, a major constituent of muscle fibers. The term “myocyte” also refers to myocytes that are de-differentiated. The term includes cells in vivo and cells cultured ex vivo regardless of whether such cells are primary or passaged. Myocytes are found in all muscle types, e.g., skeletal muscle, cardiac muscle, smooth muscle, etc. Myocytes are found and can be isolated from any vertebrate species, including, without limitation, human, orangutan, monkey, chimpanzee, dog, cat, rat, rabbit, mouse, horse, cow, pig, elephant, etc. Alternatively, the host cell can be a prokaryotic cell, e.g., a bacterial cell such as E. coli, that is used, for example, to propagate the vectors.

It may be desirable in certain circumstances to utilize myocyte progenitor cells such as mesenchymal precursor cells or myoblasts rather than fully differentiated myoblasts. Examples of tissue from which such cells can be isolated include placenta, umbilical cord, bone marrow, skin, muscle, periosteum, or perichondrium. Myocytes can be derived from such cells, for example, by inducing their differentiation in tissue culture. The present application encompasses not only myocyte precursor/progenitor cells, but also cells that can be trans-differentiated into myocytes, e.g., adipocytes and fibroblasts.

Also encompassed are expression systems comprising nucleic acid molecules described herein. Generally, the use of recombinant expression systems involves inserting a nucleic acid molecule encoding the amino acid sequence of a desired peptide into an expression system to which the molecule is heterologous (i.e., not native or not normally present). One or more desired nucleic acid molecules encoding a polypeptide described herein (e.g., AUF1) may be inserted into the vector. When multiple nucleic acid molecules are inserted, the multiple nucleic acid molecules may encode the same or different peptides. The heterologous nucleic acid molecule is inserted into the expression system or vector in proper sense (5′ to 3′) orientation relative to the promoter and any other 5′ regulatory molecules, and correct reading frame.

The preparation of the nucleic acid constructs can be carried out using standard cloning procedures well known in the art, for example, as described by Joseph Sambrook et al., MOLECULAR CLONING: A LABORATORY MANUAL (Cold Springs Harbor 2012), which is hereby incorporated by reference in its entirety.

A nucleic acid molecule encoding an AUF1 protein or functional fragment thereof may be operably linked to a promoter, for example, a constitutive promoter or a muscle specific promoter (e.g., human muscle creatine kinase (MCK) promoter and others described herein, for example in Table 2). The vector may further comprise one or more additional regulatory elements including, without limitation, a leader sequence, a suitable 3′ regulatory region to allow transcription in the host or a certain medium, intron sequences, enhancers and polyA signal sequences, and/or any additional desired component, such as reporter or marker genes. Such additional elements may be cloned into the vector of choice using standard cloning procedures in the art.

### 5.3.Gene Cassettes and Regulatory Elements

Another aspect provided herein relates to nucleic acid expression cassettes comprising a nucleic acid encoding an AUF1(including p37, p40, p42 or p45 AUF1) or a functional fragment thereof operably linked to regulatory elements, including promoter elements, and optionally enhancer elements and/or introns, to enhance or facilitate expression of the nucleic acid encoding the AUF1 or functional fragment thereof. The expression cassettes or transgenes provided herein may comprise nucleotide sequences encoding a human AUF1 protein having an amino acid sequence of SEQ ID NO: 2, 6, 10, or 14, or a functional fragment thereof (or, alternatively, for example, for mouse model studies, the expression cassette comprises a nucleotide sequence encoding a mouse AUF1 protein having an amino acid sequence of SEQ ID NO: 4, 8, 12 or 16, or a functional fragment thereof). In embodiments, the nucleotide sequence encoding the human AUF1 is SEQ ID NO: 1, 5, 9, or 13 (or the nucleotide sequence encoding mouse AUF1 is SEQ ID NO: 3, 7, 11 or 15). In certain embodiments, the AUF1 protein has no more than 1, 2, 3, 4, 5, 10, 15 amino acid substitutions, including conservative substitutions, with respect to the amino acid sequence of SEQ ID NO: 2, 6, 10, or 14, or a functional fragment thereof (or, alternatively, for example, for mouse model studies, with respect to the amino acid sequence of SEQ ID NO: 4, 8, 12 or 16), where the AUF1 protein has one or more AUF1 functions. In embodiments, the regulatory control elements include promoters and may be either constitutive or may be tissue-specific, that is, active (or substantially more active or significantly more active) only in the target cell/tissue. In particular, provided are promoter and other regulatory elements that promote muscle specific expression, such as those in Table 2 infra. In embodiments, including for use as a transgene in a recombinant AAV particle, the expression cassette or transgene is flanked by inverted terminal repeats (ITRs) (for example AAV2 ITRs), including forms of ITRs for single-stranded AAV genomes or self-complementary AAV genomes.

### 5.3.1 Promoters

### 5.3.1.1 Tissue-Specific Promoters

In specific embodiments, an expression cassette, including of an AAV vector, comprises a regulatory sequence, such as a promoter, operably linked to the transgene that allows for expression in target tissues. The promoter may be a muscle promoter. In certain embodiments, the promoter is a muscle-specific promoter. The phrase “muscle-specific”, “muscle-selective” or “muscle-directed” refers to nucleic acid elements that have adapted their activity in muscle cells or tissue due to the interaction of such elements with the intracellular environment of the muscle cells. Such muscle cells may include myocytes, myotubes, cardiomyocytes, and the like. Specialized forms of myocytes with distinct properties such as cardiac, skeletal, and smooth muscle cells are included. Various therapeutics may benefit from muscle-specific expression of a transgene. In particular, gene therapies that treat various forms of muscular dystrophy or other indications associated with muscle wasting or reduced muscle performance delivered to and enabling high transduction efficiency in muscle cells have the added benefit of directing expression of the transgene in the cells where the transgene is most needed. Cardiac tissue will also benefit from muscle-directed expression of the transgene. Muscle-specific promoters may be operably linked to the transgenes of the invention.

Adeno-associated viral (AAV) vectors disclosed herein comprise a muscle cell-specific promoter. In some embodiments, the muscle cell-specific promoter mediates cell-specific and/or tissue-specific expression of an AUF1 protein or fragment thereof. The promoter may be a mammalian promoter. For example, the promoter may be a human promoter, a murine promoter, a porcine promoter, a feline promoter, a canine promoter, an ovine promoter, a non-human primate promoter, an equine promoter, a bovine promoter, or the like.

In some embodiments, the muscle cell-specific promoter a muscle creatine kinase (MCK) promoter, a C5-12 promoter, a CK6-CK9 promoter, a dMCK promoter, a tMCK promoter (SEQ ID NO: 33), a smooth muscle 22 (SM22) promoter, a myo-3 promoter, a Spc512 promoter, a creatine kinase (CK) 8 promoter, a creatine kinase (CK) 8e promoter, a U6 promoter, a H1 promoter, a desmin promoter, a Pitx3 promoter, a skeletal alpha-actin promoter, a MHCK7 promoter, or a Sp-301 promoter. Suitable muscle cell-specific promoter sequences are well known in the art and are provided in Table 2 below (Malerba et al., “PABPN1 Gene Therapy for Oculopharyngeal Muscular Dystrophy,” Nat. Commun. 8:14848 (2017); Wang et al., “Construction and Analysis of Compact Muscle-Specific Promoters for AAV Vectors,” Gene. Ther. 15:1489-1499 (2008); Piekarowicz et al., “A Muscle Hybrid Promoter as a Novel Tool for Gene Therapy,” Mol. Ther. Methods Clin. Dev. 15:157-169 (2019); Salva et al., “Design of Tissue-Specific Regulatory Cassettes for High-Level rAAV-Mediated Expression in Skeletal and Cardiac Muscle,” Mol. Ther. 15(2):320-329 (2007); Lui et al., “Synthetic Promoter for Efficient and Muscle-Specific Expression of Exogenous Genes,” Plasmid 106:102441 (2019), which are hereby incorporated by reference in their entirety.).

TABLE 2
Muscle Specific-Promoter Sequences

| | | SEQ ID |
|---|---|---|
| Promoter | Sequence* | NO: |
| Human | AGCCAGCCTCAGTTTCCCCTCCACTCAGTCCCTAGGAGGAAGGGGCGCCCA | 17 |
| muscle | AGCGCGGGTTTCTGGGGTTAGACTGCCCTCCATTGCAATTGGTCCTTCTCC | |
| creatine | CGGCCTCTGCTTCCTCCAGCTCACAGGGTATCTGCTCCTCCTGGAGCCACA | |
| kinase | CCTTGGTTCCCCGAGGTGCCGCTGGGACTCGGGTAGGGGTGAGGGCCCAGG | |
| (MCK) | GGGCACAGGGGGAGCCGAGGGCCACAGGAAGGGCTGGTGGCTGAAGGAGAC | |
| | TCAGGGGCCAGGGGACGGTGGCTTCTACGTGCTTGGGACGTTCCCAGCCAC | |
| | CGTCCCATGTTCCCGGCGGGGGGCCAGCTGTCCCCACCGCCAGCCCAACTC | |
| | AGCACTTGGTCAGGGTATCAGCTTGGTGGGGGGGCGTGAGCCCAGCCCCTG | |
| | GGGCGGCTCAGCCCATACAAGGCCATGGGGCTGGGCGCAAAGCATGCCTGG | |
| | GTTCAGGGTGGGTATGGTGCGGGAGCAGGGAGGTGAGAGGCTCAGCTGCCC | |
| | TCCAGAACTCCTCCCTGGGGACAACCCCTCCCAGCCAATAGCACAGCCTAG | |
| | GTCCCCCTATATAAGGCCACGGCTGCTGGCCCTTCCTTT | |
| | (NCBI sequence ID No. 1158) | |
| Human | CTGAGGCTCAGGGCTAGCTCGCCCATAGACATACATGGCAGGCAGGCTTTG | 18 |
| desmin | GCCAGGATCCCTCCGCCTGCCAGGCGTCTCCCTGCCCTCCCTTCCTGCCTA | |
| | GAGACCCCCACCCTCAAGCCTGGCTGGTCTTTGCCTGAGACCCAAACCTCT | |
| | TCGACTTCAAGAGAATATTTAGGAACAAGGTGGTTTAGGGCCTTTCCTGGG | |
| | AACAGGCCTTGACCCTTTAAGAAATGACCCAAAGTCTCTCCTTGACCAAAA | |
| | AGGGGACCCTCAAACTAAAGGGAAGCCTCTCTTCTGCTGTCTCCCCTGACC | |
| | CCACTCCCCCCCACCCCAGGACGAGGAGATAACCAGGGCTGAAAGAGGCCC | |
| | GCCTGGGGGCTGCAGACATGCTTGCTGCCTGCCCTGGCGAAGGATTGGCAG | |
| | GCTTGCCCGTCACAGGACCCCCGCTGGCTGACTCAGGGGCGCAGGCCTCTT | |
| | GCGGGGGAGCTGGCCTCCCCGCCCCCACGGCCACGGGCCGCCCTTTCCTGG | |
| | CAGGACAGCGGGATCTTGCAGCTGTCAGGGGAGGGGAGGCGGGGGCTGATG | |
| | TCAGGAGGGATACAAATAGTGCCGACGGCTGGGGGCCCT | |
| | (NCBI sequence ID No. 1674) | |
| Human | GGAGTTCCAGGGGCGTAAAGGAGAGGGAGTTCGCCTTCCTTCCCTTCCTGA | 19 |
| skeletal | GACTCAGGAGTGACTGCTTCTCCAATCCTCCCAAGCCCACCACTCCACACG | |
| muscle | ACTCCCTCTTCCCGGTAGTCGCAAGTGGGAGTTTGGGGATCTGAGCAAAGA | |
| alpha | ACCCGAAGAGGAGTTGAAATATTGGAAGTCAGCAGTCAGGCACCTTCCCGA | |
| actin | GCGCCCAGGGCGCTCAGAGTGGACATGGTTGGGGAGGCCTTTGGGACAGGT | |
| acta1 | GCGGTTCCCGGAGCGCAGGCGCACACATGCACCCACCGGCGAACGCGGTGA | |
| | CCCTCGCCCCACCCCATCCCCTCCGGCGGGCAACTGGGTCGGGTCAGGAGG | |
| | GGCAAACCCGCTAGGGAGACACTCCATATACGGCCCGGCCCGCGTTACCTG | |
| | GGACCGGGCCAACCCGCTCCTTCTTTGGTCAACGCAGGGGACCCGGGCGGG | |
| | GGCCCAGGCCGCGAACCGGCCGAGGGAGGGGGCTCTAGTGCCCAACACCCA | |
| | AATATGGCTCGAGAAGGGCAGCGACATTCCTGCGGGGTGGCGCGGAGGGAA | |
| | TGCCCGCGGGCTATATAAAACCTGAGCAGAGGGACAAGC | |
| | (NCBI sequence ID No. 58) | |
| Mouse | AGAAACCTGTGGTCTAGAGGCGGGGGGGGGCCGATGGAGGCAACGCACGCC | 20 |
| muscle | CCCGCAGGCGCCCAGGCCACGCCCTCTGCCGCAGCATTCGGTGAAACCTGC | |
| creatine | GTTCCGAGAACTTCTGAAAACTTTATCTGGGGGCCTTCGAGAAGGCTCAGA | |
| kinase | CAGTAAGGGTGCATGCTGCCAATCCTGAGGAGCTGAGTTCGATCCCTGAGA | |
| (MCK) | CCTTCAGGGTGGACAGAGACGGACTCCCACATGTTGTTTTCTGACTTCTAC | |
| | ATGTGTCCAGTCATACATACACAAATATGGAATAAACAGATGGCTCATCAG | |
| | GTAAGAGTGCTGGCTGCTTTTGCAGAGGACCCAGGTTCGATTTCCAGAACC | |
| | CACATGTCGGCTCAAAATCATCTGTAATTCCAGTTCCAGGGAGATCCAGCA | |
| | CTTTCTTCCAGGGCCTCCACAGACACACATAAAATAAAGATAAAAATCTCC | |
| | AAAAAATATTGTTTTAATAATTACAACCTGAAGACCTTGCACAACTATTCC | |
| | TGGCTGAGAAGATGGTAAGGGCGCTAGCTGCCAAGCTTGACAGCCTGAGTT | |
| | TCATCTCCAAGAACCATGAAAACTGACTCCTGGGAATTA | |
| | (NCBI sequence ID No. 12715) | |
| Mouse | GGAAGCAGAAGGCCAACATTCCTCCCAAGGGAAACTGAGGCTCAGAGTTAA | 21 |
| desmin | AACCCAGGTATCAGTGATATGCATGTGCCCCGGCCAGGGTCACTCTCTGAC | |
| | TAACCGGTACCTACCCTACAGGCCTACCTAGAGACTCTTTTGAAAGGATGG | |
| | TAGAGACCTGTCCGGGCTTTGCCCACAGTCGTTGGAAACCTCAGCATTTTC | |
| | TAGGCAACTTGTGCGAATAAAACACTTCGGGGGTCCTTCTTGTTCATTCCA | |
| | ATAACCTAAAACCTCTCCTCGGAGAAAATAGGGGGCCTCAAACAAACGAAA | |
| | TTCTCTAGCCCGCTTTCCCCAGGATAAGGCAGGCATCCAAATGGAAAAAAA | |
| | GGGGCCGGCCGGGGGTCTCCTGTCAGCTCCTTGCCCTGTGAAACCCAGCAG | |
| | GCCTGCCTGTCTTCTGTCCTCTTGGGGCTGTCCAGGGGCGCAGGCCTCTTG | |
| | CGGGGGAGCTGGCCTCCCCGCCCCCTCGCCTGTGGCCGCCCTTTTCCTGGC | |
| | AGGACAGAGGGATCCTGCAGCTGTCAGGGGAGGGGCGCCGGGGGGTGATGT | |
| | CAGGAGGGCTACAAATAGTGCAGACAGCTAAGGGGCTCC | |
| | (NCBI sequence ID No. 13346) | |
| Mouse | GGGGTGATGTGTGTCAGATCTCTGGATTGGGGGAGCTTCAAAGTGGGAAAG | 22 |
| skeletal | AAAATGGAGTTCAAATGTGGGGCTTATTTTCCATCCCTACCTGGAGCCCAT | |
| muscle | GACTCCTCCCGGCTCACCTGACCACAGGGCTACCTCCCCTGAGCTTAAGCA | |
| alpha | TCAAGGCTTAGTAGTCTGAGTTAAGdAACCCATAAATGGGGTGCATTGTGG | |
| actin | CAGGTCAGCAATCGTGTGTCCAGGTGGGCAGAACTGGGGAGACCTTTCAAA | |
| acta1 | CAGGTAAATCTTGGGAAGTACAGACCAGCAGTCTGCAAAGCAGTGACCTTT | |
| | GGCCCAGCACAGCCCTTCCGTGAGCCTTGGAGCCAGTTGGGAGGGGCAGAC | |
| | AGCTGGGGATACTCTCCATATACGGCCTGGTCCGGTCCTAGCTACCTGGGC | |
| | CAGGGCCAGTCCTCTCCTTCTTTGGTCAGTGCAGGAGACCCGGGGGGGGAC | |
| | CCAGGCTGAGAACCAGCCGAAGGAAGGGACTCTAGTGCCCGACACCCAAAT | |
| | ATGGCTTGGGAAGGGCAGCAACATTCCTTCGGGGCGGTGTGGGGAGAGCTC | |
| | CCGGGACTATATAAAAACCTGTGCAAGGGGACAGGCGGTC | |
| | (NCBI sequence ID No. 11459) | |
| MCK7 | CTAGAAGCTGCATGTCTAAGCTAGACCCTTCAGATTAAAAATAACTGAGGT | 23 |
| | AAGGGCCTGGGTAGGGGAGGTGGTGTGAGACGCTCCTGTCTCTCCTCTATC | |
| | TGCCCATCGGCCCTTTGGGGAGGAGGAATGTGCCCAAGGACTAAAAAAAGG | |
| | CCATGGAGCCAGAGGGGCGAGGGCAACAGACCTTTCATGGGCAAACCTTGG | |
| | GGCCCTGCTGTCTAGCATGCCCCACTACGGGTCTAGGCTGCCCATGTAAGG | |
| | AGGCAAGGCCTGGGGACACCCGAGATGCCTGGTTATAATTAACCCAGACAT | |
| | GTGGCTGCCCCCCCCCCCCCAACACCTGCTGCCTCTAAAAATAACCCTGTC | |
| | CCTGGTGGATCCCCTGCATGCGAAGATCTTCGAACAAGGCTGTGGGGGACT | |
| | GAGGGCAGGCTGTAACAGGCTTGGGGGCCAGGGCTTATACGTGCCTGGGAC | |
| | TCCCAAAGTATTACTGTTCCATGTTCCCGGCGAAGGGCCAGCTGTCCCCCG | |
| | CCAGCTAGACTCAGCACTTAGTTTAGGAACCAGTGAGCAAGTCAGCCCTTG | |
| | GGGCAGCCCATACAAGGCCATGGGGCTGGGCAAGCTGCACGCCTGGGTCCG | |
| | GGGTGGGCACGGTGCCCGGGCAACGAGCTGAAAGCTCATCTGCTCTCAGGG | |
| | GCCCCTCCCTGGGGACAGCCCCTCCTGGCTAGTCACACCCTGTAGGCTCCT | |
| | CTATATAACCCAGGGGCACAGGGGCTGCCCTCATTCTACCACCACCTCCAC | |
| | AGCAC | |
| Spc5-12 | CGAGCTCCACCGCGGTGGCGGCCGTCCGCCCTCGGCACCATCCTCACGACA | 24 |
| | CCCAAATATGGCGACGGGTGAGGAATGGTGGGGAGTTATTTTTAGAGCGGT | |
| | GAGGAAGGTGGGCAGGCAGCAGGTGTTGGCGCTCTAAAAATAACTCCCGGG | |
| | AGTTATTTTTAGAGCGGAGGAATGGTGGACACCCAAATATGGCGACGGTTC | |
| | CTCACCCGTCGCCATATTTGGGTGTCCGCCCTCGGCCGGGGCCGCATTCCT | |
| | GGGGGCCGGGCGGTGCTCCCGCCCGCCTCGATAAAAGGCTCCGGGGCCGGC | |
| | GGCGGCCCACGAGCTACCCGGAGGAGCGGGAGGCGCCAAGCTCTAGAACTA | |
| | GTGGATCCCCCGGGCTGCAGGAATTC | |
| Truncated | CCACTACGGG TCTAGGCTGC CCATGTAAGG AGGCAAGGCC | 33 |
| MCK | TGGGGACACC CGAGATGCCT GGTTATAATT AACCCCAACA | |
| (tMCK) | CCTGCTGCCC CCCCCCCCCC AACACCTGCT GCCTGAGCCT | |
| | GAGCGGTTAC CCCACCCCGG TGCCTGGGTC TTAGGCTCTG | |
| | TACACCATGG AGGAGAAGCT CGCTCTAAAA ATAACCCTGT | |
| | CCCTGGTGGA TCCACTACGG GTCTATGCTG CCCATGTAAG | |
| | GAGGCAAGGC CTGGGGACAC CCGAGATGCC TGGTTATAAT | |
| | TAACCCCAAC ACCTGCTGCC CCCCCCCCCC CAACACCTGC | |
| | TGCCTGAGCC TGAGCGGTTA CCCCACCCCG GTGCCTGGGT | |
| | CTTAGGCTCT GTACACCATG GAGGAGAAGC TCGCTCTAAA | |
| | AATAACCCTG TCCCTGGTGG ACCACTACGG GTCTAGGCTG | |
| | CCCATGTAAG GAGGCAAGGC CTGGGGACAC CCGAGATGCC | |
| | TGGTTATAAT TAACCCCAAC ACCTGCTGCC CCCCCCCCCC | |
| | AACACCTGCT GCCTGAGCCT GAGCGGTTAC CCCACCCCGG | |
| | TGCCTGGGTC TTAGGCTCTG TACACCATGG AGGAGAAGCT | |
| | CGCTCTAAAA ATAACCCTGT CCCTGGTCCT CCCTGGGGAC | |
| | AGCCCCTCCT GGCTAGTCAC ACCCTGTAGG CTCCTCTATA | |
| | TAACCCAGGG GCACAGGGGC TGCCCCCGGG TCAC | |

In some embodiments, the muscle cell-specific promoter is a muscle creatine-kinase (“MCK”) promoter or a truncated MCK promoter. The muscle creatine kinase (MCK) gene is highly active in all striated muscles. Creatine kinase plays an important role in the regeneration of ATP within contractile and ion transport systems. It allows for muscle contraction when neither glycolysis nor respiration is present by transferring a phosphate group from phosphocreatine to ADP to form ATP. There are four known isoforms of creatine kinase: brain creatine kinase (CKB), muscle creatine kinase (MCK), and two mitochondrial forms (CKMi). MCK is the most abundant non-mitochondrial mRNA that is expressed in all skeletal muscle fiber types and is also highly active in cardiac muscle. The MCK gene is not expressed in myoblasts, but becomes transcriptionally active when myoblasts commit to terminal differentiation into myocytes. MCK gene regulatory regions display striated muscle-specific activity and have been extensively characterized in vivo and in vitro. The major known regulatory regions in the MCK gene include a muscle-specific enhancer located approximately 1.1 kb 5′ of the transcriptional start site in mouse and a 358-bp proximal promoter. Additional sequences that modulate MCK expression are distributed over 3.3 kb region 5′ of the transcriptional start site and in the 3.3-kb first intron. Mammalian MCK regulatory elements, including human and mouse promoter and enhancer elements, are described in Hauser et al., “Analysis of Muscle Creatine Kinase Regulatory Elements in Recombinant Adenoviral Vectors,” Mol. Therapy 2:16-25 (2000), which is hereby incorporated by reference in its entirety. Suitable muscle creatine kinase (MCK) promoters include, without limitation, a wild type MCK promoter, a dMCK promoter, and a tMCK promoter (Wang et al., “Construction and Analysis of Compact Muscle-Specific Promoters for AAV Vectors,” Gene Ther. 15(22):1489-1499 (2008), which is hereby incorporated by reference in its entirety).

Alternatively, the promoter may be a constitutive promoter, for example, the CB7 promoter. Additional promoters include: cytomegalovirus (CMV) promoter, Rous sarcoma virus (RSV) promoter, MMT promoter, EF-1 alpha promoter, UB6 promoter, chicken beta-actin promoter, or CAG promoter. In some embodiments, particularly where it may be desirable to turn off transgene expression, an inducible promoter is used, e.g., hypoxia-inducible or rapamycin-inducible promoter.

### 5.3.2 Introns

Certain gene expression cassettes further include an intron, for example, 5′ of the AUF1 coding sequence which may enhances proper splicing and, thus, AUF1 expression. Accordingly, in some embodiments, an intron is coupled to the 5′ end of a sequence encoding an AUF1 protein. In particular, the intron nucleotide sequence can be linked to the nucleotide sequence attached to the actin-binding domain. In other embodiments, the intron is less than 100 nucleotides in length. In embodiments, the intron is a VH4 intron. The VH4 intron nucleic acid can comprise SEQ ID NO: 25 as shown in Table 3 below.

TABLE 3
Nucleotide sequences for different introns

| | SEQ | |
|---|---|---|
| Structure | ID | Sequence |
| VH4 | 25 | GTGAGTATCTCAGGGATCCAGACATGGGGATATGGG |
| intron | | AGGTGCCTCTGATCCCAGGGCTCACTGTGGGTCTCT |
| | | CTGTTCACAG |
| Chimeric | 26 | GTAAGTATCAAGGTTACAAGACAGGTTTAAGGAGAC |
| intron | | CAATAGAAACTGGGCTTGTCGAGACAGAGAAGACTC |
| | | TTGCGTTTCTGATAGGCACCTATTGGTCTTACTGAC |
| | | ATCCACTTTGCCTTTCTCTCCACAG |
| SV40 | 27 | GTAAGTTTAGTCTTTTTGTCTTTTATTTCAGGTCCC |
| intron | | GGATCCGGTGGTGGTGCAAATCAAAGAACTGCTCCT |
| | | CAGTGGATGTTGCCTTTACTTCTAG |

### 5.3.3 Other Regulatory Elements

5.3.3.1 polyA

Another aspect of the present disclosure relates to expression cassettes comprising a polyadenylation (polyA) site downstream of the coding region of the AUF1 transgene. Any polyA site that signals termination of transcription and directs the synthesis of a polyA tail is suitable for use in AAV vectors of the present disclosure. Exemplary polyA signals are derived from, but not limited to, the following: the SV40 late gene, the rabbit P-globin gene, the bovine growth hormone (BPH) gene, the human growth hormone (hGH) gene, and the synthetic polyA (SPA) site.

5.3.4 Reporter genes

In some embodiments, the disclosed gene cassettes, and thus the adeno-associated viral vectors, comprise a nucleic acid molecule encoding a reporter protein. The reporter protein may be, e.g., β-galactosidase, chloramphenicol acetyl transferase, luciferase, or fluorescent proteins. In embodiments, the reporter gene sequence is linked to a transgene (such as an AUF1 coding sequence) through a linker, such as an IRES element, such that both the transgene and the reporter sequences are co-expressed from the viral vector.

In certain embodiments, the reporter protein is a fluorescent protein. Suitable fluorescent proteins include, without limitation, green fluorescent proteins (e.g., GFP, GFP-2, tagGFP, turboGFP, EGFP, Emerald, Azami Green, Monomeric Azami Green, CopGFP, AceGFP, ZsGreenl), yellow fluorescent proteins (e.g., YFP, EYFP, Citrine, Venus, YPet, PhiYFP, ZsYellowl), blue fluorescent proteins (e.g., EBFP, EBFP2, Azurite, mKalamal, GFPuv, Sapphire, T-sapphire), cyan fluorescent proteins (e.g., ECFP, Cerulean, CyPet, AmCyanl, Midoriishi-Cyan), red fluorescent proteins (mKate, mKate2, mPlum, DsRed monomer, mCherry, mRFP1, DsRed-Express, DsRed2, DsRed-Monomer, HcRed-Tandem, HcRedl, AsRed2, mRasberry, mStrawberry, Jred), and orange fluorescent proteins (mOrange, mKO, Kusabira-Orange, Monomeric Kusabira-Orange, mTangerine, tdTomato), or any other suitable fluorescent protein. In certain embodiments, the reporter protein is a fluorescent protein, including green fluorescent protein (GFP), enhanced green fluorescent protein (EGFP), and yellow fluorescent protein (YFP).

In some embodiments, the reporter protein is luciferase. As used herein, the term “luciferase” refers to members of a class of enzymes that catalyze reactions that result in production of light. Luciferases have been identified in and cloned from a variety of organisms including fireflies, click beetles, sea pansy (Renilla), marine copepods, and bacteria among others. Examples of luciferases that may be used as reporter proteins include, e.g., Renilla (e.g., Renilla reniformis) luciferase, Gaussia (e.g., Gaussia princeps) luciferase), Metridia luciferase, firefly (e.g., Photinus pyralis luciferase), click beetle (e.g., Pyrearinus termitilluminans) luciferase, deep sea shrimp (e.g., Oplophorus gracilirostris) luciferase). Luciferase reporter proteins include both naturally occurring proteins and engineered variants designed to have one or more altered properties relative to the naturally occurring protein, such as increased photostability, increased pH stability, increased fluorescence or light output, reduced tendency to dimerize, oligomerize, aggregate or be toxic to cells, an altered emission spectrum, and/or altered substrate utilization.

### 5.3.5 Viral Vectors

Adeno-associated viral vectors and recombinant adeno-associated virus (AAV) vectors are well known delivery vehicles that can be constructed and used to deliver a nucleic acid molecule to cells, as described in Shi et al., “Therapeutic Expression of an Anti-Death Receptor-5 Single-Chain Fixed Variable Region Prevents Tumor Growth in Mice,” Cancer Res. 66:11946-53 (2006); Fukuchi et al., “Anti-Aβ Single-Chain Antibody Delivery via Adeno-Associated Virus for Treatment of Alzheimer's Disease,” Neurobiol. Dis. 23:502-511 (2006); Chatterjee et al., “Dual-Target Inhibition of HIV-1 In Vitro by Means of an Adeno-Associated Virus Antisense Vector,” Science 258:1485-1488 (1992); Ponnazhagan et al., “Suppression of Human Alpha-globin Gene Expression Mediated by the Recombinant Adeno-associated Virus 2-based Antisense Vectors,” J. Exp. Med. 179:733-738 (1994), which are hereby incorporated by reference in their entirety. In vivo use of these vehicles is described in Flotte et al., “Stable In Vivo Expression of the Cystic Fibrosis Transmembrane Conductance Regulator With an Adeno-Associated Virus Vector,” Proc. Nat'l. Acad. Sci. 90:10613-10617 (1993), which is hereby incorporated by reference in its entirety.

Recombinant adeno-associated virus (AAV) vectors provide the ability to stably transduce and express genes with very long-term (many years) duration in skeletal muscle, and depending on the AAV vector serotype and its modification, to do so with high muscle-tropism and selectivity whether using local intramuscular injection or systemic routes of delivery (Phillips et al., “Systemic Gene Transfer to Skeletal Muscle Using Reengineered AAV Vectors,” Methods Mol. Biol. 709:141-51 (2011) and Muraine et al., “Transduction Efficiency of Adeno-Associated Virus Serotypes After Local Injection in Mouse and Human Skeletal Muscle,” Hum. Gene Ther. 31(3-4):233-240 (2020), which are hereby incorporated by reference in their entirety). Moreover, for certain AAV serotypes and engineered variants, particularly AAV8 and its engineered variants, studies in mice have been shown to be predictive of human skeletal muscle transduction and gene expression, as found in clinical trials for skeletal muscle transmission and expression (Phillips et al., “Systemic Gene Transfer to Skeletal Muscle Using Reengineered AAV Vectors,” Methods Mol. Biol. 709:141-51 (2011) and Muraine et al., “Transduction Efficiency of Adeno-Associated Virus Serotypes After Local Injection in Mouse and Human Skeletal Muscle,” Hum. Gene Ther. 31(3-4):233-240 (2020), which are hereby incorporated by reference in their entirety).

Accordingly, the transgenes or expression cassettes encoding AUF1 as described herein can be included in an AAV vector for gene therapy administration to a human subject. In some embodiments, recombinant AAV (rAAV) vectors can comprise an AAV viral capsid and a viral or artificial genome comprising an expression cassette flanked by AAV inverted terminal repeats (ITRs) wherein the expression cassette comprises an AUF1 transgene, operably linked to one or more regulatory sequences that control expression of the transgene in muscle cells to express and deliver the AUF1 protein. Provided are compositions comprising any isolated recombinant AAV particles encoding an AUF1 protein, and methods for treating a disease or disorder amenable for treatment with AUF1 in a subject in need thereof comprising the administration of any isolated recombinant AAV particles encoding AUF1 as described herein. As such, the rAAV can be of any serotype, variant, modification, hybrid, or derivative thereof, known in the art, or any combination thereof (collectively referred to as “serotype”), including for delivery and expression of the AUF1 transgene in muscle cells, including skeletal and/or cardiac muscle cells. Accordingly, in particular embodiments, the AAV serotype has a tropism for muscle tissue. The rAAV vector described herein may comprise a capsid of serotype 1 (AAV1), 2 (AAV2), 3 (AAV3), 4 (AAV4), 5 (AAV5), 6 (AAV6), 7 (AAV7), 8 (AAV8), 9 (AAV9), 10 (AAV10), 11 (AAV11) or any combination thereof.

In some embodiments, the adeno-associated viral (AAV) vector is a recombinant vector.

In one particular embodiment, the AAV vector is AAV8 serotype. AAV8 derived from macaques is very poorly immunogenic, resulting in long-term expression of the encoded transgene (for many years), and efficiently transduce skeletal muscle with high tropism and selectivity in both human and mouse (Phillips et al., “Systemic Gene Transfer to Skeletal Muscle Using Reengineered AAV Vectors,” Methods Mol. Biol. 709:141-51 (2011); Muraine et al., “Transduction Efficiency of Adeno-Associated Virus Serotypes After Local Injection in Mouse and Human Skeletal Muscle,” Hum. Gene Ther. 31(3-4):233-240 (2020); Blankinship et al., “Efficient Transduction of Skeletal Muscle Using Vectors Based on Adeno-associated Virus Serotype 6,” Mol. Ther. 10(4):671-8 (2004); and Gregorevic et al., “Viral Vectors for Gene Transfer to Striated Muscle,” Curr. Opin. Mol. Ther. 6(5):491-8 (2004), which are hereby incorporated by reference in their entirety). AAV8 shows essentially no liver tropism, is largely specific for skeletal fibers and satellite cells, and has been shown to transduce skeletal muscles throughout the body (Wang et al., “Construction and Analysis of Compact Muscle-specific Promoters for AAV Vectors,” Gene Ther. 15(22):1489-99 (2008), which is hereby incorporated by reference in its entirety).

According to one embodiment, the adeno-associated viral (AAV) vector is an AAV8 vector which has a capsid encoded by the nucleotide sequence of SEQ ID NO:28.

AAV8 Capsid Nucleotide Sequence
(SEQ ID NO: 28)
CCTGCAGGCA GCTGCGCGCT CGCTCGCTCA CTGAGGCCGC CCGGGCAAAG CCCGGGCGTC 60
GGGCGACCTT TGGTCGCCCG GCCTCAGTGA GCGAGCGAGC GCGCAGAGAG GGAGTGGCCA 120
ACTCCATCAC TAGGGGTTCC TGCGGCCTAA GGCAATTGGC CACTACGGGT CTAGGCTGCC 180
CATGTAAGGA GGCAAGGCCT GGGGACACCC GAGATGCCTG GTTATAATTA ACCCCAACAC 240
CTGCTGCCCC CCCCCCCCAA CACCTGCTGC CTGAGCCTGA GCGGTTACCC CACCCCGGTG 300
CCTGGGTCTT AGGCTCTGTA CACCATGGAG GAGAAGCTCG CTCTAAAAAT AACCCTGTCC 360
CTGGTGGATC GCCACTACGG GTCTAGGCTG CCCATGTAAG GAGGCAAGGC CTGGGGACAC 420
CCGAGATGCC TGGTTATAAT TAACCCCAAC ACCTGCTGCC CCCCCCCCCC AACACCTGCT 480
GCCTGAGCCT GAGCGGTTAC CCCACCCCGG TGCCTGGGTC TTAGGCTCTG TACACCATGG 540
AGGAGAAGCT CGCTCTAAAA ATAACCCTGT CCCTGGTGGA TCGCCACTAC GGGTCTAGGC 600
TGCCCATGTA AGGAGGCAAG GCCTGGGGAC ACCCGAGATG CCTGGTTATA ATTAACCCCA 660
ACACCTGCTG CCCCCCCCCC CCAACACCTG CTGCCTGAGC CTGAGCGGTT ACCCCACCCC 720
GGTGCCTGGG TCTTAGGCTC TGTACACCAT GGAGGAGAAG CTCGCTCTAA AAATAACCCT 780
GTCCCTGGTG GATCCCTCCC TGGGGACAGC CCCTCCTGGC TAGTCACACC CTGTAGGCTC 840
CTCTATATAA CCCAGGGGCA CAGGGGCTGC CCCCGGGTCA CCGCTAGCCA AAGCTTCTCG 900
AGGCTGGCTA GTTAAGCTAT CAACAAGTTT GTACAGAAAA GCAGGCTTTA AAGGAACCAA 960
TTCAGTCGAC GCTAGCAAGC TTGGTACCGG ATCCGAATTC CACCATGTCG GAGGAGCAGT 1020
TCGGAGGGGA CGGGGCGGCG GCGGCGGCAA CGGCGGCGGT AGGCGGCTCG GCGGGCGAGC 1080
AGGAGGGAGC CATGGTGGCG GCGGCGGCGC AGGGGCCGGC GGCGGCGGCG GGAAGCGGGA 1140
GCGGCGGCGG CGGCTCTGCG GCCGGAGGCA CCGAAGGAGG CAGCGCCGAG GCAGAGGGAG 1200
CCAAGATCGA CGCCAGTAAG AACGAGGAGG ATGAAGGCCA TTCAAACTCC TCCCCACGAC 1260
ACACTGAAGC AGCGGCGGCA CAGCGGGAAG AATGGAAAAT GTTTATAGGA GGCCTTAGCT 1320
GGGACACCAC AAAGAAAGAT CTGAAGGACT ACTTTTCCAA ATTTGGTGAA GTTGTAGACT 1380
GCACTCTGAA GTTAGATCCT ATCACAGGGC GATCAAGGGG TTTTGGCTTT GTGCTATTTA 1440
AAGAGTCGGA GAGTGTAGAT AAGGTCATGG ATCAGAAAGA ACATAAATTG AATGGGAAAG 1500
TCATTGATCC TAAAAGGGCC AAAGCCATGA AAACAAAAGA GCCTGTCAAA AAAATTTTTG 1560
TTGGTGGCCT TTCTCCAGAC ACACCTGAAG AAAAAATAAG AGAGTACTTT GGTGGTTTTG 1620
GTGAGGTTGA ATCCATAGAG CTCCCTATGG ACAACAAGAC CAATAAGAGG CGTGGGTTCT 1680
GTTTTATTAC CTTTAAGGAA GAGGAGCCAG TGAAGAAGAT AATGGAAAAG AAATACCACA 1740
ATGTTGGTCT TAGTAAATGT GAAATAAAAG TAGCCATGTC AAAGGAACAG TATCAGCAGC 1800
AGCAGCAGTG GGGATCTAGA GGAGGGTTTG CAGGCAGAGC TCGCGGAAGA GGTGGAGATC 1860
AGCAGAGTGG TTATGGGAAA GTATCCAGGC GAGGTGGACA TCAAAATAGC TACAAACCAT 1920
ACTAAGATAT CGCGGCCGCC TCGAGGACTA CAAGGATGAC GATGACAAGG ATTACAAAGA 1980
CGACGATGAT AAGGACTATA AGGATGATGA CGACAAATAA TAGCAATTCC TCGACGACTG 2040
CATAGGGTTA CCCCCCTCTC CCTCCCCCCC CCCTAACGTT ACTGGCCGAA GCCGCTTGGA 2100
ATAAGGCCGG TGTGCGTTTG TCTATATGTT ATTTTCCACC ATATTGCCGT CTTTTGGCAA 2160
TGTGAGGGCC CGGAAACCTG GCCCTGTCTT CTTGACGAGC ATTCCTAGGG GTCTTTCCCC 2220
TCTCGCCAAA GGAATGCAAG GTCTGTTGAA TGTCGTGAAG GAAGCAGTTC CTCTGGAAGC 2280
TTCTTGAAGA CAAACAACGT CTGTAGCGAC CCTTTGCAGG CAGCGGAACC CCCCACCTGG 2340
CGACAGGTGC CTCTGCGGCC AAAAGCCACG TGTATAAGAT ACACCTGCAA AGGCGGCACA 2400
ACCCCAGTGC CACGTTGTGA GTTGGATAGT TGTGGAAAGA GTCAAATGGC TCTCCTCAAG 2460
CGTATTCAAC AAGGGGCTGA AGGATGCCCA GAAGGTACCC CATTGTATGG GATCTGATCT 2520
GGGGCCTCGG TGCACATGCT TTACATGTGT TTAGTCGAGG TTAAAAAACG TCTAGGCCCC 2580
CCGAACCACG GGGACGTGGT TTTCCTTTGA AAAACACGAT GATAATGGCC ACAACTAGTG 2640
CCACCATGGT GAGCAAGGGC GAGGAGCTGT TCACCGGGGT GGTGCCCATC CTGGTCGAGC 2700
TGGACGGCGA CGTAAACGGC CACAAGTTCA GCGTGTCCGG CGAGGGCGAG GGCGATGCCA 2760
CCTACGGCAA GCTGACCCTG AAGTTCATCT GCACCACCGG CAAGCTGCCC GTGCCCTGGC 2820
CCACCCTCGT GACCACCCTG ACCTACGGCG TGCAGTGCTT CAGCCGCTAC CCCGACCACA 2880
TGAAGCAGCA CGACTTCTTC AAGTCCGCCA TGCCCGAAGG CTACGTCCAG GAGCGCACCA 2940
TCTTCTTCAA GGACGACGGC AACTACAAGA CCCGCGCCGA GGTGAAGTTC GAGGGCGACA 3000
CCCTGGTGAA CCGCATCGAG CTGAAGGGCA TCGACTTCAA GGAGGACGGC AACATCCTGG 3060
GGCACAAGCT GGAGTACAAC TACAACAGCC ACAACGTCTA TATCATGGCC GACAAGCAGA 3120
AGAACGGCAT CAAGGTGAAC TTCAAGATCC GCCACAACAT CGAGGACGGC AGCGTGCAGC 3180
TCGCCGACCA CTACCAGCAG AACACCCCCA TCGGCGACGG CCCCGTGCTG CTGCCCGACA 3240
ACCACTACCT GAGCACCCAG TCCGCCCTGA GCAAAGACCC CAACGAGAAG CGCGATCACA 3300
TGGTCCTGCT GGAGTTCGTG ACCGCCGCCG GGATCACTCT CGGCATGGAC GAGCTGTACA 3360
AGTAAGTTTA AACTCTAGAC CCAGCTTTCT TGTACAAAGT GGTTGATCTA GAGGGCCCGT 3420
AACTAGTTGA GCGGCCGCAA CTCGAGACTC TAGAGGTTAA TCGATAATCA ACCTCTGGAT 3480
TACAAAATTT GTGAAAGATT GACTGGTATT CTTAACTATG TTGCTCCTTT TACGCTATGT 3540
GGATACGCTG CTTTAATGCC TTTGTATCAT GCTATTGCTT CCCGTATGGC TTTCATTTTC 3600
TCCTCCTTGT ATAAATCCTG GTTGCTGTCT CTTTATGAGG AGTTGTGGCC CGTTGTCAGG 3660
CAACGTGGCG TGGTGTGCAC TGTGTTTGCT GACGCAACCC CCACTGGTTG GGGCATTGCC 3720
ACCACCTGTC AGCTCCTTTC CGGGACTTTC GCTTTCCCCC TCCCTATTGC CACGGCGGAA 3780
CTCATCGCCG CCTGCCTTGC CCGCTGCTGG ACAGGGGCTC GGCTGTTGGG CACTGACAAT 3840
TCCGTGGTGT TGTCGGGGAA ATCATCGTCC TTTCCTTGGC TGCTCGCCTG TGTTGCCACC 3900
TGGATTCTGC GCGGGACGTC CTTCTGCTAC GTCCCTTCGG CCCTCAATCC AGCGGACCTT 3960
CCTTCCCGCG GCCTGCTGCC GGCTCTGCGG CCTCTTCCGC GTCTTCGCCT TCGCCCTCAG 4020
ACGAGTCGGA TCTCCCTTTG GGCCGCCTCC CCGCATCGAA ACCCGCTGAC TAGACGACTG 4080
TGCCTTCTAG TTGCCAGCCA TCTGTTGTTT GCCCCTCCCC CGTGCCTTCC TTGACCCTGG 4140
AAGGTGCCAC TCCCACTGTC CTTTCCTAAT AAAATGAGGA AATTGCATCG CATTGTCTGA 4200
GTAGGTGTCA TTCTATTCTG GGGGGTGGGG TGGGGCAGGA CAGCAAGGGG GAGGATTGGG 4260
AAGACAATAG CAGGCATGCT GGGGATGCGG TGGGCTCTAT GGCCGCGGGC CGCAGGAACC 4320
CCTAGTGATG GAGTTGGCCA CTCCCTCTCT GCGCGCTCGC TCGCTCACTG AGGCCGGGCG 4380
ACCAAAGGTC GCCCGACGCC CGGGCTTTGC CCGGGCGGCC TCAGTGAGCG AGCGAGCGCG 4440
CAGCTGCCTG CAGGGGCGCC TGATGCGGTA TTTTCTCCTT ACGCATCTGT GCGGTATTTC 4500
ACACCGCATA CGTCAAAGCA ACCATAGTAC GCGCCCTGTA GCGGCGCATT AAGCGCGGCG 4560
GGTGTGGTGG TTACGCGCAG CGTGACCGCT ACACTTGCCA GCGCCCTAGC GCCCGCTCCT 4620
TTCGCTTTCT TCCCTTCCTT TCTCGCCACG TTCGCCGGCT TTCCCCGTCA AGCTCTAAAT 4680
CGGGGGCTCC CTTTAGGGTT CCGATTTAGT GCTTTACGGC ACCTCGACCC CAAAAAACTT 4740
GATTTGGGTG ATGGTTCACG TAGTGGGCCA TCGCCCTGAT AGACGGTTTT TCGCCCTTTG 4800
ACGTTGGAGT CCACGTTCTT TAATAGTGGA CTCTTGTTCC AAACTGGAAC AACACTCAAC 4860
CCTATCTCGG GCTATTCTTT TGATTTATAA GGGATTTTGC CGATTTCGGC CTATTGGTTA 4920
AAAAATGAGC TGATTTAACA AAAATTTAAC GCGAATTTTA ACAAAATATT AACGTTTACA 4980
ATTTTATGGT GCACTCTCAG TACAATCTGC TCTGATGCCG CATAGTTAAG CCAGCCCCGA 5040
CACCCGCCAA CACCCGCTGA CGCGCCCTGA CGGGCTTGTC TGCTCCCGGC ATCCGCTTAC 5100
AGACAAGCTG TGACCGTCTC CGGGAGCTGC ATGTGTCAGA GGTTTTCACC GTCATCACCG 5160
AAACGCGCGA GACGAAAGGG CCTCGTGATA CGCCTATTTT TATAGGTTAA TGTCATGATA 5220
ATAATGGTTT CTTAGACGTC AGGTGGCACT TTTCGGGGAA ATGTGCGCGG AACCCCTATT 5280
TGTTTATTTT TCTAAATACA TTCAAATATG TATCCGCTCA TGAGACAATA ACCCTGATAA 5340
ATGCTTCAAT AATATTGAAA AAGGAAGAGT ATGAGTATTC AACATTTCCG TGTCGCCCTT 5400
ATTCCCTTTT TTGCGGCATT TTGCCTTCCT GTTTTTGCTC ACCCAGAAAC GCTGGTGAAA 5460
GTAAAAGATG CTGAAGATCA GTTGGGTGCA CGAGTGGGTT ACATCGAACT GGATCTCAAC 5520
AGCGGTAAGA TCCTTGAGAG TTTTCGCCCC GAAGAACGTT TTCCAATGAT GAGCACTTTT 5580
AAAGTTCTGC TATGTGGCGC GGTATTATCC CGTATTGACG CCGGGCAAGA GCAACTCGGT 5640
CGCCGCATAC ACTATTCTCA GAATGACTTG GTTGAGTACT CACCAGTCAC AGAAAAGCAT 5700
CTTACGGATG GCATGACAGT AAGAGAATTA TGCAGTGCTG CCATAACCAT GAGTGATAAC 5760
ACTGCGGCCA ACTTACTTCT GACAACGATC GGAGGACCGA AGGAGCTAAC CGCTTTTTTG 5820
CACAACATGG GGGATCATGT AACTCGCCTT GATCGTTGGG AACCGGAGCT GAATGAAGCC 5880
ATACCAAACG ACGAGCGTGA CACCACGATG CCTGTAGCAA TGGCAACAAC GTTGCGCAAA 5940
CTATTAACTG GCGAACTACT TACTCTAGCT TCCCGGCAAC AATTAATAGA CTGGATGGAG 6000
GCGGATAAAG TTGCAGGACC ACTTCTGCGC TCGGCCCTTC CGGCTGGCTG GTTTATTGCT 6060
GATAAATCTG GAGCCGGTGA GCGTGGGTCT CGCGGTATCA TTGCAGCACT GGGGCCAGAT 6120
GGTAAGCCCT CCCGTATCGT AGTTATCTAC ACGACGGGGA GTCAGGCAAC TATGGATGAA 6180
CGAAATAGAC AGATCGCTGA GATAGGTGCC TCACTGATTA AGCATTGGTA ACTGTCAGAC 6240
CAAGTTTACT CATATATACT TTAGATTGAT TTAAAACTTC ATTTTTAATT TAAAAGGATC 6300
TAGGTGAAGA TCCTTTTTGA TAATCTCATG ACCAAAATCC CTTAACGTGA GTTTTCGTTC 6360
CACTGAGCGT CAGACCCCGT AGAAAAGATC AAAGGATCTT CTTGAGATCC TTTTTTTCTG 6420
CGCGTAATCT GCTGCTTGCA AACAAAAAAA CCACCGCTAC CAGCGGTGGT TTGTTTGCCG 6480
GATCAAGAGC TACCAACTCT TTTTCCGAAG GTAACTGGCT TCAGCAGAGC GCAGATACCA 6540
AATACTGTCC TTCTAGTGTA GCCGTAGTTA GGCCACCACT TCAAGAACTC TGTAGCACCG 6600
CCTACATACC TCGCTCTGCT AATCCTGTTA CCAGTGGCTG CTGCCAGTGG CGATAAGTCG 6660
TGTCTTACCG GGTTGGACTC AAGACGATAG TTACCGGATA AGGCGCAGCG GTCGGGCTGA 6720
ACGGGGGGTT CGTGCACACA GCCCAGCTTG GAGCGAACGA CCTACACCGA ACTGAGATAC 6780
CTACAGCGTG AGCTATGAGA AAGCGCCACG CTTCCCGAAG GGAGAAAGGC GGACAGGTAT 6840
CCGGTAAGCG GCAGGGTCGG AACAGGAGAG CGCACGAGGG AGCTTCCAGG GGGAAACGCC 6900
TGGTATCTTT ATAGTCCTGT CGGGTTTCGC CACCTCTGAC TTGAGCGTCG ATTTTTGTGA 6960
TGCTCGTCAG GGGGGCGGAG CCTATGGAAA AACGCCAGCA ACGCGGCCTT TTTACGGTTC 7020
CTGGCCTTTT GCTGGCCTTT TGCTCACATG T 7051

In certain embodiments, the rAAV particles have an AAV8 serotype capsid and AAV2 5′ and 3′ ITRs. The amino acid sequence of the AAV8 capsid and the nucleotide sequences of the AAV2 ITRs are provided in Table 4.

TABLE 4

| | SEQ | |
|---|---|---|
| Structure | ID | Sequence |
| AAV8 | 29 | MAADGYLPDW LEDNLSEGIR EWWALKPGAP KPKANQQKOD DGRGLVLPGY |
| Capsid | | KYLGPFNGLD KGEPVNAADA AALEHDKAYD QQLQAGDNPY LRYNHADAEF |
| | | QERLQEDTSF GGNLGRAVFQ AKKRVLEPLG LVEEGAKTAP GKKRPVEPSP |
| | | QRSPDSSTGI GKKGQQPARK RLNFGQTGDS ESVPDPQPLG EPPAAPSGVG |
| | | PNTMAAGGGA PMADNNEGAD GVGSSSGNWH CDSTWLGDRV ITTSTRTWAL |
| | | PTYNNHLYKQ ISNGTSGGAT NDNTYFGYST PWGYFDFNRF HCHFSPRDWQ |
| | | RLINNNWGFR PKRLSFKLEN IQVKEVTQNE GTKTIANNLT STIQVFTDSE |
| | | YQLPYVLGSA HQGCLPPFPA DVFMIPQYGY LTLNNGSQAV GRSSFYCLEY |
| | | FPSQMLRTGN NFQFTYTFED VPFHSSYAHS QSLDRLMNPL IDQYLYYLSR |
| | | TOTTGGTANT QTLGFSQGGP NTMANQAKNW LPGPCYRQQR VSTTTGQNNN |
| | | SNFAWTAGTK YHLNGRNSLA NPGIAMATHK DDEERFFPSN GILIFGKQNA |
| | | ARDNADYSDV MLTSEEEIKT TNPVATEEYG IVADNLQQON TAPQIGTVNS |
| | | QGALPGMVWQ NRDVYLQGPI WAKIPHTDGN FHPSPLMGGF GLKHPPPQIL |
| | | IKNTPVPADP PTTFNQSKLN SFITQYSTGQ VSVEIEWELQ KENSKRWNPE |
| | | IQYTSNYYKS TSVDFAVNTE GVYSEPRPIG TRYLTRNL |
| AAV2 | 30 | cgcgcgctcg ctcgctcact gaggccgccc gggcaaagcc cgggcgtcgg |
| 5′ITR | | gcgacctttg gtcgcccggc ctcagtgagc gagcgagcgc gcagagaggg |
| | | agtggccaac tccatcacta ggggttcct |
| AAV2 | 31 | aggaacccct agtgatggag ttggccactc cctctctgcg cgctcgctcg |
| 3′ITR | | ctcactgagg ccgggcgacc aaaggtcgcc cgacgcccgg gctttgcccg |
| | | ggcggcctca gtgagcgagc gagcgcgcag |

In some embodiments, rAAV particles comprise a capsid protein that has an AAV8 capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of the AAV8 capsid protein (SEQ ID NO: 29). In some embodiments, the rAAV particles have an AAV capsid serotype of AAV8 or a derivative, modification, or pseudotype thereof. In other embodiments, the rAAV is an AAV2i8 or AAV2.5 serotype or alternatively may be an AAVrh.8, AAVrh.10, AAVrh.43, or AAVrh.74 serotype.

In additional embodiments, rAAV particles comprise a pseudotyped AAV capsid. In some embodiments, the pseudotyped AAV capsids are an rAAV2/8 pseudotyped AAV capsids. Methods for producing and using pseudotyped rAAV particles are known in the art (see, e.g., Duan et al., J. Virol., 75:7662-7671 (2001); Halbert et al., J. Virol., 74:1524-1532 (2000); Zolotukhin et al., Methods 28:158-167 (2002); and Auricchio et al., Hum. Molec. Genet. 10:3075-3081, (2001).

In certain embodiments, a single-stranded AAV (ssAAV) can be used. In certain embodiments, a self-complementary vector, e.g., scAAV, can be used (see, e.g., Wu, 2007, Human Gene Therapy, 18(2):171-82, McCarty et al, 2001, Gene Therapy, Vol. 8, Number 16, Pages 1248-1254; and U.S. Pat. Nos. 6,596,535; 7,125,717; and 7,456,683, each of which is incorporated herein by reference in its entirety).

Another aspect of the present application relates to a composition comprising an adeno-associated viral (AAV) vector as described herein.

In some embodiments, the rAAV genome comprises a vector comprising the following components: (1) AAV inverted terminal repeats that flank an expression cassette; (2) regulatory control elements, such as a) promoter/enhancers, b) a poly A signal, and c) optionally an intron; and (3) nucleic acid sequences coding for AUF1. In a specific embodiment, the constructs described herein comprise the following components: (1) AAV2 or AAV8 inverted terminal repeats (ITRs) that flank the expression cassette; (2) control elements, which include a muscle-specific promoter and a poly A signal; and (3) transgene providing (e.g., coding for) a nucleic acid encoding human AUF1 as described herein (including a human p37AUF1, p40AUF1, p42AUF1 or p45AUF1). In a specific embodiment, the constructs described herein comprise the following components: (1) AAV2 or AAV8 ITRs that flank the expression cassette; (2) control elements, which include a) the muscle-specific MCK promoter, b) a poly A signal, and c) optionally, an intron sequence; and (3) AUF1 coding sequence

In a specific embodiment, the constructs described herein comprise the following components: (1) AAV2 ITRs that flank the expression cassette; (2) control elements, which include a promoter, such as the muscle-specific MCK promoter, and b) a poly A signal; and (3) the nucleic acid encoding an AUF1 (including a human p37AUF1, p40AUF1, p42AUF1 or p45AUF1). In some embodiments, constructs described herein comprising AAV ITRs flanking an AUF1 expression cassette, which includes one or more of the AUF1 sequences disclosed herein.

### 5.4. Methods of Making rAAV Particles

Provided are methods of making the rAAV particles comprising a genome with a transgene encoding an AUF1 protein. In embodiments, the rAAV particles are made by providing a nucleic acid encoding a capsid protein, such as an AAV8 capsid protein; and using a packaging cell system to prepare corresponding rAAV particles with capsid coats made up of the capsid protein. The capsid protein, coat, and rAAV particles may be produced by techniques known in the art. In some embodiments, the viral genome comprises at least one inverted terminal repeat to allow packaging into a vector. In some embodiments, the viral genome further comprises a cap gene and/or a rep gene for expression and splicing of the cap gene. In embodiments, the cap and rep genes are provided by a packaging cell and not present in the viral genome.

In some embodiments, the nucleic acid encoding the engineered capsid protein is cloned into an AAV Rep-Cap plasmid in place of the existing capsid gene. When introduced together into host cells, this plasmid helps package an rAAV genome into the engineered capsid protein as the capsid coat. Packaging cells can be any cell type possessing the genes necessary to promote AAV genome replication, capsid assembly, and packaging.

Numerous cell culture-based systems are known in the art for production of rAAV particles, any of which can be used to practice a method disclosed herein. The cell culture-based systems include transfection, stable cell line production, and infectious hybrid virus production systems which include, but are not limited to, adenovirus-AAV hybrids, herpesvirus-AAV hybrids and baculovirus-AAV hybrids. rAAV production cultures for the production of rAAV virus particles require: (1) suitable host cells, including, for example, human-derived cell lines, mammalian cell lines, or insect-derived cell lines; (2) suitable helper virus function, provided by wild type or mutant adenovirus (such as temperature-sensitive adenovirus), herpes virus, baculovirus, or a plasmid construct providing helper functions; (3) AAV rep and cap genes and gene products; (4) a transgene (such as a therapeutic transgene) flanked by AAV ITR sequences and optionally regulatory elements; and (5) suitable media and media components (nutrients) to support cell growth/survival and rAAV production.

Nonlimiting examples of host cells include: A549, WEHI, 10T1/2, BHK, MDCK, COS1, COS7, BSC 1, BSC 40, BMT 10, VERO, W138, HeLa, HEK293 and their derivatives (HEK293T cells, HEK293F cells), Saos, C2C12, L, HT1080, HepG2, primary fibroblast, hepatocyte, myoblast cells, CHO cells or CHO-derived cells, or insect-derived cell lines such as SF-9 (e.g. in the case of baculovirus production systems). For a review, see Aponte-Ubillus et al., 2018, Appl. Microbiol. Biotechnol. 102:1045-1054, which is incorporated by reference herein in its entirety for manufacturing techniques.

A skilled artisan is aware of the numerous methods by which AAV rep and cap genes, AAV helper genes (e.g., adenovirus E1a gene, E1b gene, E4 gene, E2a gene, and VA gene), and rAAV genomes (comprising one or more genes of interest flanked by inverted terminal repeats (ITRs)) can be introduced into cells to produce or package rAAV. The phrase “adenovirus helper functions” refers to a number of viral helper genes expressed in a cell (as RNA or protein) such that the AAV grows efficiently in the cell. The skilled artisan understands that helper viruses, including adenovirus and herpes simplex virus (HSV), promote AAV replication and certain genes have been identified that provide the essential functions, e.g. the helper may induce changes to the cellular environment that facilitate such AAV gene expression and replication. In some embodiments of a method disclosed herein, AAV rep and cap genes, helper genes, and rAAV genomes are introduced into cells by transfection of one or more plasmid vectors encoding the AAV rep and cap genes, helper genes, and rAAV genome.

Any combination of vectors can be used to introduce AAV rep and cap genes, AAV helper genes, and rAAV genome to a cell in which rAAV particles are to be produced or packaged. In some embodiments of a method disclosed herein, a first plasmid vector encoding an rAAV genome comprising a gene of interest flanked by AAV inverted terminal repeats (ITRs), a second vector encoding AAV rep and cap genes, and a third vector encoding helper genes can be used. In some embodiments, a mixture of the three vectors is co-transfected into a cell. In some embodiments, a combination of transfection and infection is used by using both plasmid vectors as well as viral vectors.

In some embodiments, one or more of rep and cap genes, and AAV helper genes are constitutively expressed by the cells and does not need to be transfected or transduced into the cells. In some embodiments, the cell constitutively expresses rep and/or cap genes. In some embodiments, the cell constitutively expresses one or more AAV helper genes. In some embodiments, the cell constitutively expresses Ela. In some embodiments, the cell comprises a stable transgene encoding the rAAV genome.

### 5.5.Compositions

Disclosed herein are compositions comprising one or more of the nucleic acid sequences, proteins, vectors, viral particles or cells described herein.

In some embodiments, the composition of the present application further comprises a buffer solution.

The composition of the present application may further comprise one or more targeting elements. Suitable targeting elements include, without limitation, agents such as saponins or cationic polyamides (see, e.g., U.S. Pat. Nos. 5,739,118 and 5,837,533, which are hereby incorporated by reference in their entirety); microparticles, microcapsules, liposomes, or other vesicles; lipids; cell-surface receptors; transfecting agents; peptides (e.g., one known to enter the nucleus); or ligands (such as one subject to receptor-mediated endocytosis). Suitable means for using such targeting elements include, without limitation: microparticle bombardment; coating the polynucleotide with lipids, cell-surface receptors, or transfecting agents; encapsulation of the polynucleotide in liposomes, microparticles, or microcapsules; administration of the polynucleotide linked to a peptide which is known to enter the nucleus; or administration of the polynucleotide linked to a ligand subject to receptor-mediated endocytosis (see, e.g., Wu et al., “Receptor-Mediated in vitro Gene Transformation by a Soluble DNA Carrier System,” J. Biol. Chem. 262:4429-4432 (1987), which is hereby incorporated by reference in its entirety), which can be used to target cell types specifically expressing the receptors. Alternatively, a polynucleotide-ligand complex can be formed allowing the polynucleotide to be targeted for cell specific uptake and expression in vivo by targeting a specific receptor (see, e.g., PCT Application Publication Nos. WO 92/06180, WO 92/22635, WO 92/203167, WO 93/14188, and WO 93/20221, which are hereby incorporated by reference in their entirety).

Thus, in some embodiments, the composition further includes a transfection reagent. The transfection reagent may be a positively charged transfection reagent. Suitable transfection reagents are well known in the art and include, e.g., Lipofectamine® RNAiMAX (Invitrogen™), Lipofectamine® 2000 (Invitrogen™), Lipofectamine® 3000 (Invitrogen™), Invivofectamine™ 3.0 (Invitrogen™), Lipofectamine™ MessengerMAX™ (Invitrogen™), Lipofectin™ (Invitrogen™), siLentFet™ (Bio-Rad), DharmaFECT™ (Dharmacon), HiPerFect (Qiagen), TransIT-X2® (Mirus), jetMESSENGER® (Polyplus), Trans-Hi™, JetPEI® (Polyplus), and ViaFect™ (Promega).

In some embodiments, the composition is an aqueous composition. Aqueous compositions of the present application comprise an effective amount of the vector, dissolved or dispersed in a pharmaceutically acceptable carrier or aqueous medium.

A further aspect of the present application relates to a pharmaceutical composition comprising an adeno-associated viral (AAV) vector described herein and a pharmaceutically-acceptable carrier.

The term “pharmaceutically acceptable carrier” refers to a carrier that does not cause an allergic reaction or other untoward effect in patients to whom it is administered and are compatible with the other ingredients in the formulation. Pharmaceutically acceptable carriers include, for example, pharmaceutical diluents, excipients, or carriers suitably selected with respect to the intended form of administration, and consistent with conventional pharmaceutical practices. For example, solid carriers/diluents include, but are not limited to, a gum, a starch (e.g., corn starch, pregelatinized starch), a sugar (e.g., lactose, mannitol, sucrose, dextrose), a cellulosic material (e.g., microcrystalline cellulose), an acrylate (e.g., polymethylacrylate), calcium carbonate, magnesium oxide, talc, or mixtures thereof. Pharmaceutically acceptable carriers may further comprise minor amounts of auxiliary substances such as wetting or emulsifying agents, preservatives or buffers, which enhance the shelf life or effectiveness of the nucleic acid molecule described herein.

The vector(s) (i.e., adeno-associated viral (AAV) vector and/or lentiviral vectors disclosed herein) and/or pharmaceutical composition(s) disclosed herein can be formulated according to any available conventional method. Examples of preferred dosage forms include a tablet, a powder, a subtle granule, a granule, a coated tablet, a capsule, a syrup, a troche, an inhalant, a suppository, an injectable, an ointment, an ophthalmic ointment, an eye drop, a nasal drop, an ear drop, a cataplasm, a lotion and the like. In the formulation, generally used additives such as a diluent, a binder, a disintegrant, a lubricant, a colorant, a flavoring agent, and if necessary, a stabilizer, an emulsifier, an absorption enhancer, a surfactant, a pH adjuster, an antiseptic, an antioxidant, and the like can be used.

In addition, formulating a pharmaceutical composition can be carried out by combining compositions that are generally used as a raw material for pharmaceutical formulation, according to conventional methods. Examples of these compositions include, for example, (1) an oil such as a soybean oil, a beef tallow and synthetic glyceride; (2) hydrocarbon such as liquid paraffin, squalene, and solid paraffin; (3) ester oil such as octyldodecyl myristic acid and isopropyl myristic acid; (4) higher alcohol such as cetostearyl alcohol and behenyl alcohol; (5) a silicon resin; (6) a silicon oil; (7) a surfactant such as polyoxyethylene fatty acid ester, sorbitan fatty acid ester, glycerin fatty acid ester, polyoxyethylene sorbitan fatty acid ester, a solid polyoxyethylene castor oil and polyoxyethylene polyoxypropylene block co-polymer; (8) water soluble macromolecule such as hydroxyethyl cellulose, polyacrylic acid, carboxyvinyl polymer, polyethyleneglycol, polyvinylpyrrolidone and methylcellulose; (9) lower alcohol such as ethanol and isopropanol; (10) multivalent alcohol such as glycerin, propyleneglycol, dipropyleneglycol and sorbitol; (11) a sugar such as glucose and cane sugar; (12) an inorganic powder such as anhydrous silicic acid, aluminum magnesium silicicate, and aluminum silicate; (13) purified water, and the like.

Additives for use in the above formulations may include, for example, (1) lactose, corn starch, sucrose, glucose, mannitol, sorbitol, crystalline cellulose, and silicon dioxide as the diluent; (2) polyvinyl alcohol, polyvinyl ether, methyl cellulose, ethyl cellulose, gum arabic, tragacanth, gelatine, shellac, hydroxypropyl cellulose, hydroxypropylmethyl cellulose, polyvinylpyrrolidone, polypropylene glycol-poly oxyethylene-block co-polymer, meglumine, calcium citrate, dextrin, pectin, and the like as the binder; (3) starch, agar, gelatine powder, crystalline cellulose, calcium carbonate, sodium bicarbonate, calcium citrate, dextrin, pectic, carboxymethylcellulose/calcium, and the like as the disintegrant; (4) magnesium stearate, talc, polyethyleneglycol, silica, condensed plant oil, and the like as the lubricant; (5) any colorant whose addition is pharmaceutically acceptable is adequate as the colorant; (6) cocoa powder, menthol, aromatizer, peppermint oil, cinnamon powder as the flavoring agent; (7) antioxidants whose addition is pharmaceutically accepted such as ascorbic acid or alpha-tophenol.

For parenteral administration in an aqueous solution, for example, the solution generally is suitably buffered and the liquid diluent first rendered isotonic for example with sufficient saline or glucose. Such aqueous solutions may be used, for example, for intravenous, intramuscular, subcutaneous and intraperitoneal administration. Preferably, sterile aqueous media are employed as is known to those of skill in the art, particularly in light of the present application. By way of illustration, a single dose may be dissolved in 1 ml of isotonic NaCl solution and either added to 1000 ml of hypodermoclysis fluid or injected at the proposed site of infusion, (see, for example, “Remington's Pharmaceutical Sciences” 15th Edition, pages 1035-1038 and 1570-1580, which is hereby incorporated by reference in its entirety). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety, and purity standards as required by FDA Office of Biologics standards.

### 5.6.Methods of Treatment

As described herein, advancing age and sedentary life-style promotes significant muscle loss that becomes largely irreversible with advancing age, including very severe muscle loss and atrophy with age (sarcopenia). Sarcopenia and age-related muscle loss is a significant source of morbidity and mortality in the aging and the elderly population. Only physical exercise is considered an effective strategy to improve muscle maintenance and function, but it must begin well before the onset of disease. In addition, muscle injury in traumatic wounds can be a significant obstacle to healing and full recovery. There are few effective therapeutic options. Thus, an aspect of the present application relates to a method of promoting muscle regeneration.

The Examples of the present application demonstrate AUF1 skeletal muscle gene transfer: (1) strongly enhances exercise endurance in middle-aged (12 month; equivalent to approximately 38 to 47 year old humans) and old mice (18 months; equivalent to about 56 years of age humans) to even older mice (24 months, equivalent to approximately 70 year or older) to levels of performance displayed by young mice (3 months old; equivalent to late teens, early 20's in humans) (see, e.g., Flurkey, Currer, and Harrison, 2007. ‘The mouse in biomedical research.’ in James G. Fox (ed.), American College of Laboratory Animal Medicine series (Elsevier, AP: Amsterdam; Boston, which is incorporated by reference herein in its entirety) (2) stimulates both fast and slow muscle, but specifically specifies slow muscle lineage by increasing levels of expression of the gene pgc1α (Peroxisome proliferator-activated receptor gamma co-activator 1-alpha), a major activator of mitochondrial biogenesis and slow-twitch myofiber specification; (3) significantly increases skeletal muscle mass and normal muscle fiber formation in middle age and old mice in age-related muscle loss; and (4) reduces expression of established biomarkers of muscle atrophy and muscle inflammation in age-related muscle loss.

Accordingly, provided are methods of promoting muscle regeneration or reducing or slowing the degeneration or atrophy of muscle by administering AUF1 to muscle cells in a subject in need thereof to increase muscle cell mass, increase muscle cell endurance, and/or reduce serum markers of muscle atrophy. Such administering may be systemic or direct local administration to muscles in need of treatment of the AUF1 protein or nucleic acid encoding AUF1, for example as DNA, mRNA, plasmid, or viral vector, including lentiviral vector or AAV vector. In a specific embodiments, provided are methods of contacting muscle cells with an adeno-associated viral (AAV) vector or a composition described herein under conditions effective to express exogenous AUF1 in the muscle cells to increase muscle cell mass, increase muscle cell endurance, and/or reduce serum markers of muscle atrophy.

Accordingly, provided are methods of treating sarcopenia in a subject in need thereof by administering AUF1 (including human AUF1 p37, AUF1 p40, AUF1 p42, AUF1 p45) to the muscles of the subject. The AUF1 may be administered as protein, or as nucleic acid, for example, as DNA, plasmid DNA, mRNA, or viral vector, including lentiviral vectors or AAV vectors. In specific embodiments, methods are provided of administering an rAAV particle comprising a genome comprising a nucleotide sequence encoding AUF1, operably linked to a promoter for expression of the AUF1 in muscle cells (such as, for example, a muscle creatine kinase promoter or other muscle specific promoter). The regulatory element, in embodiments, promotes expression in one or a combination of skeletal muscle, cardiac muscle, or diaphragm muscle The subject is human and may be a middle aged (from 40 to 50, from 45 to 55, from 50 to 60, from 55 to 65 years of age) or alternatively, the subject may be elderly, including subjects from 65 to 75 years of age, 70 to 80 years of age, 75 to 85 years of age, 80 to 90 years of age or even older than 90 years of age and the administration of the AUF1-encoding gene therapy results (within 2 weeks, 1 month, 2 months, 3 months, 4 months or 6 months) in increased muscle mass, muscle performance, muscle stamina and slowing or even reversal of muscle atrophy, for example, as assessed by biomarkers for muscle mass, muscle performance, muscle stamina or muscle atrophy. In alternative embodiments, the subject is a non-human mammal, including dogs, cats, horses, cows, pigs, sheep, etc. and is middle aged or elderly.

Suitable cells for use according to the methods of the present application include, without limitation, mammalian cells such as rodent (mouse or rat) cells, cat cells, dog cells, rabbit cells, horse cells, sheep cells, pig cells, cow cells, and non-human primate cells. In some embodiments the cells are human cells.

In some embodiments, the muscle cells are a myocyte, a myoblast, a skeletal muscle cell, a cardiac muscle cell, a smooth muscle cell, or a muscle stem cells (e.g., a satellite cell).

The method may be carried out in vitro or ex vivo.

In some embodiments, the method further involves culturing the muscle cells ex vivo under conditions effective to express exogenous AUF1.

In some embodiments, the method is carried out in vivo.

In some embodiments, the method further involves contacting the muscle cells with a purine-rich element binding protein β (Purβ) inhibitor. The Purβ inhibitor may be a nucleic acid molecule, a polypeptide, or a small molecule. In some embodiments, the nucleic acid molecule is an siRNA, shRNA, or miRNA. Suitable nucleic acid molecules are described in detail supra.

The dystrophin glycoprotein complex (DGC) is a specialization of cardiac and skeletal muscle membrane. This large multicomponent complex has both mechanical stabilizing and signaling roles in mediating interactions between the cytoskeleton, membrane, and extracellular matrix. The DGC links the actin cytoskeleton to the basement membrane and is thought to provide mechanical stability to the sarcolemma (see, e.g., Petrof B J (2002) Am J Phys Med Rehabil 81, S162-S174). AUF1 increases expression or stability of one or more of the components in the DGC or that interact with the DGC, which provides stability to the sarcolemma and thereby increases or improves muscle strength and/or function.

Accordingly, disclosed are methods of stabilizing sarcolemma in a subject, including a human subject, in need thereof, said method comprising administering to the subject a pharmaceutical composition comprising a therapeutically effective amount of AUF1 (including human p37AUF1, p40AUF1, p42AUF1, or p45AUF1), including by administering a vector, such as an rAAV, and a pharmaceutically acceptable carrier, wherein the vector comprises a muscle cell-specific promoter and a nucleic acid molecule encoding an AUF1 protein or a functional fragment thereof, wherein the nucleic acid molecule is operatively coupled to the muscle cell-specific promoter. Provided are methods of stabilizing the sarcolemma in a subject in need thereof, including a human subject, said method comprising administering to the subject a pharmaceutical composition comprising a therapeutically effective amount of an rAAV viral particle comprising a transgene encoding AUF1. These methods may be useful in the treatment of muscle degenerative diseases and disorders, such as dystrophinopathies, as described below.

β-dystroglycan, present in the DGC, forms a complex in skeletal muscle fibers and plays a role in linking dystrophin to the laminin in the extracellular matrix. The presence of the DGC helps strengthen muscle fibers and protect them from injury. Disclosed are methods of increasing β-dystroglycan in a DGC comprising administering to the subject a vector, including an rAAV vector, comprising a muscle cell-specific promoter and a nucleic acid molecule encoding an AUF1 protein (including human p37AUF1, p40AUF1, p42AUF1, or p45AUF1), or a functional fragment thereof, wherein the nucleic acid molecule is operatively coupled to the muscle cell-specific promoter.

β-sarcoglycan can also form a complex with the DGC to help stabilize and strengthen muscle. Disclosed are methods of increasing β-sarcoglycan in a DGC comprising administering to the subject a vector, including an rAAV vector, comprising a muscle cell-specific promoter and a nucleic acid molecule encoding an AUF1 protein or a functional fragment thereof, wherein the nucleic acid molecule is operatively coupled to the muscle cell-specific promoter. Further provided are methods of increasing utrophin participation in DGCs in a subject in need thereof by administering to the subject a vector, including an rAAV vector, comprising a muscle cell-specific promoter and a nucleic acid molecule encoding an AUF1 protein (including human p37AUF1, p40AUF1, p42AUF1, or p45AUF1), or a functional fragment thereof, wherein the nucleic acid molecule is operatively coupled to the muscle cell-specific promoter. Further methods are provided of increasing levels of and/or participation in the DGC of one or more of α-sarcoglycan, β-sarcoglycan, δ-sarcoglycan, γ-sarcoglycan, ε-sarcoglycan, ζ-sarcoglycan, α-dystroglycan, β-dystroglycan, sarcospan, α-syntrophin, β-syntrophin, α-dystrobrevin, β-dystrobrevin, Caveolin-3, or nNOS by administering AUF1, including by administering an rAAV vector, comprising a muscle cell-specific promoter and a nucleic acid molecule encoding an AUF1 protein (including human p37AUF1, p40AUF1, p42AUF1, or p45AUF1), or a functional fragment thereof, wherein the nucleic acid molecule is operatively coupled to the muscle cell-specific promoter.

A further aspect of the present application relates to a method of treating degenerative skeletal muscle loss in a subject. This method involves selecting a subject in need of treatment for skeletal muscle loss and administering to the subject a pharmaceutical composition comprising a therapeutically effective amount of AUF1 (including human p37AUF1, p40AUF1, p42AUF1, or p45AUF1), including by administering AUF1 protein or a nucleic acid encoding AUF1, such as DNA, a plasmid, mRNA, and includes administering a vector, such as an rAAV, and a pharmaceutically acceptable carrier, wherein the vector comprises a muscle cell-specific promoter and a nucleic acid molecule encoding an AUF1 protein or a functional fragment thereof, wherein the nucleic acid molecule is operatively coupled to the muscle cell-specific promoter under conditions effective to cause skeletal muscle regeneration in the selected subject. For example, the administering may be effective to activate muscle stem cells, accelerate the regeneration of mature muscle fibers (myofibers), enhance expression of muscle regeneration factors, accelerate the regeneration of injured skeletal muscle, increase regeneration of slow-twitch (Type I) and/or fast-twitch (Type II) fibers), and/or restore muscle mass, muscle strength, and create normal muscle and/or improve mitochondrial oxidative capacity, muscle exercise capacity, muscle performance, stamina and resistance to fatigue in the selected subject.

In some embodiments, the subject has a degenerative muscle condition. As used herein, the term “degenerative muscle condition” refers to conditions, disorders, diseases and injuries characterized by one or more of muscle loss, muscle degeneration or wasting, muscle weakness, and defects or deficiencies in proteins associated with normal muscle function, growth or maintenance. In certain embodiments, a degenerative muscle condition is sarcopenia or cachexia. In other embodiments, a degenerative muscle condition is one or more of muscular dystrophy, muscle injury, including acute muscle injury, resulting in loss of muscle tissue, muscle atrophy, wasting or degeneration, muscle overuse, muscle disuse atrophy, muscle disuse atrophy, denervation muscle atrophy, dysferlinopathy, AIDS/HIV, diabetes, chronic obstructive pulmonary disease, kidney disease, cancer, aging, autoimmune disease, polymyositis, and dermatomyositis. Thus, in some embodiments, the subject has a degenerative muscle condition, including sarcopenia or myopathy.

The compositions and methods described herein may be used in combination with other known treatments or standards of care for given diseases, injury, or conditions. For example, in the context of muscular dystrophy, a composition of the invention for promoting muscle satellite cell expansion can be administered in conjunction with such compounds as CT-1, pregnisone, or myostatin. The treatments (and any combination treatments provided herein) may be administered together, separately or sequentially.

The subject may have a muscle disorder mediated by functional AUF1 deficiency or a muscle disorder not mediated by functional AUF deficiency.

In some embodiments, the subject has an adult-onset myopathy or muscle disorder.

As used herein, the term “muscular dystrophy” includes, for example, Duchenne, Becker, Limb-girdle muscular dystrophy, Congenital, Facioscapulohumeral, Myotonic, Oculopharyngeal, Distal, and Emery-Dreifuss muscular dystrophies. In particular embodiments, the muscular dystrophy is characterized, at least in part, by a deficiency or dysfunction of the protein dystrophin. Such muscular dystrophies may include Duchenne muscular dystrophy (DMD) and Becker muscular dystrophy (DMD). In other embodiments, the muscular dystrophy is associated with degenerative muscle conditions such as muscle disuse atrophy, denervation muscle atrophy, dysferlinopathy, AIDS/HIV, diabetes, chronic obstructive pulmonary disease, kidney disease, cancer, aging, autoimmune disease, polymyositis, and dermatomyositis.

In some embodiments of the methods disclosed herein, the subject has Duchenne Muscular Dystrophy (DMD). As described above, DMD is an X-linked muscle wasting disease that is quite common (1/3500 live births), generally but not exclusively found in males, caused by mutations in the dystrophin gene that impair its expression for which there are few therapeutic options that have been shown to be effective. Muscle satellite cells are unresponsive in DMD and are said to be functionally exhausted, thereby limiting or preventing new muscle development and regeneration. DMD typically presents in the second year after birth and progresses over the next two to three decades to death in young men.

In some embodiments of the methods disclosed herein, the subject has Becker muscular dystrophy. As described above, Becker muscular dystrophy is a less severe form of the disease that also involves mutations that impair dystrophin function or expression but less severely. There are few therapeutic options that have been shown to be effective for Becker muscular dystrophy. There are no cures for DMD or Becker disease.

Accordingly, provided are methods of treating or ameliorating the symptoms of a dystrophinopathy, including DMD, Becker disease, or limb girdle muscular dystrophy, by administering an rAAV vector comprising a genome encoding AUF1 operably linked to a regulatory element that promotes expression of the AUF1 in muscle cells.

In embodiments, the effectiveness of the gene therapy administration to stabilize the sarcolemma, increases muscle mass, function and/or performance, to reduce muscle atrophy and to treat muscle degeneration can be assessed at, for example, 1 month, 2 months, 3 months. 4 months, 5 months or 6 months after administration relative to normal muscle (or reference normal or diseased muscle) or muscle of the subject prior (e.g. 2 weeks, 1 month or 2 months prior) to administration of the therapeutic. In embodiments, the methods disclosed herein provide for stabilization of the sarcolemma and/or reduction in muscle leakiness as reflected in 20%, 30%, 40%, 50%, 60%, 70%, 80%, 90% or 100% or greater (2 fold, 3 fold or more) reduction in markers of sarcolemma integrity, including, for example, serum creatine kinase levels, 20%, 30%, 40%, 50%, 60%, 70%, 80%, 90% or 100% or greater (2 fold, 3 fold or more), reduction in markers of muscle atrophy (for example, biomarkers as disclosed herein), 20%, 30%, 40%, 50%, 60%, 70%, 80%, 90% or 100% or greater (2 fold, 3 fold or more) increase in utrophin levels or a member of the dystrophin sarcoglycan complex, 20%, 30%, 40%, 50%, 60%, 70%, 80%, 90% or 100% or greater (2 fold, 3 fold or more) increase compared to normal muscle or muscle of the subject prior to administration of the therapeutic of muscle mass, or muscle function, or performance using methods known in the art for assessing muscle mass, muscle function or muscle performance.

In some embodiments, the administering is effective to transduce muscle cells, including skeletal muscle cells, cardiac muscle cells, and/or diaphragm muscle cells and/or provide long-term (e.g., lasting at least 1 month, 2 months, 3 months, 4 months, 5 months, 6 months, 7 months, 8 months, 9 months, 10 months, 11 months, or more) muscle cell-specific AUF1 expression in the selected subject.

In other embodiments, the administering of the rAAV encoding AUF1 (including human p37AUF1, p40AUF1, p42AUF1, or p45AUF1) operably linked to a regulatory element to promote muscle cell expression is effective to (i) activate high levels of satellite cells and myoblasts; (ii) significantly increase skeletal muscle mass and normal muscle fiber formation; and/or (iii) significantly enhanced exercise endurance in the selected subject as compared to when the administering is not carried out.

In further embodiments, the administering of the rAAV encoding AUF1 (including human p37AUF1, p40AUF1, p42AUF1, or p45AUF1) operably linked to a regulatory element to promote muscle cell expression is effective to reduce (i) biomarkers of muscle atrophy and muscle cell death; (ii) inflammatory immune cell invasion in skeletal muscle (including diaphragm); and/or (iii) muscle fibrosis and necrosis in skeletal muscle (including diaphragm) in the selected subject, as compared to when the administering is not carried out.

In certain embodiments, the administering of an rAAV encoding AUF1 (including human p37AUF1, p40AUF1, p42AUF1, or p45AUF1) operably linked to a regulatory element to promote muscle cell expression is effective to (i) increase expression of endogenous utrophin in DMD muscle cells and/or (ii) suppress expression of embryonic dystrophin, a marker of muscle degeneration in DMD in the selected subject, as compared to when the administering is not carried out. In some embodiments of the methods disclosed herein, said administering of an rAAV encoding AUF1 is effective to upregulate endogenous utrophin protein expression in the selected subject, as compared to when the administering is not carried out. In some embodiments of the methods disclosed herein, said administering and rAAV encoding AUF1 is effective to upregulate endogenous utrophin protein expression in said muscle cells, as compared to when the administering is not carried out.

In some embodiments, the administering of rAAV encoding AUF1 (including human p37AUF1, p40AUF1, p42AUF1, or p45AUF1) is effective to (i) increase normal expression of genes involved in muscle development and regeneration and/or (ii) suppress genes involved in muscle cell fibrosis, death, atrophy and muscle-expressed inflammatory cytokines in the selected subject, as compared to when the administering is not carried out.

In further embodiments, the administering does not increase muscle mass, endurance, or activate satellite cells in normal skeletal muscle (i.e., healthy skeletal muscle that does not express markers of atrophy, degeneration or loss of weight or stamina).

In some embodiments, the administering is effective to accelerate muscle gain in the selected subject, as compared to when said administering is not carried out.

In certain embodiments, the administering is effective to reduce expression of established biomarkers of muscle atrophy (for example, by 20%, 30%, 40%, 50%, 60%, 70%, 80%, 90% or 100% or more) in a subject having degenerative skeletal muscle loss relative to pre-treatment levels (for example, within 1 day, 1 weeks, 2 weeks or one month prior to therapeutic administration or an appropriate time period for assessing a baseline value of these markers). Suitable biomarkers of muscle atrophy include, without limitation, TRIM63 and Fbxo32 mRNA. In some embodiments, the administering is effective to enhance expression of established biomarkers of muscle myoblast activation, differentiation, and muscle regeneration in the selected subject. Suitable biomarkers of muscle atrophy include, without limitation, myogenin and MyoD mRNA levels, biomarkers of myoblast activation, differentiation and muscle regeneration (Zammit, “Function of the Myogenic Regulatory Factors Myf5, MyoD, Myogenin and MRF4 in Skeletal Muscle, Satellite Cells and Regenerative Myogenesis,” Semin. Cell. Dev. Biol. 72:19-32 (2017), which is hereby incorporated by reference in its entirety).

In some embodiments, the method further involves administering a purine-rich element binding protein R (Purβ) inhibitor. The Purβ inhibitor may be a nucleic acid molecule, a polypeptide, or a small molecule. In some embodiments, the nucleic acid molecule is an siRNA, shRNA, and miRNA. Suitable nucleic acid molecules are describe in detail supra.

### Traumatic Muscle Injury

A further aspect of the present application relates to a method of preventing traumatic muscle injury in a subject. This method involves selecting a subject at risk of traumatic muscle injury and administering to the selected subject an AUF1 protein, or a nucleic acid encoding AUF1, such as DNA, mRNA, plasmid or viral vector such as an AAV vector described herein, a composition described herein, or a lentiviral vector comprising a muscle cell specific promoter and a nucleic acid molecule encoding an AU-rich mRNA binding factor 1 (AUF1) protein (including human p37AUF1, p40AUF1, p42AUF1, or p45AUF1) or a functional fragment thereof, where the nucleic acid molecule is heterologous to and operatively coupled to the muscle cell-specific promoter. The administration may be systemic or local to the muscle or muscles at risk.

Still another aspect of the present application relates to a method of treating traumatic muscle injury in a subject. This method involves selecting a subject having traumatic muscle injury and administering to the selected subject AUF1, either as an AUF1 protein or nucleic acid encoding AUF1, such as DNA, mRNA, plasmid or viral vector, such as an AAV) vector described herein that encodes AUF1, operably linked to regulatory sequences that promote expression in muscle cells, a composition described herein, or a lentiviral vector comprising a muscle cell specific promoter and a nucleic acid molecule encoding an AU-rich mRNA binding factor 1 (AUF1) protein (including human p37AUF1, p40AUF1, p42AUF1, or p45AUF1) or a functional fragment thereof, where the nucleic acid molecule is operatively coupled to a muscle cell-specific promoter. The AUF1, or nucleic acid encoding AUF1 may be administered systemically, such as IV or IM, or may be administered locally to the affected muscle tissue.

In some embodiments of the methods disclosed herein, the subject has traumatic muscle injury. As used herein, the term “traumatic muscle injury” refers to a condition resulting from a wide variety of incidents, ranging from, e.g., everyday accidents, falls, sporting accidents, automobile accidents, to surgical resections to injuries on the battlefield, and many more. Non-limiting examples of traumatic muscle injuries include battlefield muscle injuries, auto accident-related muscle injuries, and sports-related muscle injuries.

Suitable subjects for treatment according to the methods of the present application include, without limitation, domesticated and undomesticated animals such as rodents (mouse or rat), cats, dogs, rabbits, horses, sheep, pigs, and non-human primates. In some embodiments the subject is a human subject. Exemplary human subjects include, without limitation, infants, children, adults, and elderly subjects.

In some embodiments, the subject is at risk of developing or is in need of treatment for a traumatic muscle injury, including a laceration, a blunt force contusion, a shrapnel wound, a muscle pull, a muscle tear, a burn, an acute strain, a chronic strain, a weight or force stress injury, a repetitive stress injury, an avulsion muscle injury, and compartment syndrome.

In some embodiments, the subject is at risk of developing or is in need of treatment for a traumatic muscle injury that involves volumetric muscle loss (“VML”). The terms “volumetric muscle loss” or “VML” refer to skeletal muscle injuries in which endogenous mechanisms of repair and regeneration are unable to fully restore muscle function in a subject. The consequences of VML are substantial functional deficits in joint range of motion and skeletal muscle strength, resulting in life-long dysfunction and disability.

In some embodiments, the administering is carried out to treat a subject having traumatic muscle injury and said administering is carried out immediately after the traumatic muscle injury (for example, within one minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 60 minutes, or any amount of time there between) of the traumatic muscle injury. In certain embodiments, said administering is carried out out within 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours of the traumatic muscle injury. In other embodiments, said administering is carried out within 1 day, 2 days, 3 days, 4 days, 5 days, 6 days, 7 days, 8 days, 9 days, 10 days, 11 days, 12 days, 13 days, or 14 days of the traumatic muscle injury. In further embodiments, said administering may be carried out within 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 52 weeks, or any amount of time after the traumatic muscle injury.

The adeno-associated viral (AAV) vector and/or the lentiviral vector for use in the methods disclosed herein may encode AUF1 isoform p37^{AUF1}, p40^{AUF1}, p42^{AUF1}, or p45^{AUF1}. Suitable AUF isoform nucleic acid and amino acid sequences are identified supra. In certain embodiments, the adeno-associated viral (AAV) vector and/or the lentiviral vector for use in the methods disclosed herein encodes AUF isoform p40^{AUF}1

In some embodiments, the rAAV is AAV8-tMCK-AUF1 or another AAV serotype including but not limited to AAV1, AAV2, AAV5, AAV6, or AAV9 vector encoding AUF1 (e.g., AUF1 isoforms p37^{AUF1}, p40^{AUF1}, p42^{AUF1}, or p45^{AUF1}). In other embodiments, the AAV is a human novel AAV capsid variant engineered for enhanced muscle-specific tropism including but not limited to AAV2i8 or AAV2.5. In yet other embodiments, the AAV vector is a non-human primate AAV vector including but not limited to AAVrh.8, AAVrh.10, AAVrh.43, or AAVrh.74.

In some embodiments, the lentiviral vector is a lentivirus p45 AUF1 vector, or a lentivirus expressing another AUF1 isoform (e.g., p37^{AUF1}, p40^{AUF1}, or p42^{AUF1}) or combinations thereof (Abbadi et al., “Muscle Development and Regeneration Controlled by AUF1-mediated Stage-specific Degradation of Fate-determining Checkpoint mRNAs,” Proc. Nat'l. Acad. Sci. USA 116:11285-90 (2019), which is hereby incorporated by reference in its entirety). Other embodiments include expression of p37^{AUF1}, p40^{AUF1}, p42^{AUF1}, p45^{AUF1}, or combinations thereof from non-human lentivirus vectors including but not limited to simian, feline, and other mammalian lentivirus gene transfer vectors. In some embodiments, the administering is effective to prevent muscle atrophy and/or muscle loss following traumatic muscle injury to the selected subject. In other embodiments, the administering is effective to activate muscle stem cells following traumatic muscle injury to the selected subject. In further embodiments, the administering is effective to accelerate the regeneration of mature muscle fibers (myofibers), enhance expression of muscle regeneration factors, accelerate the regeneration of injured muscle, increased regeneration of slow-twitch (Type I) and/or fast-twitch (Type II) fibers), and/or restore muscle mass, muscle, strength and create normal muscle following traumatic muscle injury in the selected subject.

In some embodiments, the administering is effective to accelerate muscle gain following traumatic muscle injury in the selected subject, as compared to when said administering is not carried out.

In certain embodiments, the administering is effective to reduce (for example, by 20%, 30%, 40%, 50%, 60%, 70%, 80%, 90% or 100% or more relative to pre-treatment levels of the markers in the subject) expression of established biomarkers of muscle atrophy following traumatic muscle injury to the selected subject. Suitable biomarkers of muscle atrophy include, without limitation, TRIM63 and Fbxo32 mRNA. In some embodiments, the administering is effective to enhance expression of established biomarkers of muscle myoblast activation, differentiation and muscle regeneration following traumatic muscle injury to the selected subject. Suitable biomarkers of muscle atrophy include, without limitation, myogenin and MyoD mRNA levels, biomarkers of myoblast activation, differentiation and muscle regeneration (Zammit, “Function of the Myogenic Regulatory Factors Myf5, MyoD, Myogenin and MRF4 in Skeletal Muscle, Satellite Cells and Regenerative Myogenesis,” Semin. Cell. Dev. Biol. 72:19-32 (2017), which is hereby incorporated by reference in its entirety).

In some embodiments, the administering is effective to deliver the vector or pharmaceutical composition (including the transgene protein product) described herein to a specific tissue in the subject. The tissue may be muscle tissue. For example, the muscle tissue may be all types of skeletal muscle, smooth muscle, or cardiac muscle.

Administering, according to the methods of the present application, may be carried out orally, topically, transdermally, parenterally, subcutaneously, intravenously, intramuscularly, intraperitoneally, by intranasal instillation, by intracavitary or intravesical instillation, intraocularly, intraarterially, intralesionally, or by application to mucous membranes. Thus, in some embodiments, the administering is carried out intramuscularly, intravenously, subcutaneously, orally, or intraperitoneally. In specific embodiments, the administering is carried out by intramuscular injection. In some embodiments, an rAAV vector encoding AUF1 is administered peripherally, including intramuscularly, intravenously or any other systemic administration method or any method that results in delivery of the rAAV to muscle cells. The dosage of the rAAV administered may be 1E13 vg/kg to 1E14 vg/kg, including 2E13 vg/kg, and may also include 3E13 vg/kg, 4E13 vg/kg, 5E13 vg/kg, 6E13 vg/kg, 7E13 vg/kg, 8E13 vg/kg, or 9E13 vg/kg. (Note that vector genomes (vg) and genome copy (gc) are used interchangeably herein as are EX and X10^{X}).

Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Suitable regimens for initial contacting and further doses or for sequential contacting steps may all be the same or may be variable. Appropriate regimens can be ascertained by the skilled artisan, from the disclosure of the present application, the documents cited herein, and the knowledge in the art.

A dosage unit to be administered in methods of the present application will vary depending on the vector used, the route of administration, the type of tissue and cell being targeted, and the purpose of treatment, among other parameters. Dosage for treatment can be determined by a skilled person who would know how to determine dose using methods standard in the art. A dosage unit, corresponding to genome copy number, for example, could range from about, 1×10¹ to 1×10¹¹, 1×10² to 1×10¹¹, 1×10³ to 1×10¹¹, 1×10⁴ to 1×10¹¹, 1×10⁵ to 1×10¹¹, 1×10⁶ to 1×10¹¹, 1×10⁷ to 1×10¹¹, 1×10⁸ to 1×10¹¹, 1×10⁹ to 1×10¹¹, 1×10¹⁰ to 1×10¹¹, 1×10¹ to 1×10¹⁰, 1×10² to 1×10¹⁰, 1×10³ to 1×10¹⁰, 1×10⁴ to 1×10¹⁰, 1×10⁵ to 1×10¹⁰, 1×10⁶ to 1×10¹⁰, 1×10⁷ to 1×10¹⁰, 1×10⁸ to 1×10¹⁰, 1×10⁹ to 1×10¹⁰, 1×10¹ to 1×10⁹, 1×10² to 1×10⁹, 1×10³ to 1×10⁹, 1×10⁴ to 1×10⁹, 1×10⁵ to 1×10⁹, 1×10⁶ to 1×10⁹, 1×10⁷ to 1×10⁹, 1×10⁸ to 1×10⁹, 1×10¹ to 1×10⁸, 1×10² to 1×10⁸, 1×10³ to 1×10⁸, 1×10⁴ to 1×10⁸, 1×10⁵ to 1×10⁸, 1×10⁶ to 1×10⁸, or 1×10⁷ to 1×10⁸ genome copies of a vector disclosed herein. In some embodiments, a dosage unit, corresponding to genome copy number, for example, is administered in the range of 1×10¹ to 1×10¹², 1×10² to 1×10¹², 1×10³ to 1×10¹², 1×10⁴ to 1×10¹², 1×10⁵ to 1×10¹², 1×10⁶ to 1×10¹², 1×10⁷ to 1×10¹², 1×10⁸ to 1×10¹², 1×10⁹ to 1×10¹², 1×10¹⁰ to 1×10¹², or 1×10¹¹ to 1×10¹² genome copies; 1×10¹ to 1×10¹³, 1×10² to 1×10¹³, 1×10³ to 1×10¹³, 1×10⁴ to 1×10¹³, 1×10⁵ to 1×10¹³, 1×10⁶ to 1×10¹³, 1×10⁷ to 1×10¹³, 1×10⁸ to 1×10¹³, 1×10⁹ to 1×10¹³, 1×10¹⁰ to 1×10¹³, 1×10¹¹ to 1×10¹³, or 1×10¹² to 1×10¹³ genome copies; 1×10¹ to 1×10¹⁴, 1×10² to 1×10¹⁴, 1×10³ to 1×10¹⁴, 1×10⁴ to 1×10¹⁴, 1×10⁵ to 1×10¹⁴, 1×10⁶ to 1×10¹⁴, 1×10⁷ to 1×10¹⁴, 1×10⁸ to 1×10¹⁴, 1×10⁹ to 1×10¹⁴, 1×10¹⁰ to 1×10¹⁴, 1×10¹¹ to 1×10¹⁴, 1×10¹² to 1×10¹⁴, or 1×10¹³ to 1×10¹⁴ genome copies; 1×10¹ to 1×10¹⁵, 1×10² to 1×10¹⁵, 1×10³ to 1×10¹⁵, 1×10⁴ to 1×10¹⁵, 1×10⁵ to 1×10¹⁵, 1×10⁶ to 1×10¹⁵, 1×10⁷ to 1×10¹5, 1×10⁸ to 1×10¹⁵, 1×10⁹ to 1×10¹⁵, 1×10¹⁰ to 1×10¹⁵, 1×10¹¹ to 1×10¹⁵, 1×10¹² to 1×10¹⁵, 1×10¹³ to 1×10¹⁵, or 1×10¹⁴ to 1×10¹⁵ genome copies; 1×10¹ to 1×10¹⁶, 1×10² to 1×10¹⁶, 1×10³ to 1×10¹⁶, 1×10⁴ to 1×10¹⁶, 1×10⁵ to 1×10¹⁶, 1×10⁶ to 1×10¹⁶, 1×10⁷ to 1×10¹⁶, 1×10⁸ to 1×10¹⁶, 1×10⁹ to 1×10¹⁶, 1×10¹⁰ to 1×10¹⁶, 1×10¹¹ to 1×10¹⁶, 1×10¹² to 1×10¹⁶, 1×10¹³ to 1×10¹⁶, 1×10¹⁴ to 1×10¹⁶, or 1×10¹⁵ to 1×10¹⁶ genome copies; and any amount there between. Dosage will depend on route of administration, type of tissue and cells to receive the vector, timing of administration to human subjects, whether dosage is determined based on total genome copies to be delivered, and whether administration is determined by genome copies per kilogram body weight.

### 5.7.Methods of Use for Producing Cultured or Synthetic Muscle Tubes, Fibers and Muscle

Example 9 herein discloses that expression of p40 AUF1 in cultured muscle cells, such as C2C12 cells, accelerated development of mature myofibers and increased expression of transcriptional markers of slow twitch muscle 2 to 10 fold. Accordingly, disclosed are methods for producing synthetic meat products that may be used for consumption. The present disclosure describes methods for enhancing cultured meat production, such as animal-free meat production. Technologies currently used for cultured meat production suffer from the inability to increase the presence of slow-twitch (dark) muscle fibers in cultured meat (myotubes or myofibers), and contain instead a large proportion or are entirely composed of fast-twitch myotubes or myofibers. Slow twitch muscle is generally considered more flavorful and desirable, but methods have not been developed to reliably enhance the slow twitch muscle composition in cultured muscle.

Provided are methods of culturing muscle cells in the presence of AUF1 or expressing AUF1 for the production of cultured meat, promoting the development of slow twitch muscle over that of fast twitch muscle in the culturing of muscle for animal-free meat production. The presence or expression of AUF1 in the muscle cell culture increases the proportion of slow twitch muscle by 2 fold, 5 fold, 10 fold or 100 fold. Thus, provided is a method for increasing the production of slow twitch muscle in culture by contacting the cultured muscle cells with AUF1 or expressing AUF1 in the cells in an amount sufficient to promote production of slow twitch muscle that is sustainable, scalable and can be integrated into existing platforms for cultured meat production, whether cultured in two-dimensional monolayer systems, three-dimensional complex muscle structure systems with or without consumable matrices, or bioreactors. In embodiments, the muscle cells expressing or in contact with AUF1 are cultured with cell types other than muscle such as adipocytes to increase the natural texture and composition of cultured muscle as a food product. Provided are methods to increase the composition of slow twitch muscle cells cultured muscle from practically all animal species except human, including but not restricted to sheep, goat, pig, deer, rabbit, hare, whale, kangaroo; birds such as chicken, goose, pheasant, duck, ostrich and partridge; reptiles such as frog, turtle, crocodile; fish such as tuna, eel, cod, sole, shark and herring; and shellfish such as oyster, crab, langoustine and shrimp. Mixtures or combinations of the above can also be made.

Disclosed are methods of producing synthetic meat comprising administering AUF1 to or expressing AUF1 in cultured muscle cells; and growing the muscle cells to produce synthetic meat, wherein the synthetic meat comprises an increased proportion (2 fold, 5 fold, 10 fold, 20 fold or 100 fold increase) of slow twitch muscle fibers compared to synthetic meat from cultured muscle cells not contacted with AUF1 or expressing AUF1.

Disclosed are methods of increasing slow twitch muscle fibers in synthetic meat comprising administering AUF1 to cultured muscle cells; and growing the muscle cells to produce synthetic meat comprising increased slow twitch muscle fibers.

In some aspects, AUF1 can be administered as a protein, functional protein fragment, nucleic acid encoding AUF1, or in an expression vector encoding AUF1 or in a viral particle, such as an rAAV, encoding AUF1.

In some aspects, the muscle cells can be derived from any non-human animals consumed by humans such as mammals (e.g. cattle, buffalo, pigs, sheep, deer, etc.), birds (e.g. chicken, ducks, ostrich, turkey, pheasant, etc.), fish (e.g. swordfish, salmon, tuna, sea bass, trout, catfish, etc.), invertebrates (e.g. lobster, crab, shrimp, clams, oysters, mussels, sea urchin, etc.), reptiles (e.g. snake, alligator, turtle, etc.), and amphibians (e.g. frog legs). In certain embodiments, muscle cells are derived from pluripotent embryonic mesenchymal stem cells that give rise to muscle cells, fat cells, bone cells, and cartilage cells. The muscle cells may also be derived from totipotent embryonic stem cells such as cells from the blastocyst stage, fertilized eggs, placenta, or umbilical cords of these animals.

Thus, in some aspects, the disclosed methods comprise administering AUF1 to, including expressing AUF1 in, stem cells that can be or have been differentiated into muscle cells. Therefore, any of the disclosed methods can involve a first step of differentiating stem cells to muscle cells. The differentiation can occur before administering AUF1 or expressing AUF1 in the cells or simultaneously with administering AUF1 or expressing AUF1 in the cells.

In one embodiment, muscle cells can be grown on, around, or inside a three-dimensional support structure. The support structure can be sculpted into different sizes, shapes, and forms, as desired, to provide the shape and form for the muscle cells to grow and resemble different types of muscle tissues such as steak, tenderloin, shank, chicken breast, drumstick, lamb chops, fish fillet, lobster tail, etc. The support structure can be made from natural or synthetic biomaterials that are preferably non-toxic so that they may not be harmful if ingested. Natural biomaterials may include, for example, collagen, fibronectin, laminin, or other extracellular matrices. Synthetic biomaterials may include, for example, hydroxyapatite, alginate, polyglycolic acid, polylactic acid, or their copolymers. The support structure can be formed as a solid or semisolid support

In another embodiment of the invention, regulatory factors, growth factors, or other gene products can also be introduced into the muscle cells along with the AUF1. These factors, known as myogenic regulatory factors (“MRFs”), can stimulate and regulate the growth of muscles in vivo, but may not normally be produced by muscle cells in vivo or in vitro. Thus, expressing myogenic regulatory factors in cultured muscle cells can increase the production of muscle cells in vitro.

In another embodiment of the invention, the meat products derived from muscle cells in vitro can include different derivatives of meat products. These derivatives can be prepared, for example, by grounding or shredding the muscle tissues grown in vitro and mixed with appropriate seasoning to make meatballs, fishballs, hamburger patties, etc. The derivatives can also be prepared from layers of muscle cells cut and spiced into, for example, beef jerky, ham, bologna, salami, etc. Thus, the meat products produced by the methods disclosed herein can be used to generate any kind of food product originating from the meat of an animal.

Also provided are methods of producing cultured or synthetic muscle tubes, fibers and/or muscle by methods disclosed herein for producing muscle tissue that can be used for transplantation, to repair or supplement muscle tissue and may increase the proportion of slow twitch muscle at a locus in the body of a subject.

Disclosed are methods of producing cultured or synthetic muscle tubes, fibers and/or tissue comprising administering AUF1 to or expressing AUF1 in cultured muscle cells; and growing the muscle cells to produce the cultured or synthetic muscle tubes, fibers or tissue, wherein the synthetic or cultured muscle tubes, fibers or tissue comprises an increased proportion (2 fold, 5 fold, 10 fold, 20 fold or 100 fold increase) of slow twitch muscle fibers compared to synthetic muscle tubes, fibers or tissue from cultured muscle cells not contacted with AUF1 or expressing AUF1. Such cultured or synthetic muscle tubes, fibers or tissue may be used as muscle transplant to increase or induce production of slow twitch muscle fiber content in muscle tissue.

In some aspects, AUF1 can be administered as a protein, functional protein fragment, nucleic acid encoding AUF1, or in an expression vector encoding AUF1 or in a viral particle, such as an rAAV, encoding AUF1. Thus, in some aspects, the disclosed methods comprise administering AUF1 to, including expressing AUF1 in, stem cells that can be or have been differentiated into muscle cells. Therefore, any of the disclosed methods can involve a first step of differentiating stem cells to muscle cells. The differentiation can occur before administering AUF1 or expressing AUF1 in the cells or simultaneously with administering AUF1 or expressing AUF1 in the cells.

In another embodiment of the invention, regulatory factors, growth factors, or other gene products can also be introduced into the muscle cells along with the AUF1. These factors, known as myogenic regulatory factors (“MRFs”), can stimulate and regulate the growth of muscles in vivo, but may not normally be produced by muscle cells in vivo or in vitro. Thus, expressing myogenic regulatory factors in cultured muscle cells can increase the production of muscle cells in vitro.

### 6. EXAMPLES

### 6.1 Example 1

The examples below are intended to exemplify the practice of embodiments of the present application but are by no means intended to limit the scope thereof.

### Materials and Methods for Examples 1-8

### Mice

All animal studies were approved by the NYU School of Medicine Institutional Animal Care and Use Committee (IACUC) and conducted in accordance with IACUC guidelines. All auf^{-/-} KO mice and WT mice are of the 129/B6-background, bred at the F3 and F4 generations from auf^{-/-} heterozygous mice (Pont et al., “mRNA Decay Factor AUF1 Maintains Normal Aging, Telomere Maintenance, and Suppression of Senescence by Activation of Telomerase Transcription,” Molecular Cell 47(1):5-15 (2012) and Lu et al., “Endotoxic Shock in AUF1 Knockout Mice Mediated by Failure to Degrade Proinflammatory Cytokine mRNAs,” Genes Dev. 20(22):3174-3184 (2006), which are hereby incorporated by reference in their entirety). 12 month old C57BL6 mice (Jackson) for AUF1 supplementation during AAV experiments. One month old C57BL10 and C57BL/10ScSn-Dmd^{mdx}/J mice (Jackson) were used for AAV experiments in Example 8.

### Cells

C2C12 cells were obtained from the American Type Culture Collection (ATCC), authenticated by STR profiling and routinely checked for mycoplasma contamination. C2C12 cells were maintained in DMEM (Corning), 10% FBS (Gibco), and 1% penicillin streptomycin (Life Technologies). To differentiate cells, media was switched to DMEM (Corning), 2% Horse Serum (Gibco), and 1% penicillin streptomycin (Life Technologies) during 96 hours (Panda et al., “RNA-Binding Protein AUF1 Promotes Myogenesis by Regulating MEF2C Expression Levels,” Mol. Cell Biol. 34(16): 3106-3119 (2014), which is hereby incorporated by reference in its entirety). auf1 KO C2C12 cells were created with Crispr-Cas9 methods (Abbadi et al., “Muscle Development and Regeneration Controlled by AUF1-Mediated Stage-Specific Degradation of Fate-Determining Checkpoint mRNAs,” Proc. Natl. Acad. Sci. USA 116(23):11285-11290 (2019), which is hereby incorporated by reference in its entirety). For assays performed in the presence of actinomycin D to determine mRNA stability, C2C12 myoblasts cells were treated with 0.2 g/ml of actinomycin D (Sigma). RNA immune-precipitation experiments were done in WT C2C12 before and 48 hours of differentiation using a normal IgG rabbit control or a rabbit-anti AUF1 antibody (07-260, Millipore).

### Immunofluorescence

Mice had skeletal muscles removed as indicated in the text, put in OCT, frozen in dry ice-cooled isopentane (Tissue-Tek), fixed in 4% paraformaldehyde, and blocked in 3% BSA in TBS. C2C12 cells were fixed in 4% paraformaldehyde and blocked in 3% BSA in PBS. Samples were immunostained overnight with antibodies: AUF1 (07-260, Millipore), Slow myosin (NOQ7.5.4D, Sigma), Fast myosin (MY-32, Sigma), Laminin alpha 2 (4H8-2, Sigma), and GFP (2956, Cell signaling). Slow and fast myosin staining were done using MOM kit (Vector biolabs). Alexa Fluor donkey 488 and 555 secondary antibodies were used at 1:300 and incubated for 1 hour at room temperature. Slides were sealed with Vectashield with DAPI (Vector). Images were processed using ImageJ.

### Cloning and Reporter Assays

The 3′UTR ARE region of mouse PGC1a was cloned into the vector pIS1 downstream of the Renilla luciferase cDNA using an EcoRV site. The pIS1-PGC1a-3′UTR or pIS1 control plasmids were transfected using TransIT-LT1 (Mirus) into WT and WT AUF1 overexpressing C2C12 myoblasts. Cells were lysed after 24 h and luciferase activity measured using a dual-luciferase assay kit (Promega). All studies were performed in in triplicate.

### Succinate Dehydrogenase Activity Staining

Histochemical SDH staining was used as an index of muscle fiber oxidative capacity as described. Briefly, tissue sections were incubated in SDH incubation solution (sodium succinate; 50 mM, nitroblue tetrazolium, 0.5 mg/ml and phosphate buffer, 50 mM) for 1 h at 37° C. Tissue sections were washed in distilled water and mounted with glycerol based mounting medium. Five fields chosen at random were quantified using ImageJ software.

### Microscopy, Image Processing, and Analysis

Images were acquired using a Zeiss LSM 700 confocal microscope, primarily with the 20× lens. Images were processed using ImageJ. If needed, color balance was adjusted linearly for the entire image and all images in experimental sets.

### Immunoblot Studies

C2C12 cells or muscle tissues were lysed using lysis buffer (50 mmol/L Tris-HCl, pH 7.5, 150 mmol/L NaCl, 1 mmol/L EDTA, 1 mmol/L EGTA, 1% TritonX100) supplemented with complete protease inhibitor cocktail (Complete mini, ROCHE). Equal amounts of total protein were loaded on a polyacrylamide gel, resolved and transferred to PVDF membrane. Membrane was blocked with 5% nonfat milk in TBS-Tween 20 (0.1%) for 1 hour and probed with Antibody against AUF1 (07-260, Millipore) or against PGC1alpha (Novus biologicals NBP1-04676). Bands were detected by peroxidase conjugated secondary antibodies (GE healthcare) and visualized with the ECL chemiluminescence system. The immunoblots were also probed with a rabbit antibody to β-tubulin (Cell Signaling 2146S) or GAPDH (Cell Signaling 2118S) as a control for loading. Quantification was performed by ImageJ.

### Real-Time PCR Analysis

RNA was extracted using Trizol (Invitrogen) according to the manufacturer's instructions. DNase treatment was systematically performed. Quantification of extracted RNA was assessed using Nanodrop. The cDNA was synthesized using High Capacity cDNA Reverse Transcription Kit (Applied Biosystems). mRNA was analyzed by real-time PCR using the iTaq Universal SYBR Green Supermix (Bio Rad) probe. Relative quantification was determined using the comparative CT method with data normalized to housekeeping gene and calibrated to the average of control groups.

### AAV-AUF1 Expression/AAV AUF1 Gene Transfer

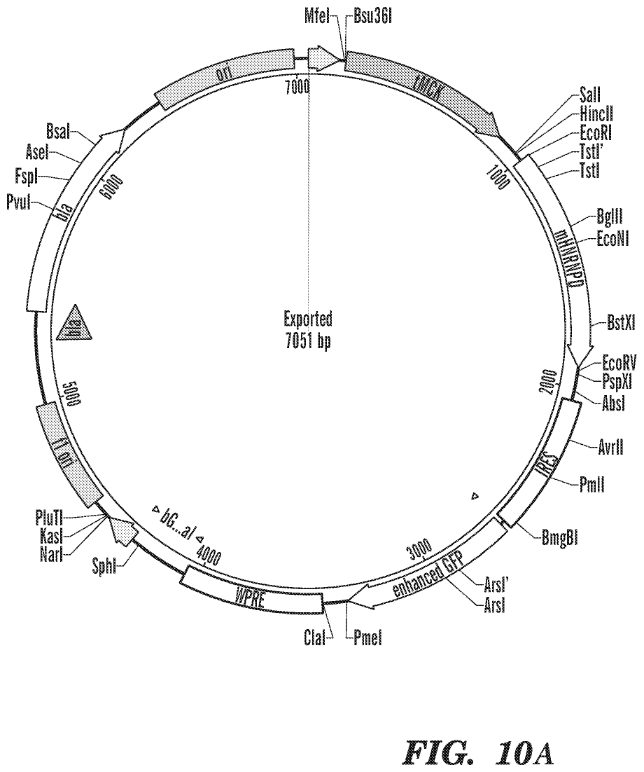

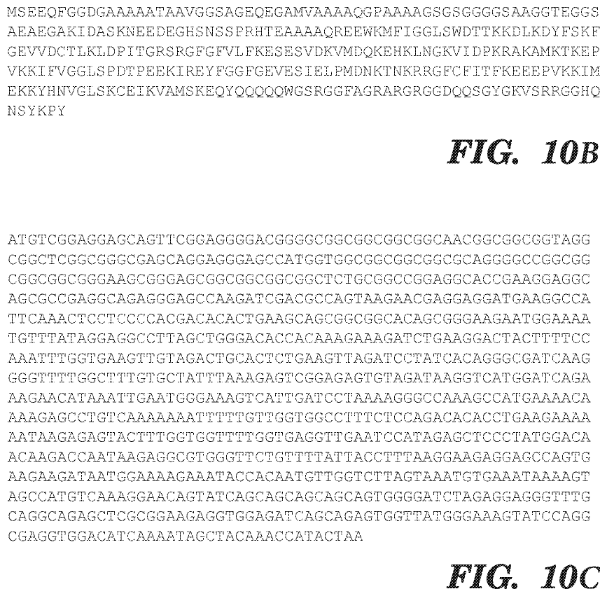

AUF1 was integrated into an AAV8 vector under the tMCK promoter (AAV8-tMCK-AUF1-IRES-eGFP) (Vector Biolabs) (FIG. 10). AAV8-tMCK-IRES-eGFP was used as a control vector. This promoter was generated by the addition of a triple tandem of 2RS5 enhancer sequences (3-Ebox) ligated to the truncated regulation region of the MCK (muscle creatine kinase) promoter, which induced high muscle specificity (Wang et al., “Construction and Analysis of Compact Muscle-Specific Promoters for AAV Vectors,” Gene Ther. 15(22):1489-1499 (2008), which is hereby incorporated by reference in its entirety). C57B16 mice were injected with a single retro-orbital injection of 50 μl (final concentration: 2.5×10¹¹ particles). Muscle function tests (grid hanging time, time and distance to exhaustion and maximum speed on a treadmill) were performed 40 days or 6 months post injection. Mice were then euthanized and tissues were collected.

### Muscle Function Tests

Grid hanging time. Mice were placed in the center of a grid, 30 cm above soft bedding to prevent injury should they fall. The grid was then inverted. Grid hanging time was measured as the amount of time mice held on before dropping off the grid. Each mouse was analyzed twice with 5 repetitions per mouse.

### Time, Distance to Exhaustion, and Maximum Speed.

After 1 week of acclimation, mice were placed on a treadmill and the speed was increased by 1 m/min every 3 minutes and the slope was increased every 9 minutes by 5 cm to a maximum of 15 cm. Mice were considered to be exhausted when they stayed on the electric grid more than 10 seconds. Based on their weight and running performance, work performance was calculated in Joules (J). Each mouse was analyzed twice with 5 repetitions per mouse.

### Strength by Grip Test (Examples 8 and 9)

In this test, mice grasp a horizon tall grid connected to a dynamometer and are pulled backwards five times by tugging on the tail. The force applied to the grid each time before the animal loses its grip is recorded in Newtons. The average of the five tests is then normalized to the whole-body weight of each mouse. Mice are typically analyzed twice with 5 repetitions per mouse.

### Dexa Muscle Mass Non-Invasive Quantitative Analysis (Example 7)

Dual energy X-ray absorptiometry (DEXA) was used to record lean muscle mass and changes in muscle mass upon injury or age previously published (Chenette et al., “Targeted mRNA Decay by RNA Binding Protein AUF1 Regulates Adult Muscle Stem Cell Fate, Promoting Skeletal Muscle Integrity,” Cell Rep. 16(5):1379-1390 (2016), which is hereby incorporated by reference in its entirety).

### Quantification of Satellite Cells (Example 7)

Muscles are excised and digested in collagenase type I. Cell numbers are quantified by flow cytometry gating for Sdc4⁺ CD45⁻ CD31⁻ Sca1⁻ satellite cell populations (Shefer et al., “Satellite-Cell Pool Size Does Matter: Defining the Myogenic Potency of Aging Skeletal Muscle,” Dev. Biol. 294(1):50-66 (2006) and Brack et al., “Pax7 is Back,” Skelet. Muscle 4(1):24 (2014), which are hereby incorporated by reference in their entirety).

### Muscle Fiber Type Analysis (Example 7)

Skeletal muscles were removed, put in OCT compound, fixed in 4% paraformaldehyde, and immunostained with antibodies to AUF1 (07-260, Millipore), slow myosin (NOQ7.5.4D, Sigma), fast myosin (MY-32, Sigma), and laminin alpha 2 membrane component (4H8-2, Sigma).

### Histological Studies and Biochemical Analysis of Muscle Tissues (Examples 7 and 8)

Muscles were removed and frozen in OCT compound, fixed in 4% paraformaldehyde, and blocked in 3% BSA in TBS. Immunofluorescence or immunochemistry (Hematoxylin and Eosin, Masson Trichome) was performed. Fibrosis was assessed by staining of muscle sections with Masson trichrome to visualize areas of collagen deposition and quantified using ImageJ software. Immunofluorescence images were acquired using a Zeiss LSM 700 confocal microscope. Images and morphometric analysis (Feret diameter, Cross sectional area) were processed using ImageJ as recently described (Abbadi et al., “Muscle Development and Regeneration Controlled by AUF1-Mediated Stage-Specific Degradation of Fate-Determining Checkpoint mRNAs,” Proc. Natl. Acad. Sci. USA 116(23):11285-11290 (2019), which is hereby incorporated by reference in its entirety). Muscles were harvested for biochemical analysis including immunoblot, RNAseq, and RT-PCR analysis.

### Evan Blue Dye Analysis (Example 7)

Evan Blue dye was used as an in vivo marker of muscle damage. It identifies permeable skeletal myofibers that have become damaged (Wooddell et al., “Myofiber Damage Evaluation by Evans Blue Dye Injection,” Curr. Protoc. Mouse Biol. 1(4):463-488 (2011), which is hereby incorporated by reference in its entirety).

### Serum Creatine Kinase (CK) Activity (Example 7)

Serum CK was evaluated at 37° C. by standard spectrophotometric analysis using a creatine kinase activity assay kit (abcam). The results are expressed in mU/mL.

### Blood Harvesting (Example 7)

Peripheral blood was harvested to quantify creatine kinase levels, and levels of cytokines, cells and inflammatory markers.

### Quantification and Statistical Analysis

All results are expressed as the mean±SEM. Two group comparisons were analyzed by the unpaired Mann-Whitney test. Multiple group comparisons were performed using one-way analysis of variance (ANOVA). The non-parametric Kruskal-Wallis test followed by the Dunn's comparison of pairs was used to analyze groups when suitable. P-values of <0.05 were considered significant. All statistical analyses were performed using GraphPad Prism (version 7) software.

### Genome-Wide Transcriptomic and Translatomic Studies and Bioinformatic Data Analysis (Example 7)

Polysome fractionation and mRNA isolation. Polysome isolation was performed by separation of ribosome-bound mRNAs by sucrose gradient centrifugation using cytoplasmic extracts as previously described (de la Parra et al., “A Widespread Alternate form of Cap-Dependent mRNA Translation Initiation,” Nat. Commun. 9(1):3068 (2018) and Badura et al., “DNA Damage and eIF4G1 in Breast Cancer Cells Reprogram Translation for Survival and DNA Repair mRNAs,” Proc. Natl. Acad. Sci. USA 109(46):18767-72 (2012), which are hereby incorporated by reference in their entirety). Post-fractionation samples were pooled based on enriched for mRNAs bound to 2-3 ribosomes and >4 ribosomes corresponding to poorly translated and well translated fractions respectively, and used for RNA sequencing (RNAseq). RNA quality was measured by a Bioanalyzer (Agilent Technologies).

RNA sequencing and data analysis. Paired-end RNA-seq was carried out by the New York University School of Medicine Genome Technology Core using the Illumina HiSeq 4000 single read. The low-quality reads (less than 20) were trimmed with Trimmomatic (Bolger et al., “Trimmomatic: A Flexible Trimmer for Illumina Sequence Data,” Bioinformatics, 30(15):2114-20 (2014), which is hereby incorporated by reference in its entirety) (version 0.36) with the reads lower than 35 nt being excluded. The resulted sequences were aligned with STAR (Dobin et al., “STAR: Ultrafast Universal RNA-Seq Aligner,” Bioinformatics 29(1):15-21 (2013), which is hereby incorporated by reference in its entirety) (version 2.6.0a) to the hg38 reference genome in the single-end mode. The alignment results were sorted with SAMtools (Li et al., “The Sequence Alignment/Map format and SAMtools,” Bioinformatics 25(16):2078-2079 (2009), which is hereby incorporated by reference in its entirety) (version 1.9), after which supplied to HTSeq (Anders et al., “HTSeq—A Python Framework to Work with High-Throughput Sequencing Data,” Bioinformatics 31(2):166-9 (2015), which is hereby incorporated by reference in its entirety) (version 0.10.0) to obtain the feature counts. The feature counts tables from different samples were concatenated with a custom R script. To examine differences in transcription and translation, total mRNA and polysome mRNA were quantile-normalized separately. Regulation by transcription and translation and accompanying statistical analysis was performed using RIVET (Ernlund et al., “RIVET: Comprehensive Graphic User Interface for Analysis and Exploration of Genome-Wide Translatomics Data,” BMC Genomics 19(1):809 (2018), which is hereby incorporated by reference in its entirety), where significant genes were identified as P<0.05 and >1 log fold change. Reactome pathway analysis was performed on genes that were up- and down-regulated by transcription and translation using Metascape (Zhou et al., “Metascape Provides a Biologist-Oriented Resource for the Analysis of Systems-Level Datasets,” Nat. Commun. 10(1):1523 (2019), which is hereby incorporated by reference in its entirety). Pathway analysis and enrichment plots of the top 100 genes that were the most regulated by transcription and/or translation were generated using DAVID (Huang da et al., “Systematic and Integrative Analysis of Large Gene Lists Using DAVID Bioinformatics Resources,” Nat. Protoc. 4(1):44-57 (2009), which is hereby incorporated by reference in its entirety) and Metascape. Prediction of transcription factors of the same list of 100 genes was performed using Enrichr (Chen et al., “Enrichr: Interactive and Collaborative HTML5 Gene List Enrichment Analysis Tool,” BMC Bioinformatics 14:128 (2013), which is hereby incorporated by reference in its entirety) (TRANSFAC and JASPER PWM program) and PASTAA (Roider et al., “Predicting Transcription Factor Affinities to DNA from a Biophysical Model,” Bioinformatics 23(2):134-41 (2007), which is hereby incorporated by reference in its entirety) online tool. Genes enriched in TFH cells was determined from GSE16697 (Johnston et al., “Bcl6 and Blimp-1 are Reciprocal and Antagonistic Regulators of T Follicular Helper Cell Differentiation,” Science 325(5943):1006-1010 (2009), which is hereby incorporated by reference in its entirety) and similar genes between datasets were determined using Venny.

### Traumatic Injury Animal Model (Example 8)

Three month old male mice, unless otherwise noted, were administered an intramuscular injection of 50 μl of filtered 1.2% BaCl₂ in sterile saline with control or with lentivirus AUF1 vector (1×10⁸ genome copy number/ml) (total volume 100 l) into the left tibialis anterior (TA) muscle. The right TA muscle remained uninjured as a control. Mice were sacrificed at 3 or 7 days post-injection. Muscles were weighed and frozen in OCT for immunofluorescence staining or put in Trizol for mRNA extraction.

### Example 1—Skeletal Muscle AUF1 Expression is Downregulated with Age

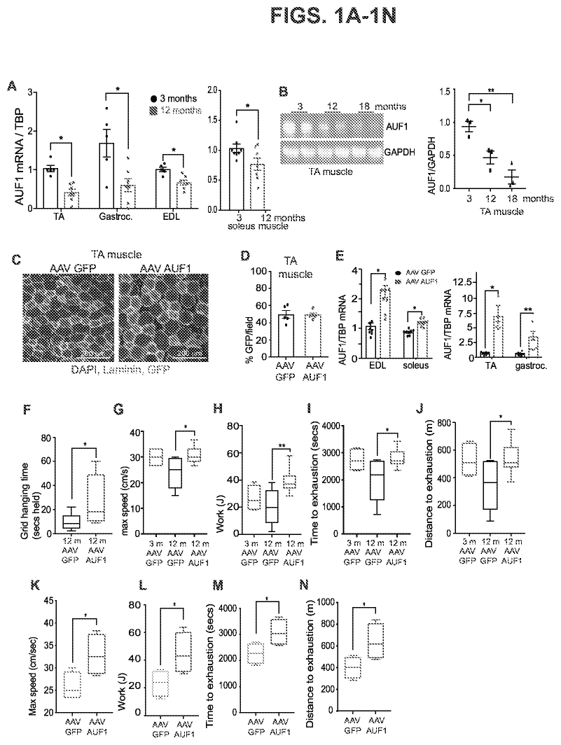

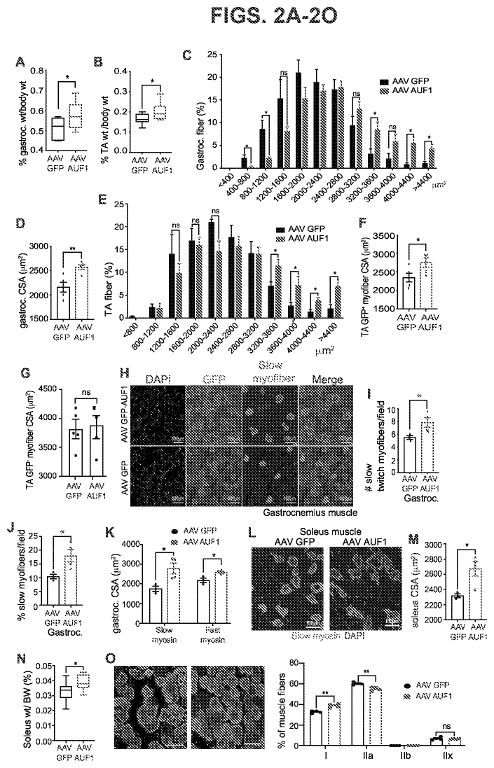

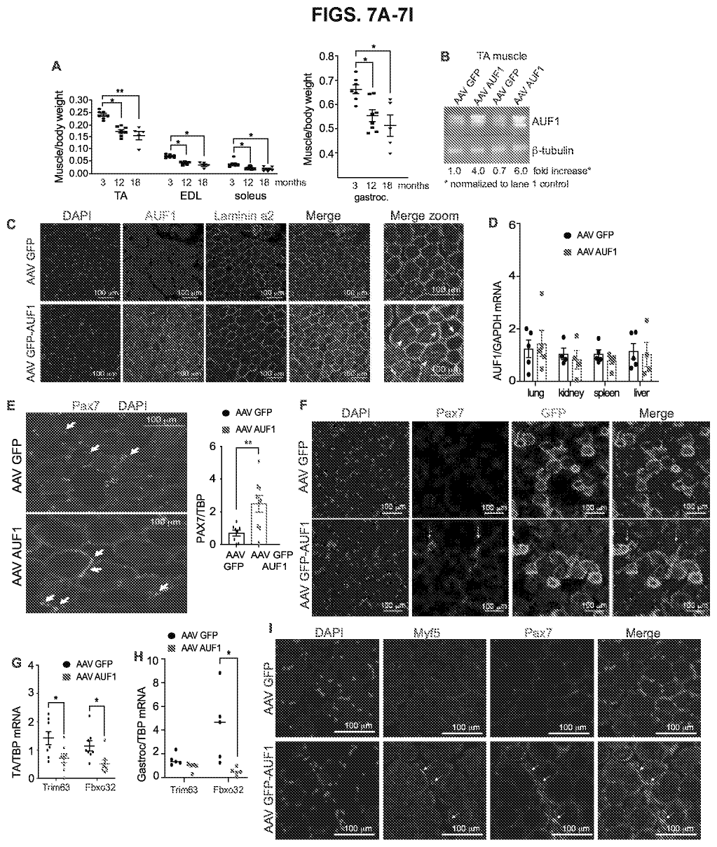

Because mice deleted in the auf1 gene undergo an accelerated loss of muscle mass (Chenette et al., “Targeted mRNA Decay by RNA Binding Protein AUF1 Regulates Adult Muscle Stem Cell Fate, Promoting Skeletal Muscle Integrity,” Cell Rep. 16(5):1379-1390 (2016); Abbadi et al., “Muscle Development and Regeneration Controlled by AUF1-Mediated Stage-Specific Degradation of Fate-Determining Checkpoint mRNAs,” Proc. Natl. Acad. Sci. USA 116(23):11285-11290 (2019); and Pont et al., “mRNA Decay Factor AUF1 Maintains Normal Aging, Telomere Maintenance, and Suppression of Senescence by Activation of Telomerase Transcription,” Molecular Cell 47(1):5-15 (2012), which are hereby incorporated by reference in their entirety), whether reduced expression of AUF1 with age occurs in wild type animals and is involved in age-related muscle atrophy was investigated. The expression of AUF1 in limb skeletal muscles of young (3 month), middle-aged (12 month) and older mice (18 month) was analyzed. Compared to 3 month young mice, auf1 mRNA expression was strongly downregulated by 12 months of age in non-exercised animals, shown in the tibialis anterior (TA), gastrocnemius, extensor digitorum longus (EDL) and soleus muscles (FIG. 1A). In all studies test mRNAs were normalized to gapdh or tbp mRNAs which were unchanged in abundance regardless of AUF1 expression. As shown in the TA muscle, AUF1 protein levels tracked mRNA levels, which were reduced ˜3-fold at 12 months and 4-fold at 18 months, normalized to muscle total protein and invariant GAPDH (FIG. 1B). Reduced skeletal muscle AUF1 expression with age in non-exercised animals was associated with a significant loss of muscle mass in limb muscles, shown in the TA, EDL, gastrocnemius and soleus muscles in 12 and 18 month old mice compared to 3 month old animals (FIG. 7A). Importantly, by 18 months of age, loss of muscle mass began to plateau from 12 month values. The TA muscle was reduced in relative mass by almost 50%, the EDL by 30%, the soleus by almost 50% and the gastrocnemius by 25%. It should be noted that there was also evident reduced absolute muscle mass at 12 and 18 months that cannot be accounted for by an increase in overall body weight in adults compared to young mice (Table 5). In 12 and 18 month old mice, the gastrocnemius was reduced 11% and 14%, and the TA muscle by 18% and 24%, respectively, which has been observed by others as well.

TABLE 5

| | | Mean | |
|---|---|---|---|
| Age | Muscle | weight (mg) | P value |
| 3 month | TA | 81.4 | |
| | EDL | 18.5 | |
| | Soleus | 13.7 | |
| | Gastroc. | 234.5 | |
| 12 month | TA | 66.5 | 0.0002 *** |
| | EDL | 17.8 | 0.65 ns |
| | Soleus | 13.7 | 0.94 ns |
| | Gastroc. | 202.4 | 0.004 ** |
| 18 month | TA | 61.8 | 0.05 * |
| | EDL | 17.1 | 0.44 ns |
| | Soleus | 10.9 | 0.22 ns |
| | Gastroc. | 208.2 | 0.25 ns |

* P < 0.05,
** P < 0.01,
*** P < 0.001;
ns, not significant by unpaired Mann-Whitney U test

### Example 2—AUF1 Skeletal Muscle Gene Transfer Enhances Exercise Endurance in Middle-Aged and Old Mice

Whether loss of skeletal muscle mass with age in mice is a result of reduced expression of AUF1 in skeletal muscle was investigated. An AAV8 (adeno-associated virus type 8) vector was developed to deliver and selectively express AUF1 in skeletal muscle. AAV vectors express AUF1 and GFP (AUF1-GFP, with GFP translated from the same mRNA as AUF1 by the HCV IRES), or as a control only GFP. Expression of both genes is controlled by the creatine kinase tMCK promoter that is selectively active in skeletal muscle cells Wang et al., “Construction and Analysis of Compact Muscle-Specific Promoters for AAV Vectors,” Gene Ther. 15(22):1489-1499 (2008), which is hereby incorporated by reference in its entirety). Mice ages 3 and 12 months were administered a single retro-orbital injection of either AAV AUF1-GFP or control AAV GFP vectors (2.0×10¹¹ genome copies). When analyzed starting at 40 days post-administration of AAV vectors, as shown in 12 month old mice, both AAV AUF1-GFP and AAV GFP control vector-treated animals displayed similar vector transduction and retention rates, shown by TA muscle GFP staining (FIGS. 1C-1D). auf1 mRNA expression in skeletal muscle was increased by AAV8 AUF1-GFP administration, on average 2.5-fold in EDL, 6-fold in TA, 2.5-fold in gastrocnemius and slightly in soleus muscle (FIG. 1E). AUF1 protein levels in gene transferred animals in skeletal muscle, as shown in the TA muscle, demonstrated 4-6 fold increased expression over endogenous levels, corresponding to auf1 mRNA levels (FIGS. 1E and 7B). Representative immunofluorescence staining also demonstrated strong uptake and expression of AUF1 localized in nuclei (white arrows) and sarcoplasm (yellow arrows) as expected in AAV AUF1-GFP infected TA muscle fibers that is not seen for control AAV GFP (FIG. 7C). There was no evidence for increased expression of AUF1 in non-muscle tissues compared to control mice receiving either vector administered either vector (kidney, lung, spleen, liver) (FIG. 7D), demonstrating strong tissue specificity for skeletal muscle expression controlled by the tMCK promoter. Importantly, Pax7 expression, a key marker for activation of muscle satellite cells and proliferating myoblasts, was also increased 3-4 fold with AAV AUF1-GFP administration (FIG. 7E). Moreover, increased expression of Pax7 was limited to cells expressing AUF1-GFP, which was not evident in cells expressing only GFP in the absence of AUF1 gene delivery (FIG. 7F). Correspondingly, markers of muscle atrophy such as trim63 and fbxo32 (Nilwik et al., “The Decline in Skeletal Muscle Mass with Aging is Mainly Attributed to a Reduction in type II muscle Fiber Size,” Exp. Gerontol. 48(5):492-498 (2013), which is hereby incorporated by reference in its entirety), were downregulated 2-3-fold in the TA muscle, and 0.5 to 4-fold (respectively) in gastrocnemius muscle in animals administered AAV AUF1-GFP but AAV GFP (FIGS. 7G and 7 H). These data indicate that AUF1 gene transfer into skeletal muscle is sufficient to reduce markers of muscle atrophy coincident with activation of satellite cells and myoblasts.

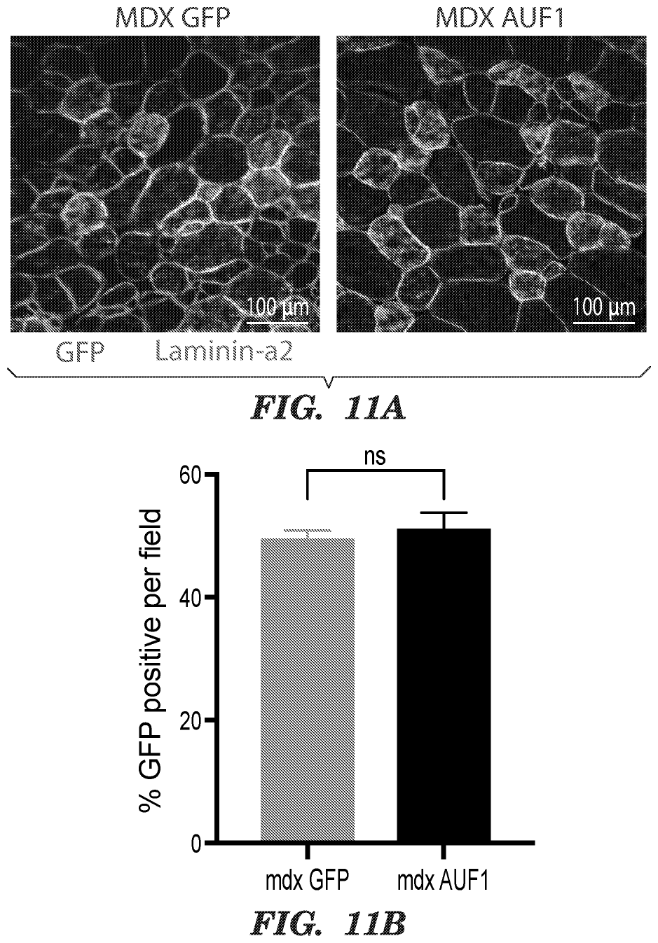

It was therefore investigated whether AUF1 gene transfer can increase physical endurance in middle aged and older sedentary mice (12 and 18 month old mice), using a number of well-established criteria. Twelve month old sedentary mice were administered AAV8 AUF1-GFP or control AAV8 GFP, then tested at 40 days post-administration. AUF1 supplemented mice showed a ˜50% improvement in grid hanging time (FIG. 1F), a measure of limb-girdle skeletal muscle strength and endurance. When tested by treadmill, AAV AUF1-GFP mice displayed 25% higher maximum speed (FIG. 1G) and 50% increase in work performance (FIG. 1H) compared to AAV GFP control mice, as well as 25% greater time to exhaustion and 30% increased distance to exhaustion (FIGS. 11 and 1J). When compared to 3 month old mice receiving control AAV GFP, 12 month old mice gained equivalent physical endurance capacity to the level of young mice. Physical endurance was also tested 6 months post AAV-AUF1 injection of 12 month old mice that were 18 months at the time and kept non-exercised until the time of testing. Maximum speed (FIG. 1K), work performed (FIG. 1L), as well as time and distance to exhaustion (FIGS. 1M and 1N) were all significantly higher in AUF1-AAV treated animals, similar to 12 month old mice at 40 days post-treatment. These results demonstrate that the enhancement of exercise endurance in older mice with muscle loss and atrophy by supplementation with AUF1 is durable at 6 months post-treatment, with no evidence for diminution. It was therefore next investigated whether the biological and molecular characteristics of AUF1 restored skeletal muscle.

### Example 3—AUF1 Gene Therapy Increases Muscle Mass and Greater Slow-Twitch than Fast-Twitch Myofibers

Skeletal muscles vary in slow- and fast-twitch myofiber composition (Type I or II, respectively). EDL, and gastrocnemius muscles are composed mostly of Type II fast-twitch myofibers (nearly 99% fast, 1% slow), the TA is ˜20% Type I and 80% Type II, whereas the soleus muscle is highly enriched in Type I slow-twitch myofibers (nearly 40% slow, 60% fast) (Augusto et al., “Skeletal Muscle Fiber Types in C57BL6J mice,” J. Morphol. Sci. 21(2):89-94 (2004), which is hereby incorporated by reference in its entirety). Analysis of the gastrocnemius and TA muscles showed that 12 month sedentary old mice gained an average total increase of ˜20% in muscle mass relative to body weight in animals administered AAV AUF1-GFP compared to AAV GFP controls (FIGS. 2A and 2B). Increased muscle fiber size (myofiber cross-sectional area, CSA) and number are established hallmarks of muscle regeneration (Schiaffino & Reggiani, “Fiber Types in Mammalian Skeletal Muscles,” Physiol. Rev. 91(4):1447-1531 (2011) and Yin et al., “Satellite Cells and the Muscle Stem Cell Niche,” Physiol. Rev. 93(1):23-67 (2013), which are hereby incorporated by reference in their entirety). In 12 month old mice, AUF1 supplemented gastrocnemius and TA muscles increased in muscle fiber size (myofiber cross-sectional area, CSA) particularly in the percentage of larger myofibers (≥3200 m²), as well as number, which largely represents increased fast twitch Type II fibers mice, (FIG. 2C-F). Increased myofiber size can be indicative of vigorous and mature muscle regeneration. In contrast to AUF1-transduced myofibers, non-transduced (GFP-) myofibers saw no increase in CSA, as shown for the TA muscle in animals administered either AAV GFP or AAV AUF1-GFP (FIG. 2G). Non-transduced myofibers tended to have a greater CSA than vector transduced fibers expressing GFP. It is possible that the largest fibers are not efficiently infected.

The gastrocnemius muscle in animals administered either control GFP or AUF1-GFP AAV8 was analyzed by co-staining for GFP and slow myosin to determine AAV8 infection levels in slow and fast-twitch myofibers (FIG. 2H). The AAV8 vector efficiently infected both slow myofibers (red and GFP stained) and fast myofibers (GFP stained only). Since Type I myofibers comprise a small percentage of most muscles, the effect of supplemental AUF1 expression specifically on slow-myofibers was investigated by co-staining with slow myosin and GFP. In the gastrocnemius muscle, AUF1 supplementation increased by more than 50% the number of Type I myofibers per field, the percentage per field, and the CSA (FIGS. 2H-2K). In the soleus muscle, which is composed primarily of ˜40% Type I fibers, the CSA was similarly increased with AUF1 supplementation, as was muscle weight normalized to body weight (FIGS. 2L-2N). Next, we immunostaining of the different myofibers in the soleus muscle was carried out at 40 d post-AUF1 gene transfer in 12 month old mice (FIG. 2O). These data show that AUF1 gene transfer results in a small increase in Type I fibers and a small reduction in Type IIa myofibers in the soleus muscle, without altering levels of IIb and IIx myofibers. These data correlate with increased endurance in mice receiving AUF1 gene transfer and that AUF1 promotes formation of Type I myofibers, either by myofiber conversion, regeneration, or both. Therefore, immunostaing of Pax7 and Myf5 in the TA muscle of 12 month old mice was carried out 40 days after AUF1 gene transfer to determine levels of satellite cell activation indicative of muscle hypertrophy and regeneration. As shown in representative images (FIG. 7I), Myf5 staining correlated with Pax7 co-staining, supporting the conclusion that AUF1 gene therapy promotes muscle hypertrophy, regeneration and fiber conversion.

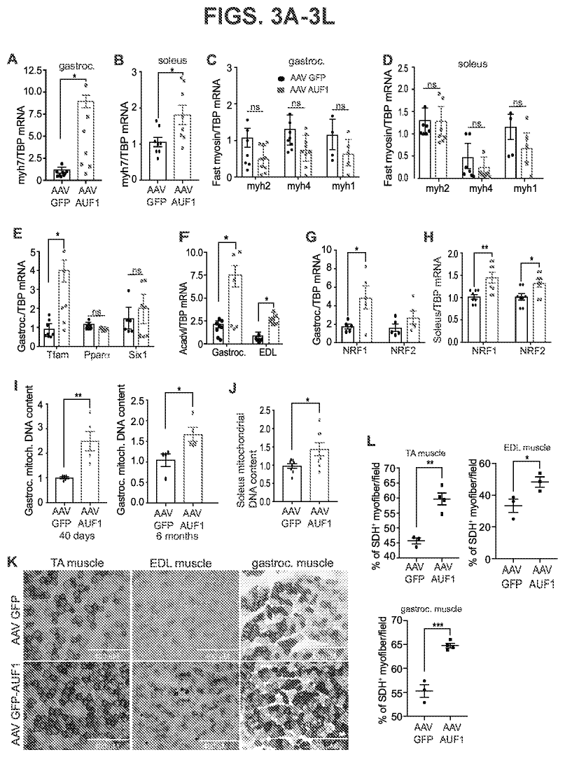

Expression levels of different myosin type mRNAs also support that AUF1 gene transfer resulted in real gain in skeletal muscle mass. The major slow-twitch myosin mRNA, myh7, was increased 6-fold in gastrocnemius and 2-fold in soleus muscle with AUF1 gene transfer (FIG. 3A, FIG. 3B), whereas fast myosin mRNAs such as myh1, myh2 and myh4 were not statistically changed (FIG. 3C, FIG. 3D).

Further evidence was obtained for increased muscle generation by AUF1 is supported by measuring the mRNA levels of several genes whose expression are hallmarks of increased myofiber regeneration, oxidative processes and mitochondrial biogenesis. Slow-twitch myofibers in particular are enriched in oxidative mitochondria (Schiaffino & Reggiani, “Fiber Types in Mammalian Skeletal Muscles,” Physiol. Rev. 91(4):1447-1531 (2011), which is hereby incorporated by reference in its entirety). The focus of these studies was on the gastrocnemius muscle because it demonstrated a median response to AUF1 gene therapy and it is not biased toward enrichment of slow-twitch myofibers. While AUF1 gene transfer had no effect on gastrocnemius mRNA levels of non-mitochondrial genes such as pparα (peroxisome proliferator-activated receptor alpha) or six1(Sineoculis homeobox homolog 1), it increased levels of mitochondrial mRNAs for tfam (mitochondria transcription factor A) by 4-fold, acadvl (acyl-CoA dehydrogenase very long chain) by 6-fold, nrf1 and nrf2 by 2-3-fold (nuclear respiratory factor) (FIGS. 3E-3H). The ratio of mitochondrial to nuclear DNA was also increased in the gastrocnemius with AUF1 gene transfer, indicative of increased mitochondrial content at both 40 days and 6 months post-gene transfer (FIG. 3I, FIG. 3J). Finally, the myofiber succinate dehydrogenase (SDH) activity, a mitochondrial membrane protein complex in muscle that is an established indicator of mitochondrial and oxidative potential, was quantified. ezawork-Geleta, A., Rohlena, J., Dong, L., Pacak, K. & Neuzil, J. (2017). Mitochondrial Complex II: At the Crossroads. Trends Biochem Sci 42, 312-325. Immunohistochemical determination of SDH activity showed that it was strongly increased in TA, gastrocnemius and EDL muscle fibers, and only in animals receiving AAV AUF1 supplementation (FIG. 2K), averaging 30% or more in all three muscles (FIG. 3L). Collectively, these results show that AUF1 promotes transition from fast to slow twitch myofiber. Collectively, these results show that AUF1 promotes transition from fast to slow twitch myofiber.

### Example 4—AUF1 Stimulates Slow-Twitch Muscle Development in Part by Increasing PGC1a Expression

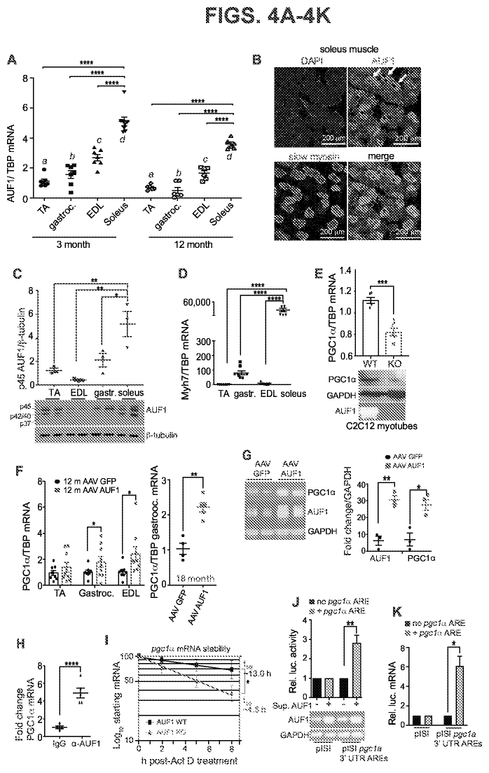

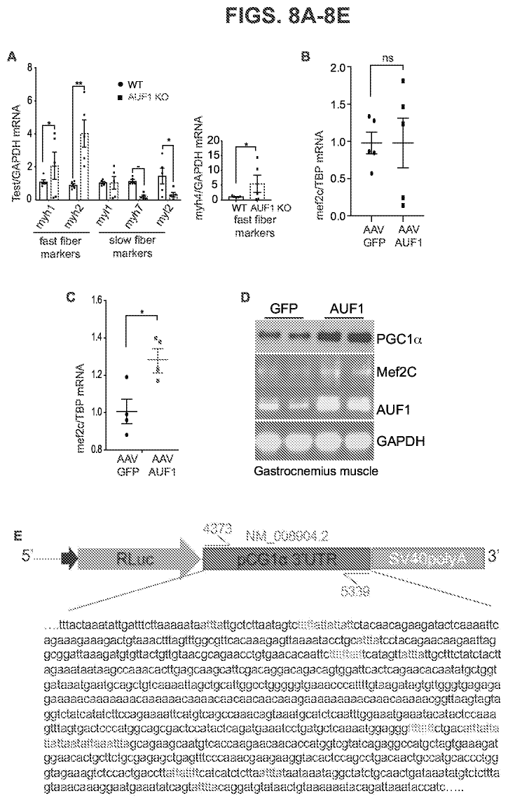

Increased levels slow-twitch Type I muscle fibers are particularly sought for combating muscle loss with age because it is associated with increased muscle endurance. A key feature of slow muscle is that it confers exercise endurance because slow-twitch myofibers have much higher oxidative capacity than fast-twitch fibers (Cartee et al., “Exercise Promotes Healthy Aging of Skeletal Muscle,” Cell Metab. 23(6):1034-1047 (2016) and Yoo et al., “Role of Exercise in Age-Related Sarcopenia,” J. Exerc. Rehabil. 14(4):551-558 (2018), which are hereby incorporated by reference in their entirety). Therefore, the level of AUF1 expression in different muscles with varying proportions of slow- and fast myofibers was characterized. There was a notable 2-4 fold higher level of expression of auf1 mRNA and AUF1 protein levels in the soleus muscle of 3 month and sedentary 12 month old untreated mice compared to other muscle types with fewer slow-twitch myofibers (FIGS. 4A-4C). Accordingly, of the lower limb skeletal muscles, the soleus muscle is the most endurant, the most enriched in slow-twitch myofibers (Schiaffino & Reggiani, “Fiber Types in Mammalian Skeletal Muscles,” Physiol. Rev. 91(4):1447-1531 (2011) and Augusto et al., “Skeletal Muscle Fiber Types in C57BL6J mice,” J. Morphol. Sci. 21(2):89-94 (2004), which are hereby incorporated by reference in their entirety), and expresses much higher levels of myh7(FIG. 4D), the main slow-twitch myofiber myosin. Therefore, the role of AUF1 in expression of different levels of myosin mRNAs was assessed by deletion of AUF1 in C2C12 mouse myoblasts. Deletion of AUF1 increased the expression of fast-twitch myh2 mRNA levels, while slow myosin mRNAs, such as myh7 or myl2, were decreased (FIG. 8A), consistent with AUF1 greater specification of slow-twitch myofiber development. MEF2c can activate or repress different myogenic transcriptional programs and its increased expression is also consistent with increased generation of Type I slow-twitch muscle (Lin et al., “Transcriptional Co-Activator PGC-1 Alpha Drives the Formation of Slow-Twitch Muscle Fibres,” Nature 418 (6899):797-801 (2002), which is hereby incorporated by reference in its entirety), suggesting involvement in AUF1-mediated specification of slow-twitch muscle. MEF2C levels were assessed because AUF1 was previously shown to promote mef2c ARE-mRNA translation without altering its mRNA stability. Lin, J. et al. (2002). Transcriptional co-activator PGC-1 alpha drives the formation of slow-twitch muscle fibres. Nature 418, 797-801; and Panda, A. C. et al. (2014). RNA-binding protein AUF1 promotes myogenesis by regulating MEF2C expression levels. Mol Cell Biol 34, 3106-3119. mef2c mRNA levels were not increased at 40 days post-AUF1 supplementation, and showed only a slight increase at 6 months (FIGS. 8B and 8C). MEF2C protein levels were moderately increased at 40 d post-supplementation (FIG. 8D), whereas PGC1α protein levels were increased strongly at 40 d post-supplementation. As shown later (FIG. 4G), increased PGC1α protein levels were sustained at 6 months post-AUF1 supplementation.

The MEF2c protein stimulates expression of PGC1α (Peroxisome proliferator-activated receptor gamma coactivator 1 alpha) which drives the specification and development of slow-twitch myofibers (Lin et al., “Transcriptional Co-Activator PGC-1 Alpha Drives the Formation of Slow-Twitch Muscle Fibres,” Nature 418 (6899):797-801 (2002), which is hereby incorporated by reference in its entirety). Deletion of the auf1 gene in C2C12 myoblasts induced to differentiate to myotubes decreased pgc1α mRNA levels by half and protein levels by 4-fold (FIG. 4E), suggesting that AUF1 acts to increase PGC1α protein and mRNA expression. Accordingly, AAV8-AUF1 gene transfer in mice showed that pgc1α mRNA levels were increased 2-3 fold in the gastrocnemius and EDL muscles, and trended toward upregulation in the TA muscle in 12 month old mice (FIG. 4F). AUF1 gene transfer in 18 month old sedentary mice also strongly increased pgc1α mRNA levels ˜2.5-fold, as shown in the gastrocnemius muscle (FIG. 4F), which corresponded to an average 5-fold increase in PGC1α protein levels (FIG. 4G).

The pgc1α mRNA contains a 3′ UTR with multiple ARE motifs that could be potential AUF1-binding sites (Lai et al., “Effect of Chronic Contractile Activity on mRNA Stability in Skeletal Muscle,” Am. J. Physiol. Cell. Physiol. 299(1):C155-163 (2010), which is hereby incorporated by reference in its entirety). Therefore, AUF1 was immunoprecipitated from WT C2C12 myoblasts 48 hours after differentiation when AUF1 is expressed, with control IgG or anti-AUF1 antibodies, followed by qRT-PCR to quantify the levels of bound pgc1α mRNA (FIG. 4G). AUF1 bound strongly to the pgc1a mRNA in differentiating C2C12 cells. The effect of AUF1 expression on the pgc1a mRNA half-life was then determined using WT and AUF1 KO C2C12 cells by addition of actinomycin D to block new transcription (FIG. 4H). Surprisingly, in the absence of AUF1, pgc1α mRNA displayed an almost 3-fold reduced stability. Studies next determined whether AUF1 acts on the pgc1α 3′UTR AU-rich elements. The pgc1α 3′UTR AU-rich region was inserted into the 3′UTR of a luciferase reporter (FIG. 8E) and compared control luciferase activity and mRNA levels to luciferase with the pgc1α ARE in transfected C2C12 myoblasts, or myoblasts stably transfected with p40 AUF1 to increase AUF1 expression. Three-fold increased expression of AUF1 increased activity (expression) of luciferase by -3-fold from the mRNA containing the pgc1α AREs, and luc-ARE mRNA levels by 6-fold (FIGS. 4J and 4K). The pgc1α mRNA therefore belongs to the class of ARE-mRNAs that are stabilized rather than destabilized by AUF1, accounting in part for increased levels of PGC1α protein and increased specification of slow-twitch fiber formation by AUF1. The effect of AUF1 expression was investigated specifically on slow-twitch muscle loss and atrophy.

### Example 5—Loss of AUF1 Expression Selectively Accelerates Atrophy of Slow-Twitch Muscle in Young Mice

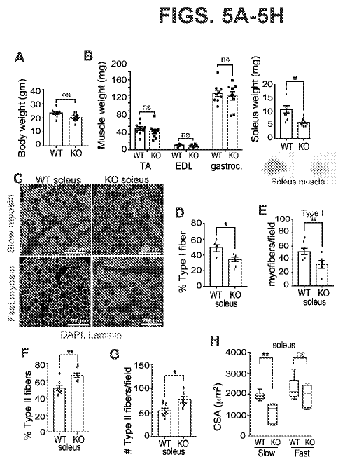

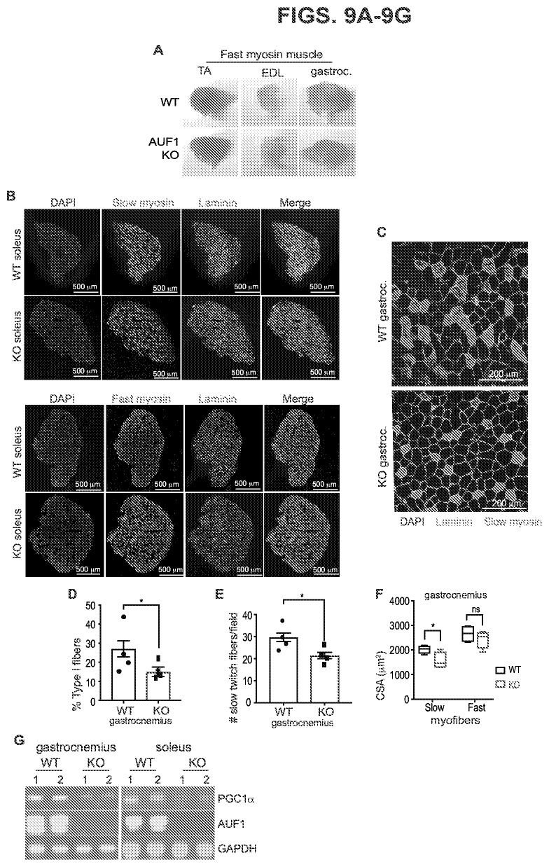

To better understand the role of AUF1 gene therapy in the formation and maintenance of slow-twitch myofibers, slow-twitch myofibers in WT and AUF1 KO mice were investigated at 3 months of age, before the onset of dystrophy (Chenette et al., “Targeted mRNA Decay by RNA Binding Protein AUF1 Regulates Adult Muscle Stem Cell Fate, Promoting Skeletal Muscle Integrity,” Cell Rep. 16(5):1379-1390 (2016), which is hereby incorporated by reference in its entirety). At 3 months, WT and auf1 KO mice have similar body weights (FIG. 5A). While deletion of auf1 did not change the size, color (mitochondrial density, myoglobin content) or weight of the TA, EDL or gastrocnemius muscles, it did reduce the size and weight of the soleus muscle by half at 3 months, which was much paler, indicative of loss of mitochondrial and myoglobin-rich Type I myofibers (FIG. 5B; FIG. 9A). The proportion and number per field of slow myosin myofibers in the AUF1 KO mouse soleus muscle was reduced 40-50% (FIGS. 5C-5E; FIG. 9B). In contrast, both the proportion and number of fast-myosin-expressing myofibers was increased by 25% or more in the absence of AUF1 expression (FIGS. 5C-5E and FIG. 9B). Reduced expression of slow myosin was also seen in the gastrocnemius muscle with auf1 deletion in auf1 KO mice (FIGS. 9C-9E). In addition, the mean CSA was reduced by 2-fold in slow-twitch myofibers, as shown in the soleus and gastrocnemius muscles, but was unchanged in fast-twitch myofibers (FIG. 5H; FIG. 9F). Consistent with these data, AUF1 KO mice at 3 months expressed 3-4 fold lower levels of PGC1α protein than WT mice, as shown in the gastrocnemius and soleus muscles (FIG. 9G). AUF1 therefore specifies regeneration and maintenance of slow-twitch muscle.

### Example 6—Reduced Expression of AUF1 in Adult Mice Accelerates Atrophy and Decline of Both Slow- and Fast-Twitch Muscle

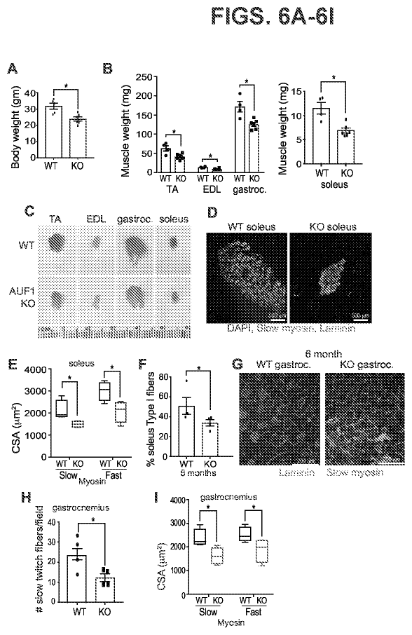

At 6 months of age, auf1 KO mice show a 20% loss of body weight, which is largely a result of loss of skeletal muscle mass (FIGS. 6A and 6B). Unlike 3 month old mice where the slow-twitch rich soleus muscle was the only muscle showing significant atrophy in the absence of AUF1 expression, in 6 month old mice both fast-twitch rich and slow-twitch rich muscles demonstrate significant atrophy. The size and weight of the TA, EDL and gastrocnemius muscles were reduced by -25% in auf1 KO compared to WT animals, and the soleus muscle was reduced by almost 50% (FIG. 6B). In addition, auf1 KO mouse skeletal muscles were paler than control WT mice, consistent with greater loss of mitochondrial-dense, slow-twitch myofibers (FIG. 6C). Accordingly, the mean CSA of both slow- and fast-twitch myofibers, as shown in the soleus and gastrocnemius muscles, showed a striking reduction at 6 months in auf1 KO mice compared to WT, indicative of overall myofiber atrophy (FIG. 6D, FIG. 6E). As seen in 3 month old mice, AUF1 deficiency reduced by half the percentage and number of slow-twitch myofibers per field in the soleus and gastrocnemius muscles (FIGS. 6F-6I). Thus, while AUF1 specifies development of slow-twitch muscle, its additional activities are essential for maintenance and regeneration of both slow- and fast-twitch muscle, consistent with the ability of AUF1 gene transfer to promote increased overall muscle mass and function in sedentary animals that have undergone muscle loss and atrophy during aging.

### Discussion of Examples 1-6

These examples report four important sets of findings: (1) AUF1 expression in skeletal muscle is diminished in adult compared to young mice, which contributes to a reduction in muscle mass and function; (2) AUF1 gene transfer might provide a therapeutic intervention to delay or possibly reverse the loss of muscle mass and strength with age; and (3) AUF1 is required for the maintenance of both slow and fast myofibers; and (4) AUF1 promotes a transition from fast to slow muscle phenotype by increasing PGC1α levels through stabilization of its mRNA. AUF1 generally promotes rapid decay of ARE-containing mRNAs but can stabilize a subset of other ARE-mRNAs (Moore et al., “Physiological Networks and Disease Functions of RNA-Binding Protein AUF1,” Wiley Interdiscip. Rev. RNA 5(4):549-564 (2014), which is hereby incorporated by reference in its entirety). During muscle regeneration, AUF1 therefore regulates satellite cell maintenance and differentiation in part by programming each stage of myogenesis through selective degradation of short-lived myogenic checkpoint ARE-mRNAs (Chenette et al., “Targeted mRNA Decay by RNA Binding Protein AUF1 Regulates Adult Muscle Stem Cell Fate, Promoting Skeletal Muscle Integrity,” Cell Rep. 16(5):1379-1390 (2016) and Abbadi et al., “Muscle Development and Regeneration Controlled by AUF1-Mediated Stage-Specific Degradation of Fate-Determining Checkpoint mRNAs,” Proc. Natl. Acad. Sci. USA 116(23):11285-11290 (2019), which are hereby incorporated by reference in their entirety). In addition, as shown here, by increasing AUF1 expression levels in skeletal muscles in mice using AAV gene transfer, AUF1 increases the expression of slow myosins and oxidative mitochondrial genes which mediate slow myofiber formation and oxidative phenotype. There is also evidence for reduced AUF1 expression in human skeletal muscle with aging (Masuda et al., “Tissue- and Age-Dependent Expression of RNA-Binding Proteins that Influence mRNA Turnover and Translation,” Aging (Albany NY) 1:681-698 (2009), which is hereby incorporated by reference in its entirety), although the general inability to obtain serial age-related but otherwise normal muscle specimens limits the ability to expand this finding.

Gene therapy of skeletal muscle with AUF1 by AAV8-AUF1 significantly promoted new muscle mass and exercise endurance in 12 and 18 month old non-exercised mice that had significant muscle loss and atrophy and increased muscle decline and atrophy compared to 3 month old young mice. Notably, in a rat model designed to characterize skeletal muscle markers of increased physical exercise endurance, two major factors that were found to be increased in expression were AUF1 and PGC1α (Lai et al., “Effect of Chronic Contractile Activity on mRNA Stability in Skeletal Muscle,” Am. J. Physiol. Cell. Physiol. 299(1):C155-163 (2010), which is hereby incorporated by reference in its entirety). Moreover, an exercise study in mice found that while one week of exercise induced increased levels of PGC1α, after four weeks of exercise AUF1 increased as much as 50% without changes in other ARE-binding proteins (Matravadia et al., “Exercise Training Increases the Expression and Nuclear Localization of mRNA Destabilizing Proteins in Skeletal Muscle,” Am. J. Physiol. Regul. Integr. Comp. Physiol. 305(7):R822-831 (2013), which is hereby incorporated by reference by its entirety).

Interestingly, pgc1α, tfam and nrf2 mRNAs all contain AREs in their 3′UTRs, which may be subject to regulation by ARE-binding proteins, including AUF1 (D'Souza et al., “mRNA Stability as a Function of Striated Muscle Oxidative Capacity,” Am. J. Physiol. Regul. Integr. Comp. Physiol. 303(4):R408-417 (2012), which is hereby incorporated by reference in its entirety). While perplexing at the time, AUF1 was then only known to cause ARE-mRNA decay, not stabilization. These findings, when combined with the results disclosed herein, suggests that AUF1 programs a feed-forward mechanism to promote muscle regeneration through stabilization of pgc1αmRNA and, through other AUF1 activities as well (Chenette et al., “Targeted mRNA Decay by RNA Binding Protein AUF1 Regulates Adult Muscle Stem Cell Fate, Promoting Skeletal Muscle Integrity,” Cell Rep. 16(5):1379-1390 (2016) and Abbadi et al., “Muscle Development and Regeneration Controlled by AUF1-Mediated Stage-Specific Degradation of Fate-Determining Checkpoint mRNAs,” Proc. Natl. Acad. Sci. USA 116(23):11285-11290 (2019), which are hereby incorporated by reference in their entirety). Consistent with this conclusion, the AUF1 KO mice used herein present at a young age a reduction of slow twitch myofiber size and a decreased level of PGC1α expression.

That AUF1 muscle supplementation increases PGC1α protein levels suggesting an important additional level of AUF1 activity in promoting myogenesis. PGC1α activates expression of downstream factors such as NRFs and Tfam that promote mitochondrial biogenesis, which are essential for the formation of slow-twitch muscle fibers, reduced fatigability of muscle and greater oxidative metabolism (Lin et al., “Transcriptional Co-Activator PGC-1 Alpha Drives the Formation of Slow-Twitch Muscle Fibres,” Nature 418(6899):797-801 (2002), which is hereby incorporated by reference in its entirety). These findings, along with enhanced mitochondrial DNA content observed with AUF1 supplementation, suggest that AUF1 is responsible for key activities in slow-twitch myofiber maintenance and increased exercise endurance in mice. Previous studies have shown the benefit of increased PGC1a expression in muscle damage repair and angiogenesis (Wiggs, M. P., “Can Endurance Exercise Preconditioning Prevention Disuse Muscle Atrophy?,” Front. Physiol. 6:63 (2015); Wing et al., “Proteolysis in Illness-Associated Skeletal Muscle Atrophy: From Pathways to Networks,” Crit. Rev. Clin. Lab. Sci. 48(2):49-70 (2011); Bost & Kaminski, “The Metabolic Modulator PGC-1alpha in Cancer,” Am. J. Cancer Res. 9(2):198-211 (2019); Dos Santos et al., “The Effect of Exercise on Skeletal Muscle Glucose Uptake in type 2 Diabetes: An Epigenetic Perspective,” Metabolism 64(12):1619-1628 (2015); Haralampieva et al., “Human Muscle Precursor Cells Overexpressing PGC-1alpha Enhance Early Skeletal Muscle Tissue Formation,” Cell Transplant 26(6):1103-1114 (2017); and Janice Sanchez et al., “Depletion of HuR in Murine Skeletal Muscle Enhances Exercise Endurance and Prevents Cancer-Induced Muscle Atrophy,” Nat. Commun. 10(1):4171 (2019), which are hereby incorporated by reference in their entirety).

AUF1 skeletal muscle gene transfer is therefore beneficial in countering muscle loss and atrophy because it is required to enable multiple key steps in myogenesis. AUF1 stimulates greater muscle development and physical exercise capacity in aging sedentary muscle, which in turn likely further stimulates AUF1 expression as a result of exercise itself. Moreover, the effects of AUF1 gene transfer appear to be long-lasting. Improved exercise endurance in the studies disclosed herein was found to be sustained for at least 6 months beyond the time of gene transfer (the last time point tested) with no evidence for reduction in AUF1 expression or efficacy. In this regard, AUF1 supplementation also increased levels of Pax7⁺ activated satellite cells and myoblasts, suggesting gene transfer into muscle stem cells and an active myogenesis process.

Apart from AUF1, other ARE RNA-binding proteins have also been shown to be involved in the myogenesis process. Of particular relevance to the studies disclosed herein, HuR was recently found to destabilize pgc1α mRNA, leading to the formation of type II myofibers (Janice Sanchez et al., “Depletion of HuR in Murine Skeletal Muscle Enhances Exercise Endurance and Prevents Cancer-Induced Muscle Atrophy,” Nat. Commun. 10(1):4171 (2019), which is hereby incorporated by reference in its entirety). It is noteworthy that AUF1 and HuR often have opposite effects on ARE-mRNA stability, in accord with the findings disclosed herein, and both are essential for the maintenance of myofiber specification. AUF1 can also interact with HuR although the potential functional consequence is unknown, and AUF1 can also compete for binding to AREs with TIA-1, which blocks AUF1-mediated mRNA decay ARE-mRNA translation (Pullmann et al., “Analysis of Turnover and Translation Regulatory RNA-Binding Protein Expression Through Binding to Cognate mRNAs,” Mol. Cell Biol. 27(18):6265-6278 (2007), which is hereby incorporated by reference in its entirety). Clearly, the role of ARE-binding proteins in myogenesis is complex and further investigation into their combined activities is needed to better understand this complexity. How muscle homeostasis is regulated by AUF1 with the other ARE-binding proteins remains to be discovered.

Finally, it is important to note that while AUF1 specifies Type I slow-twitch myofiber development, it also promotes and reprograms the overall myogenesis regeneration program (Abbadi et al., “Muscle Development and Regeneration Controlled by AUF1-Mediated Stage-Specific Degradation of Fate-Determining Checkpoint mRNAs,” Proc. Natl. Acad. Sci. USA 116(23):11285-11290 (2019), which is hereby incorporated by reference in its entirety), evidenced by the fact that AUF l skeletal muscle gene transfer did not result in abnormal muscle development, abnormal balance of muscle fiber types or muscle overgrowth.

### Example 7—AUF1 Restores Skeletal Muscle Mass and Function in Duchenne Muscular Dystrophy (DMD) Mice

To examine the effect of AUF1 gene therapy on skeletal muscle mass and function in a mouse model of Duchenne muscular dystrophy (DMD), the cDNA for full-length p45 AUF1 isoform, which carries out all AUF1 functions, was cloned into an AAV8 vector under the control of the tMCK promoter (AAV8-tMCK-AUF1-IRES-eGFP) (Vector Biolabs), with the AAV8-tMCK-IRES-eGFP 2RS5 enhancer sequences (3-Ebox) ligated to the truncated regulation region of the MCK (muscle creatine kinase) promoter, which provides high skeletal muscle specificity.

The transduction frequency of AAV8 AUF1-GFP and AAV8 GFP control vectors was evaluated in mdx mice by tibialis and muscle GFP staining (FIG. 11A). No statistical differences in transduction efficiency was observed between control AAV8 GFP and treatment AAV8 AUF1 GFP groups (FIG. 11B).

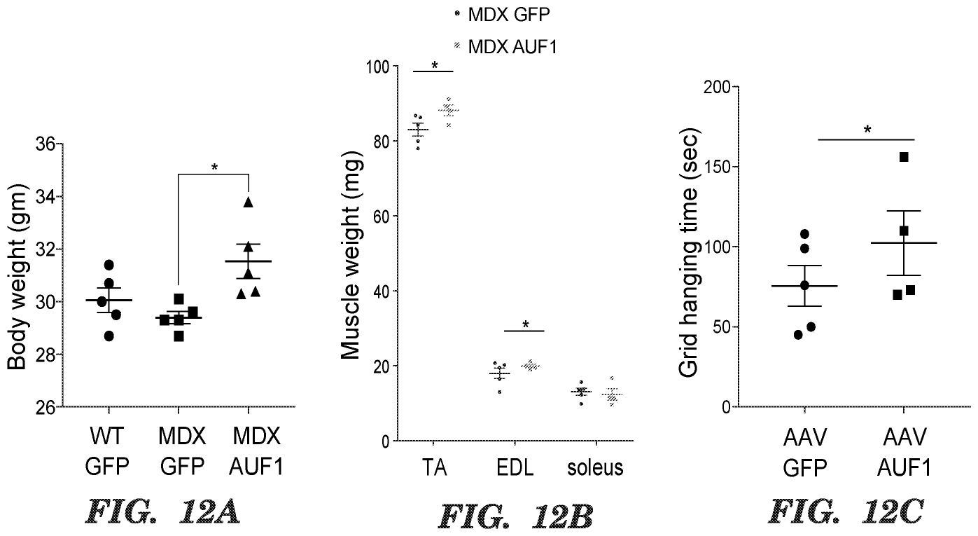

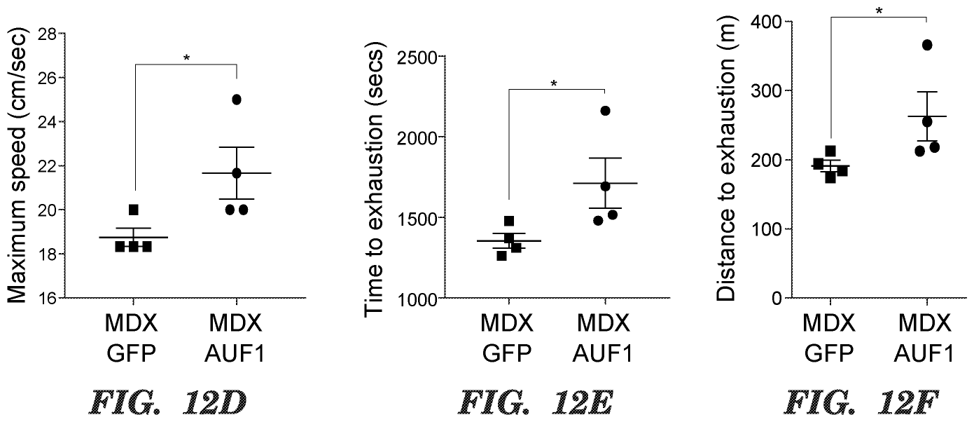

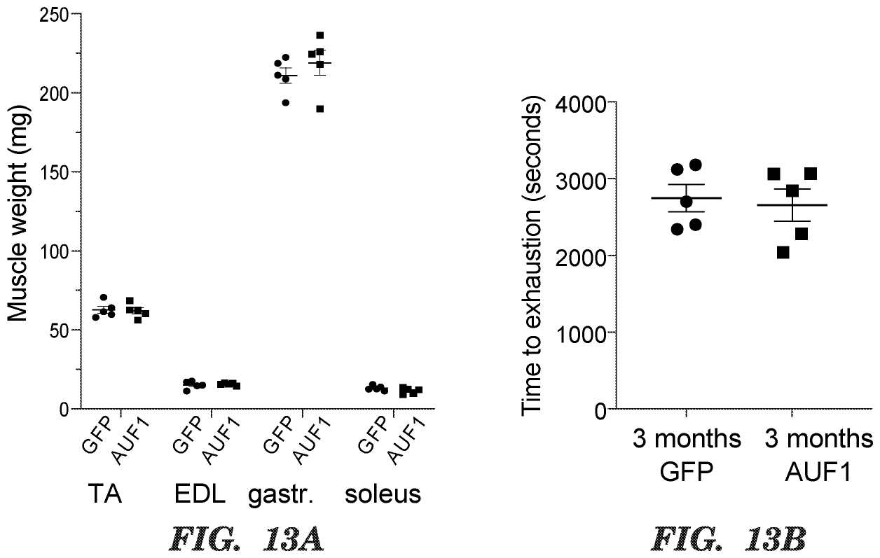

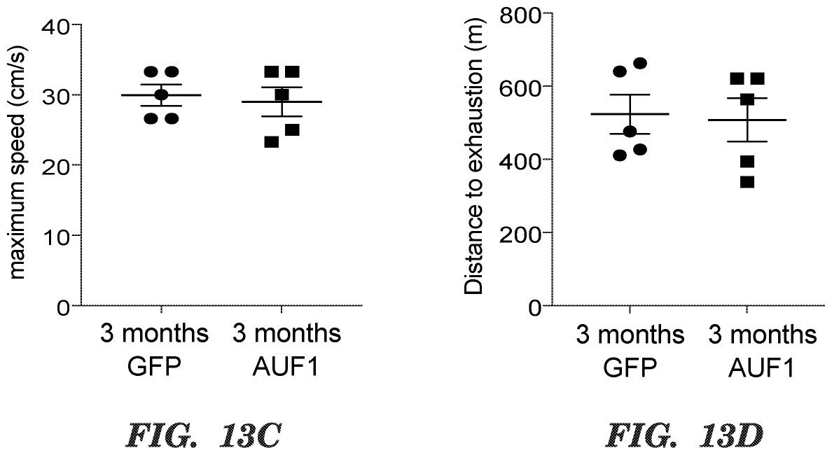

To determine whether AUF1 supplementation enhances muscle mass and/or endurance in mdx mice, one month old C57B110 and mdx mice were administered AAV8-AUF1-GFP or control AAV8-GFP vectors at 2×10¹¹ genome copies by retro-orbital injection (FIGS. 12A-12F and FIGS. 13A-13D). Mice were weighted and monitored for 2 months. AAV8 AUF1-GFP supplemented mdx mice had a significant increase in average body weight, as compared to control mdx mice (FIG. 12A). Moreover, AAV8 AUF1-GFP treated mdx mice demonstrated a 10% increase in TA muscle mass and an 11% increase in extensor digitorum longus (EDL) muscle mass (FIG. 12B), as compared to control AAV8 GFP treated mdx mice. Compared to control AAV8 GFP treated mdx mice, AUF1 supplemented mdx mice showed a ˜40% improvement in grid hanging time, a measure of limb-girdle skeletal muscle strength and endurance (FIG. 12C). When tested by treadmill, AAV AUF1-GFP mdx mice displayed 16% higher maximum speed (FIG. 12D), a 35% greater time to exhaustion (FIG. 12E), and a 37% increased distance to exhaustion (FIG. 12F). These data demonstrate a substantial and statistically significant increase in exercise performance and endurance in mdx mice as a result of AUF1 gene transfer. In contrast to the mdx mice, there was no significant increase in body weight (FIG. 13A), treadmill time to exhaustion (FIG. 13B), maximum speed (FIG. 13C), or distance to exhaustion (FIG. 13D) in AAV8 AUF1-GFP treated WT mice as compared to control AAV8 GFP treated mice of the same genetic background.

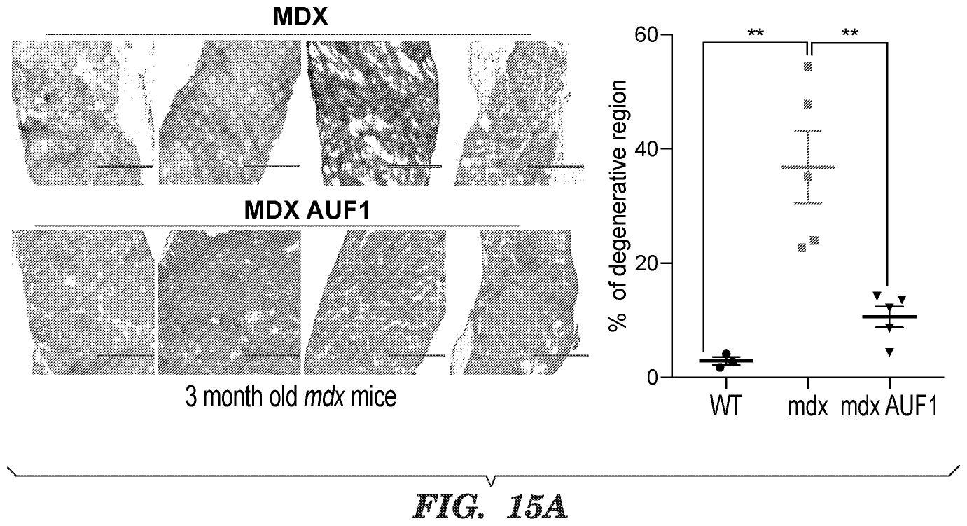

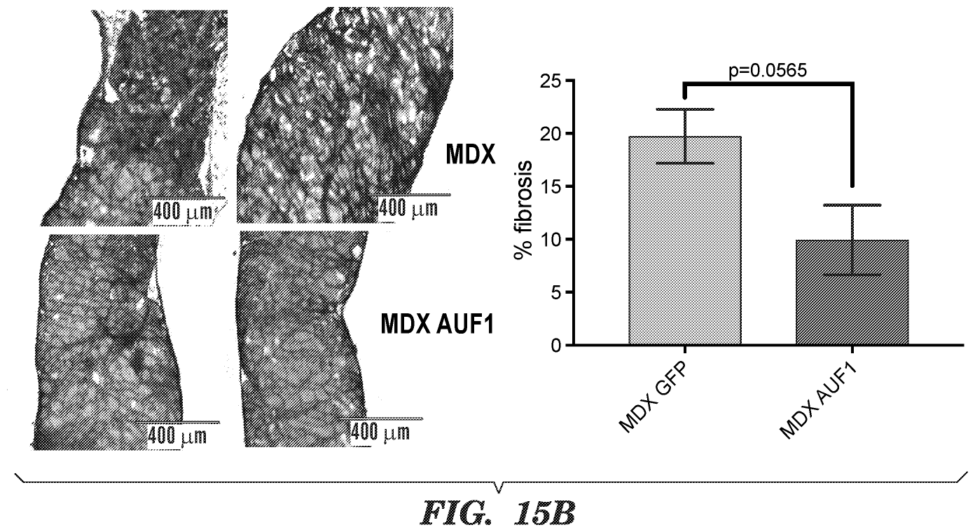

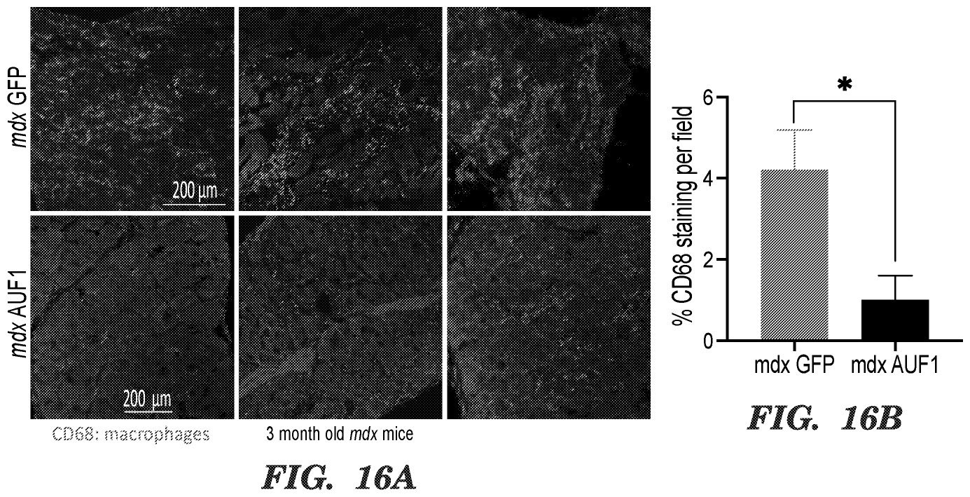

AUF1 overexpression in mdx mice also ameliorated the diaphragm dystrophic phenotype (FIGS. 15A-15B). The percent degenerative diaphragm muscle was reduced by 74% in AAV8 AUF1-GFP treated mdx mice as compared to control AAV8 GFP treated mdx mice (FIG. 15A). AUF1 gene transfer also significantly reduced diaphragm fibrosis (FIG. 15B) and macrophage infiltration (FIGS. 16A-16B) in AAV8 AUF1-GFP treated mdx mice, as compared to control AAV8 GFP treated mdx mice.

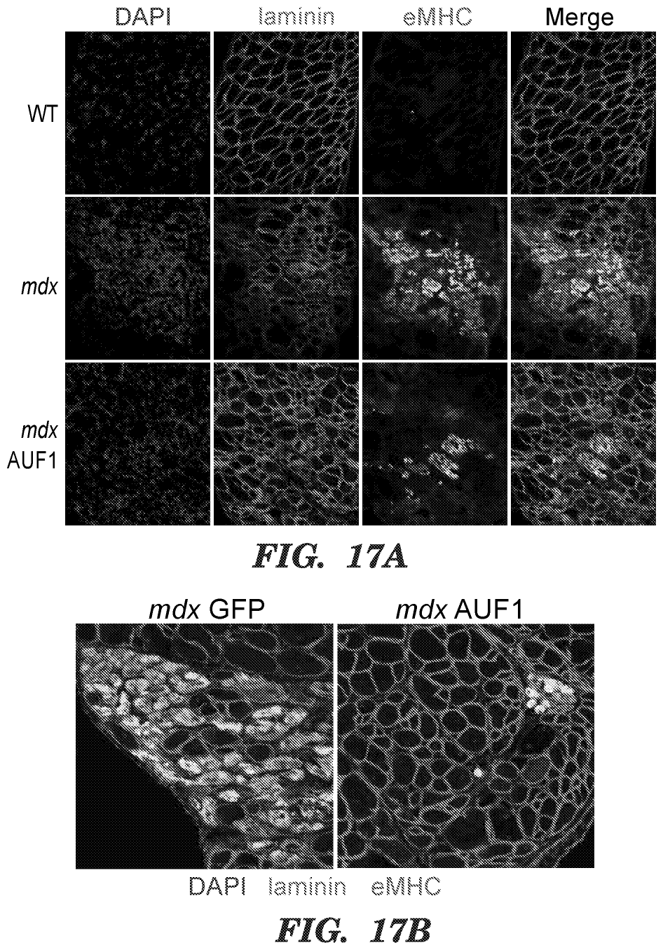

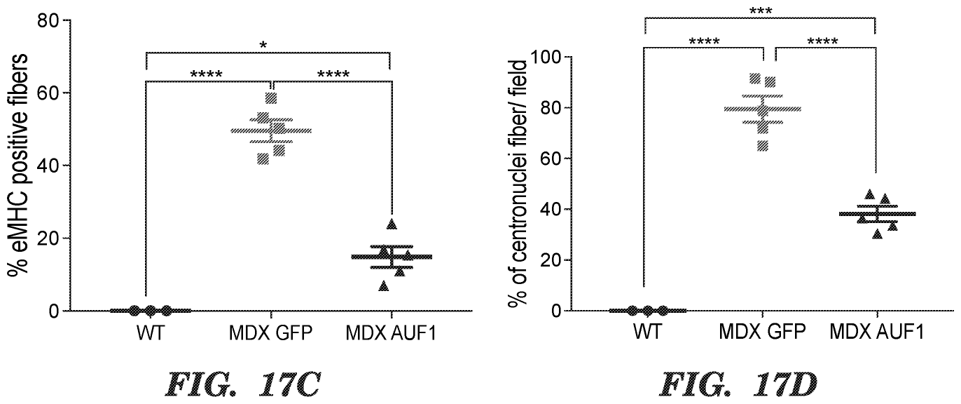

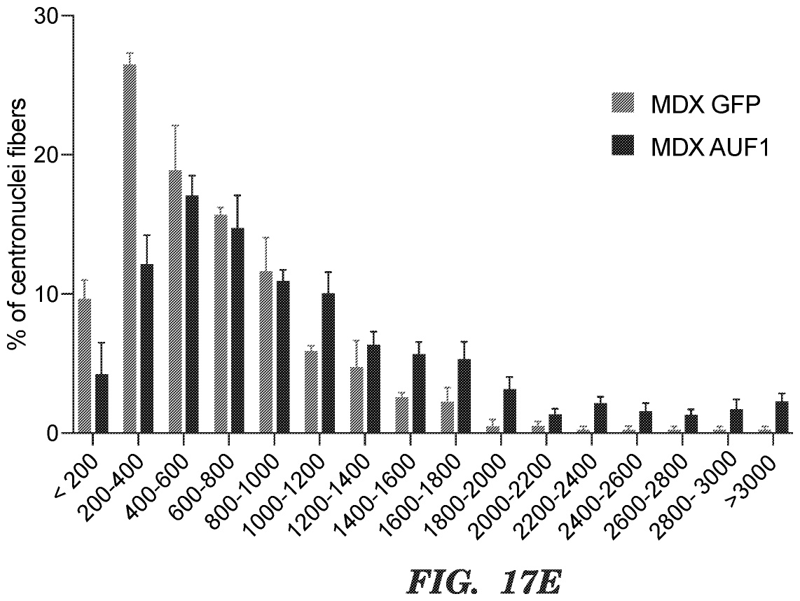

Histological signs of muscular dystrophy, including myofiber centro-nucleation and embryonic myosin heavy chain (eMHC) expression were tested. The percent of centro-nuclei and eMHC positive fibers found increased in mdx mice were highly downregulated upon AUF1 supplementation (FIGS. 17A-17D). The size of centro-nuclei myofibers was also increased upon AUF1 supplementation (FIG. 17E).

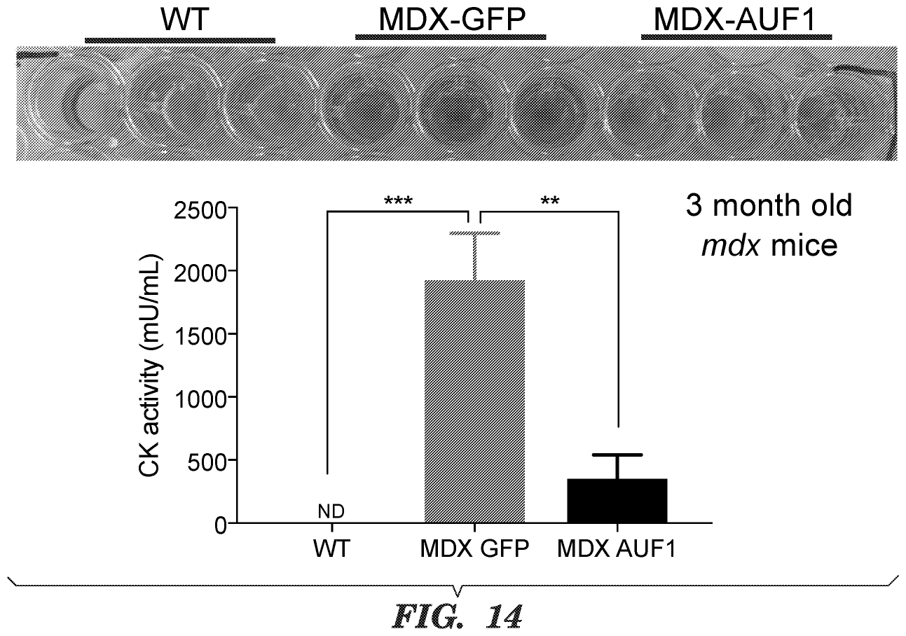

Serological level of creatine kinase (CK) activity, a measure of sarcolemma leakiness used to aid diagnosis of DMD is found increased in control mdx mice, however CK activity was highly decreased upon AUF1 supplementation in mdx mice (FIG. 14).

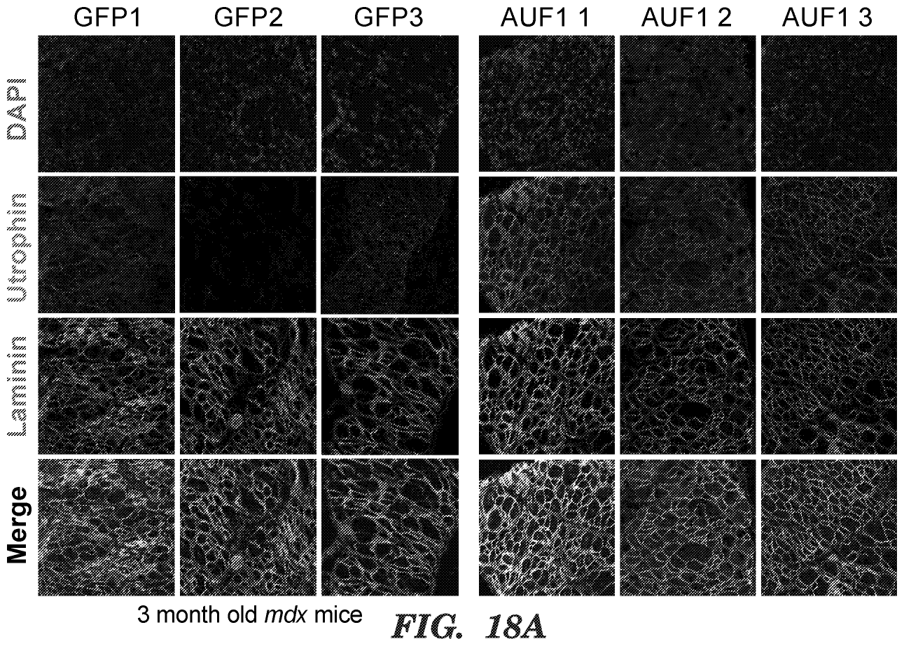

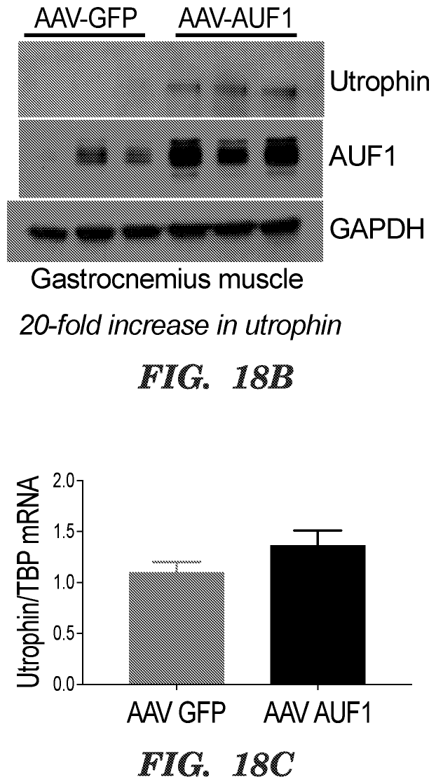

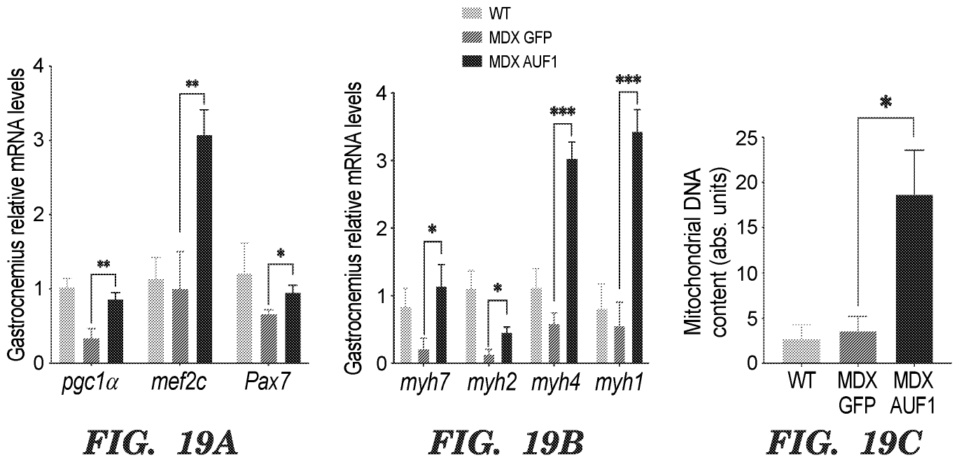

Utrophin expression was also assessed in vitro and in vivo. In vitro, only WT C2C12 myoblasts differentiated into myotubes present an increase of utrophin mRNA and protein. AUF 1 gene therapy strongly increased expression of utrophin and showed evidence for normalization of myofiber integrity in mdx mice, relative to control mdx mice receiving vector alone (FIGS. 18A-18C). AAV8 AUF1 gene transfer increased expression of satellite cell activation gene Pax7 (FIG. 19A), key muscle regeneration genes pgc1α and mef2c (FIG. 19A), slow twitch determination genes (FIG. 19B), and mitochondrial DNA content (FIG. 19C) in mdx mice, relative to control mdx mice receiving vector alone.

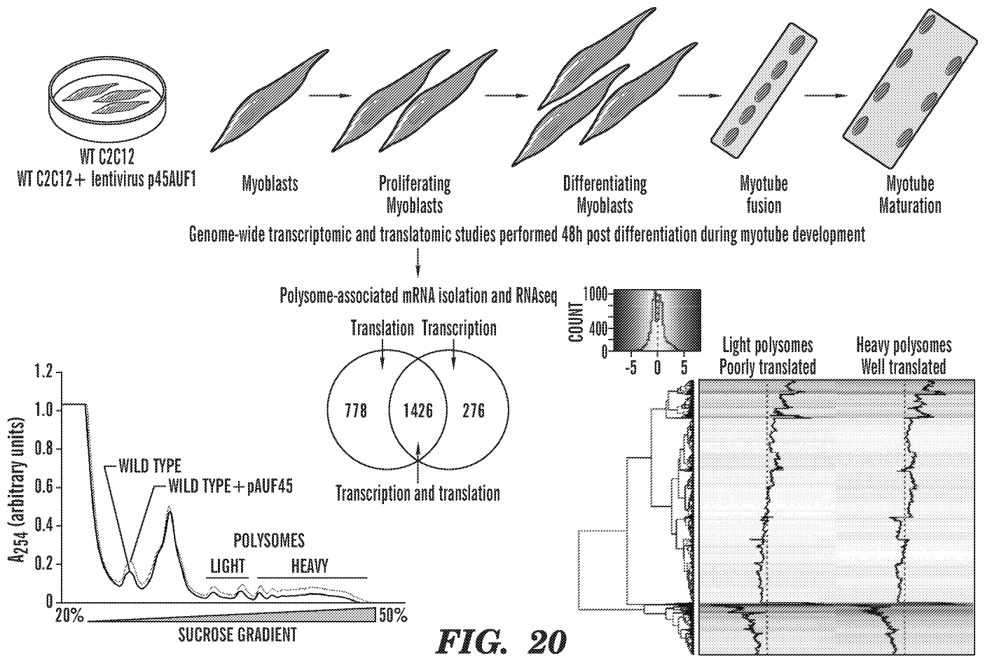

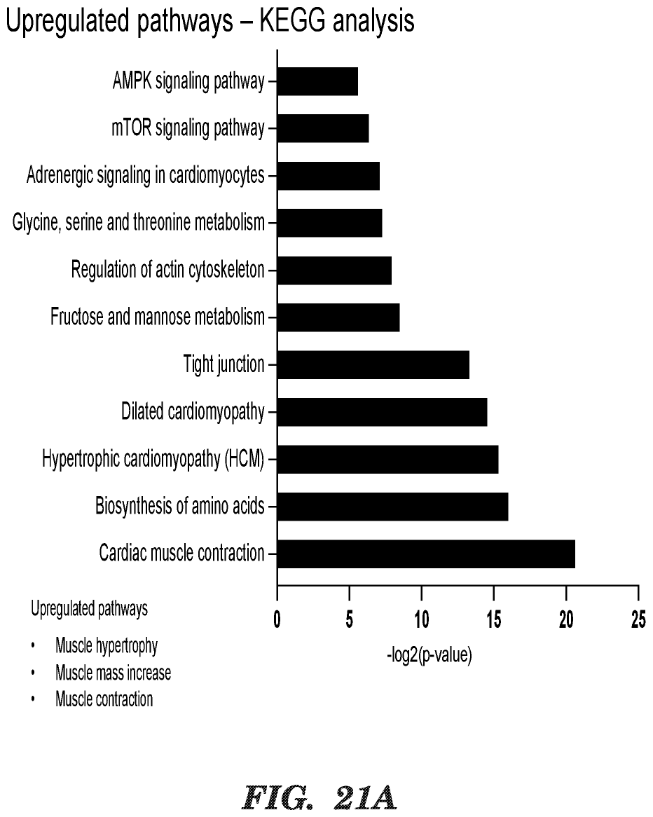

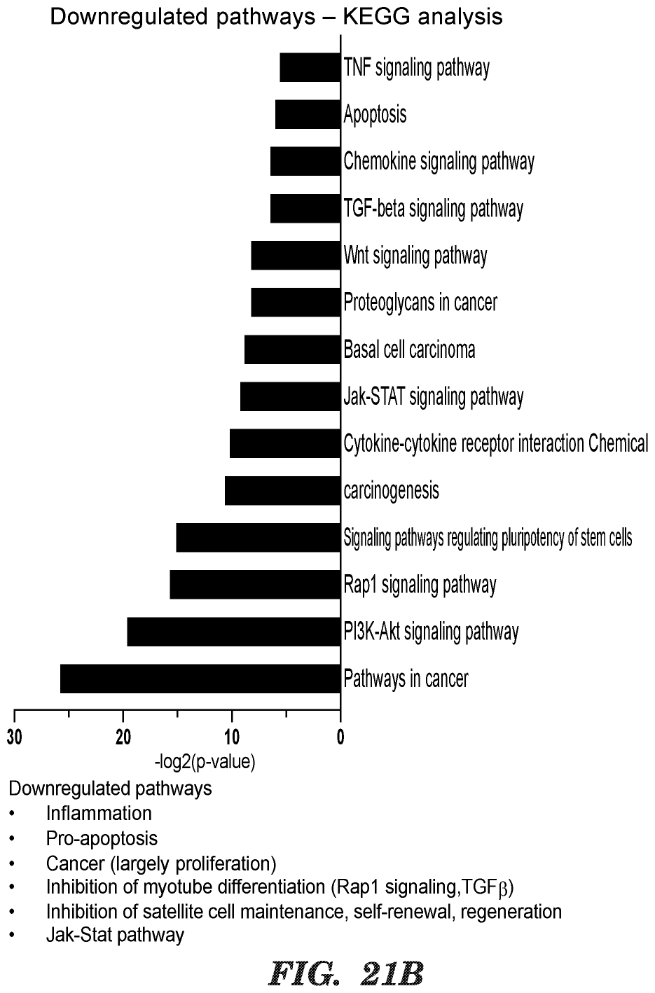

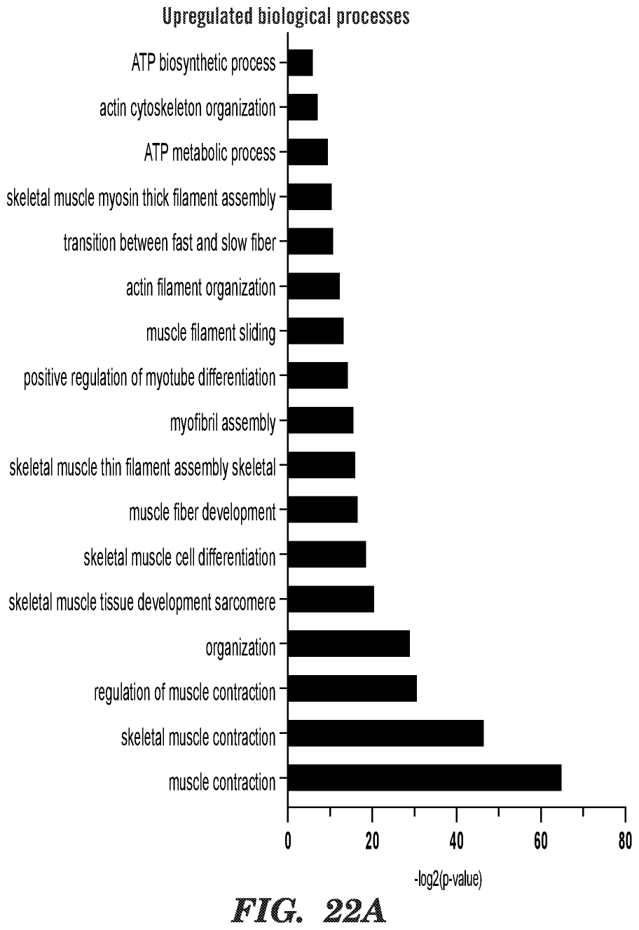

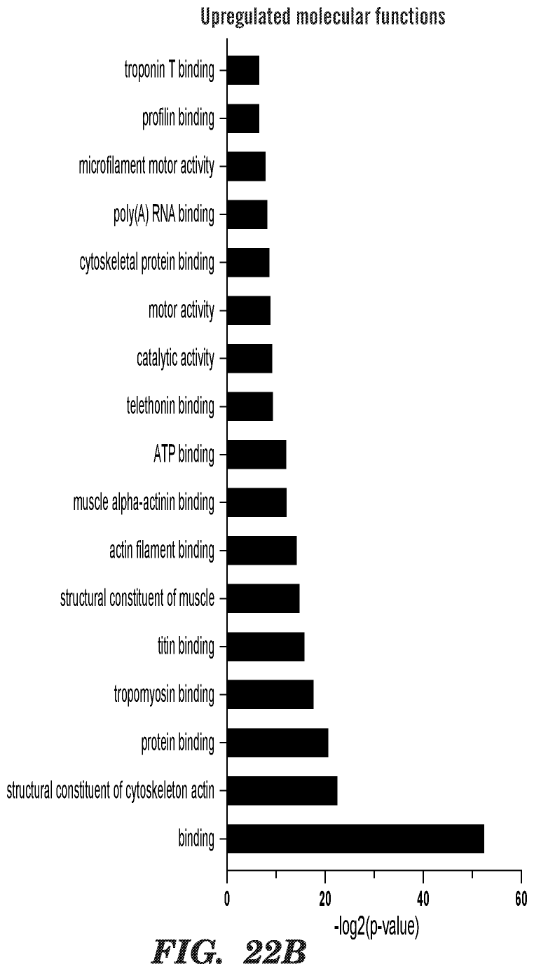

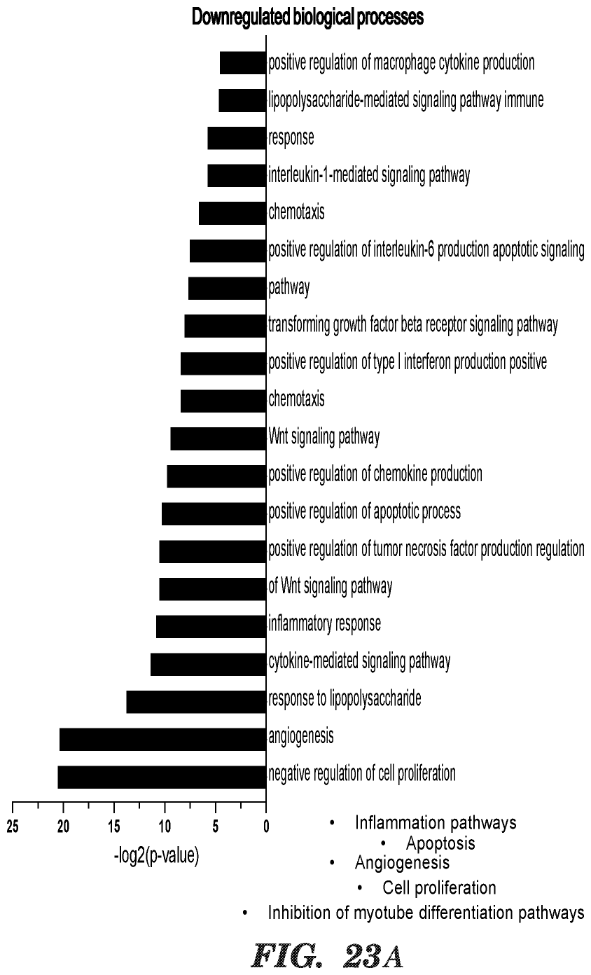

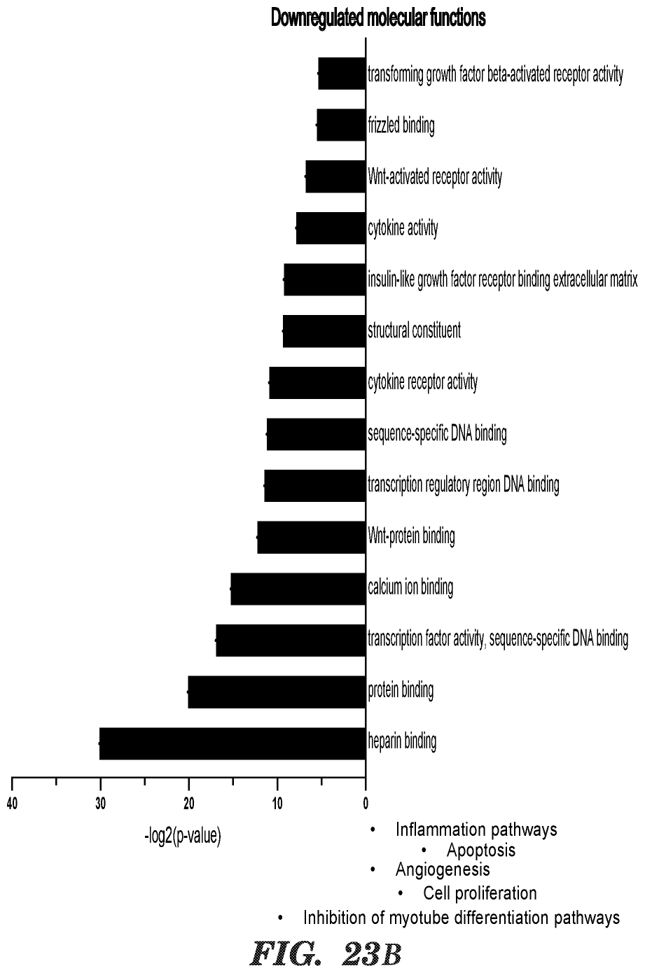

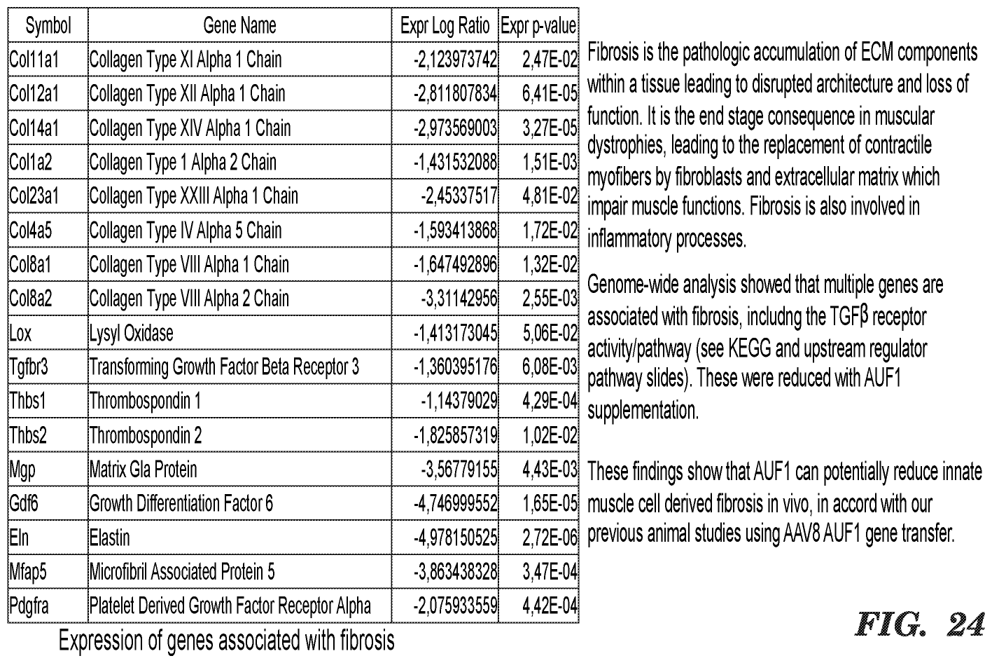

Genome-wide transcriptomic and translatomic studies were carried out to evaluate whether AUF1 activation of C2C12 activates myoblast muscle fiber development (FIG. 20). These studies demonstrate that AUF1 supplementation (i) stimulates expression of major muscle development pathways and decreases expression of inflammatory cytokine, inflammation, cell proliferation, cell death, and anti-muscle regeneration pathways (FIGS. 21A-21B); (ii) upregulates pathways for major biological processes and molecular functions in muscle development and regeneration (FIGS. 22A-22B); (iii) decreases muscle inflammation, inflammatory cytokine, and signaling pathways that oppose muscle regeneration (FIGS. 23A-23B); and (iv) decreases expression of muscle genes associated with development of fibrosis (FIG. 24).

### Discussion of Example 7

### Dystrophin Gene Therapy

As described above, DMD is caused by mutations in the dystrophin gene, resulting in a near-absence of expression of the protein, which plays a key role in stabilization of muscle cell membranes (Bonilla et al., “Duchenne Muscular Dystrophy: Deficiency of Dystrophin at the Muscle Cell Surface,” Cell 54(4):447-452 (1988) and Hoffman et al., “Dystrophin: The Protein Product of the Duchenne Muscular Dystrophy Locus,” Cell 51(6):919-928 (1987), which is hereby incorporated by reference in its entirety). Since the dystrophin gene is very large, it is impossible to reintroduce the entire gene by gene therapy. Thus, current gene therapy attempts involve introducing by gene transfer “mini” and “micro” dystrophin genes, i.e., small pieces of the dystrophin gene packaged in AAV vectors. Since dystrophin is mutated in DMD, there is currently intense interest in finding ways to increase expression of the dystrophin homolog known as utrophin that has overlapping function. To date, this has not been achieved at therapeutic levels that can be shown to be effective.

### DMD mdx Mouse Model

The most widely used DMD mdx mouse (C57BL/10 background) has a spontaneous genetic mutation resulting in a nonsense mutation (premature stop codon) in exon 23 of the very large dystrophin mRNA, similar to the occurrence in roughly 13% of DMD males (Bulfield et al., “X Chromosome-Linked Muscular Dystrophy (mdx) in the Mouse,” Proc. Natl. Acad. Sci. USA 81(4):1189-1192 (1984), which is hereby incorporated by reference in its entirety). This mdx mouse model has been used extensively for DMD investigations and therapeutics research, and is considered the “gold standard” animal model for study of DMD. The C57BL/10 mdx mice are as susceptible to physical muscle damage as are humans and reflects human disease in certain tissues (diaphragm, cardiac muscles), although they are less susceptible to damage in skeletal muscle (Moens et al., “Increased Susceptibility of EDL Muscles from mdx Mice to Damage Induced by Contractions with Stretch,” J. Muscle Res. Cell. Motil. 14(4):446-451 (1993), which is hereby incorporated by reference in its entirety). As in humans, the disease progresses in skeletal muscle with age in mdx mice (Moens et al., “Increased Susceptibility of EDL Muscles from mdx Mice to Damage Induced by Contractions with Stretch,” J. Muscle Res. Cell. Motil. 14(4):446-451 (1993), which is hereby incorporated by reference in its entirety). Equally important, the diaphragm as a target for myo-pathogenesis in mdx mice has been shown to very precisely reproduce the level and rate of damage seen in humans and is an excellent readout for effectiveness of therapeutic intervention (Stedman et al., “The mdx Mouse Diaphragm Reproduces the Degenerative Changes of Duchenne Muscular Dystrophy,” Nature 352(6335):536-539 (1991), which is hereby incorporated by reference in its entirety), and will be studied here.

Importantly, both mdx mice and DMD patients deplete their satellite cells after cycles of necrosis and regeneration of myofibers which promotes disease progression (Manning & O'Malley, “What has the mdx Mouse Model of Duchenne Muscular Dystrophy Contributed to our Understanding of this Disease?” J. Muscle Res. Cell Motil. 36(2):155-167 (2015) and Coley et al., “Effect of Genetic Background on the Dystrophic Phenotype in mdx Mice,” Hum. Mol. Genet. 25(1):130-145 (2016), which are hereby incorporated by reference in their entirety). Moreover, mdx mice and DMD patients both develop an inflammatory response that increases with disease progression (Manning & O'Malley, “What has the mdx Mouse Model of Duchenne Muscular Dystrophy Contributed to our Understanding of this Disease?” J. Muscle Res. Cell Motil. 36(2):155-167 (2015) and Coley et al., “Effect of Genetic Background on the Dystrophic Phenotype in mdx Mice,” Hum. Mol. Genet. 25(1):130-145 (2016), which are hereby incorporated by reference in their entirety).

Despite the fact that skeletal muscle dystrophic disease is generally milder in the mdx mouse than in humans, it still provides a predictive model for pharmacologic response, particularly when coupled with progression of disease in diaphragm. Thus, the mdx mouse provides a reliable, well-established and predictive model in which to follow disease progression and treatment response in animals that has been proven to be useful in development of strategies for interventional agents for DMD clinical trial (Fairclough et al., “Davies, Pharmacologically Targeting the Primary Defect and Downstream Pathology in Duchenne Muscular Dystrophy,” Curr. Gene Ther. 12(3):206-244 (2012) and Stedman et al., “The mdx Mouse Diaphragm Reproduces the Degenerative Changes of Duchenne Muscular Dystrophy,” Nature 352(6335):536-539 (1991), which are hereby incorporated by reference in their entirety). Moreover, studies have also shown that allowing mdx mice to participate in voluntary exercise (wheel running, treadmill) increases skeletal muscle disease due to the introduction of micro-tears from physical stress, similar to human (Smythe et al., “Voluntary Wheel Running in Dystrophin-Deficient (mdx) Mice: Relationships Between Exercise Parameters and Exacerbation of the Dystrophic Phenotype,” PLoS Curr. 3:RRN1295 (2011); Nakae et al., “Quantitative Evaluation of the Beneficial Effects in the mdx Mouse of Epigallocatechin Gallate, an Antioxidant Polyphenol from Green Tea,” Histochem. Cell Biol. 137(6):811-27 (2012); and Archer et al., “Persistent and Improved Functional Gain in mdx Dystrophic Mice after Treatment with L-Arginine and Deflazacort,” FASEB J. 20(6):738-740 (2006), which are hereby incorporated by reference in their entirety). Thus, there are readily available methods for producing a representative human skeletal muscle form of disease in mdx mice that constitute a model for therapeutic assessment and clinical development.

### AUF1 Gene Therapy

The results of Example 7 demonstrate that muscle cell-specific AUF1 gene therapy restores skeletal muscle mass and function in a mouse model of Duchenne muscular dystrophy. In particular, evaluation of muscle cell-specific gene therapy in the DMD mdx model provided evidenced that AAV8 vectored AUF1 gene therapy: (1) efficiently transduced skeletal muscle including cardiac diaphragm and to provide long-duration AUF1 expression without evidence of loss of expression over 6 months (the longest time point tested); (2) activated high levels of satellite cells and myoblasts; (3) significantly increased skeletal muscle mass and normal muscle fiber formation; (4) significantly enhanced exercise endurance; (5) strongly reduced biomarkers or muscle atrophy and muscle cell death in DMD mice; (6) strongly reduced inflammatory immune cell invasion in skeletal muscle including diaphragm; (7) strongly reduced muscle fibrosis and necrosis in skeletal muscle including diaphragm; (8) strongly increased expression of endogenous utrophin in DMD muscle cells while suppressing expression of embryonic dystrophin, a marker of muscle degeneration in DMD; (9) increased normal expression of a large group of genes all of which are involved in muscle development and regeneration, and to suppress genes involved in muscle cell fibrosis, death and muscle-expressed inflammatory cytokines; and (10) did not increase muscle mass, endurance or activate satellite cells in normal skeletal muscle. No aberrant effects of AUF1 skeletal muscle specific gene therapy were observed.

### Example 8—AUF1 Gene Therapy Accelerates Skeletal Muscle Regeneration in Muscle-Injured Mice

A mouse model of BaCl₂ induced necrosis (Garry et al., “Cardiotoxin Induced Injury and Skeletal Muscle Regeneration,” Methods Mol. Biol. 1460:61-71 (2016) and Tierney et al., “Inducing and Evaluating Skeletal Muscle Injury by Notexin and Barium Chloride,” Methods Mol. Biol. 1460:53-60 (2016), which are hereby incorporated by reference in their entirety) was used to examine whether AUF1 gene therapy accelerates skeletal muscle regeneration.

In this study, three month old male mice were administered an intramuscular injection of 50 μl of filtered 1.2% BaCl₂ in sterile saline with control lentivirus vector or with lentivirus p45 AUF1 vector (Abbadi et al., “Muscle Development and Regeneration Controlled by AUF1-mediated Stage-specific Degradation of Fate-determining Checkpoint mRNAs,” Proc. Nat'l. Acad. Sci. USA 116:11285-90 (2019), which is hereby incorporated by reference in its entirety) into the left tibialis anterior (TA) muscle. The right TA muscle remained uninjured as a control.

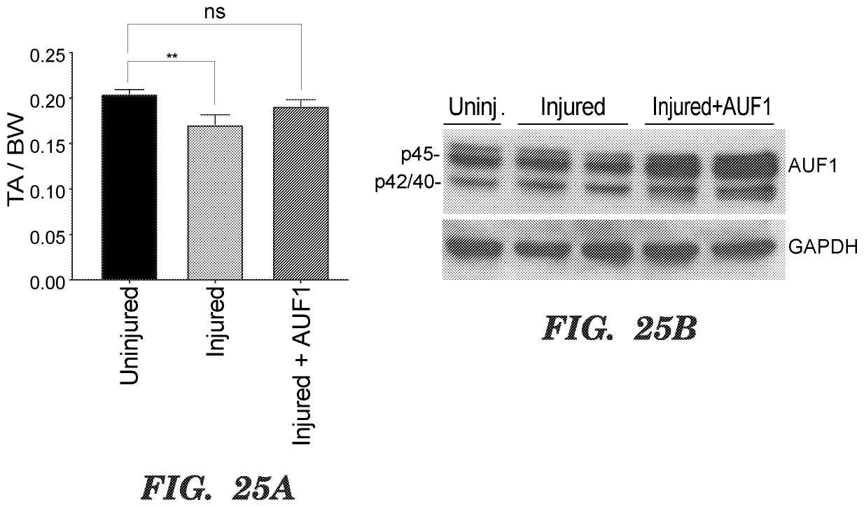

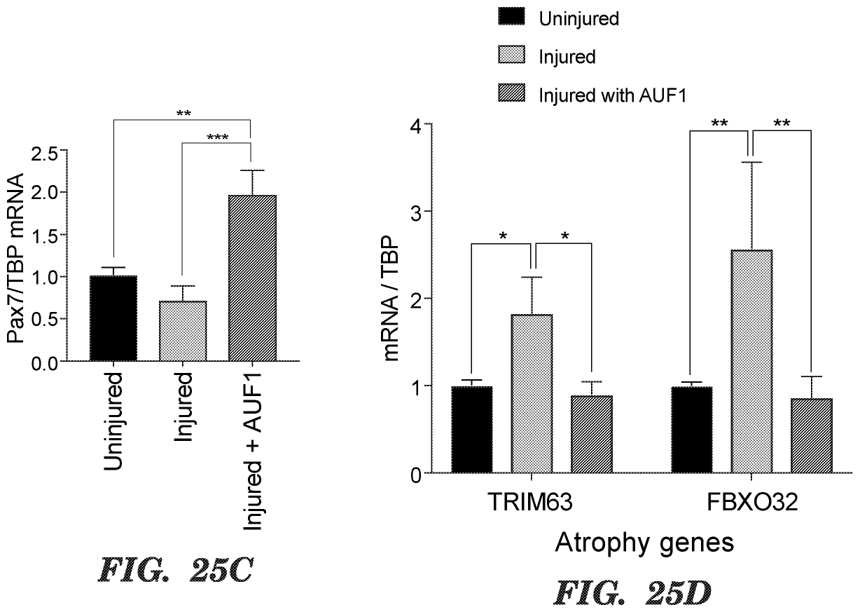

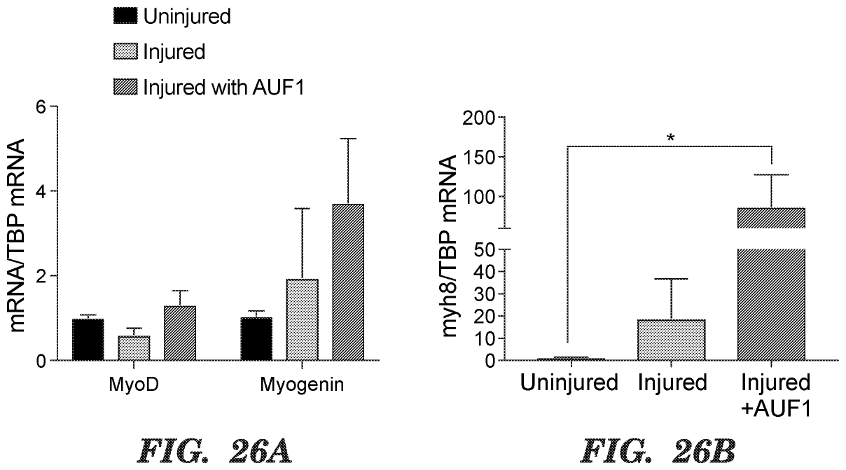

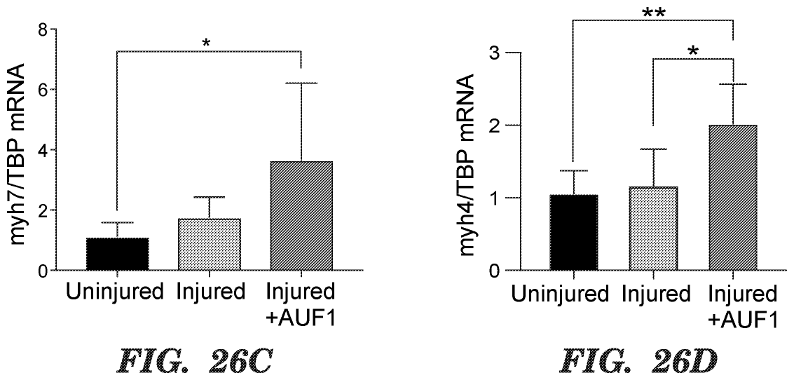

Muscle atrophy was determined by weight of excised TA muscle. In mice sacrificed at 7 days post-injection, TA injury reduced TA weight by 27% which was restored to near-uninjured levels by concurrent AUF1 gene therapy (FIG. 25A). p45 AUF1 gene transfer increased AUF1 expression by several fold in lentivirus transduced muscle (FIG. 25B), which was associated with reduced expression of TRIM63 and Fbxo32, two established biomarkers of muscle atrophy, that were strongly increased following muscle injury but reduced to near non-injured levels with AUF1 gene transfer (FIG. 25D). Strong muscle regeneration correlated with strong activation of the PAX7, gene consistent with satellite cell activation in the TA muscle (FIG. 25C). p45 AUF1 gene transfer also significantly enhanced expression of muscle regeneration factors (MRFs) such as MyoD and myogenin (FIG. 26A), myh8 (FIG. 26B), myh7 (FIG. 26C), and myh4 (FIG. 26D).

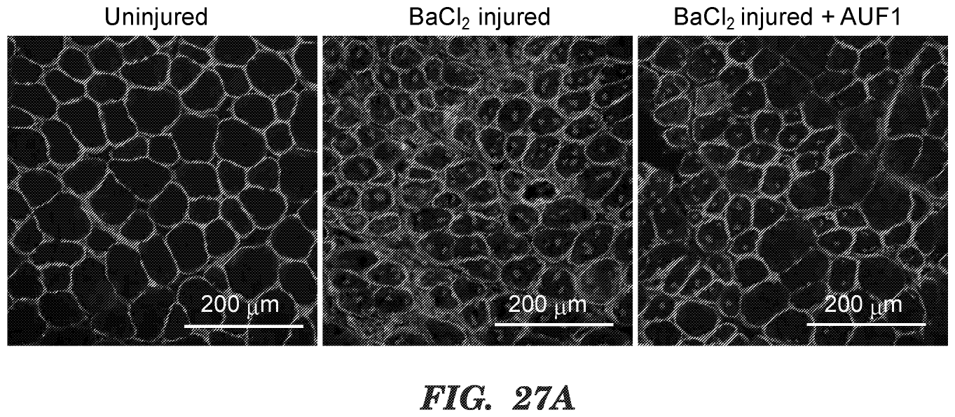

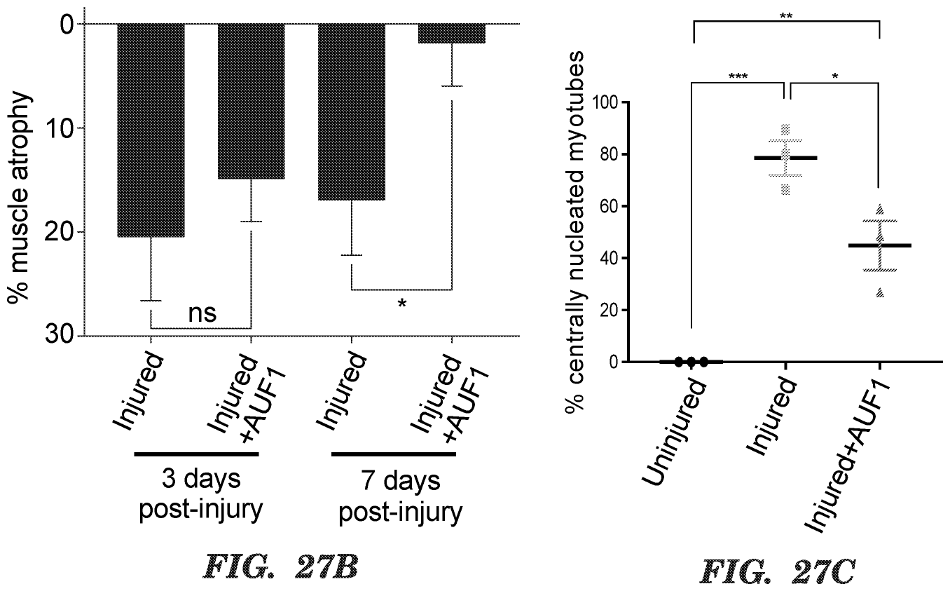

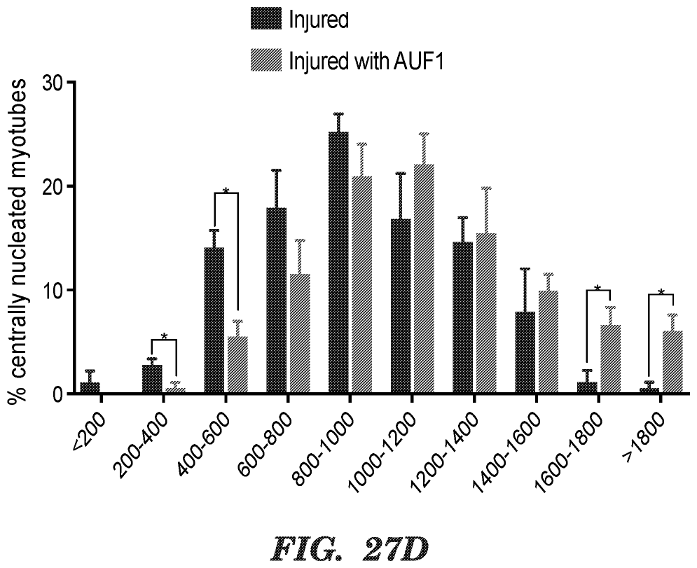

Images of muscle fibers provide further evidence for accelerated but normal muscle regeneration of myofibers in animals administered lentiviral AUF1 that was not seen in control vector mice. A disrupted myofiber architecture and high level of central nuclei in the vector alone TA muscle was observed compared to lenti-AUF1 supplementation (FIG. 27A). Likewise, injured TA muscle receiving sham gene therapy sustained a 20% loss in mass by day 3 following injury, which only very slightly improved by day 7 (FIG. 27B). In contrast, injured TA muscle receiving AUF1 gene therapy showed a trend to less atrophy by day 3, which was almost fully recovered by day 7, demonstrating near normal mass (FIG. 27B). Accelerated muscle regeneration produced mature myofibers, as shown by the striking increase in CSA and reduced central nuclei per myofiber (FIGS. 27C-27D).

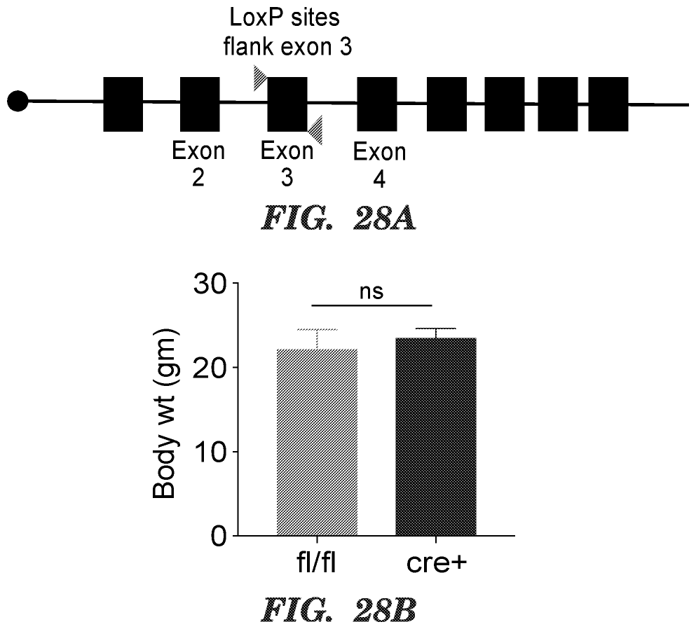

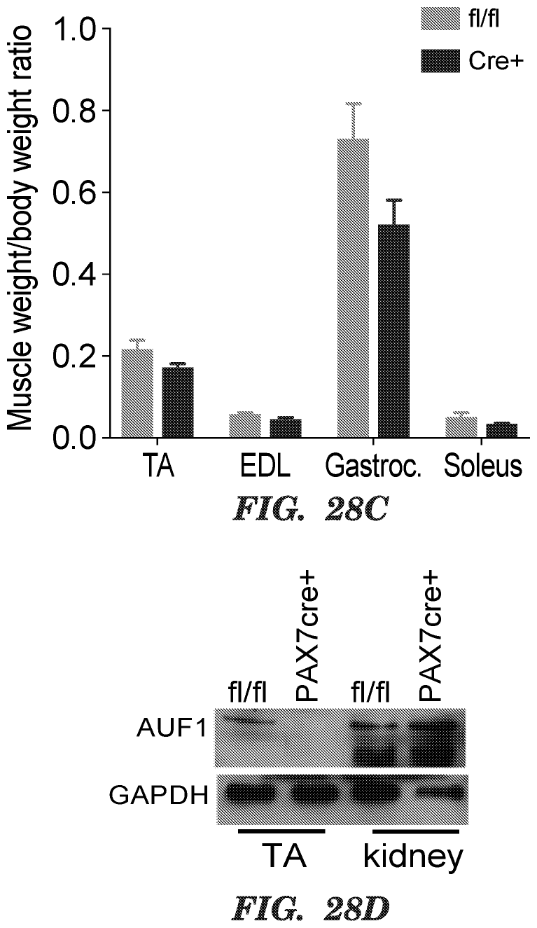

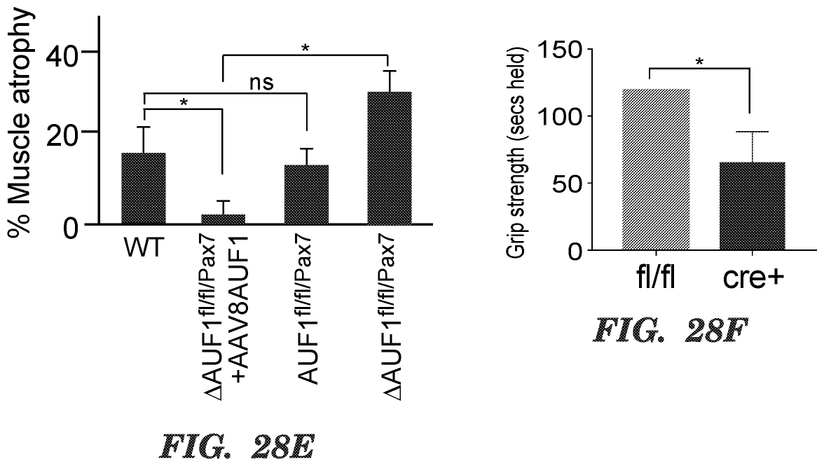

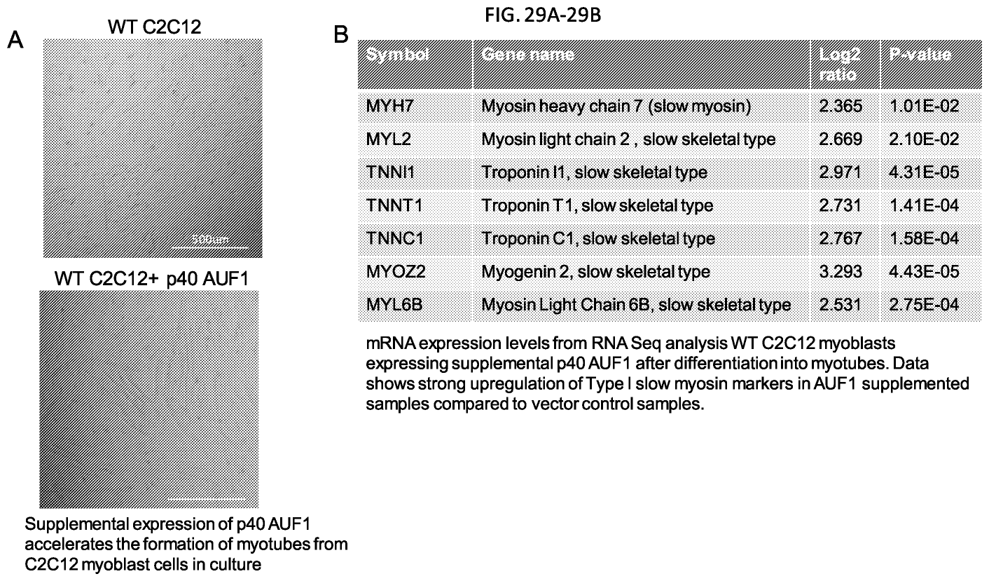

Finally, using an inducible AUF1 conditional knockout mouse (FIGS. 28A-28D) developed as party of the technology described herein, selective AUF1 deletion only in skeletal muscle demonstrated the essential requirement for AUF1 expression to promote regeneration of muscle following traumatic injury (FIG. 28E), and the ability to protect muscle from extensive injury when delivered as AAV8 AUF1 gene therapy (FIG. 28E). In particular, TA muscle from mice injured by 1.2% BaCl₂ injection were evaluated for muscle atrophy at 7 days injection. TA muscle of AUF1^{Flox/Flox}×PAX7^{cre}ERT2 mice expressing AUF1 and WT mice expressing AUF1 (not induced for cre) showed 16-18% atrophy that was not statistically different (FIG. 28E). In contrast, deletion of the AUF1 gene caused strongly increased atrophy of the TA muscle, doubling atrophy levels to 35% (FIG. 28E). However, animals deleted for the AUF1 gene but prophylactically administered AAV8 AUF1 gene therapy demonstrated dramatically reduced levels of TA muscle atrophy, averaging ˜3% (FIG. 28E). AUF1 deleted mice were tested at 5 months for grip strength, a measure of limb-girdle skeletal muscle strength and endurance. AUF1 deleted mice showed a ˜50% reduction in grip strength (FIG. 28F).

Collectively, these data demonstrate that AUF1 is essential for maintenance of muscle strength and muscle regeneration following injury, and that AUF1 gene therapy provides a remarkable ability to promote muscle regeneration and protect muscle from extensive damage despite traumatic injury.

### Discussion of Example 8

Large, severe, or traumatic muscle injuries can result in volumetric muscle loss (VML) in which the conventional muscle repair mechanisms of the body that innately repair and regenerate muscle are overwhelmed, resulting in permanent muscle injury, poor ability to repair muscle, muscle loss, and functional impairment (Grogan et al., “Volumetric Muscle Loss,” J. Am. Acad. Orthop. Surg. 19(Suppl 1):S35-7 (2011); Sicherer et al., “Recent Trends in Injury Models to Study Skeletal Muscle Regeneration and Repair,” Bioengineering (Basel) 7 (2020); Qazi et al., “Cell Therapy to Improve Regeneration of Skeletal Muscle Injuries,” J. Cachexia Sarcopenia Muscle 10:501-16 (2019); and Garg et al., “Volumetric Muscle Loss: Persistent Functional Deficits Beyond Frank Loss of Tissue,” J. Orthop. Res. 33:40-6 (2015), which are hereby incorporated by reference in their entirety). Traumatic skeletal muscle injuries are the most common injuries whether in military service, sports or just accidents in everyday life (Copland et al., “Evidence-Based Treatment of Hamstring Tears,” Curr. Sports Med. Rep. 8:308-14 (2009), which is hereby incorporated by reference in its entirety). Traumatic injuries typically result in muscle necrosis and chronic inflammation, and if they proceed to VML, they can irreparably deplete muscle by 20% or more, which is replaced by fibrotic scar tissue and sets in and persistently long-term disability (Copland et al., “Evidence-Based Treatment of Hamstring Tears,” Curr. Sports Med. Rep. 8:308-14 (2009) and Jarvinen et al., “Muscle Injuries: Biology and Treatment,” Am. J. Sports Med. 33:745-64 (2005), which are hereby incorporated by reference in their entirety). In fact, open bone fractures resulting from accidents or military injuries, of which there are more than 150,000 a year in the civilian population alone in the United States, are responsible for the majority (65%) of severe and poorly healing muscle injuries, in many cases resulting in permanent functional disabilities in as much as 8% of the population (Owens et al., “Characterization of Extremity Wounds in Operation Iraqi Freedom and Operation Enduring Freedom,” J. Orthop. Trauma 21:254-7 (2007); Corona et al., “Volumetric Muscle Loss Leads to Permanent Disability Following Extremity Trauma,” J. Rehabil. Res. Dev. 52:785-92 (2015); and Court-Brown et al., “The Epidemiology of Tibial Fractures,” J. Bone Joint Surg. Br. 77:417-21 (1995), which are hereby incorporated by reference in their entirety).

With skeletal muscle injury, normally quiescent muscle satellite cells are released from their niche in the basal lamina, become activated and begin proliferating (Dumont et al., “Intrinsic and Extrinsic Mechanisms Regulating Satellite Cell Function,” Development 142:1572-81 (2015), which is hereby incorporated by reference in its entirety). Typically, activation of quiescent satellite cells results from micro-damage to muscle fibers (Murphy et al., “Satellite Cells, Connective Tissue Fibroblasts and their Interactions are Crucial for Muscle Regeneration,” Development 138:3625-37 (2011); Carlson et al., “Loss of Stem Cell Regenerative Capacity within Aged Niches,” Aging Cell 6:371-82 (2007); Collins et al., “Stem Cell Function, Self-Renewal, and Behavioral Heterogeneity of Cells from the Adult Muscle Satellite Cell Niche,” Cell 122:289-301 (2005); Gopinath et al., “Stem Cell Review Series: Aging of the Skeletal Muscle Stem Cell Niche,” Aging Cell 7:590-8 (2008); Seale et al., “A New Look at the Origin, Function, and “Stem-Cell” Status of Muscle Satellite Cells,” Dev Biol 218:115-24 (2000); and Dumont et al., “Intrinsic and Extrinsic Mechanisms Regulating Satellite Cell Function,” Development 142:1572-81 (2015), which are hereby incorporated by reference in their entirety) but with extensive damage there is chronic release and activation of satellite cells which can become functionally exhausted and even depleted in such circumstances.

Satellite cells are a small population of muscle cells comprising ˜2-4% of adult skeletal muscle cells. Only a small number of satellite cells self-renew and return to quiescence, while the rest differentiate into muscle progenitor cells called myoblasts. Myoblasts undergo myogenesis (muscle development), a program that includes fusing with existing damaged muscle fibers (myofibers), thereby repairing and regenerating new muscle (Gunther et al., “Myf5-Positive Satellite Cells Contribute to Pax7-Dependent Long-Term Maintenance of Adult Muscle Stem Cells,” Cell Stem Cell 13:590-601 (2013), which is hereby incorporated by reference in its entirety). However, traumatic muscle injury can easily exceed the ability of the myogenesis program to repair injured muscle fibers.

The newly generated myofibers fall into one of two categories: slow-twitch (Type I) or fast-twitch (Type II) fibers, defined according to their speed of movement, type of metabolism, and myosin gene expression. Type II myofibers are the first to atrophy in response to traumatic damage, whereas slow-twitch myofibers are more resilient (Arany, Z. “PGC-1 Coactivators and Skeletal Muscle Adaptations in Health and Disease,” Curr. Opin. Genet. Dev. 18:426-34 (2008) and Wang et al., “Mechanisms for Fiber-Type Specificity of Skeletal Muscle Atrophy,” Curr. Opin. Clin. Nutr. Metab. Care 16:243-50 (2013), which are hereby incorporated by reference in their entirety). The ability to stimulate skeletal muscle regeneration in general, and to selectively promote more resilient slow-twitch muscle in particular, has been a long-standing goal of regenerative muscle biology and clinical practice, as it could potentially be an effective therapy for traumatic muscle injury and various forms of muscular dystrophies (Ljubicic et al., “The Therapeutic Potential of Skeletal Muscle Plasticity in Duchenne Muscular Dystrophy: Phenotypic Modifiers as Pharmacologic Targets,” FASEB J. 28:548-68 (2014), which is hereby incorporated by reference in its entirety). As satellite cells age, or with traumatic muscle injuries that result in chronic cycles of necro-regeneration, satellite cells lose their regenerative capacity and are difficult to reactivate (Bernet et al., “p38 MAPK Signaling Underlies a Cell-Autonomous Loss of Stem Cell Self-Renewal in Skeletal Muscle of Aged Mice,” Nat. Med. 20:265-71 (2014); Dumont et al., “Intrinsic and Extrinsic Mechanisms Regulating Satellite Cell Function,” Development 142:1572-81 (2015); Kudryashova et al., “Satellite Cell Senescence Underlies Myopathy in a Mouse Model of Limb-Girdle Muscular Dystrophy 2H,” J. Clin. Invest. 122:1764-76 (2012); and Silva et al., “Inhibition of Stat3 Activation Suppresses Caspase-3 and the Ubiquitin-Proteasome System, Leading to Preservation of Muscle Mass in Cancer Cachexia,” J. Biol. Chem. 290: 1177-87 (2015), which are hereby incorporated by reference in their entirety).

The cycles of muscle degeneration and regeneration in large or traumatic injuries can lead to functional exhaustion and even loss of muscle stem cells that are essential for muscle regeneration and repair (Carlson et al., “Loss of Stem Cell Regenerative Capacity within Aged Niches,” Aging Cell 6:371-82 (2007); Shefer et al., “Satellite-Cell Pool Size does Matter: Defining the Myogenic Potency of Aging Skeletal Muscle,” Dev. Biol. 294:50-66 (2006); Bernet et al., “p38 MAPK Signaling Underlies a Cell-Autonomous Loss of Stem Cell Self-Renewal in Skeletal Muscle of Aged Mice,” Nat. Med. 20:265-71 (2014); and Dumont et al., “Intrinsic and Extrinsic Mechanisms Regulating Satellite Cell Function,” Development 142:1572-81 (2015), which are hereby incorporated by reference in their entirety), resulting in severe loss of muscle regenerative capacity, permanent muscle loss and chronic disability (Brack, A. S., “Pax7 is Back,” Skelet Muscle 4:24 (2014), which is hereby incorporated by reference in its entirety). Consequently, there are few therapeutic options to increase de novo muscle regeneration, mass and strength available for individuals with severe skeletal muscle injuries, and little evidence that any approaches are very particularly effective (Corona et al., “Pathophysiology of Volumetric Muscle Loss Injury,” Cells Tissues Organs 202:180-88 (2016), which is hereby incorporated by reference in its entirety).

Physical rehabilitation approaches have not been found to be effective in increasing existing muscle mass, muscle regeneration or strength in individuals who have VML injuries (Garg et al., “Volumetric Muscle Loss: Persistent Functional Deficits Beyond Frank Loss of Tissue,” J. Orthop. Res. 33:40-6 (2015) and Mase et al., “Clinical Application of an Acellular Biologic Scaffold for Surgical Repair of a Large, Traumatic Quadriceps Femoris Muscle Defect,” Orthopedics 33:511 (2010), which are hereby incorporated by reference in their entirety). Muscle regeneration approaches that are focused on attenuating the underlying inflammatory response resulting from injury fail to promote effective regeneration of new muscle mass or strength (Corona et al., “Pathophysiology of Volumetric Muscle Loss Injury,” Cells Tissues Organs 202:180-88 (2016) and Qazi et al., “Cell Therapy to Improve Regeneration of Skeletal Muscle Injuries,” J. Cachexia Sarcopenia Muscle 10:501-16 (2019), which are hereby incorporated by reference in their entirety).

Surgical treatments for individuals with chronic muscle injury are also not very effective and have significant limitations. Surgical intervention normally involves surgical reconstruction of injured muscle using autologous muscle transplant and engraftment from healthy muscle elsewhere in the body, which has a high rate of graft degeneration and failure, re-injury, and itself can cause traumatic injury of the resident healthy donor muscle and loss of function (Whiteside, L. A., “Surgical Technique: Gluteus Maximus and Tensor Fascia Lata Transfer for Primary Deficiency of the Abductors of the Hip,” Clin. Orthop. Relat. Res. 472:645-53 (2014); Dziki et al., “An Acellular Biologic Scaffold Treatment for Volumetric Muscle Loss: Results of a 13-Patient Cohort Study,” NPJ Regen. Med. 1:16008 (2016); Sicari et al., “An Acellular Biologic Scaffold Promotes Skeletal Muscle Formation in Mice and Humans with Volumetric Muscle Loss,” Sci. Transl. Med. 6:234ra58 (2014); Hurtgen et al., “Autologous Minced Muscle Grafts Improve Endogenous Fracture Healing and Muscle Strength after Musculoskeletal Trauma,” Physiol. Rep. 5 (2017); and Qazi et al., “Cell Therapy to Improve Regeneration of Skeletal Muscle Injuries,” J. Cachexia Sarcopenia Muscle 10:501-16 (2019), which are hereby incorporated by reference in its entirety). Other surgical approaches that use experimental scaffolds and muscle organoids to promote increased muscle regeneration are technically complex and have also not shown consistent efficacy in model systems (Gholobova et al., “Vascularization of Tissue-Engineered Skeletal Muscle Constructs,” Biomaterials 235:119708 (2020) and Sicherer et al., “Recent Trends in Injury Models to Study Skeletal Muscle Regeneration and Repair,” Bioengineering (Basel) 7 (2020), which are hereby incorporated by reference in their entirety).

Molecular approaches to treat skeletal traumatic injuries generally consist of growth factor therapies, including intramuscular administration or release from implanted biomaterials of hepatocyte growth factor (HGF), insulin-like growth factor (IGF), vascular endothelial growth factor (VEGF) and fibroblast growth factor (FGF) among others (Syverud et al., “Growth Factors for Skeletal Muscle Tissue Engineering,” Cells Tissues Organs 202:169-79 (2016); Pawlikowski et al., “Regulation of Skeletal Muscle Stem Cells by Fibroblast Growth Factors,” Dev. Dyn. 246:359-67 (2017); Menetrey et al., “Growth Factors Improve Muscle Healing in vivo,” J. Bone Joint Surg. Br. 82:131-7 (2000); Rodgers et al., “mTORC1 Controls the Adaptive Transition of Quiescent Stem Cells from G0 to G(Alert),” Nature 510:393-6 (2014); Allen et al., “Hepatocyte Growth Factor Activates Quiescent Skeletal Muscle Satellite Cells in vitro,” J. Cell Physiol. 165:307-12 (1995); Miller et al., “Hepatocyte Growth Factor Affects Satellite Cell Activation and Differentiation in Regenerating Skeletal Muscle,” Am. J. Physiol. Cell Physiol. 278:C174-81 (2000); Grasman et al., “Biomimetic Scaffolds for Regeneration of Volumetric Muscle Loss in Skeletal Muscle Injuries,” Acta Biomater. 25:2-15 (2015); and Cezar et al., “Timed Delivery of Therapy Enhances Functional Muscle Regeneration,” Adv. Healthc. Mater. 6 (2017), which are hereby incorporated by reference in their entirety). These approaches suffer from the limitation of administration of a single muscle growth promoting factor, and that these factors are short-lived, whereas muscle regeneration is complex and requires many factors that must act in concert with each other in a precise spatial and temporal manner over time to effect muscle repair and regeneration. It is therefore not surprising that administration of growth factors, even in combinations, have not shown significant muscle regenerative effects even in experimental models of traumatic muscle injury (Pumberger et al., “Synthetic Niche to Modulate Regenerative Potential of MSCs and Enhance Skeletal Muscle Regeneration,” Biomaterials 99:95-108 (2016), which is hereby incorporated by reference in its entirety).

Most cellular therapies attempt to repopulate muscle regenerative stem (satellite) cells, and reduce necro-inflammation by using transplanted muscle satellite cells or other cells of myogenic origin. However, there are significant impediments to this approach. First, the cells employed must be freshly isolated allogeneic, which means harvesting them from existing surgically removed healthy muscle, in the case of individuals with traumatic and VML injuries. Second, the stem and myogenic cells need to be cultured and expanded, which is technically difficult and not scalable given the magnitude of unmet need. Thus, autologous muscle cell therapies are not clinically feasible for treatment of the majority of patients in need (Qazi et al., “Cell Therapy to Improve Regeneration of Skeletal Muscle Injuries,” J. Cachexia Sarcopenia Muscle 10:501-16 (2019), which is hereby incorporated by reference in its entirety).

The therapeutic options currently available for the treatment of large and/or traumatic muscle injury (e.g., cell therapies, surgical therapies, growth factor and hormonal therapies, molecular therapies, and gene therapies) aim to increase muscle regeneration, muscle mass, and muscle strength for severe skeletal muscle injuries. However, most of the available treatment options work only very poorly, if at all. The results of Example 8 demonstrate that AUF1 gene therapy (e.g., by lentivirus vector delivery directly to muscle or systemic delivery of AUF1 by AAV8 vector) is effective to: (1) activate muscle stem (satellite) cells; (2) reduce expression of established biomarkers of muscle atrophy; (3) accelerated the regeneration of mature muscle fibers (myofibers); (4) enhanced expression of muscle regeneration factors; (5) strongly accelerate the regeneration of injured muscle; (6) increase regeneration of both major types of muscle (i.e., slow-twitch (Type I) or fast-twitch (Type II) fibers); and restore muscle mass, muscle strength, and create normal muscle.

### Example 9: AUF1 Supplementation Increases Development of Slow Muscle Myotubes in Cultured Cells

Without being bound by theory or limited by any specific representative example, certain aspects of the invention employ immortalized pluripotent stem cells that can be differentiated to muscle cells using standard techniques in the literature, muscle cells, muscle stem cells or muscle myoblasts, as shown by example immortalized murine C2C12 myoblast cells.

Transcriptionally active DNA maybe delivered into cells or tissue in culture, e.g., C2C12 cells or other immortalized muscle progenitor or myoblast cells, immortalized muscle cells, being treated using transfection methods including, but not limited to, electroporation, microinjection, calcium phosphate coprecipitation, DEAE dextran facilitated transfection, cationic liposomes, and retroviruses. AUF1 can be delivered into cells in culture expressed from a lentivirus vector, an AAV vector, other viral vectors, from a plasmid vector, with or without selection. In certain embodiments, the DNA to be transfected is cloned into a vector. The AUF1 transgene can be constitutively expressed or expressed from a regulated promoter for inducible expression.

In the present example, C2C12 cells were maintained in DMEM (Corning), 20% FBS (Gibco), and 1% penicillin streptomycin (Life Technologies). To differentiate cells, media was switched to DMEM (Corning), 2% Horse Serum (Gibco), and 1% penicillin streptomycin (Life Technologies) during 96 h. Proliferating wild type C2C12 mouse cardiac myoblasts were stably infected with lentivirus control vector or a lentivirus vector expressing the p40 isoform of AUF1 under the control of the CMV promoter at 1×108 transforming units (TU) per ml. While wild type C2C12 cells express endogenous AUF1 (all four isoforms), supplementation of C2C12 cells with exogenous expressed p40 AUF1 from the lentivirus vector accelerated development of mature myofibers as shown at 48 hours in the phase contrast images, whereas normal maturation typically requires up to 96 hours (Abbadi, D., Yang, M., Chenette, D. M., Andrews, J. J. and Schneider, R. J. (2019). Muscle development and regeneration controlled by AUF1-mediated stage-specific degradation of fate-determining checkpoint mRNAs. Proc. Nat'l. Acad. Sci. USA 116:11285-11290). RNAseq gene expression analysis of vector control and p40 AUF1 lentivirus vector expressing C2C12 myotubes demonstrates that supplementation with AUF1 strongly increased expression of slow myosin mRNAs ranging from 5 to 10 fold (log 2 data shown), providing compelling evidence for development of increased levels of slow muscle myotubes compared to vector control myotubes by additional expression of AUF1.

### Example 10: Prophylactic Administration of AUF1 Gene Therapy Significantly Decreases the Percent of Muscle Atrophy after Injury

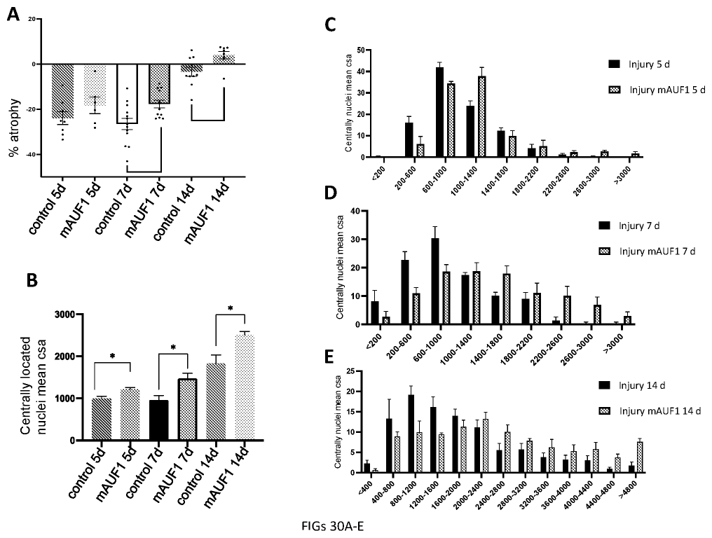

1.2% BaCl₂ was injected into the tibialis anterior (TA) muscle of WT mice at 2 months post-administration of 2E13 vg/kg AAV8-mAUF1. TA muscle was analyzed for percent atrophy (FIG. 30A). Results shows that prophylactic administration of mAUF1 significantly decreases the percent of muscle atrophy compared to WT control mice measured at 7 d and 14 d post-BaCl₂ induction of muscle necrosis. In fact, at 14 days, AAV8-mAUF1-administered WT mice demonstrated strong muscle regeneration not seen in WT control mice.

FIG. 30B is a graph plotting centrally located nuclei mean csa. Increased central nuclei and larger muscle fiber are a measure of mature muscle fiber. Results show greatest central nuclei with greatest csa muscle in AAV8-mAUF-administered animals at 14 d. FIGS. 30C-E are raw data plots used to derive the summary histogram in FIG. 30B. It shows centrally nuclei mean CSA at 5 days (C), 7 days (D) and 14 days (E) post-injury. *, P<0.05; **, P<0.01 by ANOVA. If not marked, not significant. Control=WT mice, no mAUF1 administration.

1.2% BaCl₂ was injected into the tibialis anterior (TA) muscle of WT mice 1 month post-administration of 2E13 vg/kg AAV8-mAUF1. TA muscles were harvested and stained with H&E at 7 and 14 d post-injury from injured WT mice and injured WT mice that had received AAV8-mAUF1. Inspection of results from two mice shows that prophylactic administration of AAV8-mAUF1 significantly decreased muscle degeneration (detected by darker staining) compared to WT mice that did not receive mAUF1 (data not shown). After staining for laminin to highlight muscle morphology, embryonic myosin heavy chain (eMHC) indicative of muscle regeneration, and DAPI for nucleic, results showed a strong reduction of eMHC (successful muscle regeneration) and improved muscle fiber morphology at 7 d in the AAV8-mAUF1 prophylaxed animals (data not shown). Staining with Pax7 (marker of satellite cells and active myoblasts, DAPI and laminin also showed significant improvement in muscle fiber morphology at 14 d only in the AAV8-mAUF1 prophylaxed TA muscle following injury (data not shown).

Although the invention is described in detail with reference to specific embodiments thereof, it will be understood that variations which are functionally equivalent are within the scope of this invention. Indeed, various modifications of the invention in addition to those shown and described herein will become apparent to those skilled in the art from the foregoing description and accompanying drawings. Such modifications are intended to fall within the scope of the appended claims. Those skilled in the art will recognize or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference in their entireties.

The discussion herein provides a better understanding of the nature of the problems confronting the art and should not be construed in any way as an admission as to prior art nor should the citation of any reference herein be construed as an admission that such reference constitutes “prior art” to the instant application.

1-23. (canceled)

24. A method of stabilizing the sarcolemma, increasing β-sarcoglycan in a dystrophin glycoprotein complex (DGC), increasing muscle mass, treating sarcopenia, treating a dystrophinopathy, increasing utrophin in a dystrophin glycoprotein complex (DGC), or healing traumatic muscle injury in a subject in need thereof, comprising administering to the subject a therapeutically effective amount of a vector comprising a nucleic acid molecule encoding an AUF 1 protein or a functional fragment thereof operatively coupled to a differentiated muscle cell-specific promoter.

25. The method of claim 24, wherein said administration increases muscle mass, increases muscle strength, reduces expression of biomarkers of muscle atrophy, enhances muscle performance, increases muscle stamina, increases muscle resistance to fatigue and/or increases proportion of slow twitch fibers to fast twitch fibers relative to pre-treatment levels.

26. The method of claim 24, wherein the differentiated muscle cell-specific promoter is a muscle creatine kinase (MCK) promoter, a smooth muscle 22 (SM22) promoter, a creatine kinase (CK) 8 promoter, a H1 promoter, a desmin promoter, a Pitx3 promoter, or a skeletal alpha-actin promoter.

27. The method of claim 24, wherein the differentiated muscle cell-specific promoter is a tMCK promoter

28. The method of claim 24, wherein the nucleic acid molecule encodes one or more of p37^{AUF1}, p40^{AUF1}, p42^{AUF1}, or p45^{AUF1}.

29. The method of claim 24, wherein the vector is a viral vector.

30. The method of claim 29, wherein the viral vector is a recombinant adeno-associated vector (rAAV) particle.

31. The method of claim 30, wherein the rAAV is an AAV8 serotype.

32. The method of claim 30, wherein the therapeutically effective amount of the rAAV particle is administered at dose of 5×10¹³ to 1×10¹⁵ genome copies/kg.

33. The method of claim 32, wherein the therapeutically effective amount of the rAAV particle is administered intravenously or intramuscularly.

34. A method of producing synthetic meat comprising:
contacting cultured muscle cells with AUF 1 or a nucleic acid encoding and expressing AUF1, wherein the AUF l is present in an amount sufficient to induce slow twitch muscle fibers in the cultured muscle cells; and
growing the muscle cells under conditions and for a time sufficient to produce synthetic meat,
wherein the synthetic meat comprises a greater proportion of slow twitch muscle fibers than synthetic meat produced in the absence of AUF 1.

35. A method of increasing slow twitch muscle fibers in synthetic meat comprising
contacting cultured muscle cells cultured muscle cells with AUF 1 or a nucleic acid encoding and expressing AUF1, wherein the AUF l is present in an amount sufficient to induce slow twitch muscle fibers in the cultured muscle cells; and
growing the muscle cells under conditions and for a time sufficient to produce synthetic meat having a greater proportion of slow twitch muscle fibers than synthetic meat produced in the absence of AUF1.

36. The method of claim 34, wherein the muscle cells are sheep, goat, pig, cow, buffalo, chicken, duck, or goose muscle cells.

37. The method of claim 34, wherein the muscle cells are cultured in two-dimensional monolayer systems or three-dimensional complex muscle structure systems

38. The method of claim 34, wherein the cells are cultured on a consumable matrix.

39. The method of claim 24, wherein the differentiated muscle cell-specific promoter is a C5-12 promoter, a CK6-CK9 promoter, a dMCK promoter, a tMCK promoter, a Spc512 promoter, a creatine kinase (CK) 8e promoter, a MHCK7 promoter, or a Sp-301 promoter.

40. The method of claim 24, wherein the dystrophinopathy is Duchenne muscular dystrophy (DMD), Becker muscular dystrophy (BMD), or X-linked dilated cardiomyopathy.

41. The method of claim 26, wherein the nucleic acid molecule encodes one or more of p37^{AUF1}, p40^{AUF1}, p42^{AUF1}, or p45^{AUF1}.

42. The method of claim 39, wherein the nucleic acid molecule encodes one or more of p37^{AUF1}, p40^{AUF1}, p42^{AUF1}, or p45^{AUF1}.

43. The method of claim 31, wherein the therapeutically effective amount of the rAAV8 particle is administered intravenously or intramuscularly.
